SEQUENCE LISTING (380 sequences)

SEQ ID NO: 1 - DNA, 2154 bp, Homo sapiens
atggtgtggtgcctcggcctggccgtcctcagcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgcttcattggcctgccacgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctccagcagccccacccagtcccttgccaggtcctggacccctcctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagtgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtga

SEQ ID NO: 2 - DNA, 3570 bp, Homo sapiens
gaggggctgggtgagcaggccagctgggctatggtgtggtgcctcggcctggccgtcctcagcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggccggcgtggtgggcggagtctgctttctgggagtggccgtccttgtgagcatcctggccggctgcctcctgaaccggcgcagggctgcccgccgccgccgcaagcgcctccgccaagatccacctcttatcttctctccgaccgggaagtcagctgcaccctctgctctgggctcaggcagtcctgacagcgtggcgaagctgaagctccagggatccccagtccccagcctgcgccagagtctgctctggggggatcctgccggaactcccagcccccacccggatcctccatctagccggggacccttacctctggagcccatttgccggggcccagacgggcgctttgtgatggggcccactgtggcggccccccaggaaaggtcaggccgggagcaggcagaacctcggactccagcccagcgtctggcccggtcctttgactgtagcagcagcagccccagtggggcaccccagcccctctgcattgaagacatcagccctgtggcaccccctccagcagccccacccagtcccttgccaggtcctggacccctgctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagtgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtga

SEQ ID NO: 3 - DNA, 3492 bp, Homo sapiens
atggtgtggtgcctcggcctggccgtcctcagcctggtcatcagccagggggctgacggtcgagggaagcctgaggtggtatcggtggtgggccgggctgaggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgcccagcaatggcctcctgcatccaccctcagcctctgcctacctcactgtgctctgcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggccggcgtggtgggcggagtctgctttctgggagtggccgtccttgtgagcatcctggccggctgcctcctgaaccggcgcagggctgcccgccgccgccgcaagcgcctccgccaagatccacctcttatcttctctccgaccgggaagtcagctgcaccctctgctctgggctcaggcagtcctgacagcgtggcgaagctgaagctccagggatccccagtccccagcctgcgccagagtctgctctggggggatcctgccggaactcccagcccccacccggatcctccatctagccggggacccttacctctggagcccatttgccggggcccagacgggcgctttgtgatggggcccactgtggcggccccccaggaaaggtcaggccgggagcaggcagaacctcggactccagcccagcgtctggcccggtcctttgactgtagcagcagcagccccagtggggcaccccagcccctctgcattgaagacatcagccctgtggcaccccctccagcagccccacccagtcccttgccaggtcctggacccctgctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagcgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagacagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtga

SEQ ID NO: 4 - DNA, 174 bp, Homo sapiens
ctggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcc

SEQ ID NO: 5 - DNA, 189 bp, Homo sapiens
tcccaggatgcttcattggcctgccacgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgccc

SEQ ID NO: 6 - DNA, 183 bp, Homo sapiens
ggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccc

SEQ ID NO: 7 - DNA, 267 bp, Homo sapiens
cctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagc

SEQ ID NO: 8 - DNA, 255 bp, Homo sapiens
ctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagc

SEQ ID NO: 9 - DNA, 174 bp, Homo sapiens; identical to SEQ ID NO: 4

SEQ ID NO: 10 - DNA, 189 bp, Homo sapiens
tcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgccc

SEQ ID NO: 11 - DNA, 183 bp, Homo sapiens; identical to SEQ ID NO: 6

SEQ ID NO: 12 - DNA, 168 bp, Homo sapiens
gggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgcc

SEQ ID NO: 13 - DNA, 267 bp, Homo sapiens; identical to SEQ ID NO: 7

SEQ ID NO: 14 - DNA, 255 bp, Homo sapiens; identical to SEQ ID NO: 8

SEQ ID NO: 15 - DNA, 174 bp, Homo sapiens; identical to SEQ ID NO: 4

SEQ ID NO: 16 - DNA, 189 bp, Homo sapiens
tcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgccc

SEQ ID NO: 17 - DNA, 168 bp, Homo sapiens; identical to SEQ ID NO: 12

SEQ ID NO: 18 - DNA, 462 bp, Homo sapiens
tggagactttgcacctgggcttggccagctcccggctcagacctgaagctgagacagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtgaacatccctaatgtgaggctgtgaaaaggcatatggacctgcaaaggaggcccccaaccagacagacctagtttcaaacgagggcactgcccctgcctgcccctttggtgcccaggcacagaccctgatagtgggtttgggtcaccttggtatggaatgtatgtgctgaccccctaggtgagtctggggattggaacagggatcttaggtctgcctct

SEQ ID NO: 19 - DNA, 25 bp, Homo sapiens
aggccactctgctgtgaacatccct

SEQ ID NO: 20 - DNA, 25 bp, Homo sapiens
tggagactttgcacctgggcttggc

SEQ ID NO: 21 - DNA, 25 bp, Homo sapiens
tagtttcaaacgagggcactgcccc

SEQ ID NO: 22 - DNA, 25 bp, Homo sapiens
agctaggtgtgaagactccagagga

SEQ ID NO: 23 - DNA, 25 bp, Homo sapiens
actgcccatgttactggccctgagg

SEQ ID NO: 24 - DNA, 25 bp, Homo sapiens
catatggacctgcaaaggaggcccc

SEQ ID NO: 25 - DNA, 25 bp, Homo sapiens
cctgatagtgggtttgggtcacctt

SEQ ID NO: 26 - DNA, 25 bp, Homo sapiens
gctgtgctgcccttcgggaggaatt

SEQ ID NO: 27 - DNA, 25 bp, Homo sapiens
gaacagggatcttaggtctgcctct

SEQ ID NO: 28 - DNA, 25 bp, Homo sapiens
gagatgctactagggctcggctacc

SEQ ID NO: 29 - DNA, 25 bp, Homo sapiens
ggtcaccttggtatggaatgtatgt

SEQ ID NO: 30 - DNA, 3492 bp, Homo sapiens
atggtgtggtgcctcggcctggccgtcctcagcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctgcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggccggcgtggtgggcggagtctgctttctgggagtggccgtccttgtgagcatcctggccggctgcctcctgaaccggcgcagggctgcccgccgccgccgcaagcgcctccgccaagatccacctcttatcttctctccgaccgggaagtcagctgcaccctctgctctgggctcaggcagtcctgacagcgtggcgaagctgaagctccagggatccccagtccccagcctgcgccagagtctgctctggggggatcctgccggaactcccagcccccacccggatcctccatctagccggggacccttacctctggagcccatttgccggggcccagacgggcgctttgtgatggggcccactgtggcggccccccaggaaaggtcaggccgggagcaggcagaacctcggactccagcccagcgtctggcccggtcctttgactgtagcagcagcagccccagtggggcaccccagcccctctgcattgaagacatcagccctgtggcaccccctccagcagccccacccagtcccttgccaggtcctggacccctgctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagtgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtga

SEQ ID NO: 31 - DNA, 771 bp, Homo sapiens
ggtccgccccctattgtgtagcggcgagagtggagccgagcggtgcggagcagatctggtggttctccggagagcagcttcctcgggtgttacatgagccaagccctcactgtacagaagagtgagagctgaaacctgttccctgagctgatcagaaggacatcccttggcccctccatctgggctcctgtggataggaggggctgggtgagcaggccagctgggctatggtgtggtgcctcggcctggccgtcctcagcctggtcatcagccagggggctgacggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgttt

SEQ ID NO: 32 - DNA, 18 bp, Homo sapiens
gtgccgcgtgttcttcct

SEQ ID NO: 33 - DNA, 22 bp, Homo sapiens
agccgttagcaaaatcgtcttc

SEQ ID NO: 34 - DNA, 12 bp, Homo sapiens
ccagcacatccc

SEQ ID NO: 35 - DNA, 19 bp, Homo sapiens
tcctcagcctggtcatcag

SEQ ID NO: 36 - DNA, 21 bp, Homo sapiens
agaccctcaatctggagagag

SEQ ID NO: 37 - DNA, 24 bp, Homo sapiens
ctgacggacgagtccggctgcaga

SEQ ID NO: 38 - DNA, 18 bp, Homo sapiens
tggctgcgctttggattc

SEQ ID NO: 39 - DNA, 22 bp, Homo sapiens
ccacgtaatcagggtcaattcg

SEQ ID NO: 40 - DNA, 16 bp, Homo sapiens
cttcccatcttcatcc

SEQ ID NO: 41 - DNA, 2166 bp, Homo sapiens
cagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggcc

SEQ ID NO: 42 - DNA, 2118 bp, Homo sapiens
cagggggctgacggtcgagggaagcctgaggtggtatcggtggtgggccgggctgaggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgcccagcaatggcctcctgcatccaccctcagcctctgcctacctcactgtgctctgcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggcc

SEQ ID NO: 43 - DNA, 867 bp, Homo sapiens
gttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggcc

SEQ ID NO: 44 - DNA, 267 bp, Homo sapiens
cctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgcta

SEQ ID NO: 45 - DNA, 228 bp, Homo sapiens
ccagtcatcgtggtgccccccaagaacagcacagtcaatgcctcccaggatgcttcattggcctgccacgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtg

SEQ ID NO: 46 - DNA, 369 bp, Homo sapiens
agcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaat

SEQ ID NO: 47 - DNA, 285 bp, Homo sapiens
cccccagtcatcgtggtgccccccaagaacagcacagtcaatgcctcccaggatgcttcattggcctgccacgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctc

SEQ ID NO: 48 - DNA, 267 bp, Homo sapiens; identical to SEQ ID NO: 44

SEQ ID NO: 49 - DNA, 228 bp, Homo sapiens
ccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtg

SEQ ID NO: 50 - DNA, 369 bp, Homo sapiens; identical to SEQ ID NO: 46

SEQ ID NO: 51 - DNA, 285 bp, Homo sapiens
cccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctc

SEQ ID NO: 52 - DNA, 267 bp, Homo sapiens; identical to SEQ ID NO: 44

SEQ ID NO: 53 - DNA, 228 bp, Homo sapiens
ccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtg

SEQ ID NO: 54 - DNA, 285 bp, Homo sapiens
cccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgcccagcaatggcctcctgcatccaccctcagcctctgcctacctcactgtgctc

SEQ ID NO: 55 - protein, 717 aa, Homo sapiens [sequence omitted]

SEQ ID NO: 56 - protein, 1189 aa, Homo sapiens
EGLGEQASWAMVWCLGLAVLSLVISQGADGRGKPEVVSVVGRAGESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVRILVDGSLRLLATQPDDAGCYTCVPSNGLLHPPSASAYLTVLYPAQVTAMPPETPLPIGMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLAGVVGGVCFLGVAVLVSILAGCLLNRRRAARRRRKRLRQDPPLIFSPTGKSAAPSALGSGSPDSVAKLKLQGSPVPSLRQSLLWGDPAGTPSPHPDPPSSRGPLPLEPICRGPDGRFVMGPTVAAPQERSGREQAEPRTPAQRLARSFDCSSSSPSGAPQPLCIEDISPVAPPPAAPPSPLPGPGPLLQYLSLPFFREMNVDGDWPPLEEPSPAAPPDYMDTRRCPTSSFLRSPETPPVSPRESLPGAVVGAGATAEPPYTALADWTLRERLLPGLLPAAPRGSLTSQSSGRGSASFLRPPSTAPSAGGSYLSPAPGDTSSWASGPERWPRREHVVTVSKRRNTSVDENYEWDSEFPGDMELLETLHLGLASSRLRPEAEPELGVKTPEEGCLLNTAHVTGPEARCAALREEFLAFRRRRDATRARLPAYRQPVPHPEQATLL

SEQ ID NO: 57 - protein, 1163 aa, Homo sapiens
MVWCLGLAVLSLVISQGADGRGKPEVVSVVGRAEESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVQILVDGSLRLLATQPDDAGCYTCVPSNGLLHPPSASAYLTVLCMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLAGVVGGVCFLGVAVLVSILAGCLLNRRRAARRRRKRLRQDPPLIFSPTGKSAAPSALGSGSPDSVAKLKLQGSPVPSLRQSLLWGDPAGTPSPHPDPPSSRGPLPLEPICRGPDGRFVMGPTVAAPQERSGREQAEPRTPAQRLARSFDCSSSSPSGAPQPLCIEDISPVAPPPAAPPSPLPGPGPLLQYLSLPFFREMNVDGDWPPLEEPSPAAPPDYMDTRRCPTSSFLRSPETPPVSPRESLPGAVVGAGATAEPPYTALADWTLRERLLPGLLPAAPRGSLTSQSSGRGSASFLRPPSTAPSAGGSYLSPAPGDTSSWASGPERWPRREHVVTVSKRRNTSVDENYEWDSEFPGDMELLETLHLGLASSRLRPEAETELGVKTPEEGCLLNTAHVTGPEARCAALREEFLAFRRRRDATRARLPAYRQPVPHPEQATLL

SEQ ID NO: 58 - protein, 58 aa, Homo sapiens
LEPVTLRCVARGSPLPHVTWKLRGKDLGQGQGQVQVQNGTLRIRRVERGSSGVYTCQA

SEQ ID NO: 59 - protein, 63 aa, Homo sapiens
SQDASLACHAEAYPANLTYSWFQDNINVFHISRLQPRVRILVDGSLRLLATQPDDAGCYTCVP

SEQ ID NO: 60 - protein, 61 aa, Homo sapiens
GMPGVIRCPVRANPPLLFVSWTKDGKALQLDKFPGWSQGTEGSLIIALGNEDALGEYSCTP

SEQ ID NO: 61 - protein, 89 aa, Homo sapiens
PHVVTNVSVVALPKGANVSWEPGFDGGYLQRFSVWYTPLAKRPDRMHHDWVSLAVPVGAAHLLVPGLQPHTQYQFSVLAQNKLGSGPFS

SEQ ID NO: 62 - protein, 85 aa, Homo sapiens
LSPPRGLVAVRTPRGVLLHWDPPELVPKRLDGYVLEGRQGSQGWEVLDPAVAGTETELLVPGLIKDVLYEFRLVAFAGSFVSDPS

SEQ ID NO: 63 - protein, 58 aa, Homo sapiens; identical to SEQ ID NO: 58

SEQ ID NO: 64 - protein, 63 aa, Homo sapiens
SQDVSLACHAEAYPANLTYSWFQDNINVFHISRLQPRVRILVDGSLRLLATQPDDAGCYTCVP

SEQ ID NO: 65 - protein, 61 aa, Homo sapiens; identical to SEQ ID NO: 60

SEQ ID NO: 66 - protein, 56 aa, Homo sapiens
GRELLIPCSAQGDPPPVVSWTKVGRGLQGQAQVDSNSSLILRPLTKEAHGHWECSA

SEQ ID NO: 67 - protein, 89 aa, Homo sapiens; identical to SEQ ID NO: 61

SEQ ID NO: 68 - protein, 85 aa, Homo sapiens; identical to SEQ ID NO: 62

SEQ ID NO: 69 - protein, 58 aa, Homo sapiens; identical to SEQ ID NO: 58

SEQ ID NO: 70 - protein, 63 aa, Homo sapiens
SQDVSLACHAEAYPANLTYSWFQDNINVFHISRLQPRVQILVDGSLRLLATQPDDAGCYTCVP

SEQ ID NO: 71 - protein, 56 aa, Homo sapiens; identical to SEQ ID NO: 66

SEQ ID NO: 72 - protein, 722 aa, Homo sapiens
QGADGRGKPEVVSVVGRAGESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVRILVDGSLRLLATQPDDAGCYTCVPSNGLLHPPSASAYLTVLYPAQVTAMPPETPLPIGMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLA

SEQ ID NO: 73 - protein, 706 aa, Homo sapiens
QGADGRGKPEVVSVVGRAEESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVQILVDGSLRLLATQPDDAGCYTCVPSNGLLHPPSASAYLTVLCMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLA

SEQ ID NO: 74 - protein, 289 aa, Homo sapiens
VVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLA

SEQ ID NO: 75 - protein, 16 aa, Homo sapiens
PAQVTAMPPETPLPIG

SEQ ID NO: 76 - protein, 6 aa, Homo sapiens
LTVLCM

SEQ ID NO: 77 - protein, 6 aa, Homo sapiens
TVLCMP

SEQ ID NO: 78 - protein, 6 aa, Homo sapiens
VLCMPG

SEQ ID NO: 79 - protein, 6 aa, Homo sapiens
LCMPGV

SEQ ID NO: 80 - protein, 6 aa, Homo sapiens
CMPGVI

SEQ ID NO: 81 - protein, 89 aa, Homo sapiens
PQFQETPPAVLEVQELEPVTLRCVARGSPLPHVTWKLRGKDLGQGQGQVQVQNGTLRIRRVERGSSGVYTCQASSTEGSATHATQLLVL

SEQ ID NO: 82 - protein, 76 aa, Homo sapiens
PVIVVPPKNSTVNASQDASLACHAEAYPANLTYSWFQDNINVFHISRLQPRVRILVDGSLRLLATQPDDAGCYTCV

SEQ ID NO: 83 - protein, 123 aa, Homo sapiens
SLVISQGADGRGKPEVVSVVGRAGESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVN

SEQ ID NO: 84 - protein, 95 aa, Homo sapiens
PPVIVVPPKNSTVNASQDASLACHAEAYPANLTYSWFQDNINVFHISRLQPRVRILVDGSLRLLATQPDDAGCYTCVPSNGLLHPPSASAYLTVL

SEQ ID NO: 85 - protein, 89 aa, Homo sapiens; identical to SEQ ID NO: 81

SEQ ID NO: 86 - protein, 76 aa, Homo sapiens
PVIVVPPKNSTVNASQDVSLACHAEAYPANLTYSWFQDNINVFHISRLQPRVRILVDGSLRLLATQPDDAGCYTCV

SEQ ID NO: 87 - protein, 123 aa, Homo sapiens; identical to SEQ ID NO: 83

SEQ ID NO: 88 - protein, 95 aa, Homo sapiens
PPVIVVPPKNSTVNASQDVSLACHAEAYPANLTYSWFQDNINVFHISRLQPRVRILVDGSLRLLATQPDDAGCYTCVPSNGLLHPPSASAYLTVL

SEQ ID NO: 89 - protein, 89 aa, Homo sapiens; identical to SEQ ID NO: 81

SEQ ID NO: 90 - protein, 76 aa, Homo sapiens
PVIVVPPKNSTVNASQDVSLACHAEAYPANLTYSWFQDNINVFHISRLQPRVQILVDGSLRLLATQPDDAGCYTCV

SEQ ID NO: 91 - protein, 95 aa, Homo sapiens
PPVIVVPPKNSTVNASQDVSLACHAEAYPANLTYSWFQDNINVFHISRLQPRVQILVDGSLRLLATQPDDAGCYTCVPSNGLLHPPSASAYLTVL

SEQ ID NO: 92 - DNA, 2692 bp, Homo sapiens
cccacgcgtccggcgagagtggagccgagcggtgcggagcagatctggtggttctccggagagcagcttcctcgggtgttacatgagccaagccctcactgtacagaagagtgagagctgaaacctgttccctgagctgatcagaaggacatcccttggcccctccatctgggctcctgtggataggaggggctgggtgagcaggccagctgggctatggtgtggtgcctcggcctggccgtcctcagcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgcttcattggcctgccacgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctccagcagccccacccagtcccttgccaggtcctggacccctcctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagtgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtgaacgtccctaatgtgaggctgtgaaaaggcatatggacctgcaaaggaggcccccaaccagacagacctagtttcaaacgagggcactgcccctgcctgcccctttggtgcccaggcacagaccctgatagtgggtttgggtcaccttggtatggaatgtatgtgctgaccccctaggtgagtctggggattggaacagggatcttaggtctgcctctctctctctctctctctctctctctctctgtgtgtgtgtgtgtgtgtgtgtgtgtgtgaagttttttacaggtgaataaacaaagtttgaaagataaaaaaaaaaa

SEQ ID NO: 93 - DNA, 4036 bp, Homo sapiens
gtggagccgagcggtgcggagcagatctggtggttctccggagagcagcttccttgggtgttacatgagccaagccctcactgtacagaagagtgagagctgaaacctgttccctgagctgatcagaaggacatcccttggcccctccatctgggctcctgtggataggaggggctgggtgagcaggccagctgggctatggtgtggtgcctcggcctggccgtcctcagcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggccggcgtggtgggcggagtctgctttctgggagtggccgtccttgtgagcatcctggccggctgcctcctgaaccggcgcagggctgcccgccgccgccgcaagcgcctccgccaagatccacctcttatcttctctccgaccgggaagtcagctgcaccctctgctctgggctcaggcagtcctgacagcgtggcgaagctgaagctccagggatccccagtccccagcctgcgccagagtctgctctggggggatcctgccggaactcccagcccccacccggatcctccatctagccggggacccttacctctggagcccatttgccggggcccagacgggcgctttgtgatggggcccactgtggcggccccccaggaaaggtcaggccgggagcaggcagaacctcggactccagcccagcgtctggcccggtcctttgactgtagcagcagcagccccagtggggcaccccagcccctctgcattgaagacatcagccctgtggcaccccctccagcagccccacccagtcccttgccaggtcctggacccctgctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagtgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtgaacatccctaatgtgaggctgtgaaaaggcatatggacctgcaaaggaggcccccaaccagacagacctagtttcaaacgagggcactgcccctgcctgcccctttggtgcccaggcacagaccctgatagtgggtttgggtcaccttggtatggaatgtatgtgctgaccccctaggtgagtctggggattggaacagggatcttaggtctgcctctctctctctctctctctctctgtgtgtgtgtgtgtgtgtgtgtgaagttttttacaggtgaataaacaaagtttgaaagatg

SEQ ID NO: 94 - DNA, 4024 bp, Homo sapiens
ggcacgagggtggagccgagcggtgcggagcagatctggtggttctccggagagcagcttccttgggtgttacatgagccaagccctcactgtacagaagagtgagagctgaaacctgttccctgagctgatcagaaggacatcccttggcccctccatctgggctcctgtggataggaggggctgggtgagcaggccagctgggctatggtgtggtgcctcggcctggccgtcctcagcctggtcatcagccagggggctgacggtcgagggaagcctgaggtggtatcggtggtgggccgggctgaggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgcccagcaatggcctcctgcatccaccctcagcctctgcctacctcactgtgctctgcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggccggcgtggtgggcggagtctgctttctgggagtggccgtccttgtgagcatcctggccggctgcctcctgaaccggcgcagggctgcccgccgccgccgcaagcgcctccgccaagatccacctcttatcttctctccgaccgggaagtcagctgcaccctctgctctgggctcaggcagtcctgacagcgtggcgaagctgaagctccagggatccccagtccccagcctgcgccagagtctgctctggggggatcctgccggaactcccagcccccacccggatcctccatctagccggggacccttacctctggagcccatttgccggggcccagacgggcgctttgtgatggggcccactgtggcggccccccaggaaaggtcaggccgggagcaggcagaacctcggactccagcccagcgtctggcccggtcctttgactgtagcagcagcagccccagtggggcaccccagcccctctgcattgaagacatcagccctgtggcaccccctccagcagccccacccagtcccttgccaggtcctggacccctgctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagcgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagacagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtgaacatccctaatgtgaggctgtgaaaaggcatatggacctgcaaaggaggcccccaaccagacagacctagtttcaaacgagggcactgcccctgcctgcccctttggtgcccaggcacagaccctgatagtgggtttgggtcaccttggtatggaatgtatgtgctgaccccctaggtgagtctggggattggaacagggatcttaggtctgcctctctctctctctctctctctctctctctctctctgtgtgtgtgtgtgtgtgtgaagttttttacaggtgaataaacaaagtttgaaagaaaaaaaaaaaaaaaaaaaaaa

SEQ ID NO: 95 - DNA, 1533 bp, Homo sapiens
atgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctga

SEQ ID NO: 96 - DNA, 1533 bp, Homo sapiens
atgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgatggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctga

SEQ ID NO: 97 - DNA, 1533 bp, Homo sapiens
atgcccctgtccctgggagccgagatgtgggggcctgaggcctggctgctgctgctgctactgctggcatcatttacaggccggtgccccgcgggtgagctggggacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctga

SEQ ID NO: 98 - DNA, 255 bp, Homo sapiens
ggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtc

SEQ ID NO: 99 - DNA, 165 bp, Homo sapiens
agagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtc

SEQ ID NO: 100 - DNA, 255 bp, Homo sapiens; identical to SEQ ID NO: 98

SEQ ID NO: 101 - DNA, 165 bp, Homo sapiens; identical to SEQ ID NO: 99

SEQ ID NO: 102 - DNA, 255 bp, Homo sapiens; identical to SEQ ID NO: 98

SEQ ID NO: 103 - DNA, 165 bp, Homo sapiens; identical to SEQ ID NO: 99

SEQ ID NO: 104 - DNA, 504 bp, Homo sapiens
gtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtat

SEQ ID NO: 105 - DNA, 25 bp, Homo sapiens
aggtggagactgtggctcagaccca

SEQ ID NO: 106 - DNA, 25 bp, Homo sapiens
tcagacccaggtgtgcgggcatagc

SEQ ID NO: 107 - DNA, 25 bp, Homo sapiens
ttcttgccctttccattagttgtat

SEQ ID NO: 108 - DNA, 25 bp, Homo sapiens
aagcaggtattttctcagaccccag

SEQ ID NO: 109 - DNA, 25 bp, Homo sapiens
aagtgtgaagcagccagtccctggg

SEQ ID NO: 110 - DNA, 25 bp, Homo sapiens
cagaccccagagcagtattaatgat

SEQ ID NO: 111 - DNA, 25 bp, Homo sapiens
gccgggagcttcttgcaggaatact

SEQ ID NO: 112 - DNA, 25 bp, Homo sapiens
gcaacactgtcagggtttggcgtgt

SEQ ID NO: 113 - DNA, 25 bp, Homo sapiens
gaatactgctccgaatcacttttaa

SEQ ID NO: 114 - DNA, 25 bp, Homo sapiens
gtccgtggtgtgtattatgctgtca

SEQ ID NO: 115 - DNA, 25 bp, Homo sapiens
gtgtgagggaacctgtctcctacca

SEQ ID NO: 116 - DNA, 423 bp, Homo sapiens
ggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctg

SEQ ID NO: 117 - DNA, 423 bp, Homo sapiens
ggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgatg

SEQ ID NO: 118 - DNA, 423 bp, Homo sapiens
ggagccgagatgtgggggcctgaggcctggctgctgctgctgctactgctggcatcatttacaggccggtgccccgcgggtgagctggggacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctg

SEQ ID NO: 119 - DNA, 954 bp, Homo sapiens
ggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcg

SEQ ID NO: 120 - DNA, 954 bp, Homo sapiens
ggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgatggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcg

SEQ ID NO: 121 - DNA, 954 bp, Homo sapiens
ggtgagctggggacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcg

SEQ ID NO: 122 - DNA, 63 bp, Homo sapiens
aaactgccctgcttctaccgaggggactccggcgagcaagtggggcaagtggcatgggctcgg

SEQ ID NO: 123 - DNA, 18 bp, Homo sapiens
aaactgccctgcttctac

SEQ ID NO: 124 - DNA, 18 bp, Homo sapiens
ctgccctgcttctaccga

SEQ ID NO: 125 - DNA, 18 bp, Homo sapiens
ccctgcttctaccgaggg

SEQ ID NO: 126 - DNA, 18 bp, Homo sapiens
tgcttctaccgaggggac

SEQ ID NO: 127 - DNA, 18 bp, Homo sapiens
ttctaccgaggggactcc

SEQ ID NO: 128 - DNA, 18 bp, Homo sapiens
taccgaggggactccggc

SEQ ID NO: 129 - DNA, 18 bp, Homo sapiens
cgaggggactccggcgag

SEQ ID NO: 130 - DNA, 18 bp, Homo sapiens
ggggactccggcgagcaa

SEQ ID NO: 131 - DNA, 18 bp, Homo sapiens
gactccggcgagcaagtg

SEQ ID NO: 132 - DNA, 18 bp, Homo sapiens
tccggcgagcaagtgggg

SEQ ID NO: 133 - DNA, 18 bp, Homo sapiens
ggcgagcaagtggggcaa

SEQ ID NO: 134 - DNA, 18 bp, Homo sapiens
gagcaagtggggcaagtg

SEQ ID NO: 135 - DNA, 18 bp, Homo sapiens
caagtggggcaagtggca

SEQ ID NO: 136 - DNA, 18 bp, Homo sapiens
gtggggcaagtggcatgg

SEQ ID NO: 137 - DNA, 18 bp, Homo sapiens
gggcaagtggcatgggct

SEQ ID NO: 138 - DNA, 18 bp, Homo sapiens
caagtggcatgggctcgg

SEQ ID NO: 139 - DNA, 84 bp, Homo sapiens
cttcatgtgagcccggcttacgagggccgcgtggagcagccgccgcccccacgcaaccccctggacggctcagtgctcctgcgc

SEQ ID NO: 140 - DNA, 18 bp, Homo sapiens
cttcatgtgagcccggct

SEQ ID NO: 141 - DNA, 18 bp, Homo sapiens
catgtgagcccggcttac

SEQ ID NO: 142 - DNA, 18 bp, Homo sapiens
gtgagcccggcttacgag

SEQ ID NO: 143 - DNA, 18 bp, Homo sapiens
agcccggcttacgagggc

SEQ ID NO: 144 - DNA, 18 bp, Homo sapiens
ccggcttacgagggccgc

SEQ ID NO: 145 - DNA, 18 bp, Homo sapiens
gcttacgagggccgcgtg

SEQ ID NO: 146 - DNA, 18 bp, Homo sapiens
tacgagggccgcgtggag

SEQ ID NO: 147 - DNA, 18 bp, Homo sapiens
gagggccgcgtggagcag

SEQ ID NO: 148 - DNA, 18 bp, Homo sapiens
ggccgcgtggagcagccg

SEQ ID NO: 149 - DNA, 18 bp, Homo sapiens
cgcgtggagcagccgccg

SEQ ID NO: 150 - DNA, 18 bp, Homo sapiens
gtggagcagccgccgccc

SEQ ID NO: 151 - DNA, 18 bp, Homo sapiens
gagcagccgccgccccca

SEQ ID NO: 152 - DNA, 18 bp, Homo sapiens
cagccgccgcccccacgc

SEQ ID NO: 153 - DNA, 18 bp, Homo sapiens
ccgccgcccccacgcaac

SEQ ID NO: 154 - DNA, 18 bp, Homo sapiens
ccgcccccacgcaacccc

SEQ ID NO: 155 - DNA, 18 bp, Homo sapiens
cccccacgcaaccccctg

SEQ ID NO: 156 - DNA, 18 bp, Homo sapiens
ccacgcaaccccctggac

SEQ ID NO: 157 - DNA, 18 bp, Homo sapiens
cgcaaccccctggacggc

SEQ ID NO: 158 - DNA, 18 bp, Homo sapiens
aaccccctggacggctca

SEQ ID NO: 159 - DNA, 18 bp, Homo sapiens
cccctggacggctcagtg

SEQ ID NO: 160 - DNA, 18 bp, Homo sapiens
ctggacggctcagtgctc

SEQ ID NO: 161 - DNA, 18 bp, Homo sapiens
gacggctcagtgctcctg

SEQ ID NO: 162 - DNA, 18 bp, Homo sapiens
ggctcagtgctcctgcgc

SEQ ID NO: 163 - DNA, 270 bp, Homo sapiens
cctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacc

SEQ ID NO: 164 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 165 - protein, 510 aa, Homo sapiens [sequence omitted]

SEQ ID NO: 166 - protein, 510 aa, Homo sapiens
MPLSLGAEMWGPEAWLLLLLLLASFTGRCPAGELGTSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 167 - protein, 85 aa, Homo sapiens
GQDAKLPCFYRGDSGEQVGQVAWARVDAGEGAQELALLHSKYGLHVSPAYEGRVEQPPPPRNPLDGSVLLRNAVQADEGEYECRV

SEQ ID NO: 168 - protein, 55 aa, Homo sapiens
REGAMLKCLSEGQPPPSYNWTRLDGPLPSGVRVDGDTLGFPPLTTEHSGIYVCHV

SEQ ID NO: 169 - protein, 85 aa, Homo sapiens; identical to SEQ ID NO: 167

SEQ ID NO: 170 - protein, 55 aa, Homo sapiens; identical to SEQ ID NO: 168

SEQ ID NO: 171 - protein, 85 aa, Homo sapiens; identical to SEQ ID NO: 167

SEQ ID NO: 172 - protein, 55 aa, Homo sapiens; identical to SEQ ID NO: 168

SEQ ID NO: 173 - protein, 141 aa, Homo sapiens
GAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVL

SEQ ID NO: 174 - protein, 141 aa, Homo sapiens
GAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVM

SEQ ID NO: 175 - protein, 141 aa, Homo sapiens
GAEMWGPEAWLLLLLLLASFTGRCPAGELGTSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVL

SEQ ID NO: 176 - protein, 318 aa, Homo sapiens
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSAS

SEQ ID NO: 177 - protein, 318 aa, Homo sapiens
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVMVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSAS

SEQ ID NO: 178 - protein, 318 aa, Homo sapiens
GELGTSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSAS

SEQ ID NO: 179 - protein, 21 aa, Homo sapiens
KLPCFYRGDSGEQVGQVAWAR

SEQ ID NO: 180 - protein, 6 aa, Homo sapiens
KLPCFY

SEQ ID NO: 181 - protein, 6 aa, Homo sapiens
LPCFYR

SEQ ID NO: 182 - protein, 6 aa, Homo sapiens
PCFYRG

SEQ ID NO: 183 - protein, 6 aa, Homo sapiens
CFYRGD

SEQ ID NO: 184 - protein, 6 aa, Homo sapiens
FYRGDS

SEQ ID NO: 185 - protein, 6 aa, Homo sapiens
YRGDSG

SEQ ID NO: 186 - protein, 6 aa, Homo sapiens
RGDSGE

SEQ ID NO: 187 - protein, 6 aa, Homo sapiens
GDSGEQ

SEQ ID NO: 188 - protein, 6 aa, Homo sapiens
DSGEQV

SEQ ID NO: 189 - protein, 6 aa, Homo sapiens
SGEQVG

SEQ ID NO: 190 - protein, 6 aa, Homo sapiens
GEQVGQ

SEQ ID NO: 191 - protein, 6 aa, Homo sapiens
EQVGQV

SEQ ID NO: 192 - protein, 6 aa, Homo sapiens
QVGQVA

SEQ ID NO: 193 - protein, 6 aa, Homo sapiens
VGQVAW

SEQ ID NO: 194 - protein, 6 aa, Homo sapiens
GQVAWA

SEQ ID NO: 195 - protein, 6 aa, Homo sapiens
QVAWAR

SEQ ID NO: 196 - protein, 28 aa, Homo sapiens
LHVSPAYEGRVEQPPPPRNPLDGSVLLR

SEQ ID NO: 197 - protein, 6 aa, Homo sapiens
LHVSPA

SEQ ID NO: 198 - protein, 6 aa, Homo sapiens
HVSPAY

SEQ ID NO: 199 - protein, 6 aa, Homo sapiens
VSPAYE

SEQ ID NO: 200 - protein, 6 aa, Homo sapiens
SPAYEG

SEQ ID NO: 201 - protein, 6 aa, Homo sapiens
PAYEGR

SEQ ID NO: 202 - protein, 6 aa, Homo sapiens
AYEGRV

SEQ ID NO: 203 - protein, 6 aa, Homo sapiens
YEGRVE

SEQ ID NO: 204 - protein, 6 aa, Homo sapiens
EGRVEQ

SEQ ID NO: 205 - protein, 6 aa, Homo sapiens
GRVEQP

SEQ ID NO: 206 - protein, 6 aa, Homo sapiens
RVEQPP

SEQ ID NO: 207 - protein, 6 aa, Homo sapiens
VEQPPP

SEQ ID NO: 208 - protein, 6 aa, Homo sapiens
EQPPPP

SEQ ID NO: 209 - protein, 6 aa, Homo sapiens
QPPPPR

SEQ ID NO: 210 - protein, 6 aa, Homo sapiens
PPPPRN

SEQ ID NO: 211 - protein, 6 aa, Homo sapiens
PPPRNP

SEQ ID NO: 212 - protein, 6 aa, Homo sapiens
PPRNPL

SEQ ID NO: 213 - protein, 6 aa, Homo sapiens
PRNPLD

SEQ ID NO: 214 - protein, 6 aa, Homo sapiens
RNPLDG

SEQ ID NO: 215 - protein, 6 aa, Homo sapiens
NPLDGS

SEQ ID NO: 216 - protein, 6 aa, Homo sapiens
PLDGSV

SEQ ID NO: 217 - protein, 6 aa, Homo sapiens
LDGSVL

SEQ ID NO: 218 - protein, 6 aa, Homo sapiens
DGSVLL

SEQ ID NO: 219 - protein, 6 aa, Homo sapiens
GSVLLR

SEQ ID NO: 220 - protein, 90 aa, Homo sapiens
PPLPSLNPGPALEEGQGLTLAASCTAEGSPAPSVTWDTEVKGTTSSRSFKHSRSAAVTSEFHLVPSRSMNGQPLTCVVSHPGLLQDQRIT

SEQ ID NO: 221 - DNA, 2278 bp, Homo sapiens
cacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtgg

SEQ ID NO: 222 - DNA, 2166 bp, Homo sapiens
gggtcgacccacgcgtccgggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgatggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtgg

SEQ ID NO: 223 - DNA, 2287 bp, Homo sapiens
gttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggggacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtgg

SEQ ID NO: 224 - DNA, 879 bp, Homo sapiens
atgctgggagcctgggcggttgagggaaccgctgtggcgctcctgcgactgctgctgctgctgctgccgccggcgatccggggacccgggctcggcgtggccggcgtggccggcgcggcgggggccgggctgcccgagagcgtcatttgggcggtcaacgcgggtggagaggcgcatgtggacgtgcacgggatccacttccgcaaggaccctttggaaggccgggtgggccgagcctcagactatggcatgaaactgccaatcctgcgttccaaccctgaggaccagatcctgtatcaaactgagcggtacaatgaggagacctttggctacgaagtgcccatcaaagaggagggggactacgtgctggtcttgaaatttgcagaggtctactttgcacagtcccagcaaaaggtatttgatgtacgattgaatggccacgtcgtggtgaaggacttggatatctttgatcgtgttgggcatagcacagctcacgatgaaattatacctatgagcatcagaaaggggaagctgagtgtccagggggaggtgtccaccttcacagggaaactctacattgagtttgtcaaggggtactatgacaatcccaaggtctgtgcactctacatcatggctgggacagtggatgatgtaccaaagcttcagcctcatccgggattggagaagaaagaagaggaagaagaagaagaagaatatgatgaagggtctaatctcaaaaaacagaccaataagaaccgggtgcagtcaggcccccgcacacccaacccctatgcctcggacaacagcagcctcatgtttcccatcctggtggccttcggagtcttcattccaaccctcttctgcctctgccggttgtga

SEQ ID NO: 225 - DNA, 726 bp, Homo sapiens
cccgggctcggcgtggccggcgtggccggcgcggcgggggccgggctgcccgagagcgtcatttgggcggtcaacgcgggtggagaggcgcatgtggacgtgcacgggatccacttccgcaaggaccctttggaaggccgggtgggccgagcctcagactatggcatgaaactgccaatcctgcgttccaaccctgaggaccagatcctgtatcaaactgagcggtacaatgaggagacctttggctacgaagtgcccatcaaagaggagggggactacgtgctggtcttgaaatttgcagaggtctactttgcacagtcccagcaaaaggtatttgatgtacgattgaatggccacgtcgtggtgaaggacttggatatctttgatcgtgttgggcatagcacagctcacgatgaaattatacctatgagcatcagaaaggggaagctgagtgtccagggggaggtgtccaccttcacagggaaactctacattgagtttgtcaaggggtactatgacaatcccaaggtctgtgcactctacatcatggctgggacagtggatgatgtaccaaagcttcagcctcatccgggattggagaagaaagaagaggaagaagaagaagaagaatatgatgaagggtctaatctcaaaaaacagaccaataagaaccgggtgcagtcaggcccccgcacacccaacccctatgcctcggacaacagcagc

SEQ ID NO: 226 - DNA, 531 bp, Homo sapiens
gggactacgtgctggtcttgaaatttgcagaggtctactttgcacagtcccagcaaaaggtatttgatgtacgattgaatggccacgtcgtggtgaaggacttggatatctttgatcgtgttgggcatagcacagctcacgatgaaattatacctatgagcatcagaaaggggaagctgagtgtccagggggaggtgtccaccttcacagggaaactctacattgagtttgtcaaggggtactatgacaatcccaaggtctgtgcactctacatcatggctgggacagtggatgatgtaccaaagcttcagcctcatccgggattggagaagaaagaagaggaagaagaagaagaagaatatgatgaagggtctaatctcaaaaaacagaccaataagaaccgggtgcagtcaggcccccgcacacccaacccctatgcctcggacaacagcagcctcatgtttcccatcctggtggccttcggagtcttcattccaaccctcttctgcctctgccggttgtgagaacaaa

SEQ ID NO: 227 - DNA, 25 bp, Homo sapiens
atgacaatcccaaggtctgtgcact

SEQ ID NO: 228 - DNA, 25 bp, Homo sapiens
agaggtctactttgcacagtcccag

SEQ ID NO: 229 - DNA, 25 bp, Homo sapiens
agcttcagcctcatccgggattgga

SEQ ID NO: 230 - DNA, 25 bp, Homo sapiens
gatgatgtaccaaagcttcagcctc

SEQ ID NO: 231 - DNA, 25 bp, Homo sapiens
gatatctttgatcgtgttgggcata

SEQ ID NO: 232 - DNA, 25 bp, Homo sapiens
gttgggcatagcacagctcacgatg

SEQ ID NO: 233 - DNA, 25 bp, Homo sapiens
gtgtccaccttcacagggaaactct

SEQ ID NO: 234 - DNA, 25 bp, Homo sapiens
gtggccttcggagtcttcattccaa

SEQ ID NO: 235 - DNA, 25 bp, Homo sapiens
gggactacgtgctggtcttgaaatt

SEQ ID NO: 236 - DNA, 25 bp, Homo sapiens
tgtgcactctacatcatggctggga

SEQ ID NO: 237 - DNA, 25 bp, Homo sapiens
tgcctctgccggttgtgagaacaaa

SEQ ID NO: 238 - DNA, 671 bp, Homo sapiens
ggctgagaagaaggaggcctgagagcgacatgtccccggcggctcaggcggagcggcccgtggcgctgtttttctgagtccggggtggcctggcagccggccgaggacgagggtcggcgggggctgcccccgtggtggtggccgccatgctgggagcctgggcggttgagggaaccgctgtggcgctcctgcgactgctgctgctgctgctgccgccggcgatccggggacccgggctcggcgtggccggcgtggccggcgcggcgggggccgggctgcccgagagcgtcatttgggcggtcaacgcgggtggagaggcgcatgtggacgtgcacgggatccacttccgcaaggaccctttggaaggccgggtgggccgagcctcagactatggcatgaaactgccaatcctgcgttccaaccctgaggaccagatcctgtatcaaactgagcggtacaatgaggagacctttggctacgaagtgcccatcaaagaggagggggactacgtgctggtcttgaaatttgcagaggtctactttgcacagtcccagcaaaaggtatttgatgtacgattgaatggccacgtcgtggtgaaggacttggatatctttgatcgtgttgggcatagcacagctcacgatgaaattatacctatgagcatcagaaag

SEQ ID NO: 239 - DNA, 704 bp, Homo sapiens
gtgggaggcggtacccgtggctgagaagaaggaggcctgagagcgacatgtccccggcggctcaggcggagcggcccgtggcgctgtttttctgagtccggggtggcctggcagccggccgaggacgagggtcggcgggggctgcccccgtggtggtggccgccatgctgggagcctgggcggttgagggaaccgctgtggcgctcctgcgactgctgctgctgctgctgccgccggcgatccggggacccgggctcggcgtggccggcgtggccggcgcggcgggggccgggctgcccgagagcgtcatttgggcggtcaacgcgggtggagaggcgcatgtggacgtgcacgggatccacttccgcaaggaccctttggaaggccgggtgggccgaggtgagagtccccctgccgagccgcgggatccagggcctgctgtgctgggcgcagccggccgggggctgcgggccccgagcccctttcgaccctggggccgcgtctctggagcgaagtttctctctgcagcttcttcgggggcccgctctgagctcagggcctggcactggctccaagcctcagactatggcatgaaactgccaatcctgcgttccaaccctgaggaccagatcctgtatcaaactgagcggtacaatgaggagacctttggctacgaagtgcccatcaaagaggagggggactac

SEQ ID NO: 240 - DNA, 19 bp, Homo sapiens
ggatccacttccgcaagga

SEQ ID NO: 241 - DNA, 19 bp, Homo sapiens
ggttggaacgcaggattgg

SEQ ID NO: 242 - DNA, 13 bp, Homo sapiens
tgggccgagcctc

SEQ ID NO: 243 - DNA, 23 bp, Homo sapiens
cgaagtttctctctgcagcttct

SEQ ID NO: 244 - DNA, 19 bp, Homo sapiens
gggttggaacgcaggattg

SEQ ID NO: 245 - DNA, 17 bp, Homo sapiens
tccaagcctcagactat

SEQ ID NO: 246 - protein, 292 aa, Homo sapiens
MLGAWAVEGTAVALLRLLLLLLPPAIRGPGLGVAGVAGAAGAGLPESVIWAVNAGGEAHVDVHGIHFRKDPLEGRVGRASDYGMKLPILRSNPEDQILYQTERYNEETFGYEVPIKEEGDYVLVLKFAEVYFAQSQQKVFDVRLNGHVVVKDLDIFDRVGHSTAHDEIIPMSIRKGKLSVQGEVSTFTGKLYIEFVKGYYDNPKVCALYIMAGTVDDVPKLQPHPGLEKKEEEEEEEEYDEGSNLKKQTNKNRVQSGPRTPNPYASDNSSLMFPILVAFGVFIPTLFCLCRL

SEQ ID NO: 247 - protein, 242 aa, Homo sapiens
PGLGVAGVAGAAGAGLPESVIWAVNAGGEAHVDVHGIHFRKDPLEGRVGRASDYGMKLPILRSNPEDQILYQTERYNEETFGYEVPIKEEGDYVLVLKFAEVYFAQSQQKVFDVRLNGHVVVKDLDIFDRVGHSTAHDEIIPMSIRKGKLSVQGEVSTFTGKLYIEFVKGYYDNPKVCALYIMAGTVDDVPKLQPHPGLEKKEEEEEEEEYDEGSNLKKQTNKNRVQSGPRTPNPYASDNSS

SEQ ID NO: 248 - DNA, 1505 bp, Homo sapiens
ctgagagcgacatgtccccggcggctcaggcggagcggcccgtggcgctgtttttctgagtccggggtggcctggcagccggccgaggacgagggtcggcgggggctgcccccgtggtggtggccgccatgctgggagcctgggcggttgagggaaccgctgtggcgctcctgcgactgctgctgctgctgctgccgccggcgatccggggacccgggctcggcgtggccggcgtggccggcgcggcgggggccgggctgcccgagagcgtcatttgggcggtcaacgcgggtggagaggcgcatgtggacgtgcacgggatccacttccgcaaggaccctttggaaggccgggtgggccgagcctcagactatggcatgaaactgccaatcctgcgttccaaccctgaggaccagatcctgtatcaaactgagcggtacaatgaggagacctttggctacgaagtgcccatcaaagaggagggggactacgtgctggtcttgaaatttgcagaggtctactttgcacagtcccagcaaaaggtatttgatgtacgattgaatggccacgtcgtggtgaaggacttggatatctttgatcgtgttgggcatagcacagctcacgatgaaattatacctatgagcatcagaaaggggaagctgagtgtccagggggaggtgtccaccttcacagggaaactctacattgagtttgtcaaggggtactatgacaatcccaaggtctgtgcactctacatcatggctgggacagtggatgatgtaccaaagcttcagcctcatccgggattggagaagaaagaagaggaagaagaagaagaagaatatgatgaagggtctaatctcaaaaaacagaccaataagaaccgggtgcagtcaggcccccgcacacccaacccctatgcctcggacaacagcagcctcatgtttcccatcctggtggccttcggagtcttcattccaaccctcttctgcctctgccggttgtgagaacaaatgactatcctgaacagggtggaggggtgtgggaaagaaaccagccatattggttttggtttctgtatttttcacaatgattaatgaacaaaaacaaagagaaaaaaaacacacatcaattaaaggagacaaaaagaggcagagcgagtagagagcagccctcattcaccacctggtcccagacgtgcttcagtcctcgtcctctctttgtggctggctcccagccttctctttcctcttgaggatacttagggtaaactggatccttcctgctcaaggatcctcatttgtatacctagtggaaaggactctgaactcagaggagtcactgttcctttttttaggttagaaattaacagcagggaaatgccatcttattacctgagacgaccagcactgggagttaggtacggtctgaagttatgtctagataagacttcagacgtcctgggattgaaagaatgtgtgtgaaggggtagaatttgtgcggta

SEQ ID NO: 249 - DNA, 2514 bp, Homo sapiens
atgctgcgcaccgcgatgggcctgaggagctggctcgccgccccatggggcgcgctgccgcctcggccaccgctgctgctgctcctgctgctgctgctcctgctgcagccgccgcctccgacctgggcgctcagcccccggatcagcctgcctctgggctctgaagagcggccattcctcagattcgaagctgaacacatctccaactacacagcccttctgctgagcagggatggcaggaccctgtacgtgggtgctcgagaggccctctttgcactcagtagcaacctcagcttcctgccaggcggggagtaccaggagctgctttggggtgcagacgcagagaagaaacagcagtgcagcttcaagggcaaggacccacagcgcgactgtcaaaactacatcaagatcctcctgccgctcagcggcagtcacctgttcacctgtggcacagcagccttcagccccatgtgtacctacatcaacatggagaacttcaccctggcaagggacgagaaggggaatgtcctcctggaagatggcaagggccgttgtcccttcgacccgaatttcaagtccactgccctggtggttgatggcgagctctacactggaacagtcagcagcttccaagggaatgacccggccatctcgcggagccaaagccttcgccccaccaagaccgagagctccctcaactggctgcaagacccagcttttgtggcctcagcctacattcctgagagcctgggcagcttgcaaggcgatgatgacaagatctactttttcttcagcgagactggccaggaatttgagttctttgagaacaccattgtgtcccgcattgcccgcatctgcaagggcgatgagggtggagagcgggtgctacagcagcgctggacctccttcctcaaggcccagctgctgtgctcacggcccgacgatggcttccccttcaacgtgctgcaggatgtcttcacgctgagccccagcccccaggactggcgtgacacccttttctatggggtcttcacttcccagtggcacaggggaactacagaaggctctgccgtctgtgtcttcacaatgaaggatgtgcagagagtcttcagcggcctctacaaggaggtgaaccgtgagacacagcagtggtacaccgtgacccacccggtgcccacaccccggcctggagcgtgcatcaccaacagtgcccgggaaaggaagatcaactcatccctgcagctcccagaccgcgtgctgaacttcctcaaggaccacttcctgatggacgggcaggtccgaagccgcatgctgctgctgcagccccaggctcgctaccagcgcgtggctgtacaccgcgtccctggcctgcaccacacctacgatgtcctcttcctgggcactggtgacggccggctccacaaggcagtgagcgtgggcccccgggtgcacatcattgaggagctgcagatcttctcatcgggacagcccgtgcagaatctgctcctggacacccacagggggctgctgtatgcggcctcacactcgggcgtagtccaggtgcccatggccaactgcagcctgtacaggagctgtggggactgcctcctcgcccgggacccctactgtgcttggagcggctccagctgcaagcacgtcagcctctaccagcctcagctggccaccaggccgtggatccaggacatcgagggagccagcgccaaggacctttgcagcgcgtcttcggttgtgtccccgtcttttgtaccaacaggggagaagccatgtgagcaagtccagttccagcccaacacagtgaacactttggcctgcccgctcctctccaacctggcgacccgactctggctacgcaacggggcccccgtcaatgcctcggcctcctgccacgtgctacccactggggacctgctgctggtgggcacccaacagctgggggagttccagtgctggtcactagaggagggcttccagcagctggtagccagctactgcccagaggtggtggaggacggggtggcagaccaaacagatgagggtggcagtgtacccgtcattatcagcacatcgcgtgtgagtgcaccagctggtggcaaggccagctggggtgcagacaggtcctactggaaggagttcctggtgatgtgcacgctctttgtgctggccgtgctgctcccagttttattcttgctctaccggcaccggaacagcatgaaagtcttcctgaagcagggggaatgtgccagcgtgcaccccaagacctgccctgtggtgctgccccctgagacccgcccactcaacggcctagggccccctagcaccccgctcgatcaccgagggtaccagtccctgtcagacagccccccggggtcccgagtcttcactgagtcagagaagaggccactcagcatccaagacagcttcgtggaggtatccccagtgtgcccccggccccgggtccgccttggctcggagatccgtgactctgtggtgtga

SEQ ID NO: 250 - DNA, 1143 bp, Homo sapiens
atgaaggatgtgcagagagtcttcagcggcctctacaaggaggtgaaccgtgagacacagcagtggtacaccgtgacccacccggtgcccacaccccggcctggagcgtgcatcaccaacagtgcccgggaaaggaagatcaactcatccctgcagctcccagaccgcgtgctgaactttctcaaggaccacttcctgatggacgggcaggtccgaagccgcatgctgctgctgcagccccaggctcgctaccagcgcgtggctgtacaccgcgtccctggcctgcaccacacctacgatgtcctcttcctgggcactggtgacggccggctccacaaggcagtgagcgtgggcccccgggtgcacatcattgaggagctgcagatcttctcatcgggacagcccgtgcagaatctgctcctggacacccacagggggctgctgtatgcggcctcacactcgggcgtagtccaggtgcccatggccaactgcagcctgtacaggagctgtggggactgcctcctcgcccgggacccctactgtgcttggagcggctccagctgcaagcacgtcagcctctaccagcctcagctggccaccaggccgtggatccaggacatcgagggagccagcgccaaggacctttgcagcgcgtcttcggttgtgtccccgtcttttgtaccaacaggggagaagccatgtgagcaagtccagttccagcccaacacagtgaacactttggcctgcccgctcctctccaacctggcgacccgactctggctacgcaacggggcccccgtcaatgcctcggcctcctgccacgtgctacccactggggacctgctgctggtgggcacccaacagctgggggagttccagtgctggtcactagaggagggcttccagcagctggtagccagctactgcccagaggtggtggaggacggggtggcagaccaaacagatgagggtggcagtgtacccgtcattatcagcacatcgcgtgtgagtgcaccagctggtggcaaggccagctggggtgcagacaggtcctgggctcggacccaactcctggacctttccagcctgtatcaggctgtggccacacgagaggacagcgcgagctcaggagagatttcgtga

SEQ ID NO: 251 - DNA, 18 bp, Homo sapiens
ggtgcagacaggtcctgg

SEQ ID NO: 252 - DNA, 18 bp, Homo sapiens
gcagacaggtcctgggct

SEQ ID NO: 253 - DNA, 18 bp, Homo sapiens
gacaggtcctgggctcgg

SEQ ID NO: 254 - DNA, 18 bp, Homo sapiens
aggtcctgggctcggacc

SEQ ID NO: 255 - DNA, 18 bp, Homo sapiens
tcctgggctcggacccaa

SEQ ID NO: 256 - DNA, 72 bp, Homo sapiens
tactttttcttcagcgagactggccaggaatttgagttctttgagaacaccattgtgtcccgcattgcccgc

SEQ ID NO: 257 - DNA, 18 bp, Homo sapiens
tactttttcttcagcgag

SEQ ID NO: 258 - DNA, 18 bp, Homo sapiens
tttttcttcagcgagact

SEQ ID NO: 259 - DNA, 18 bp, Homo sapiens
ttcttcagcgagactggc

SEQ ID NO: 260 - DNA, 18 bp, Homo sapiens
ttcagcgagactggccag

SEQ ID NO: 261 - DNA, 18 bp, Homo sapiens
agcgagactggccaggaa

SEQ ID NO: 262 - DNA, 18 bp, Homo sapiens
gagactggccaggaattt

SEQ ID NO: 263 - DNA, 18 bp, Homo sapiens
actggccaggaatttgag

SEQ ID NO: 264 - DNA, 18 bp, Homo sapiens
ggccaggaatttgagttc

SEQ ID NO: 265 - DNA, 18 bp, Homo sapiens
caggaatttgagttcttt

SEQ ID NO: 266 - DNA, 18 bp, Homo sapiens
gaatttgagttctttgag

SEQ ID NO: 267 - DNA, 18 bp, Homo sapiens
tttgagttctttgagaac

SEQ ID NO: 268 - DNA, 18 bp, Homo sapiens
gagttctttgagaacacc

SEQ ID NO: 269 - DNA, 18 bp, Homo sapiens
ttctttgagaacaccatt

SEQ ID NO: 270 - DNA, 18 bp, Homo sapiens
tttgagaacaccattgtg

SEQ ID NO: 271 - DNA, 18 bp, Homo sapiens
gagaacaccattgtgtcc

SEQ ID NO: 272 - DNA, 18 bp, Homo sapiens
aacaccattgtgtcccgc

SEQ ID NO: 273 - DNA, 18 bp, Homo sapiens
accattgtgtcccgcatt

SEQ ID NO: 274 - DNA, 18 bp, Homo sapiens
attgtgtcccgcattgcc

SEQ ID NO: 275 - DNA, 18 bp, Homo sapiens
gtgtcccgcattgcccgc

SEQ ID NO: 276 - DNA, 75 bp, Homo sapiens
ctcaaggcccagctgctgtgctcacggcccgacgatggcttccccttcaacgtgctgcaggatgtcttcacgctg

SEQ ID NO: 277 - DNA, 18 bp, Homo sapiens
ctcaaggcccagctgctg

SEQ ID NO: 278 - DNA, 18 bp, Homo sapiens
aaggcccagctgctgtgc

SEQ ID NO: 279 - DNA, 18 bp, Homo sapiens
gcccagctgctgtgctca

SEQ ID NO: 280 - DNA, 18 bp, Homo sapiens
cagctgctgtgctcacgg

SEQ ID NO: 281 - DNA, 18 bp, Homo sapiens
ctgctgtgctcacggccc

SEQ ID NO: 282 - DNA, 18 bp, Homo sapiens
ctgtgctcacggcccgac

SEQ ID NO: 283 - DNA, 18 bp, Homo sapiens
tgctcacggcccgacgat

SEQ ID NO: 284 - DNA, 18 bp, Homo sapiens
tcacggcccgacgatggc

SEQ ID NO: 285 - DNA, 18 bp, Homo sapiens
cggcccgacgatggcttc

SEQ ID NO: 286 - DNA, 18 bp, Homo sapiens
cccgacgatggcttcccc

SEQ ID NO: 287 - DNA, 18 bp, Homo sapiens
gacgatggcttccccttc

SEQ ID NO: 288 - DNA, 18 bp, Homo sapiens
gatggcttccccttcaac

SEQ ID NO: 289 - DNA, 18 bp, Homo sapiens
ggcttccccttcaacgtg

SEQ ID NO: 290 - DNA, 18 bp, Homo sapiens
ttccccttcaacgtgctg

SEQ ID NO: 291 - DNA, 18 bp, Homo sapiens
cccttcaacgtgctgcag

SEQ ID NO: 292 - DNA, 18 bp, Homo sapiens
ttcaacgtgctgcaggat

SEQ ID NO: 293 - DNA, 18 bp, Homo sapiens
aacgtgctgcaggatgtc

SEQ ID NO: 294 - DNA, 18 bp, Homo sapiens
gtgctgcaggatgtcttc

SEQ ID NO: 295 - DNA, 18 bp, Homo sapiens
ctgcaggatgtcttcacg

SEQ ID NO: 296 - DNA, 18 bp, Homo sapiens
caggatgtcttcacgctg

SEQ ID NO: 297 - DNA, 63 bp, Homo sapiens
cttttctatggggtcttcacttcccagtggcacaggggaactacagaaggctctgccgtctgt

SEQ ID NO: 298 - DNA, 18 bp, Homo sapiens
cttttctatggggtcttc

SEQ ID NO: 299 - DNA, 18 bp, Homo sapiens
ttctatggggtcttcact

SEQ ID NO: 300 - DNA, 18 bp, Homo sapiens
tatggggtcttcacttcc

SEQ ID NO: 301 - DNA, 18 bp, Homo sapiens
ggggtcttcacttcccag

SEQ ID NO: 302 - DNA, 18 bp, Homo sapiens
gtcttcacttcccagtgg

SEQ ID NO: 303 - DNA, 18 bp, Homo sapiens
ttcacttcccagtggcac

SEQ ID NO: 304 - DNA, 18 bp, Homo sapiens
acttcccagtggcacagg

SEQ ID NO: 305 - DNA, 18 bp, Homo sapiens
tcccagtggcacagggga

SEQ ID NO: 306 - DNA, 18 bp, Homo sapiens
cagtggcacaggggaact

SEQ ID NO: 307 - DNA, 18 bp, Homo sapiens
tggcacaggggaactaca

SEQ ID NO: 308 - DNA, 18 bp, Homo sapiens
cacaggggaactacagaa

SEQ ID NO: 309 - DNA, 18 bp, Homo sapiens
aggggaactacagaaggc

SEQ ID NO: 310 - DNA, 18 bp, Homo sapiens
ggaactacagaaggctct

SEQ ID NO: 311 - DNA, 18 bp, Homo sapiens
actacagaaggctctgcc

SEQ ID NO: 312 - DNA, 18 bp, Homo sapiens
acagaaggctctgccgtc

SEQ ID NO: 313 - DNA, 18 bp, Homo sapiens
gaaggctctgccgtctgt

SEQ ID NO: 314 - protein, 837 aa, Homo sapiens
MLRTAMGLRSWLAAPWGALPPRPPLLLLLLLLLLLQPPPPTWALSPRISLPLGSEERPFLRFEAEHISNYTALLLSRDGRTLYVGAREALFALSSNLSFLPGGEYQELLWGADAEKKQQCSFKGKDPQRDCQNYIKILLPLSGSHLFTCGTAAFSPMCTYINMENFTLARDEKGNVLLEDGKGRCPFDPNFKSTALVVDGELYTGTVSSFQGNDPAISRSQSLRPTKTESSLNWLQDPAFVASAYIPESLGSLQGDDDKIYFFFSETGQEFEFFENTIVSRIARICKGDEGGERVLQQRWTSFLKAQLLCSRPDDGFPFNVLQDVFTLSPSPQDWRDTLFYGVFTSQWHRGTTEGSAVCVFTMKDVQRVFSGLYKEVNRETQQWYTVTHPVPTPRPGACITNSARERKINSSLQLPDRVLNFLKDHFLMDGQVRSRMLLLQPQARYQRVAVHRVPGLHHTYDVLFLGTGDGRLHKAVSVGPRVHIIEELQIFSSGQPVQNLLLDTHRGLLYAASHSGVVQVPMANCSLYRSCGDCLLARDPYCAWSGSSCKHVSLYQPQLATRPWIQDIEGASAKDLCSASSVVSPSFVPTGEKPCEQVQFQPNTVNTLACPLLSNLATRLWLRNGAPVNASASCHVLPTGDLLLVGTQQLGEFQCWSLEEGFQQLVASYCPEVVEDGVADQTDEGGSVPVIISTSRVSAPAGGKASWGADRSYWKEFLVMCTLFVLAVLLPVLFLLYRHRNSMKVFLKQGECASVHPKTCPVVLPPETRPLNGLGPPSTPLDHRGYQSLSDSPPGSRVFTESEKRPLSIQDSFVEVSPVCPRPRVRLGSEIRDSVV

SEQ ID NO: 315 - protein, 380 aa, Homo sapiens
MKDVQRVFSGLYKEVNRETQQWYTVTHPVPTPRPGACITNSARERKINSSLQLPDRVLNFLKDHFLMDGQVRSRMLLLQPQARYQRVAVHRVPGLHHTYDVLFLGTGDGRLHKAVSVGPRVHIIEELQIFSSGQPVQNLLLDTHRGLLYAASHSGVVQVPMANCSLYRSCGDCLLARDPYCAWSGSSCKHVSLYQPQLATRPWIQDIEGASAKDLCSASSVVSPSFVPTGEKPCEQVQFQPNTVNTLACPLLSNLATRLWLRNGAPVNASASCHVLPTGDLLLVGTQQLGEFQCWSLEEGFQQLVASYCPEVVEDGVADQTDEGGSVPVIISTSRVSAPAGGKASWGADRSWARTQLLDLSSLYQAVATREDSASSGEIS

SEQ ID NO: 316 - protein, 6 aa, Homo sapiens
GADRSW

SEQ ID NO: 317 - protein, 6 aa, Homo sapiens
ADRSWA

SEQ ID NO: 318 - protein, 6 aa, Homo sapiens
DRSWAR

SEQ ID NO: 319 - protein, 6 aa, Homo sapiens
RSWART

SEQ ID NO: 320 - protein, 6 aa, Homo sapiens
SWARTQ

SEQ ID NO: 321 - protein, 24 aa, Homo sapiens
YFFFSETGQEFEFFENTIVSRIAR

SEQ ID NO: 322 - protein, 6 aa, Homo sapiens
YFFFSE

SEQ ID NO: 323 - protein, 6 aa, Homo sapiens
FFFSET

SEQ ID NO: 324 - protein, 6 aa, Homo sapiens
FFSETG

SEQ ID NO: 325 - protein, 6 aa, Homo sapiens
FSETGQ

SEQ ID NO: 326 - protein, 6 aa, Homo sapiens
SETGQE

SEQ ID NO: 327 - protein, 6 aa, Homo sapiens
ETGQEF

SEQ ID NO: 328 - protein, 6 aa, Homo sapiens
TGQEFE

SEQ ID NO: 329 - protein, 6 aa, Homo sapiens
GQEFEF

SEQ ID NO: 330 - protein, 6 aa, Homo sapiens
QEFEFF

SEQ ID NO: 331 - protein, 6 aa, Homo sapiens
EFEFFE

SEQ ID NO: 332 - protein, 6 aa, Homo sapiens
FEFFEN

SEQ ID NO: 333 - protein, 6 aa, Homo sapiens
EFFENT

SEQ ID NO: 334 - protein, 6 aa, Homo sapiens
FFENTI

SEQ ID NO: 335 - protein, 6 aa, Homo sapiens
FENTIV

SEQ ID NO: 336 - protein, 6 aa, Homo sapiens
ENTIVS

SEQ ID NO: 337 - protein, 6 aa, Homo sapiens
NTIVSR

SEQ ID NO: 338 - protein, 6 aa, Homo sapiens
TIVSRI

SEQ ID NO: 339 - protein, 6 aa, Homo sapiens
IVSRIA

SEQ ID NO: 340 - protein, 6 aa, Homo sapiens
VSRIAR

SEQ ID NO: 341 - protein, 25 aa, Homo sapiens
LKAQLLCSRPDDGFPFNVLQDVFTL

SEQ ID NO: 342 - protein, 6 aa, Homo sapiens
LKAQLL

SEQ ID NO: 343 - protein, 6 aa, Homo sapiens
KAQLLC

SEQ ID NO: 344 - protein, 6 aa, Homo sapiens
AQLLCS

SEQ ID NO: 345 - protein, 6 aa, Homo sapiens
QLLCSR

SEQ ID NO: 346 - protein, 6 aa, Homo sapiens
LLCSRP

SEQ ID NO: 347 - protein, 6 aa, Homo sapiens
LCSRPD

SEQ ID NO: 348 - protein, 6 aa, Homo sapiens
CSRPDD

SEQ ID NO: 349 - protein, 6 aa, Homo sapiens
SRPDDG

SEQ ID NO: 350 - protein, 6 aa, Homo sapiens
RPDDGF

SEQ ID NO: 351 - protein, 6 aa, Homo sapiens
PDDGFP

SEQ ID NO: 352 - protein, 6 aa, Homo sapiens
DDGFPF

SEQ ID NO: 353 - protein, 6 aa, Homo sapiens
DGFPFN

SEQ ID NO: 354 - protein, 6 aa, Homo sapiens
GFPFNV

SEQ ID NO: 355 - protein, 6 aa, Homo sapiens
FPFNVL

SEQ ID NO: 356 - protein, 6 aa, Homo sapiens
PFNVLQ

SEQ ID NO: 357 - protein, 6 aa, Homo sapiens
FNVLQD

SEQ ID NO: 358 - protein, 6 aa, Homo sapiens
NVLQDV

SEQ ID NO: 359 - protein, 6 aa, Homo sapiens
VLQDVF

SEQ ID NO: 360 - protein, 6 aa, Homo sapiens
LQDVFT

SEQ ID NO: 361 - protein, 6 aa, Homo sapiens
QDVFTL

SEQ ID NO: 362 - protein, 21 aa, Homo sapiens
LFYGVFTSQWHRGTTEGSAVC

SEQ ID NO: 363 - protein, 6 aa, Homo sapiens
LFYGVF

SEQ ID NO: 364 - protein, 6 aa, Homo sapiens
FYGVFT

SEQ ID NO: 365 - protein, 6 aa, Homo sapiens
YGVFTS

SEQ ID NO: 366 - protein, 6 aa, Homo sapiens
GVFTSQ

SEQ ID NO: 367 - protein, 6 aa, Homo sapiens
VFTSQW

SEQ ID NO: 368 - protein, 6 aa, Homo sapiens
FTSQWH

SEQ ID NO: 369 - protein, 6 aa, Homo sapiens
TSQWHR

SEQ ID NO: 370 - protein, 6 aa, Homo sapiens
SQWHRG

SEQ ID NO: 371 - protein, 6 aa, Homo sapiens
QWHRGT

SEQ ID NO: 372 - protein, 6 aa, Homo sapiens
WHRGTT

SEQ ID NO: 373 - protein, 6 aa, Homo sapiens
HRGTTE

SEQ ID NO: 374 - protein, 6 aa, Homo sapiens
RGTTEG

SEQ ID NO: 375 - protein, 6 aa, Homo sapiens
GTTEGS

SEQ ID NO: 376 - protein, 6 aa, Homo sapiens
TTEGSA

SEQ ID NO: 377 - protein, 6 aa, Homo sapiens
TEGSAV

SEQ ID NO: 378 - protein, 6 aa, Homo sapiens
EGSAVC

SEQ ID NO: 379 - DNA, 3297 bp, Homo sapiens
atttccggggacgccgcgccggactgaggctgtgcgcccgagactccgggtccccaggggctgcgccgggccggcctggcaagggggacgagtcagtggacactccaggaagagcggccccgcggggggcgatgaccgtgcgctgaccctgactcactccaggtccggaggcgggggcccccggggcgactcgggggcggaccgcggggcggagctgccgcccgtgagtccggccgagccacctgagcccgagccgcgggacaccgtcgctcctgctctccgaatgctgcgcaccgcgatgggcctgaggagctggctcgccgccccatggggcgcgctgccgcctcggccaccgctgctgctgctcctgctgctgctgctcctgctgcagccgccgcctccgacctgggcgctcagcccccggatcagcctgcctctgggctctgaagagcggccattcctcagattcgaagctgaacacatctccaactacacagcccttctgctgagcagggatggcaggaccctgtacgtgggtgctcgagaggccctctttgcactcagtagcaacctcagcttcctgccaggcggggagtaccaggagctgctttggggtgcagacgcagagaagaaacagcagtgcagcttcaagggcaaggacccacagcgcgactgtcaaaactacatcaagatcctcctgccgctcagcggcagtcacctgttcacctgtggcacagcagccttcagccccatgtgtacctacatcaacatggagaacttcaccctggcaagggacgagaaggggaatgtcctcctggaagatggcaagggccgttgtcccttcgacccgaatttcaagtccactgccctggtggttgatggcgagctctacactggaacagtcagcagcttccaagggaatgacccggccatctcgcggagccaaagccttcgccccaccaagaccgagagctccctcaactggctgcaagacccagcttttgtggcctcagcctacattcctgagagcctgggcagcttgcaaggcgatgatgacaagatctactttttcttcagcgagactggccaggaatttgagttctttgagaacaccattgtgtcccgcattgcccgcatctgcaagggcgatgagggtggagagcgggtgctacagcagcgctggacctccttcctcaaggcccagctgctgtgctcacggcccgacgatggcttccccttcaacgtgctgcaggatgtcttcacgctgagccccagcccccaggactggcgtgacacccttttctatggggtcttcacttcccagtggcacaggggaactacagaaggctctgccgtctgtgtcttcacaatgaaggatgtgcagagagtcttcagcggcctctacaaggaggtgaaccgtgagacacagcagtggtacaccgtgacccacccggtgcccacaccccggcctggagcgtgcatcaccaacagtgcccgggaaaggaagatcaactcatccctgcagctcccagaccgcgtgctgaacttcctcaaggaccacttcctgatggacgggcaggtccgaagccgcatgctgctgctgcagccccaggctcgctaccagcgcgtggctgtacaccgcgtccctggcctgcaccacacctacgatgtcctcttcctgggcactggtgacggccggctccacaaggcagtgagcgtgggcccccgggtgcacatcattgaggagctgcagatcttctcatcgggacagcccgtgcagaatctgctcctggacacccacagggggctgctgtatgcggcctcacactcgggcgtagtccaggtgcccatggccaactgcagcctgtacaggagctgtggggactgcctcctcgcccgggacccctactgtgcttggagcggctccagctgcaagcacgtcagcctctaccagcctcagctggccaccaggccgtggatccaggacatcgagggagccagcgccaaggacctttgcagcgcgtcttcggttgtgtccccgtcttttgtaccaacaggggagaagccatgtgagcaagtccagttccagcccaacacagtgaacactttggcctgcccgctcctctccaacctggcgacccgactctggctacgcaacggggcccccgtcaatgcctcggcctcctgccacgtgctacccactggggacctgctgctggtgggcacccaacagctgggggagttccagtgctggtcactagaggagggcttccagcagctggtagccagctactgcccagaggtggtggaggacggggtggcagaccaaacagatgagggtggcagtgtacccgtcattatcagcacatcgcgtgtgagtgcaccagctggtggcaaggccagctggggtgcagacaggtcctactggaaggagttcctggtgatgtgcacgctctttgtgctggccgtgctgctcccagttttattcttgctctaccggcaccggaacagcatgaaagtcttcctgaagcagggggaatgtgccagcgtgcaccccaagacctgccctgtggtgctgccccctgagacccgcccactcaacggcctagggccccctagcaccccgctcgatcaccgagggtaccagtccctgtcagacagccccccggggtcccgagtcttcactgagtcagagaagaggccactcagcatccaagacagcttcgtggaggtatccccagtgtgcccccggccccgggtccgccttggctcggagatccgtgactctgtggtgtgagagctgacttccagaggacgctgccctggcttcaggggctgtgaatgctcggagagggtcaactggacctcccctccgctctgctcttcgtggaacacgaccgtggtgcccggcccttgggagccttggggccagctggcctgctgctctccagtcaagtagcgaagctcctaccacccagacacccaaacagccgtggccccagaggtcctggccaaatatgggggcctgcctaggttggtggaacagtgctccttatgtaaactgagccctttgtttaaaaaacaattccaaatgtgaaactagaatgagagggaagagatagcatggcatgcagcacacacggctgctccagttcatggcctcccaggggtgctggggatgcatccaaagtggttgtctgagacagagttggaaaccctcaccaactggcctcttcaccttccacattatcccgctgccaccggctgccctgtctcactgcagattcaggaccagct

SEQ ID NO: 380 - DNA, 1712 bp, Homo sapiens
cccgcatctgcaagggcgatgagggtggagagcgggtgctacagcagcgctggacctccttcctcaaggcccagctgctgtgctcacggcccgacgatggcttccccttcaacgtgctgcaggatgtcttcacgctgagccccagcccccaggactggcgtgacacccttttctatggggtcttcacttcccagtggcacaggggaactacagaaggctctgccgtctgtgtcttcacaatgaaggatgtgcagagagtcttcagcggcctctacaaggaggtgaaccgtgagacacagcagtggtacaccgtgacccacccggtgcccacaccccggcctggagcgtgcatcaccaacagtgcccgggaaaggaagatcaactcatccctgcagctcccagaccgcgtgctgaactttctcaaggaccacttcctgatggacgggcaggtccgaagccgcatgctgctgctgcagccccaggctcgctaccagcgcgtggctgtacaccgcgtccctggcctgcaccacacctacgatgtcctcttcctgggcactggtgacggccggctccacaaggcagtgagcgtgggcccccgggtgcacatcattgaggagctgcagatcttctcatcgggacagcccgtgcagaatctgctcctggacacccacagggggctgctgtatgcggcctcacactcgggcgtagtccaggtgcccatggccaactgcagcctgtacaggagctgtggggactgcctcctcgcccgggacccctactgtgcttggagcggctccagctgcaagcacgtcagcctctaccagcctcagctggccaccaggccgtggatccaggacatcgagggagccagcgccaaggacctttgcagcgcgtcttcggttgtgtccccgtcttttgtaccaacaggggagaagccatgtgagcaagtccagttccagcccaacacagtgaacactttggcctgcccgctcctctccaacctggcgacccgactctggctacgcaacggggcccccgtcaatgcctcggcctcctgccacgtgctacccactggggacctgctgctggtgggcacccaacagctgggggagttccagtgctggtcactagaggagggcttccagcagctggtagccagctactgcccagaggtggtggaggacggggtggcagaccaaacagatgagggtggcagtgtacccgtcattatcagcacatcgcgtgtgagtgcaccagctggtggcaaggccagctggggtgcagacaggtcctgggctcggacccaactcctggacctttccagcctgtatcaggctgtggccacacgagaggacagcgcgagctcaggagagatttcgtgacaatgtacgcctttccctcagaattcagggaagagactgtcgcctgccttcctccgttgttgcgtgagaacccgtgtgccccttcccaccatatccaccctcgctccatctttgaactcaaacacgaggaactaactgcaccctggtcctctccccagtccccagttcaccctccatccctcaccttcctccactctaagggatatcaacactgcccagcacaggggccctgaatttatgtggtttttatacattttttaataagatgcactttatgtcattttttaataaagtctgaagaattactgtttaaaaaaaaaaaaaaaaaaa

(19) United States
(12) Patent Application Publication
(10) Pub. No.: US 2009/0214517 A1
(43) Pub. Date: Aug. 27, 2009

(54) Compositions and methods of use for modulators of nectin 4, semaphorin 4b, igsf9, and kiaa0152 in treating disease

(76) Inventors: Justin Wong, Oakland, CA (US); Kevin Hestir, Kensington, CA (US); Ernestine Lee, Kensington, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/658,412

(22) PCT Filed: Jul. 27, 2005
(86) PCT No.: PCT/US2005/026561

§ 371 Date: Mar. 9, 2009

Related U.S. Application Data
(60) Provisional application No. 60/591,527, filed on Jul. 27, 2004.

(51) Int. Cl. A61K 39/395; A61P 35/00

(52) U.S. Cl. 424/130.1; 424/178.1; 424/183.1

(57) ABSTRACT

Microarray analysis, confirmed by RT-PCT, demonstrated that mRNA derived from cancerous tissues hybridized specifically and preferentially to human nectin 4, semaphorin 4b, IgSF9, and KIAA0152. Microarray analysis also demonstrated that RNA from malignant bladder, pancreas, and stomach tissue hybridized specifically to human nectin 4, semaphorin 4b, IgSF9, and KIAA0152, all of which are transmembrane proteins that provide a therapeutic target for treating cancer. Modulators of nectin 4, semaphorin 4b, IgSF9, and KIAA0152 are provided for the diagnosis and treatment of proliferative disorders such as cancer and psoriasis. The invention further provides methods of treating cancer with therapeutic agents directed toward nectin 4, semaphorin 4b, IgSF9, and KIAA0152.

This application claims the benefit of priority to U.S. application No. 60/591,527, “Targets for Treating Proliferative and Immune Disorders and Modulators Thereof,” filed Jul. 27, 2004, which is incorporated by reference in its entirety. This application is related to PCT/US04/002655, “Lung-Expressed Polypeptides,” filed under the Patent Cooperation Treaty on Jan. 30, 2004, and “Compositions and Methods of Use for ADAM12 Antagonists in Treating Disease, filed under the Patent Cooperation Treaty on Jul. 26, 2005, both of which are incorporated by reference in their entireties.

### TECHNICAL FIELD

This invention relates to human nectin 4, semaphorin 4b, IgSF9, and KIAA0152 polynucleotides, and their encoded polypeptides, which are highly expressed in cancer tissues, including lung, colon, rectal, stomach, prostate, and pancreatic cancers. The invention also relates to modulators of such polynucleotides and polypeptides, for example, antibodies, that specifically bind to and/or interfere with the activity of these polypeptides, polynucleotides, their fragments, variants, and antagonists. The invention further relates to compositions containing such polypeptides, polynucleotides, or modulators thereof and uses of such compositions in methods of treating or preventing proliferative disorders, including cancer and psoriasis, by detecting these polynucleotides, polypeptides, or antibodies thereto in patient samples. The invention provides diagnostic tests which identify nectin 4, semaphorin 4b, IgSF9, and KIAA0152 polypeptides and polynucleotides that correlate with particular disorders.

### BACKGROUND ART

The American Cancer Society estimates that approximately 1,400,000 new cases of cancer were diagnosed in the United States in 2004, and that approximately 570,000 cancer patients have died of the disease. An estimated 173,000 of these new cases were diagnosed as lung cancer, and an estimated 163,000 patients died of lung cancer in 2004. Lung cancer is the leading cause of cancer death in both men and women and carries an especially poor prognosis. While the five year survival rate for all cancers combined is 64%, the five year survival rate for lung cancer is only 15%. This is because most lung cancers are not detected until the disease has reached an advanced stage. Tumor stage is the most significant determinant of survival. When lung cancer is detected at an early stage, the five year survival rate climbs to 49% (American Cancer Society, 2005).

An estimated 147,000 of the newly diagnosed cancers were diagnosed as cancer, like lung cancer, of the colon or rectum, or colorectal cancer, and an estimated 57,000 patients will have died of this disease in 2004. In its early stages, colorectal cancer usually also causes no symptoms. When it is detected at an early, localized, stage the five year survival rate is 90%; however, only 38% of colorectal cancers are discovered at this stage (American Cancer Society, 2005). Therefore, diagnostic markers for both early stage lung and colorectal cancer will have a significant impact on cancer morbidity and mortality.

Detection of cancer cell-specific biomarkers provides an effective screening strategy for a number of cancers. Their early detection provides not only early diagnosis, but also the ability to screen for and detect post-operative residual tumor cells, and for occult metastases, an early indicator of tumor recurrence. Early detection can thus improve survival in patients before diagnosis, while undergoing treatment, and while in remission.

It would be desirable to provide novel methods and compositions for the treatment and prevention of cancers, such as lung, prostate, colon and other cancers, and other proliferative and immune-related diseases that are more efficacious and have a better safety profile than the currently available treatment modalities. It would also be desirable to provide better diagnostic tests for such diseases.

### BRIEF DESCRIPTION OF THE DRAWINGS AND TABLES

### Brief Description of the Drawings

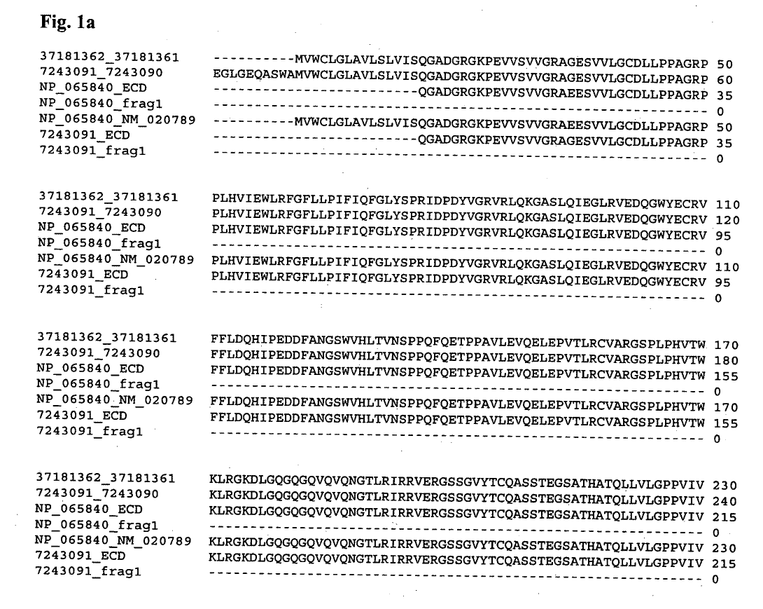

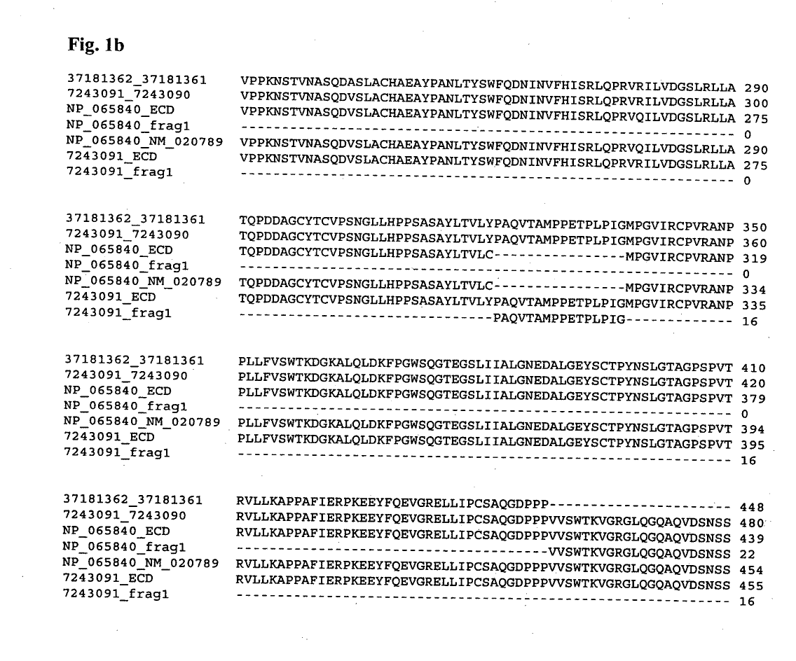

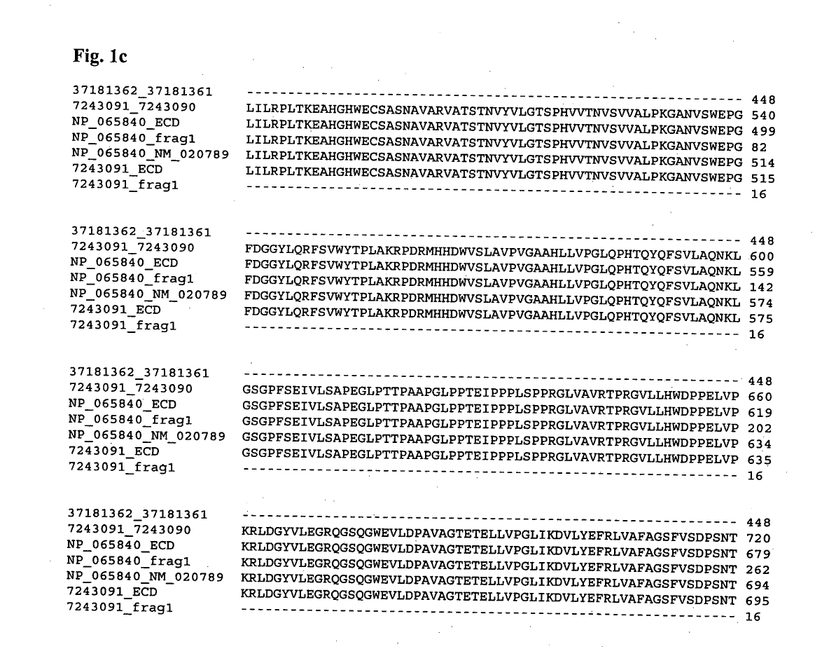

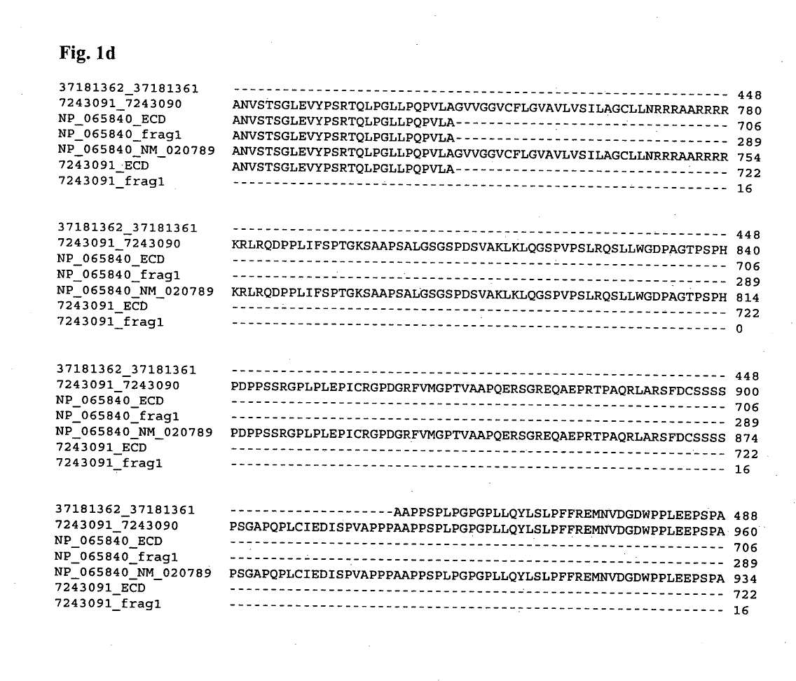

FIG. 1 compares the amino acid sequences of National Center for Biotechnology Information (NCBI) sequences belonging to cluster 192303, IgSF9. A cluster is an internally devised mechanism for grouping human cDNA clones which map to a single locus on the human chromosome. Cluster 192303, IgSF9, was identified by microarray hybridization to probe PRB103989_s_at. Sequences were aligned using clustal format for T-COFFEE Version_{—}1.37 with the parameters CPU=0.00 sec, SCORE=75, Nseq=7, Len=1198. The NCBI accession numbers 37181362_{—}37181361, 7243091_{—}7243090, and NP_{—}065840_NM020789 are provided to the left of the sequences. NP_{—}065840_ECD and 7243091_ECD denote the extracellular domains of clone NP_{—}065840 and 7243091. NP_{—}065840_ECD lacks a 16 amino acid internal sequence compared to 7243091_ECD. This 16 amino acid sequence is represented by 7243091_frag1. Asterisks (*) indicate amino acid residues shared among all the sequences; colons (:) indicate conservative amino acid changes; and dashes (−) indicate absent amino acids.

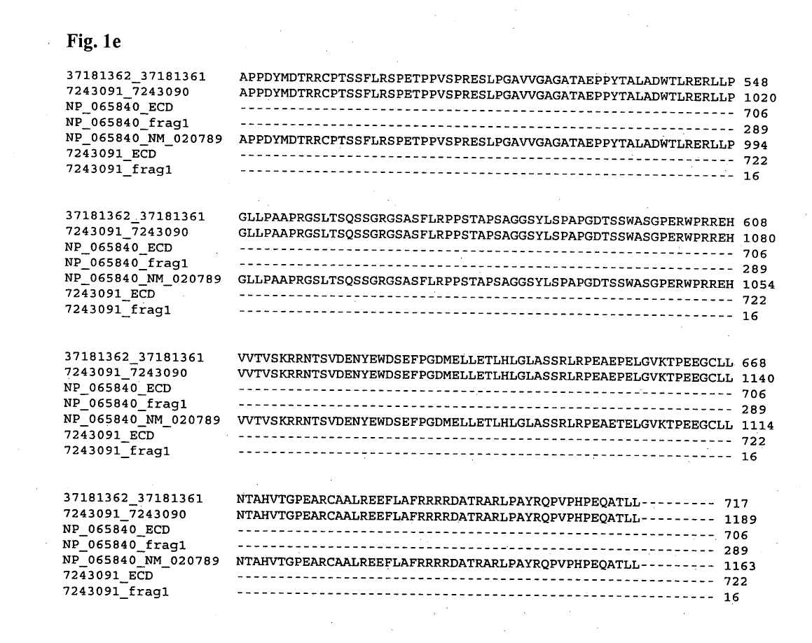

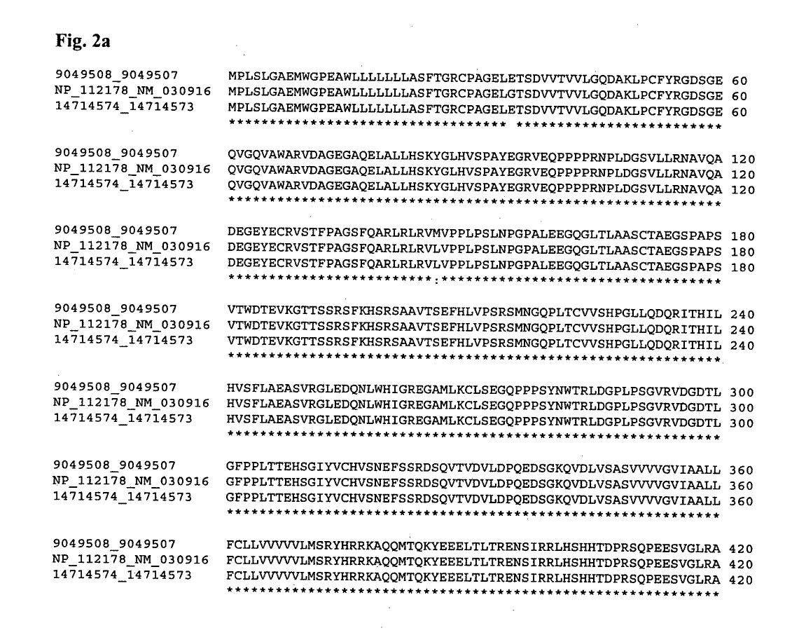

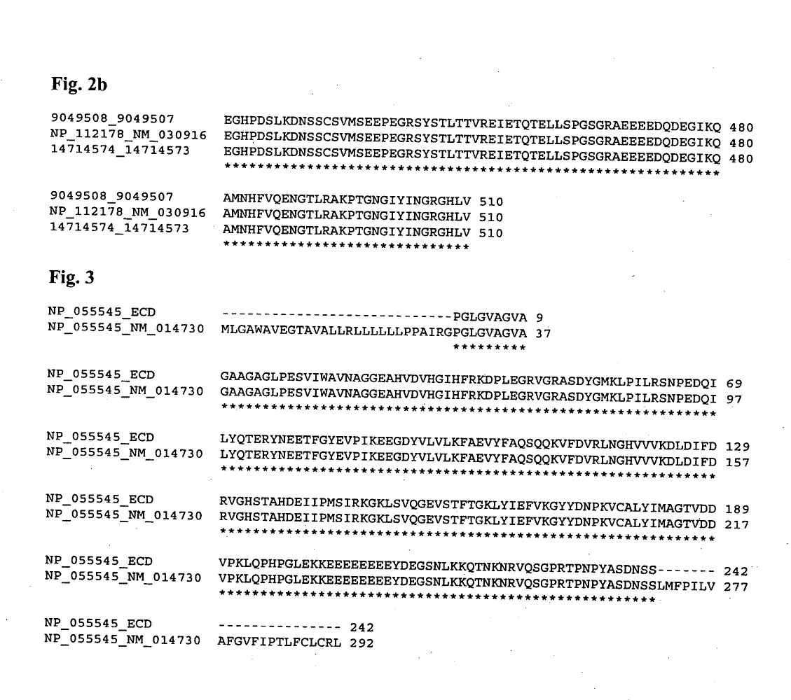

FIG. 2 compares the amino acid sequences of NCBI sequences belonging to cluster 301014, nectin 4. Cluster 301014, nectin 4, was identified by microarray hybridization to probe PRB103018_s_at. Sequences were aligned using clustal format for T-COFFEE Version_{—}1.37 with the parameters CPU=0.00 sec, SCORE=99, Nseq=3, Len=510. The NCBI accession numbers 9049508_{—}9049507, NP_{—}112178_NM_{—}030916, and 14714574_{—}14714573 are provided to the left of the sequences. Asterisks (*) indicate amino acid residues shared among all the sequences; colons (:) indicate conservative amino acid changes; and dashes (−) indicate absent amino acids.

FIG. 3 compares the amino acid sequences of NCBI sequences belonging to cluster 206895, KIAA0152. Cluster 206895, KIAA0152, was identified by microarray hybridization to probe PRB105610_at. Sequences were aligned using clustal format for T-COFFEE Version_{—}1.37 with the parameters CPU=0.00 sec, SCORE=95, Nseq=3, Len=315. The NCBI accession number NP_{—}055545_NM_{—}014730 is provided to the left of the sequences. Asterisks (*) indicate amino acid residues shared among all the sequences; colons (:) indicate conservative amino acid changes; and dashes (−) indicate absent amino acids.

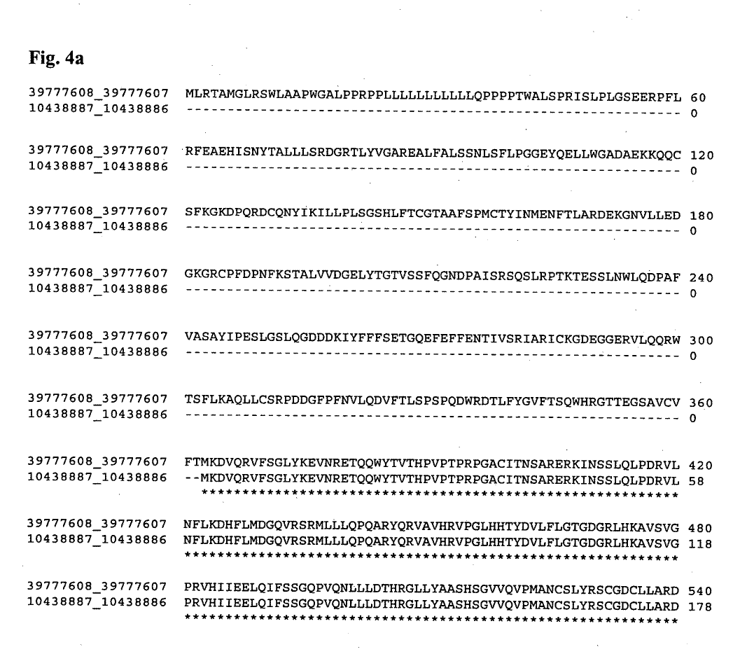

FIG. 4 compares the amino acid sequences of NCBI sequences belonging to cluster 181658, semaphorin 4B. Cluster 181658, semaphorin 4B, was identified by microarray hybridization to probe PRB101227_at. Sequences were aligned using clustal format for T-COFFEE Version_{—}1.37 with the parameters CPU=0.00 sec, SCORE=100, Nseq=2, Len=837. The NCBI accession numbers 39777608_{—}39777607 and 10438887_{—}10438886 are provided to the left of the sequences. Asterisks (*) indicate amino acid residues shared among all the sequences; colons (:) indicate conservative amino acid changes; and dashes (−) indicate absent amino acids.

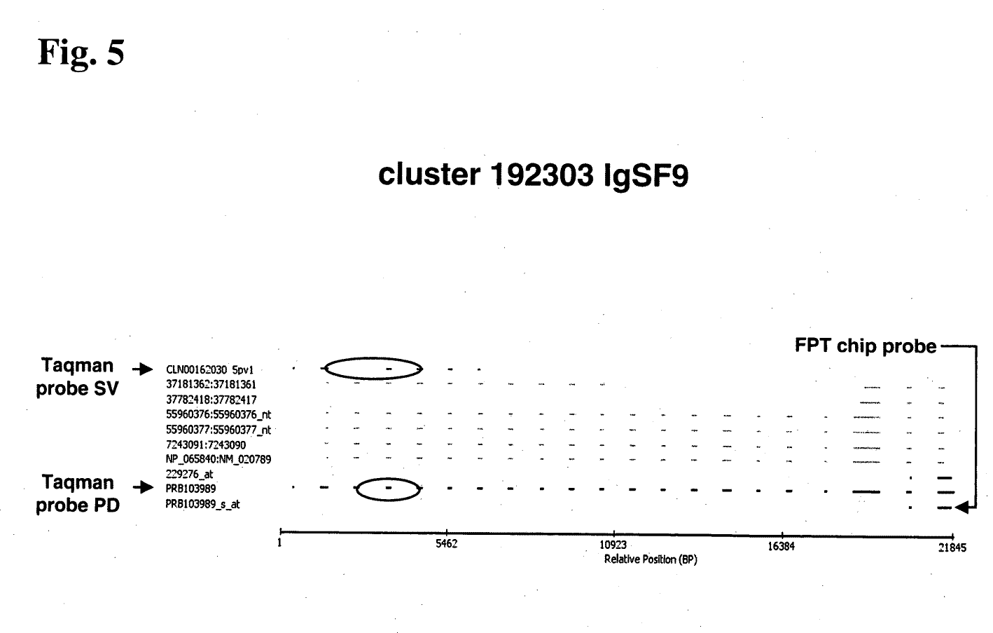

FIG. 5 shows an exon map of public and proprietary clones in cluster 192303, IgSF9; the location of microarray hybridization probe PRB103989_s_at; and the relative location of the RT-PCR primers and probes for CLN00162030 5pv1 (Taqman probe SV and Taqman probe PD). The horizontal axis is a scaled version of the genome which considers all introns to have equal lengths.

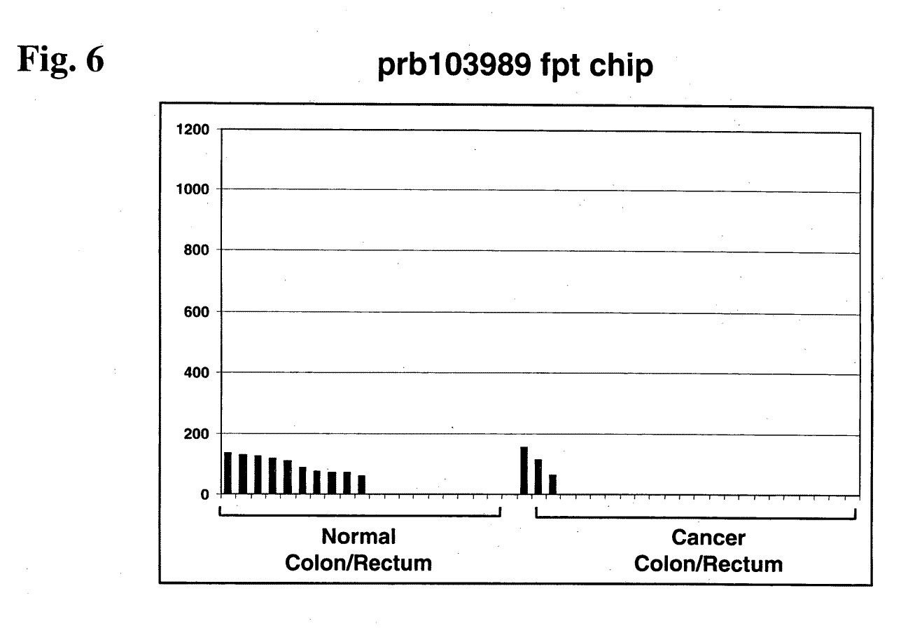

FIG. 6 shows the expression level of IgSF9 as detected by PRB103989 (black bars) and measured by microarray hybridization, using a Five Prime chip, of 23 colorectal adenocarcinoma samples, and 19 normal human colorectal specimens. The results show that IgSF9 was expressed in three tumor samples and 10 normal samples.

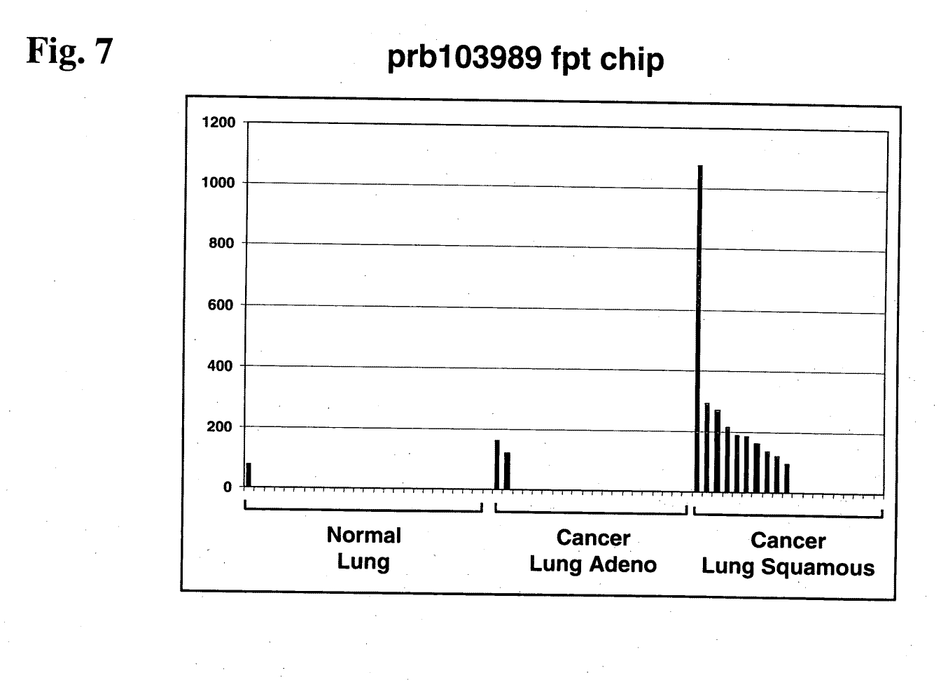

FIG. 7 shows the expression level of IgSF9 as detected by PRB103989 (black bars) and measured by microarray hybridization, using a Five Prime chip, of 19 lung squamous cell cancer samples, 19 human lung adenocarcinoma samples, and 24 normal lung samples. The results show that IgSF9 was expressed in 10 of 19 lung squamous carcinoma samples, two of 19 lung adenocarcinoma samples, and one of 24 normal lung samples.

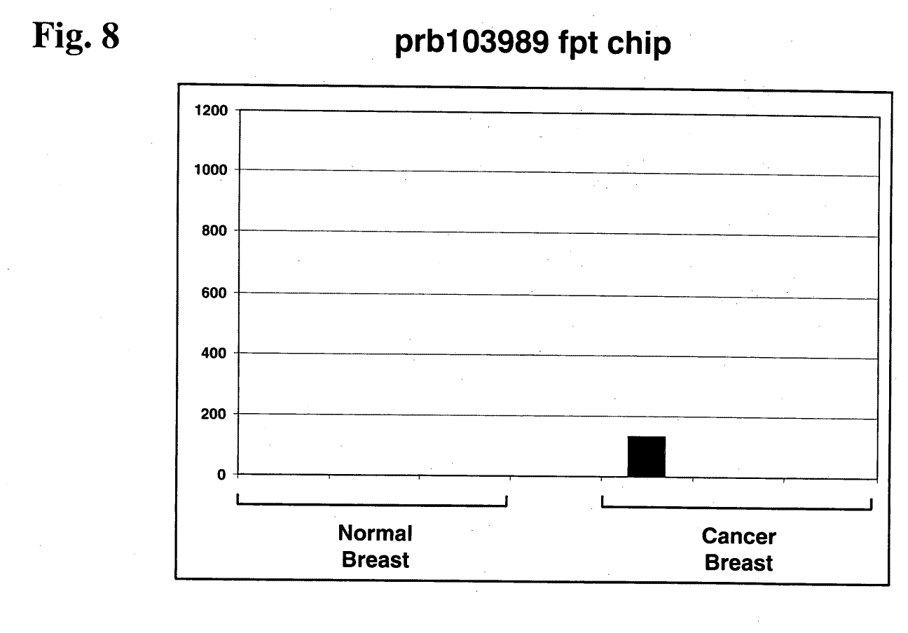

FIG. 8 shows the expression level of IgSF9 as detected by PRB103989 (black bars) and measured by microarray hybridization, using a Five Prime chip, of three malignant breast samples, and three normal human breast samples. The results show that IgSF9 was absent in the normal samples. IgSF9 was expressed in one of the tumor samples.

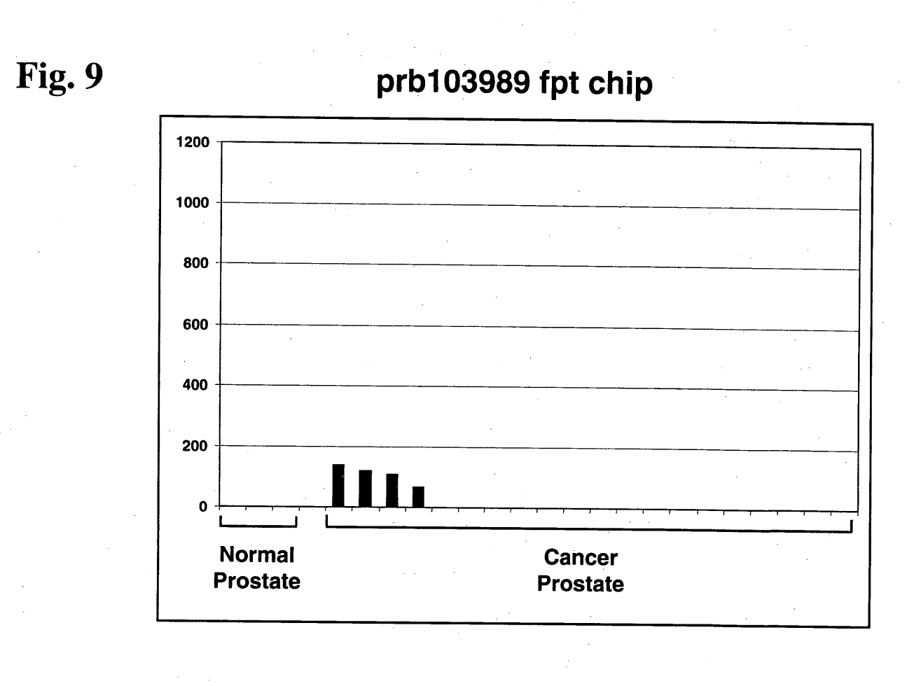

FIG. 9 shows the expression level of IgSF9 as detected by PRB103989 (black bars) and measured by microarray hybridization, using a Five Prime chip, of 20 prostate cancer samples, and three normal prostate samples. The results show that IgSF9 was expressed in four maligant prostate cancer samples and absent in all normal tissue samples.

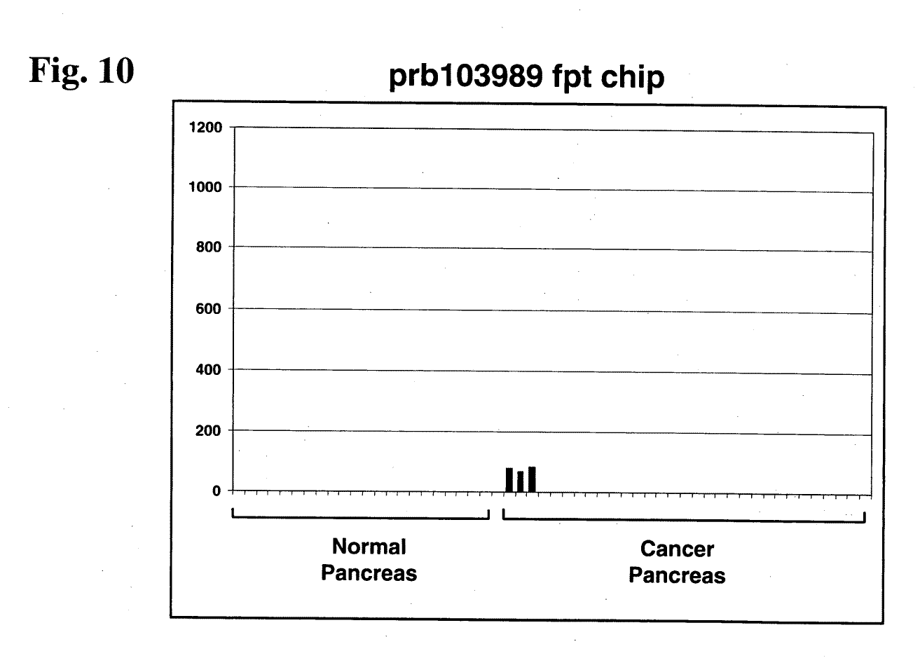

FIG. 10 shows the expression level of IgSF9 as detected by PRB103989 (black bars) and measured by microarray hybridization, using a Five Prime chip, of 31 cancerous pancreas samples, and 22 normal human pancreas specimens. The results show that IgSF9 was expressed in three cancer samples and was absent in all normal samples.

FIG. 11 shows the expression level of IgSF9 as detected by PRB103989 (black bars) and measured by microarray hybridization, using a Five Prime chip, on normal tissue specimens. FIG. 11a shows IgSF9 expression in normal adrenal, B-cell, bladder, bone marrow, CD4⁺ T-cell, CD8⁺ T-cell, duodenum, fallopian tube, gallbladder, heart, and jejunum. FIG. 11b shows IgSF9 expression in normal kidney, liver, lymph node, monocyte, myometrium, NK cell, omentum, ovary, parotid gland, pituitary, and placenta. FIG. 11c shows IgSF9 expression in normal skeletal muscle, skin, small intestine, soft tissues, spleen, stem cell, adipose tissue, testis, thymus, thyroid, uterus, and white blood cells (WBC).

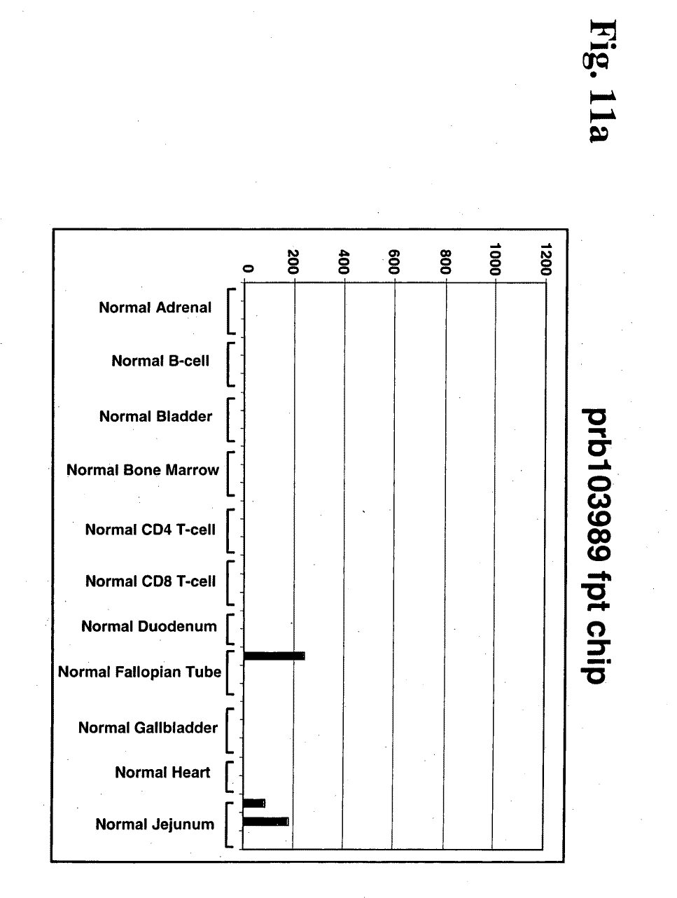

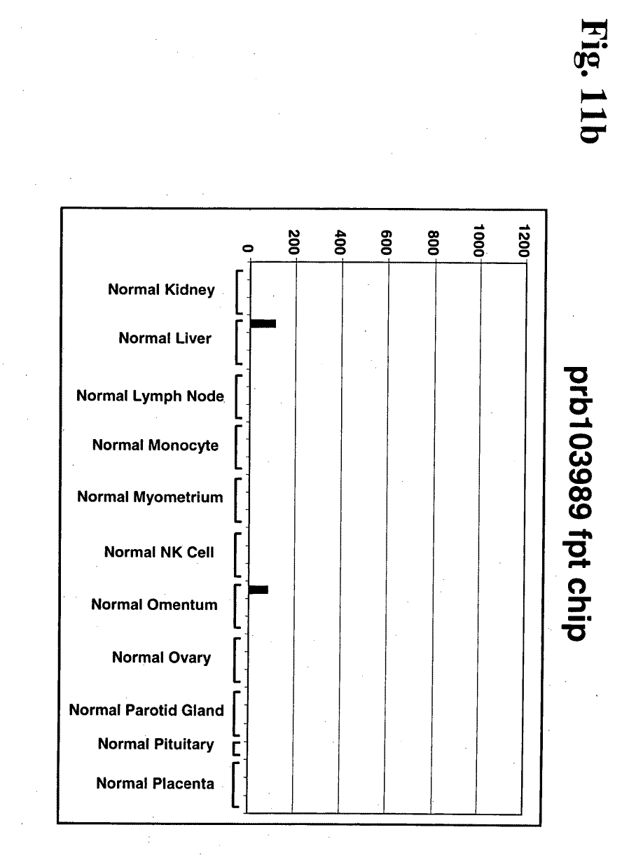

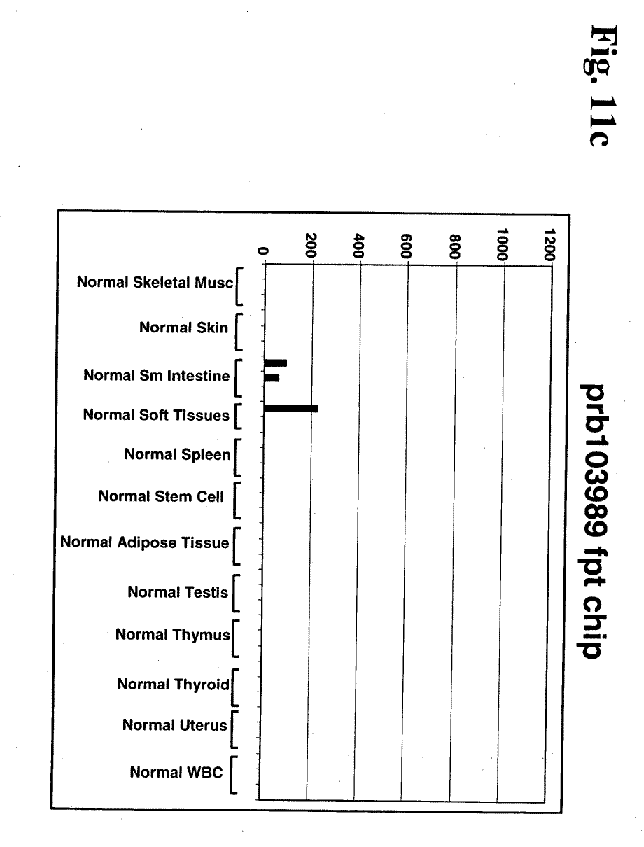

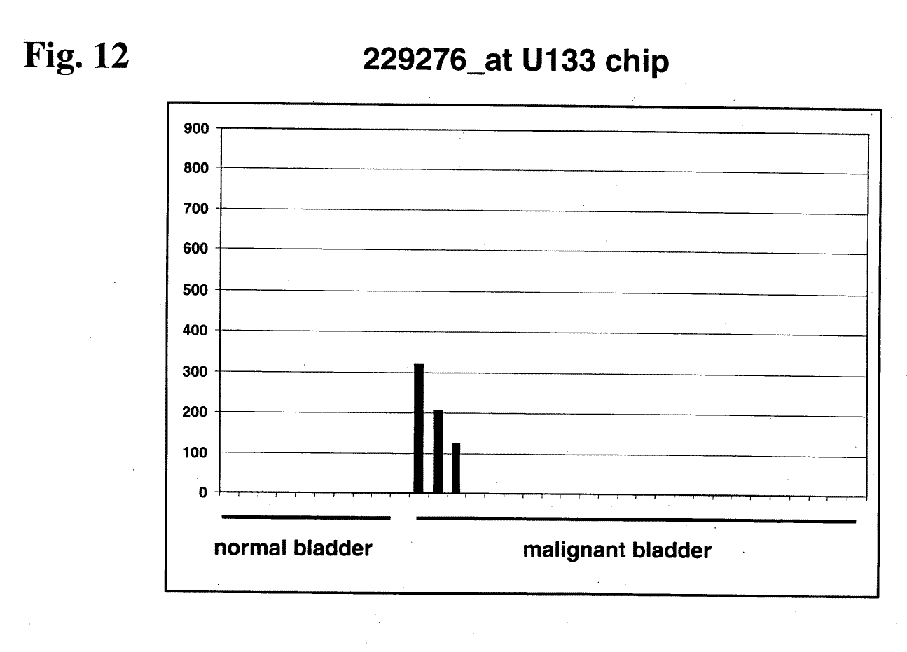

FIG. 12 shows the results of interrogating a proprietary oncology database from GeneLogic by probing an Affymetrix U133 chip with a probe 229276_at corresponding to IgSF9 in order to determine the expression of the sequences in normal and malignant bladder tissues. IgSF9 was expressed in three of the 23 malignant bladder tissues examined and none of the nine normal bladder tissues examined.

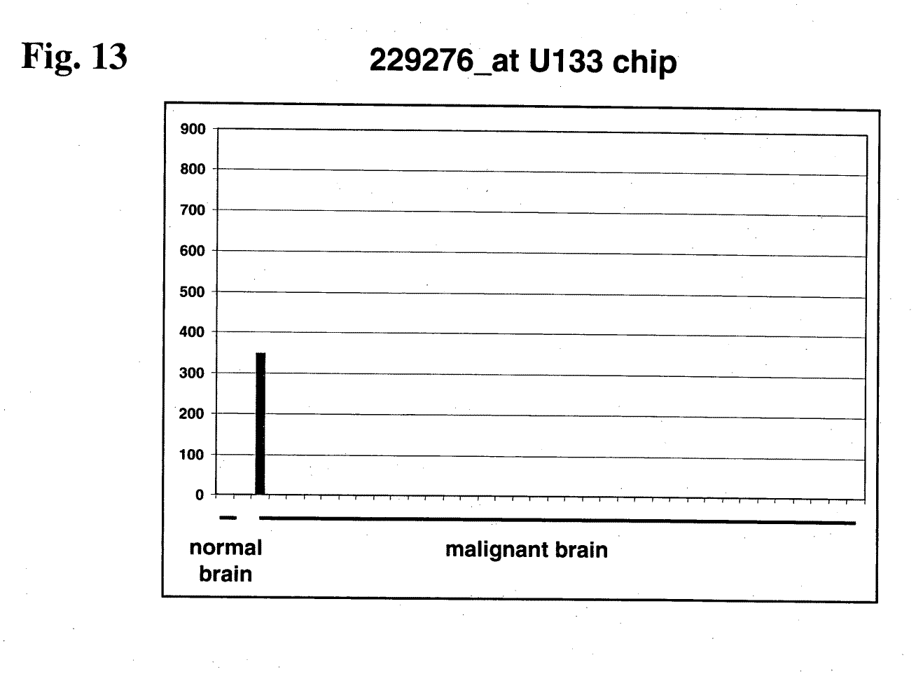

FIG. 13 shows the results of interrogating the GeneLogic database as described in FIG. 12. IgSF9 was expressed in one of the 35 malignant brain tissues examined and was absent in the normal brain specimen examined.

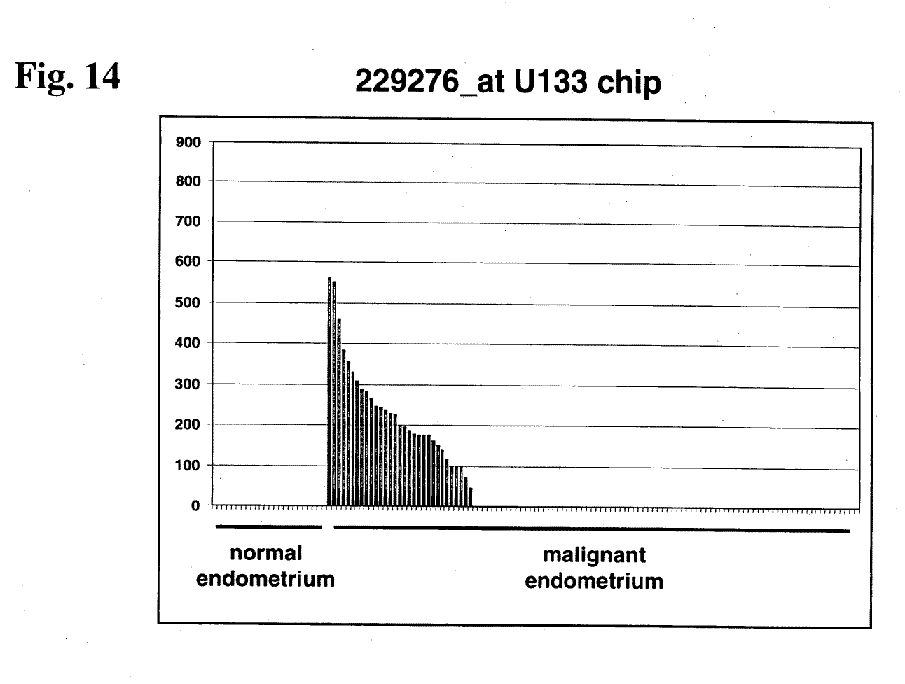

FIG. 14 shows the results of interrogating the GeneLogic database as described in FIG. 12. IgSF9 was expressed in 31 of the 112 malignant endometrium tissues examined and absent in the 23 normal endometrium tissues examined.

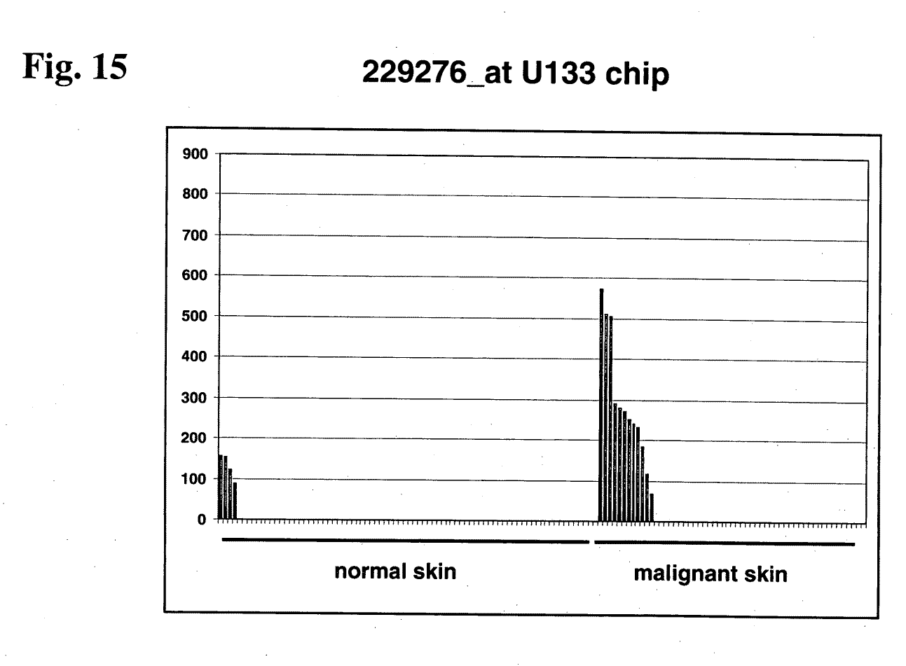

FIG. 15 shows the results of interrogating the GeneLogic database as described in FIG. 12. IgSF9 was expressed in four of the 79 normal and 12 of the 56 malignant skin tissues examined.

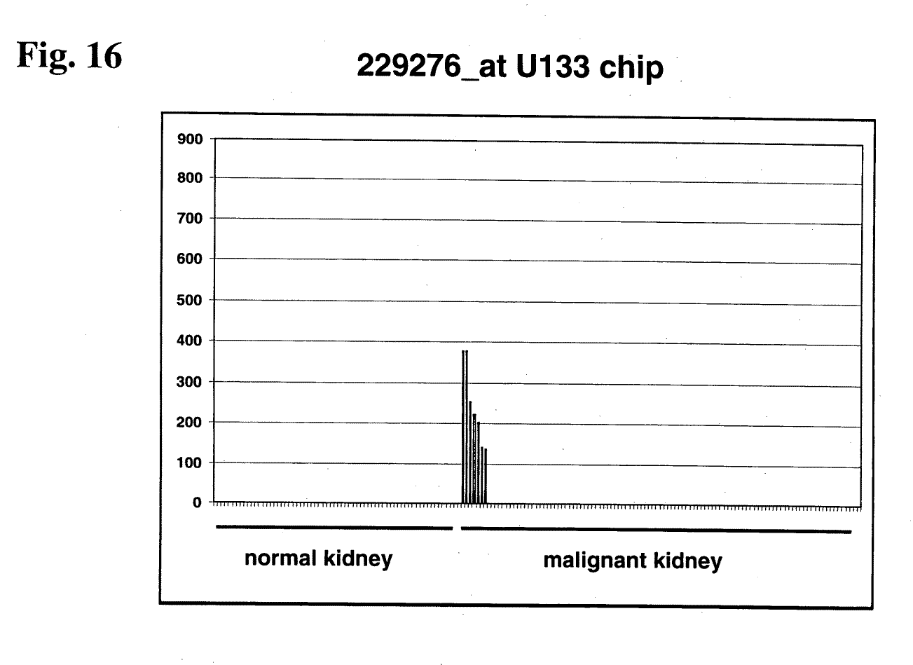

FIG. 16 shows the results of interrogating the GeneLogic database as described in FIG. 12. IgSF9 was expressed in seven of the 106 malignant kidney tissues examined and absent in all of the 65 normal kidney tissues examined.

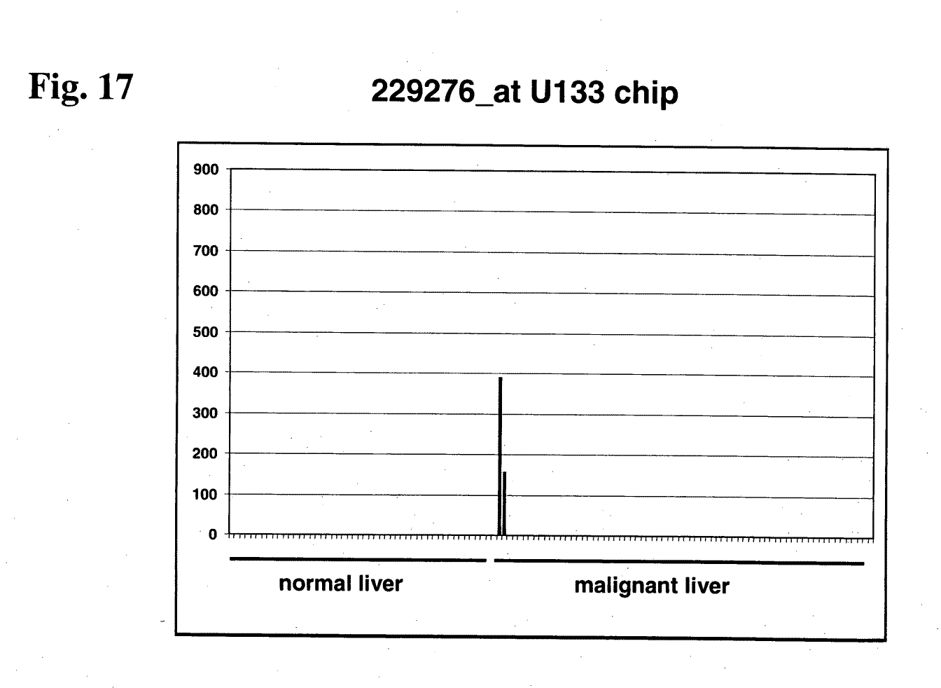

FIG. 17 shows the results of interrogating the GeneLogic database as described in FIG. 12. IgSF9 was expressed in two of the 70 malignant liver tissues examined and absent in all of the 49 normal liver tissues examined.

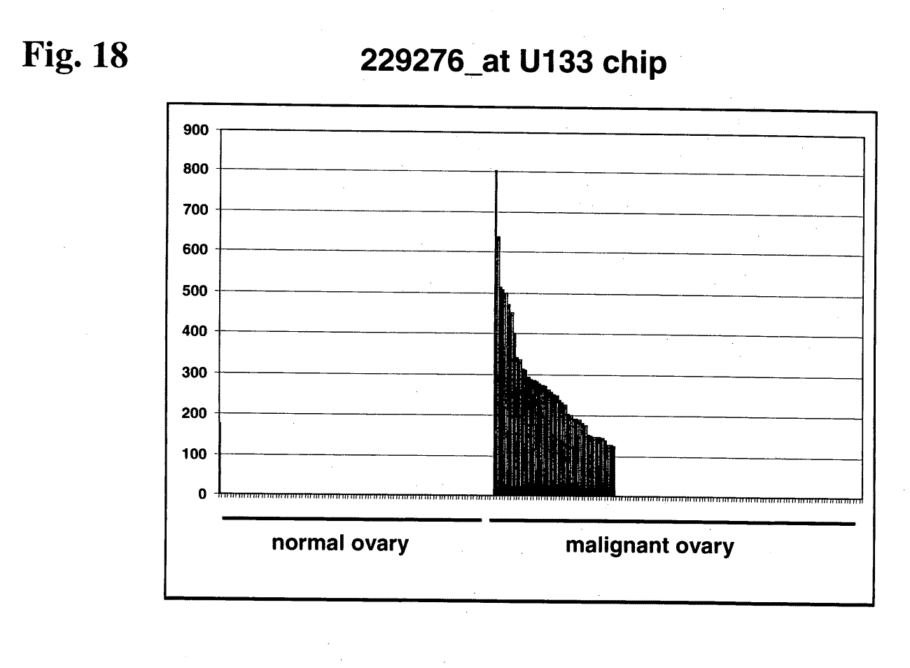

FIG. 18 shows the results of interrogating the GeneLogic database as described in FIG. 12. IgSF9 was expressed in 48 of the 128 malignant ovary tissues examined and in one of the 95 normal ovary tissues examined.

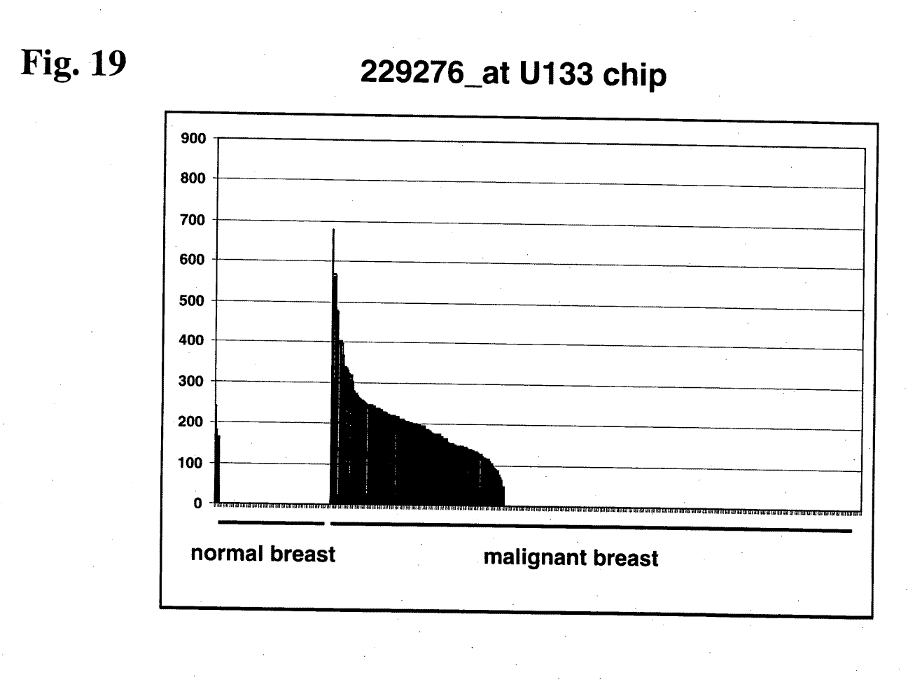

FIG. 19 shows the results of interrogating the GeneLogic database as described in FIG. 12. IgSF9 was expressed in 115 of the 348 malignant breast tissues examined and in three of the 75 normal breast tissues examined.

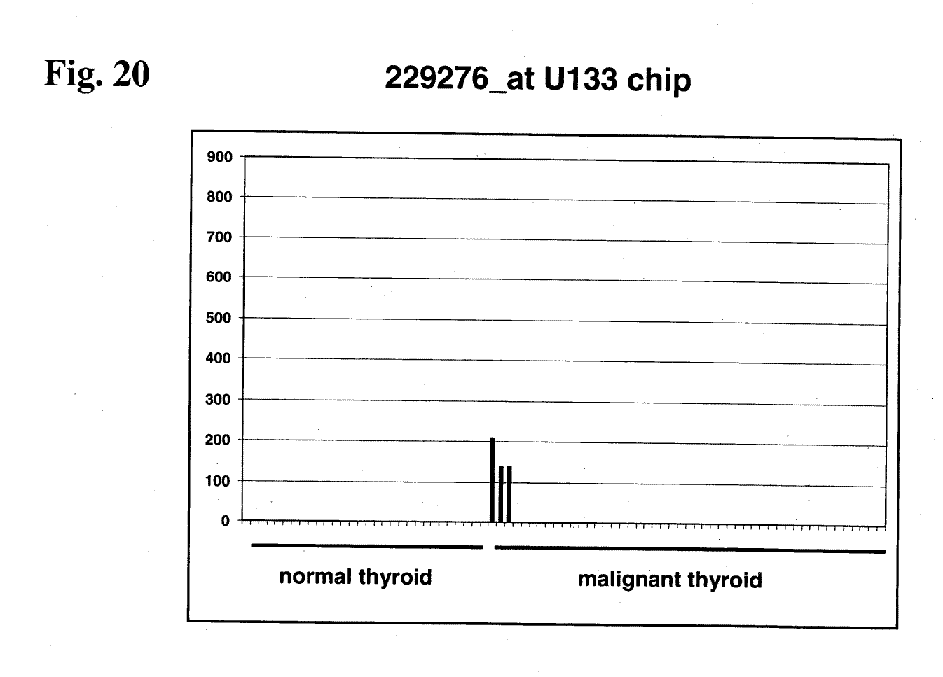

FIG. 20 shows the results of interrogating the GeneLogic database as described in FIG. 12. IgSF9 was expressed in three of the 49 malignant thyroid tissues examined and absent in all of the 29 normal thyroid tissues examined.

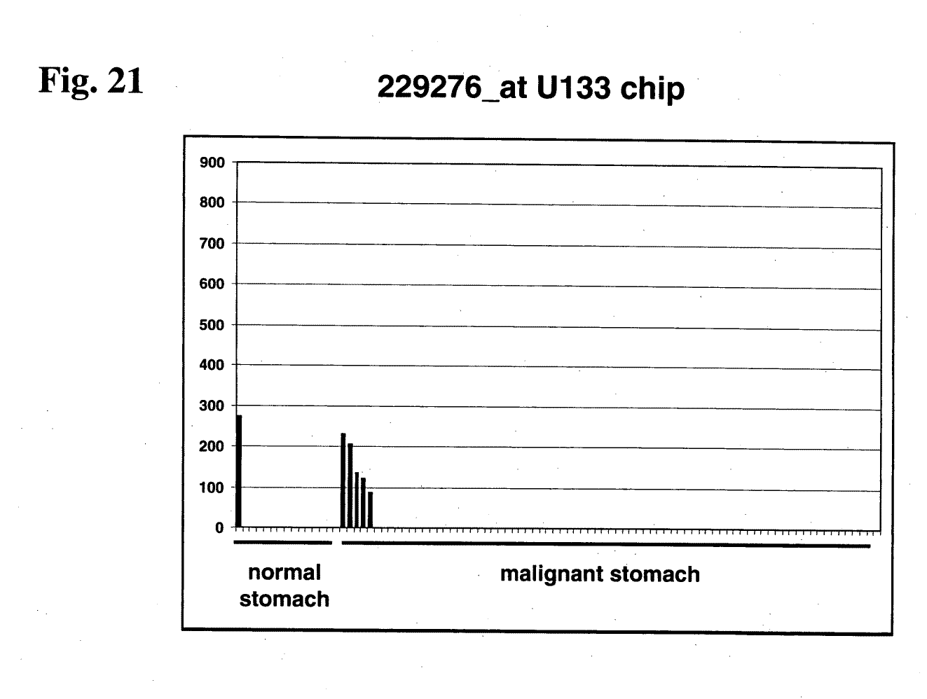

FIG. 21 shows the results of interrogating the GeneLogic database as described in FIG. 12. IgSF9 was expressed in five of the 78 malignant stomach tissues examined and in one of the 14 normal stomach tissues examined.

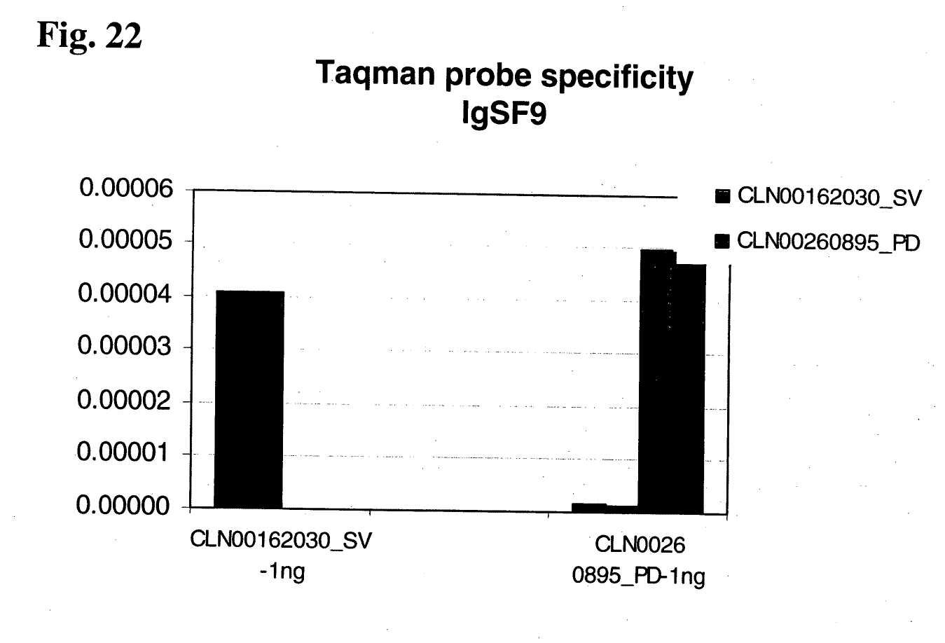

FIG. 22 shows the specificity of real-time polymerase chain reaction (RT-PCR) primers/probes for CLN00162030_SV and CLN00260895_PD. These primers/probes were designed for use in RT-PCR to specifically detect CLN00162030_SV and CLN00260895_PD, and were used in a Taqman primer test. The results show that primer/probe CLN00162030_SV detected clone CLN00162030_SV but not clone CLN00260895_PD, and primer/probe CLN00260895_PD detected clone CLN00260895_PD but not clone CLN00162030_SV.

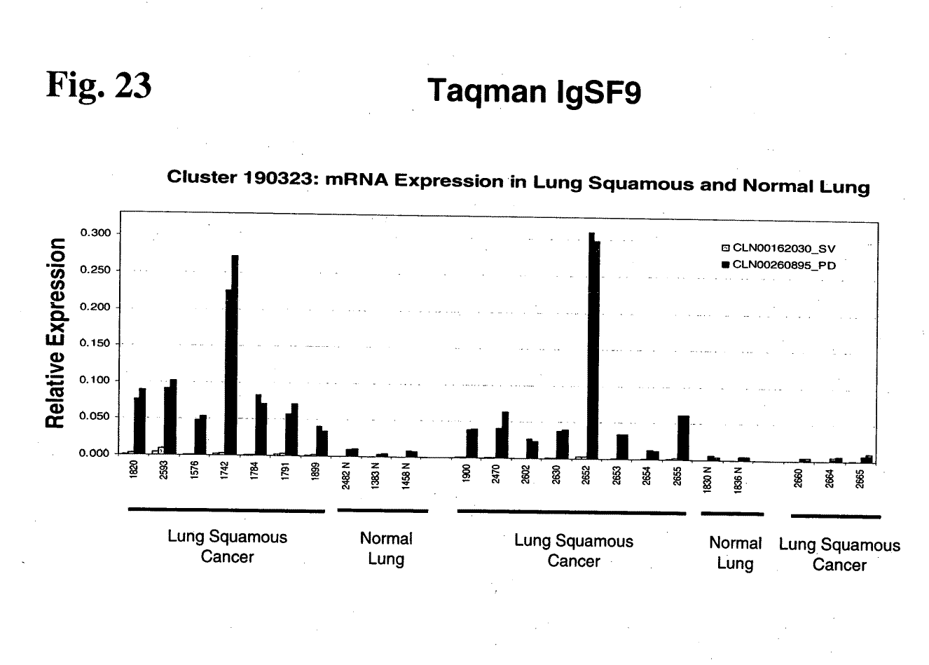

FIG. 23 shows the relative expression of CLN00162030_SV and CLN00260895_PD in lung squamous cell carcinoma and normal adjacent RNA specimens, as determined by Taqman RT-PCR. These results show CLN00260895_PD, but not CLN00162060_SV was overexpressed in the majority of lung squamous cell carcinoma tissues compared to normal lung tissues.

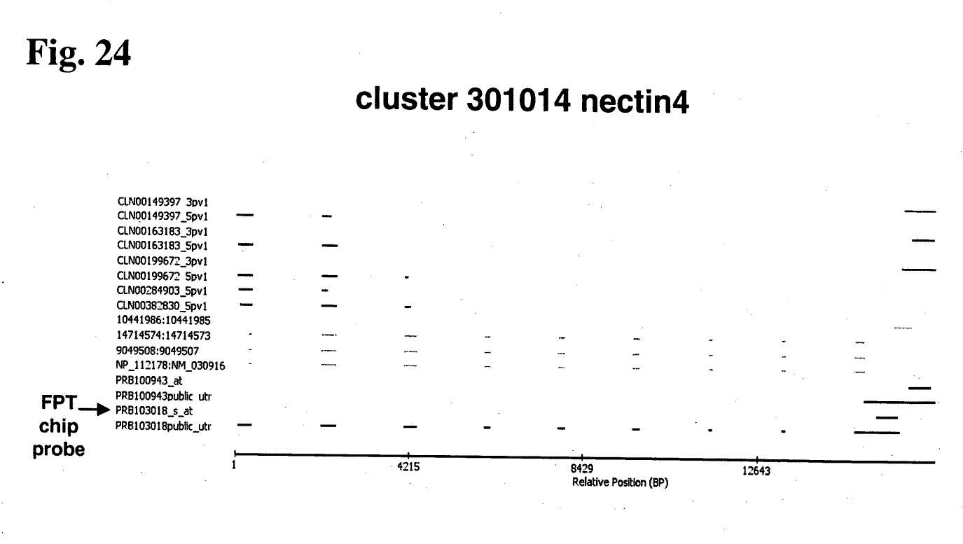

FIG. 24 shows an exon map of public and proprietary clones in cluster 301014, nectin 4; and the location of the microarray hybridization probe PRB103018_s_at. The horizontal axis is a scaled version of the genome which considers all introns to have equal lengths.

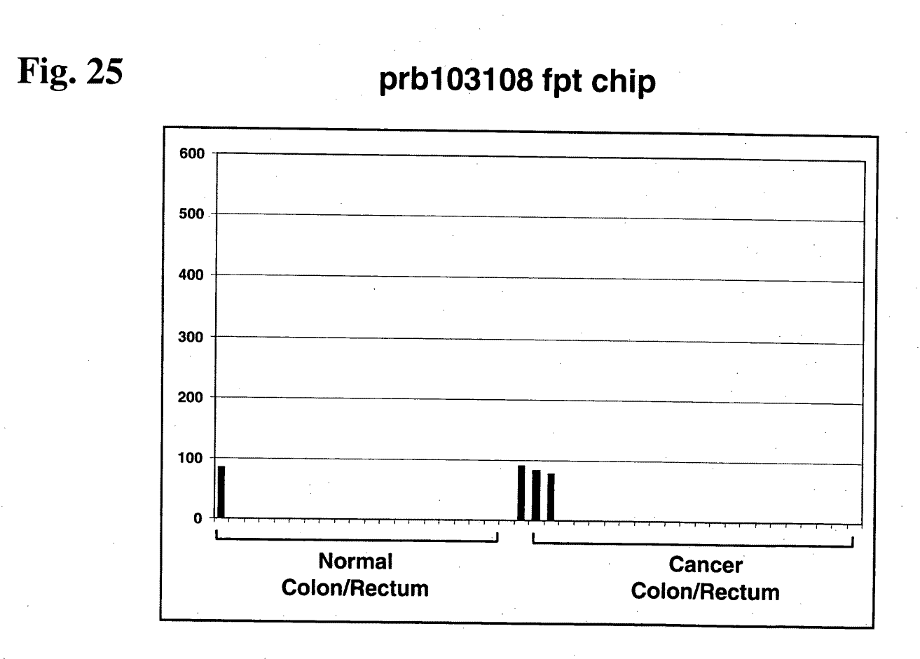

FIG. 25 shows the expression level of nectin 4 as detected by PRB103108 (black bars) and measured by microarray hybridization, using a Five Prime chip, of 23 colorectal adenocarcinoma samples, and 19 normal human colorectal specimens. The results show that nectin 4 was expressed in one normal sample and in three tumor samples.

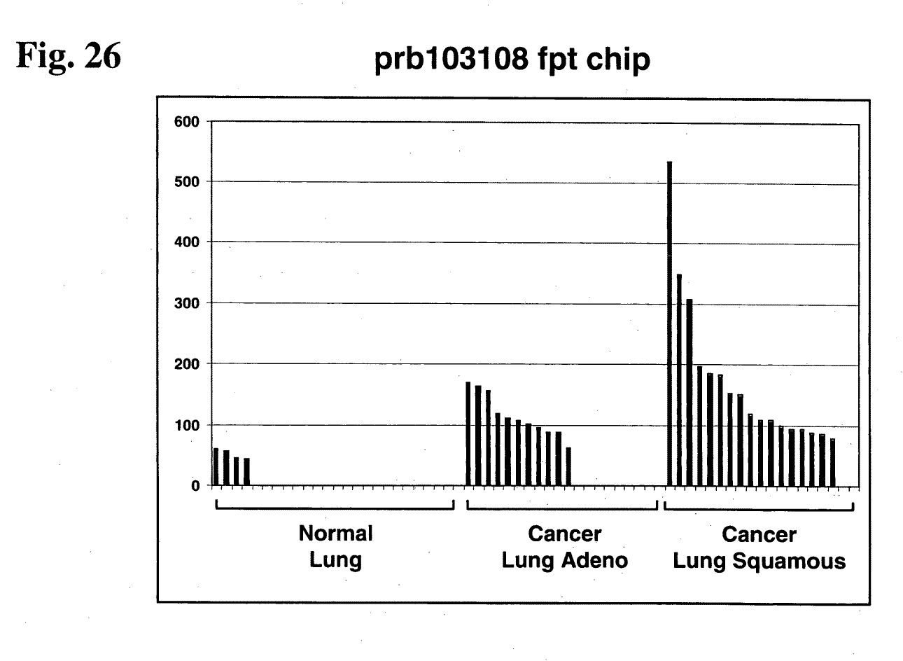

FIG. 26 shows the expression level of nectin 4 as detected by PRB103108 (black bars) and measured by microarray hybridization, using a Five Prime chip, of lung squamous cell cancer samples, lung adenocarcinoma samples, and normal lung samples. The results show that nectin 4 was expressed in 17 of 19 lung squamous carcinoma samples, 11 of 19 lung adenocarcinoma samples, and 4 of 24 normal lung samples. High-level expression (above levels in normal tissue) of nectin 4 was observed in about 17 of 19 lung squamous carcinoma samples and about 10 of 19 lung adenocarcinoma samples.

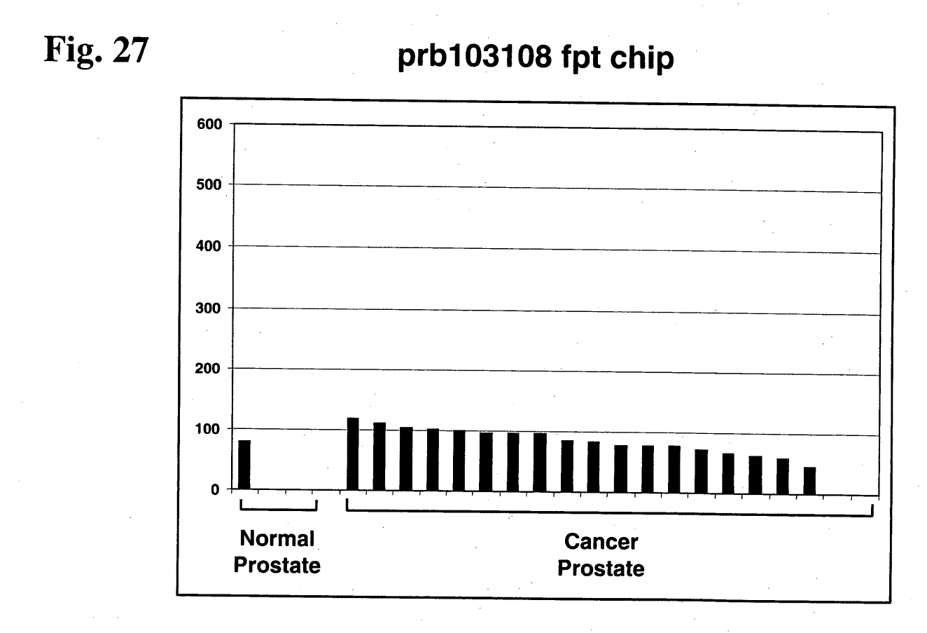

FIG. 27 shows the expression level of nectin 4 as detected by PRB103108 (black bars) and measured by microarray hybridization, using a Five Prime chip, of prostate cancer specimen and normal prostate specimen. The results show that nectin 4 was expressed in 18 of the 20 prostate cancer samples and in one of the three normal prostate samples.

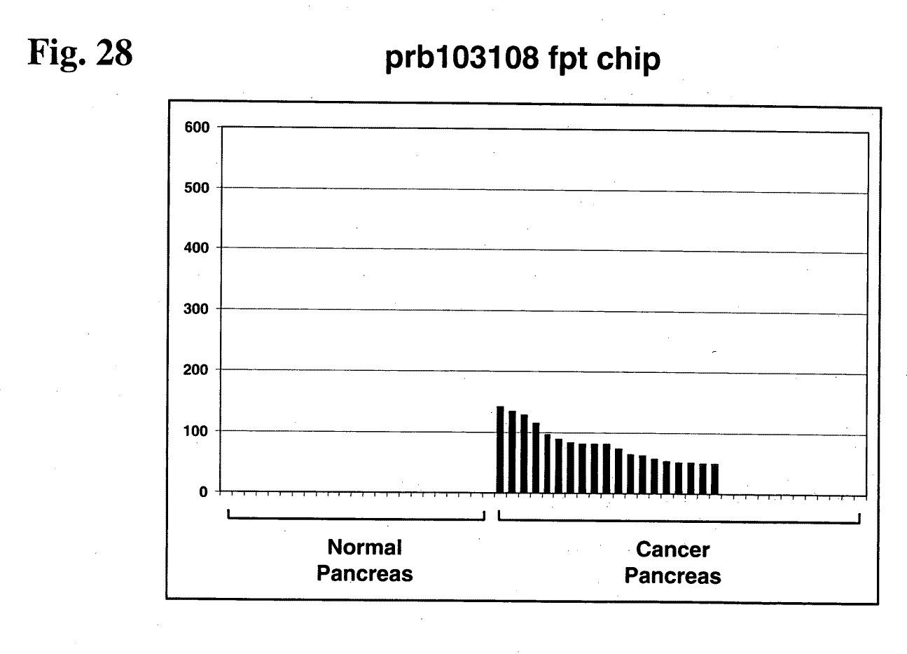

FIG. 28 shows the expression level of nectin 4 as detected by PRB103108 (black bars) and measured by microarray hybridization, using a Five Prime chip, of pancreatic cancer samples and normal pancreas samples. The results show that nectin 4 was expressed in 19 of the 31 pancreatic cancer samples and was absent in all of the 22 normal pancreas samples.

FIG. 29 shows the expression level of nectin 4 as detected by PRB103108 (black bars) and measured by microarray hybridization, using a Five Prime chip, of normal tissue specimens. FIG. 29a shows nectin 4 expression in normal adrenal, B-cell, bladder, bone marrow, CD4⁺ T-cell, CD8⁺ T-cell, duodenum, fallopian tube, gallbladder, heart, and jejunum. FIG. 29b shows nectin 4 expression in normal kidney, liver, lymph node, monocyte, myometrium, NK cell, omentum, ovary, parotid gland, pituitary, and placenta. FIG. 29c shows nectin 4 expression in normal skeletal muscle, skin, small intestine, soft tissues, spleen, stem cell, adipose tissue, testis, thymus, thyroid, uterus, and white blood cells (WBC).

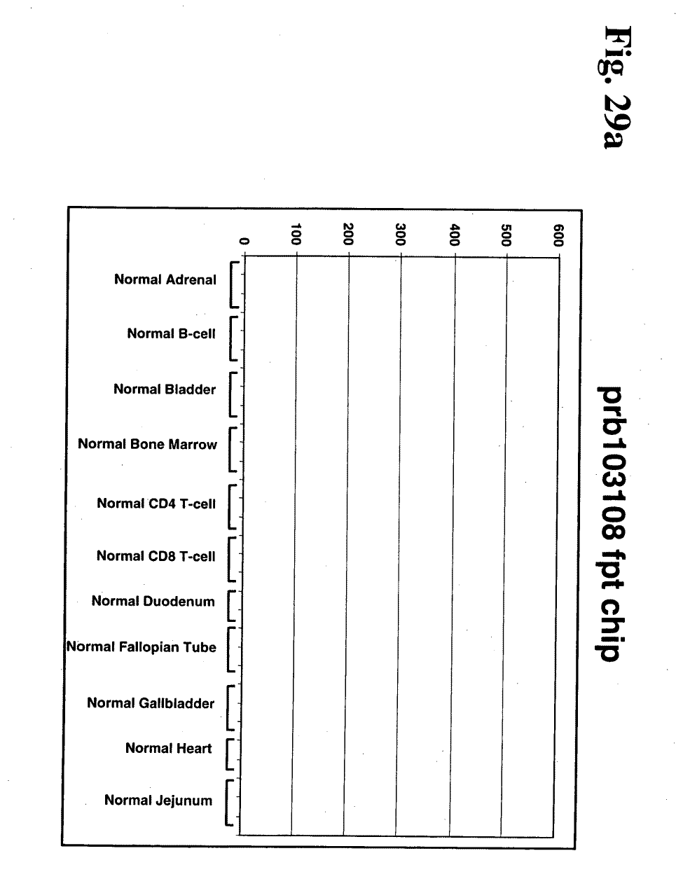

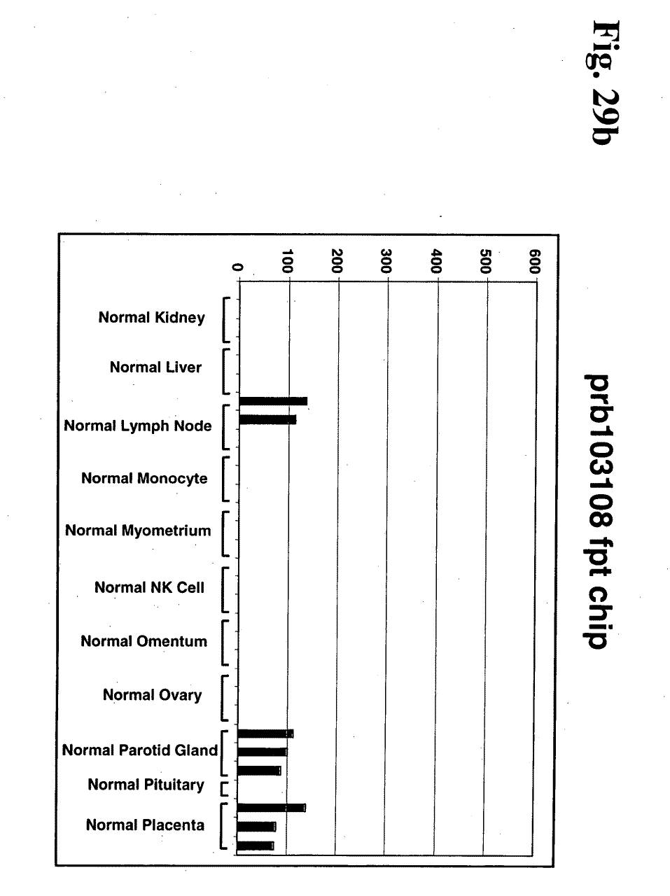

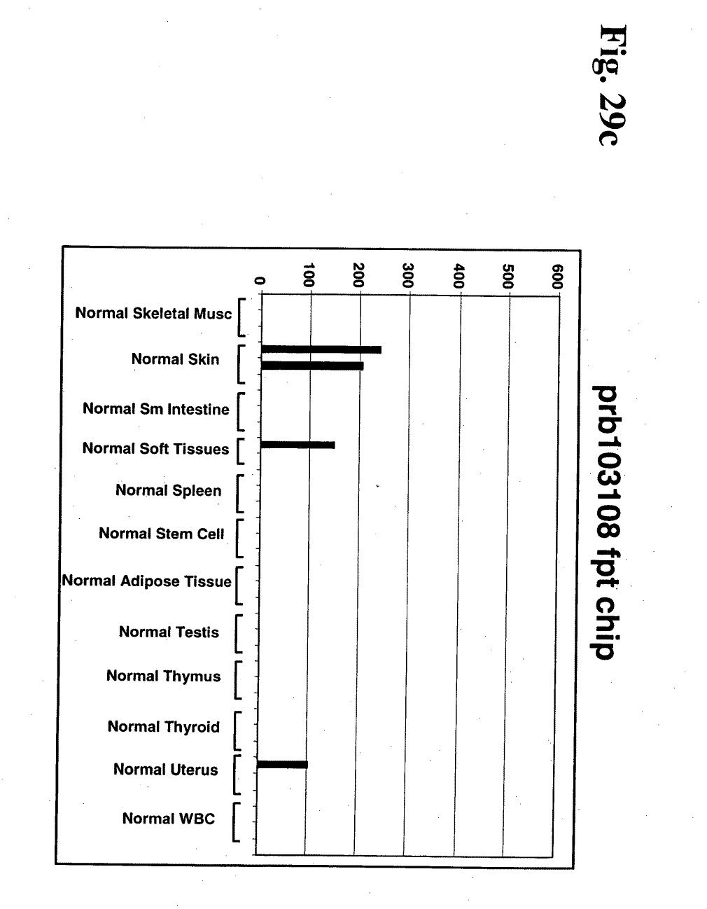

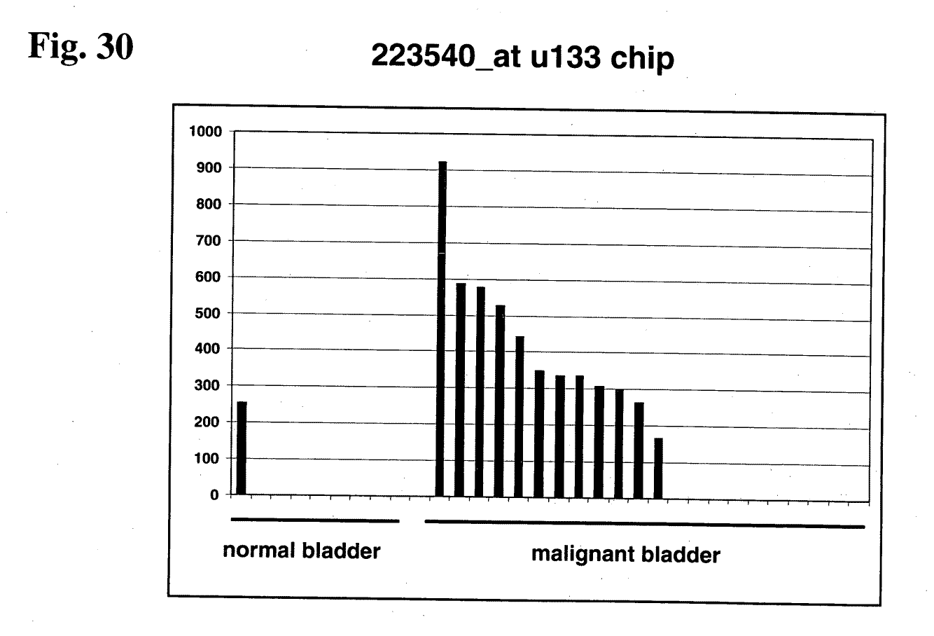

FIG. 30 shows the results of interrogating a proprietary oncology database from GeneLogic by probing an Affymetrix U133 chip with a probe 223540_at corresponding to nectin 4 in order to determine the expression of the sequences in normal and malignant bladder tissues. Nectin 4 was expressed in 12 of the 23 malignant bladder tissues examined and in one of the nine normal bladder tissues examined.

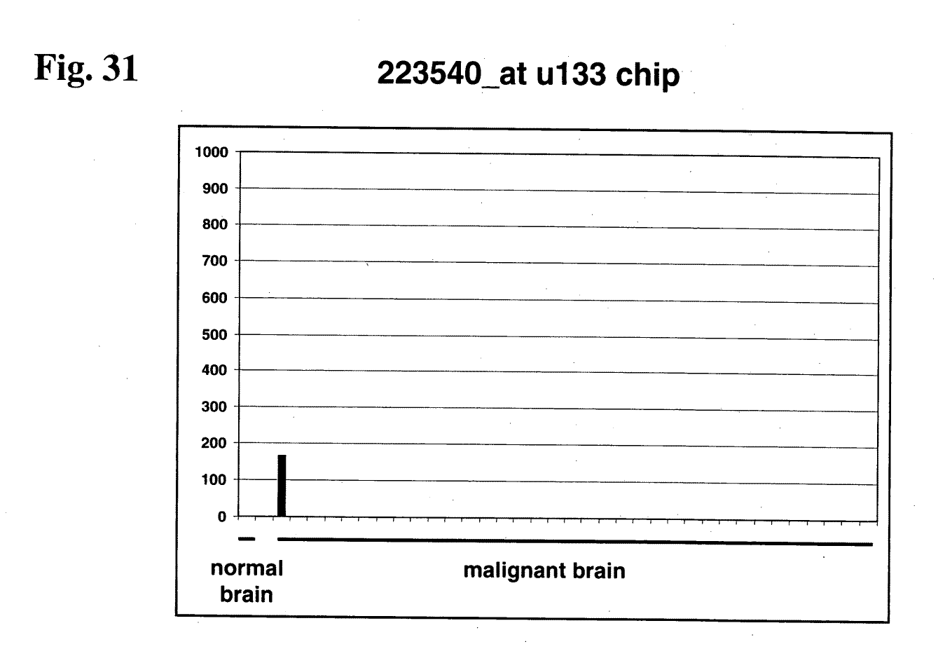

FIG. 31 shows the results of interrogating the GeneLogic database as described in FIG. 30. Nectin 4 was expressed in one of the 35 malignant brain tissues examined and was not expressed in the normal brain specimen examined.

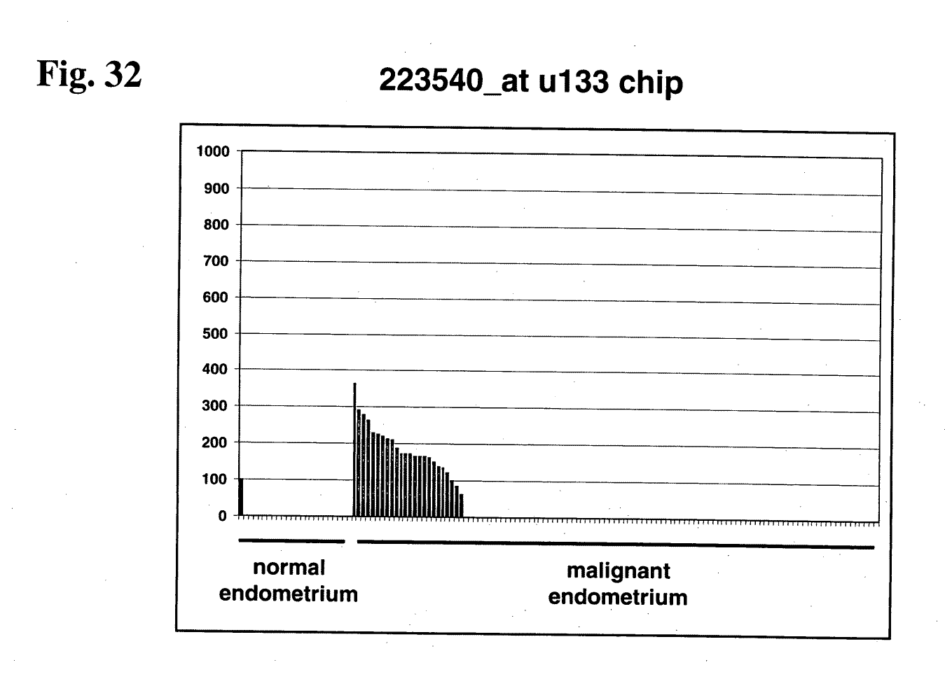

FIG. 32 shows the results of interrogating the GeneLogic database as described in FIG. 30. Nectin 4 was expressed in 24 of the 112 malignant endometrium tissues examined and in one of the 23 normal endometrium tissues examined.

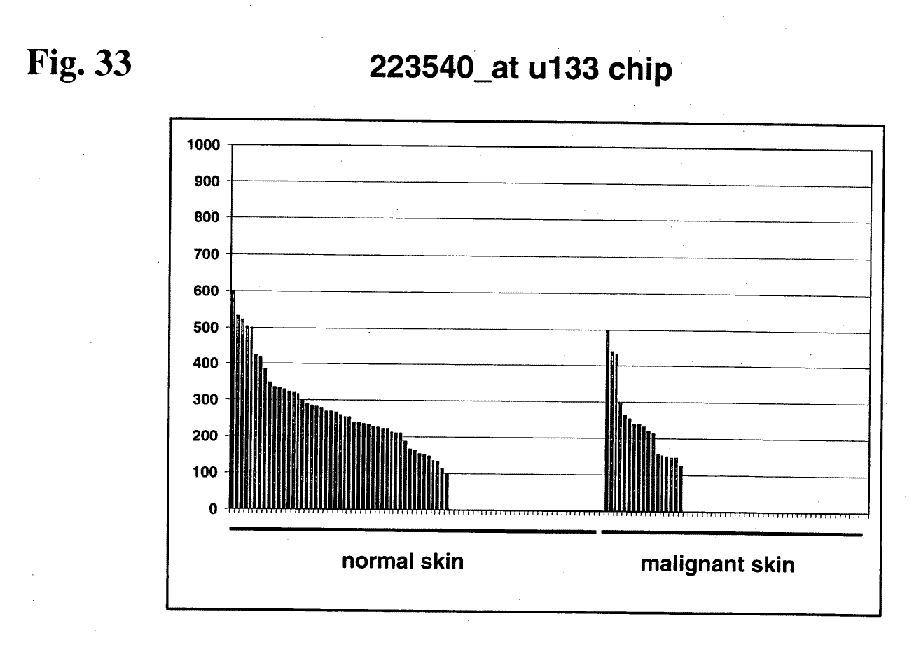

FIG. 33 shows the results of interrogating the GeneLogic database as described in FIG. 30. Nectin 4 was expressed in both normal and malignant skin tissue.

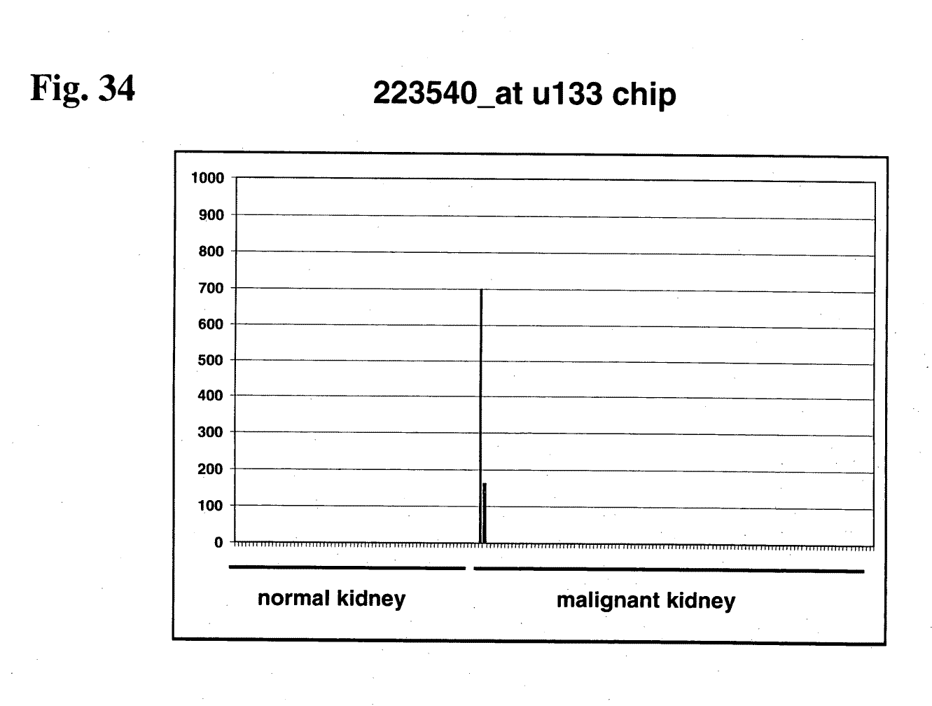

FIG. 34 shows the results of interrogating the GeneLogic database as described in FIG. 30. Nectin 4 was expressed in two of the 106 malignant kidney tissues examined and none of the 65 normal kidney tissues examined.

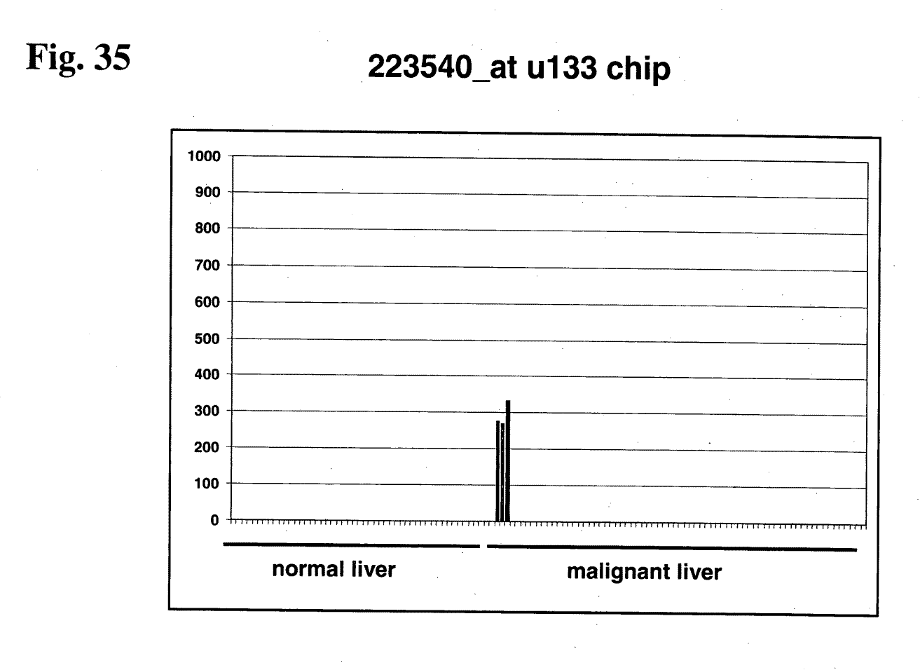

FIG. 35 shows the results of interrogating the GeneLogic database as described in FIG. 30. Nectin 4 was expressed in three of the 70 malignant liver tissues examined and none of the 49 normal liver tissues examined.

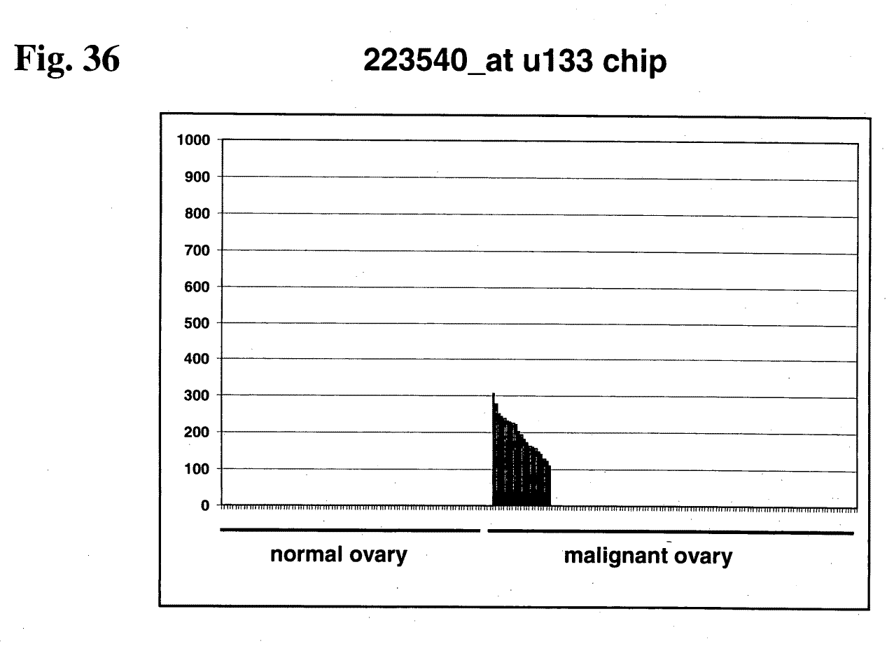

FIG. 36 shows the results of interrogating the GeneLogic database as described in FIG. 30. Nectin 4 was expressed in 21 of the 128 malignant ovary tissues examined and none of the 95 normal ovary tissues examined.

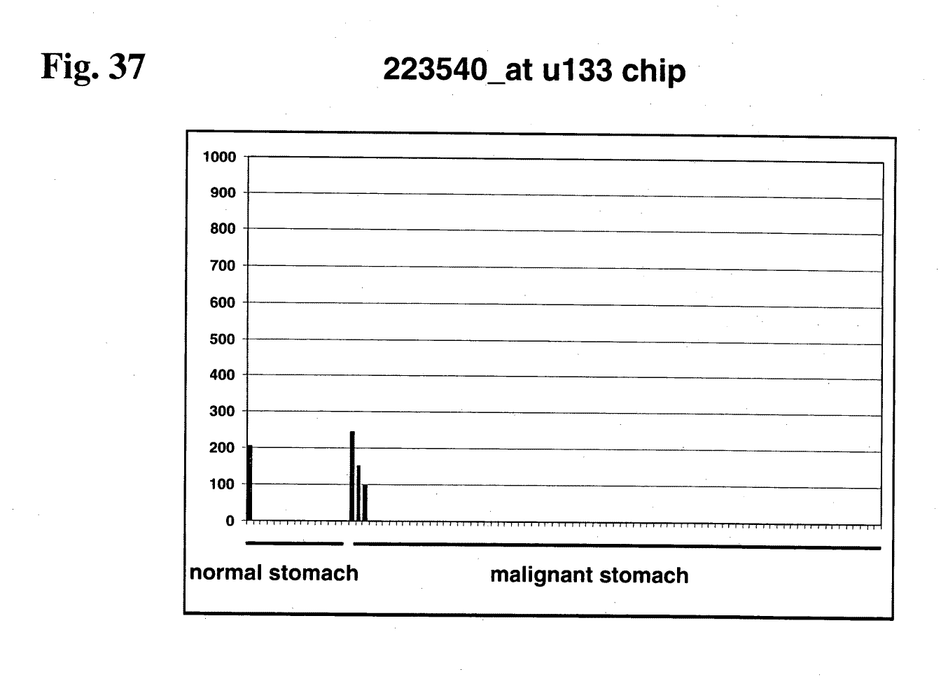

FIG. 37 shows the results of interrogating the GeneLogic database as described in FIG. 30. Nectin 4 was expressed in three of the malignant stomach samples examined and in one of the normal stomach samples examined.

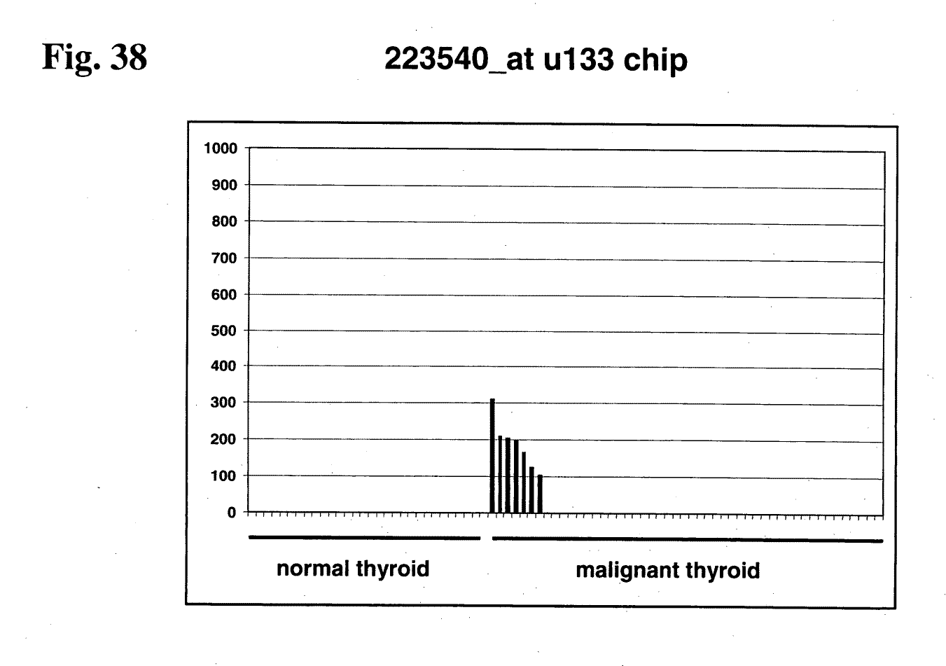

FIG. 38 shows the results of interrogating the GeneLogic database as described in FIG. 30. Nectin 4 was expressed in seven of the malignant thyroid tissues examined and in none of the normal thyroid tissues examined.

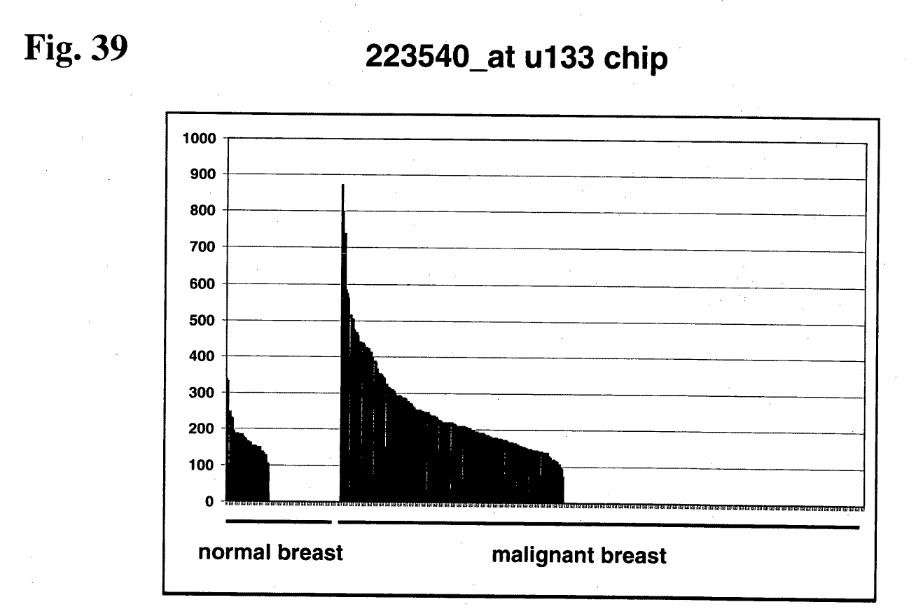

FIG. 39 shows the results of interrogating the GeneLogic database as described in FIG. 30. Nectin 4 was expressed in 150 of the 348 malignant breast tissues examined and in 29 of the 75 normal breast tissues examined.

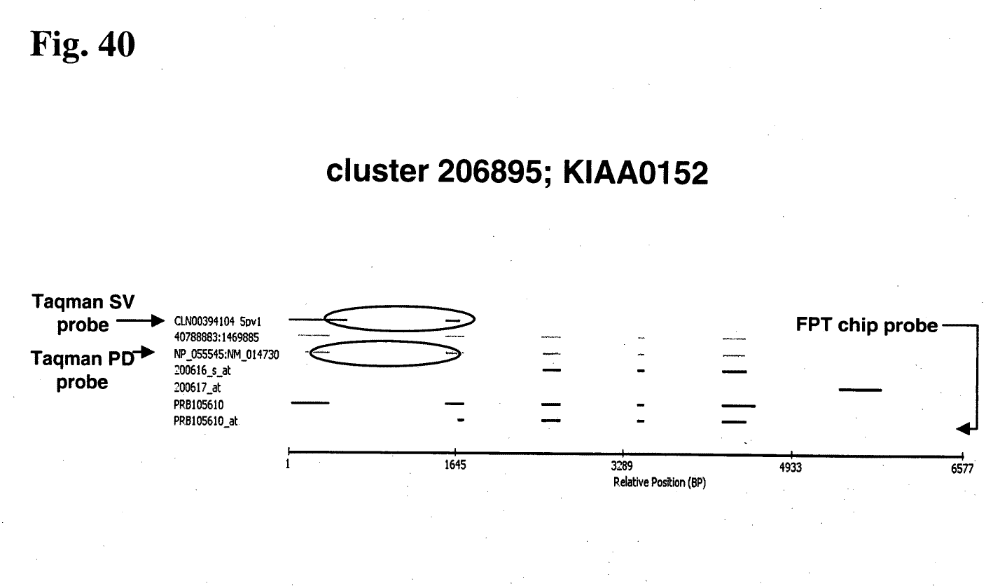

FIG. 40 shows an exon map of public and proprietary clones in cluster 206895, KIAA0152; the microarray hybridization probe location of PRB105610_s_at; and the relative location of the RT-PCR primers and probes for probe CLN00009706_PD and probe CLN00394104_SV. The horizontal axis is a scaled version of the genome which considers all introns to have equal lengths.

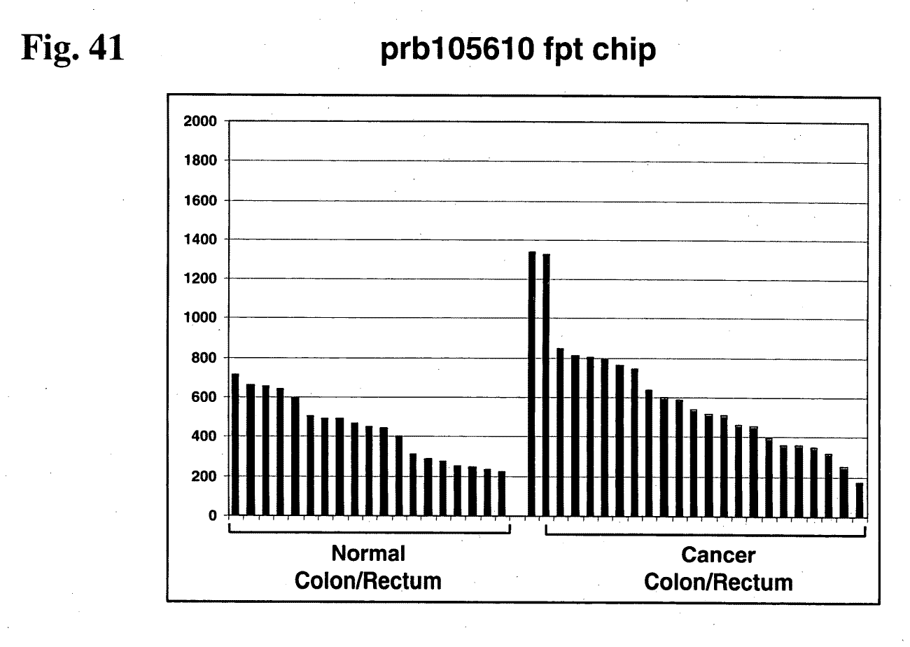

FIG. 41 shows the expression level of KIAA0152 as detected by PRB105610 (black bars) and measured by microarray hybridization, using a Five Prime chip, of colorectal adenocarcinoma samples, and normal human colorectal specimens. The results show that KIAA0152 was expressed in all 19 normal samples and in all 23 tumor samples.

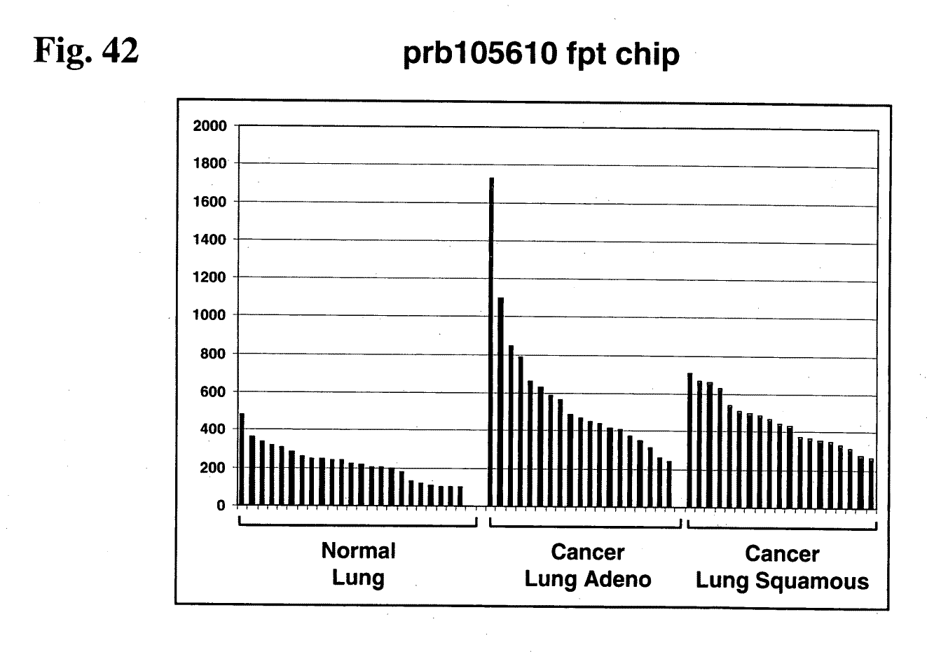

FIG. 42 shows the expression level of KIAA0152 as detected by PRB105610 (black bars) and measured by microarray hybridization, using a Five Prime chip, of lung squamous cell cancer samples, human lung adenocarcinoma samples, and normal lung samples The results show that KIAA0152 was expressed in all 19 lung squamous carcinoma samples, in all 19 lung adenocarcinoma samples, and in 23 of 24 normal lung samples.

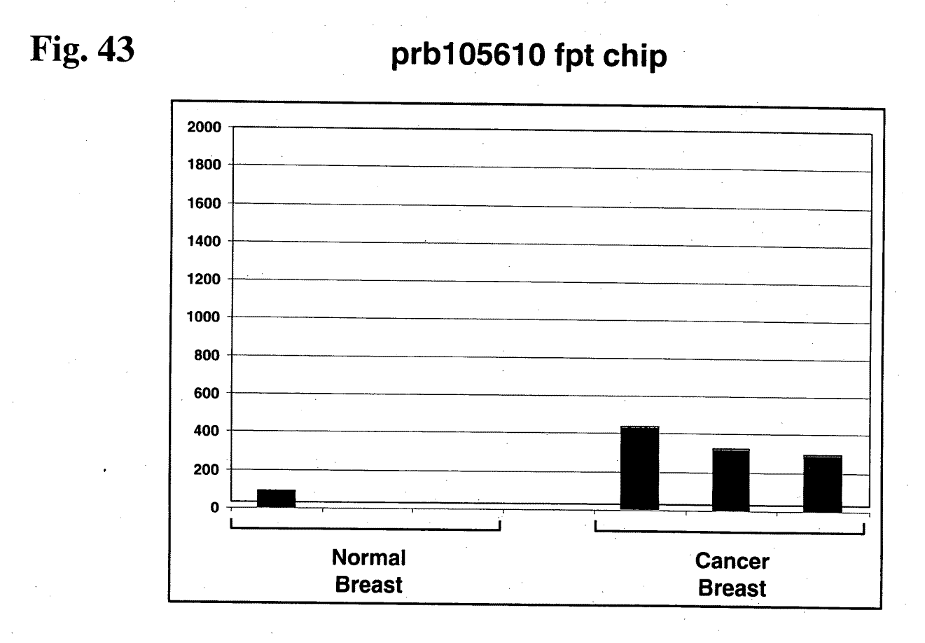

FIG. 43 shows the expression level of KIAA0152 as detected by PRB105610 (black bars) and measured by microarray hybridization, using a Five Prime chip, of breast cancer specimen and normal breast specimen. The results show that KIAA0152 was expressed in three of the three breast cancer samples and in one of the three normal breast samples.

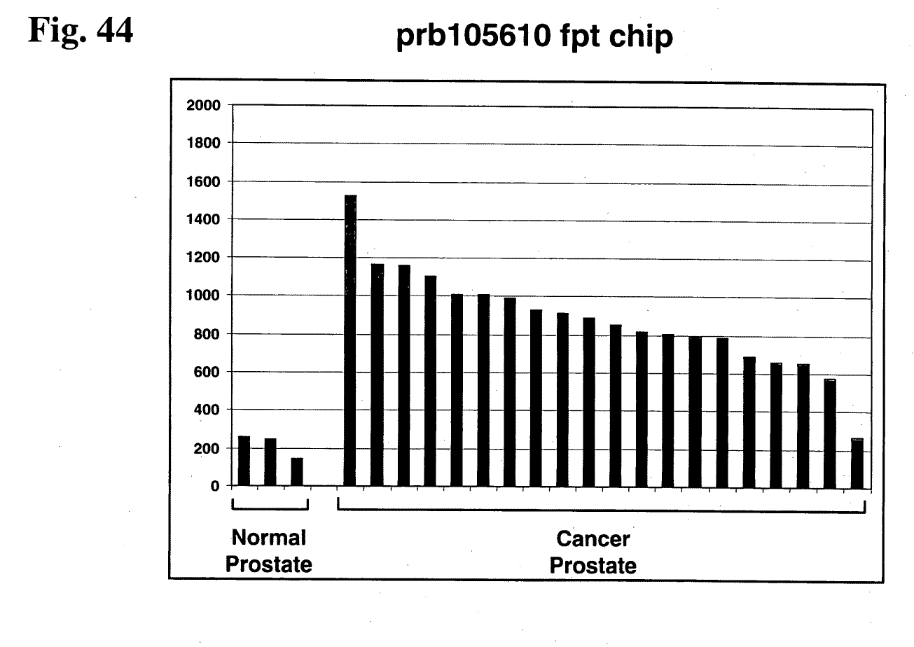

FIG. 44 shows the expression level of KIAA0152 as detected by PRB105610 (black bars) and measured by microarray hybridization, using a Five Prime chip, of prostate cancer samples and normal prostate samples. The results show that KIAA0152 was expressed in all 20 prostate cancer samples and in all three normal prostate samples.

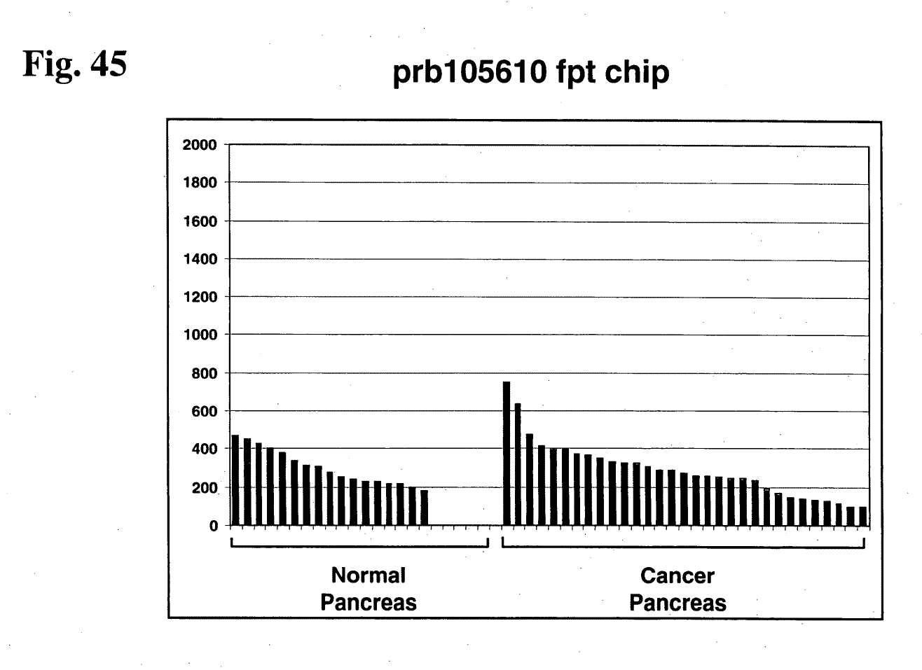

FIG. 45 shows the expression level of KIAA0152 as detected by PRB105610 (black bars) and measured by microarray hybridization, using a Five Prime chip, of pancreatic cancer samples and normal pancreas samples. The results show that KIAA0152 was expressed in all 31 pancreatic cancer samples and in 17 of the 22 normal pancreas samples.

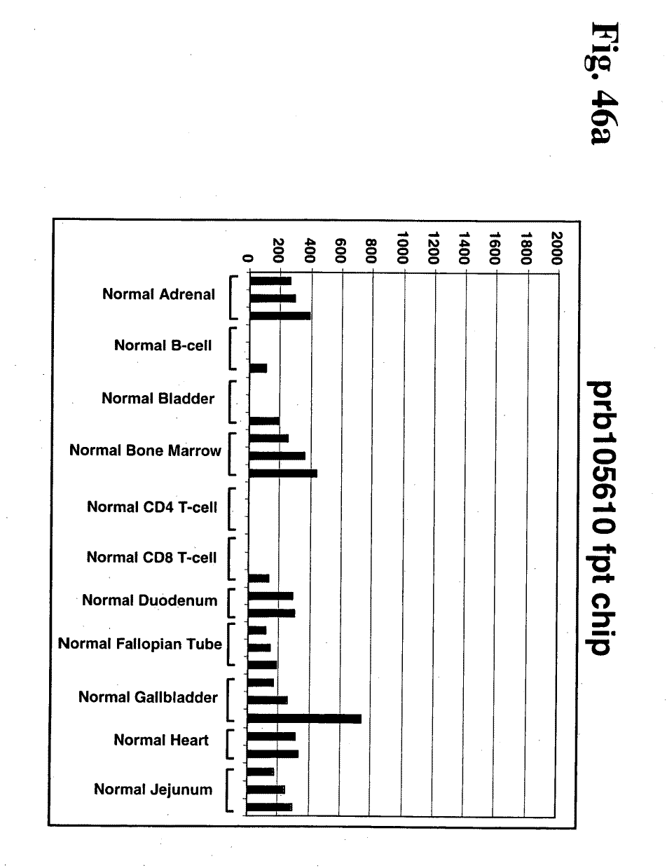

FIG. 46 shows the expression level of KIAA0152 as detected by PRB105610 (black bars) and measured by microarray hybridization, using a Five Prime chip, of normal tissue specimens. FIG. 46a shows KIAA0152 expression in normal adrenal, B-cell, bladder, bone marrow, CD4⁺ T-cell, CD8⁺ T-cell, duodenum, fallopian tube, gallbladder, heart, and jejunum. FIG. 46b shows KIAA0152 expression in normal kidney, liver, lymph node, monocyte, myometrium, NK cell, omentum, ovary, parotid gland, pituitary, and placenta. FIG. 46c shows KIAA0152 expression in normal skeletal muscle, skin, small intestine, soft tissues, spleen, stem cell, adipose tissue, testis, thymus, thyroid, uterus, and white blood cells (WBC).

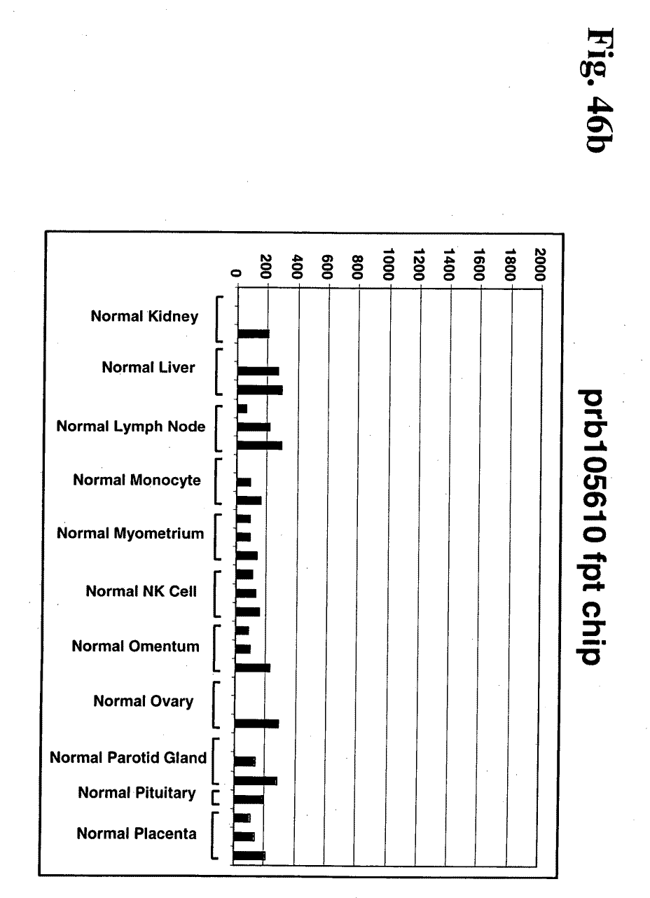

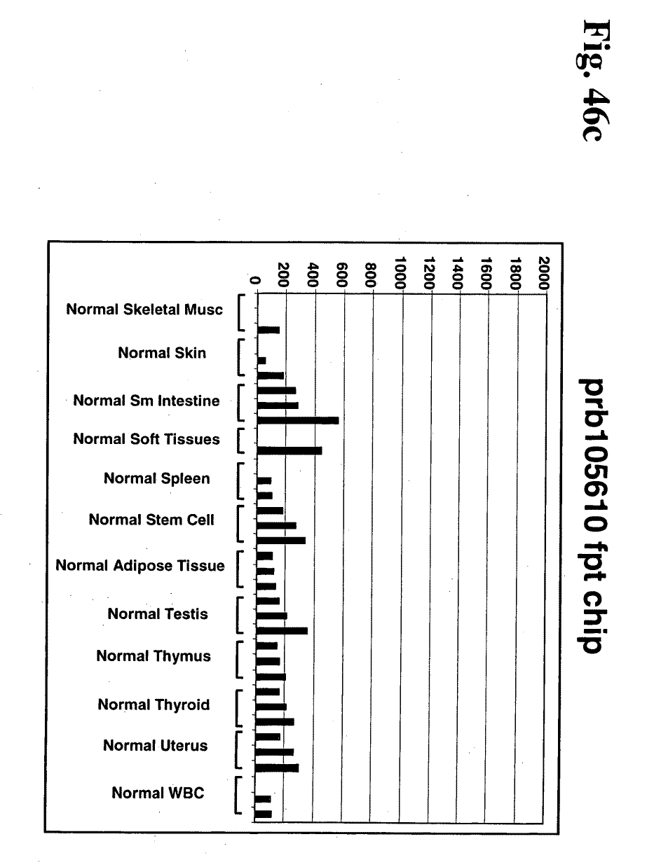

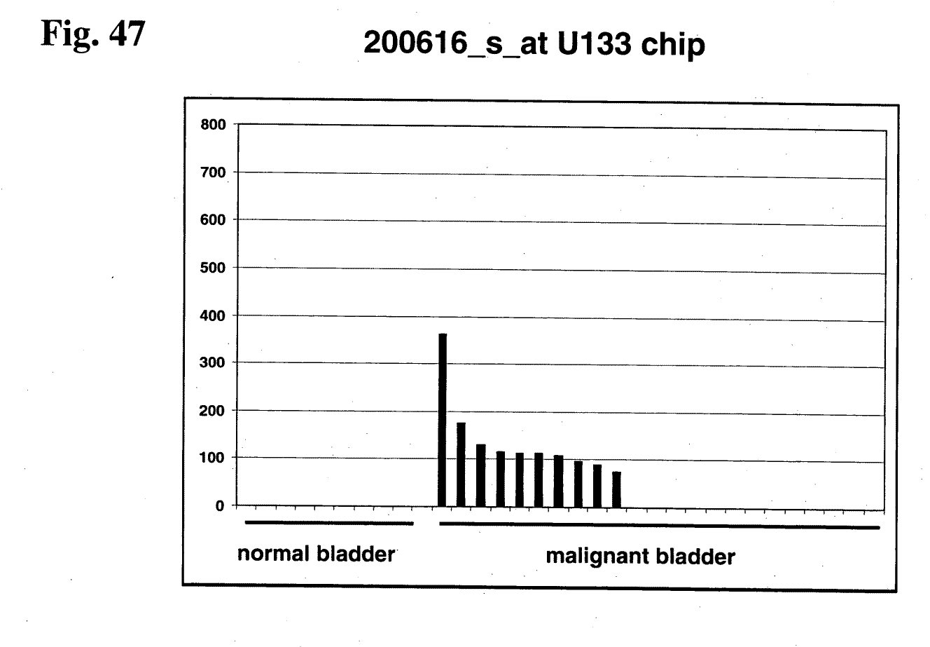

FIG. 47 shows the results of interrogating a proprietary oncology database from GeneLogic by probing an Affymetrix U133 chip with a probe 200616_s_at corresponding to KIAA0152 in order to determine the expression of the sequences in normal and malignant bladder tissues. KIAA0152 was expressed in 10 of the 23 malignant bladder tissues examined and none of the 9 normal bladder tissues examined.

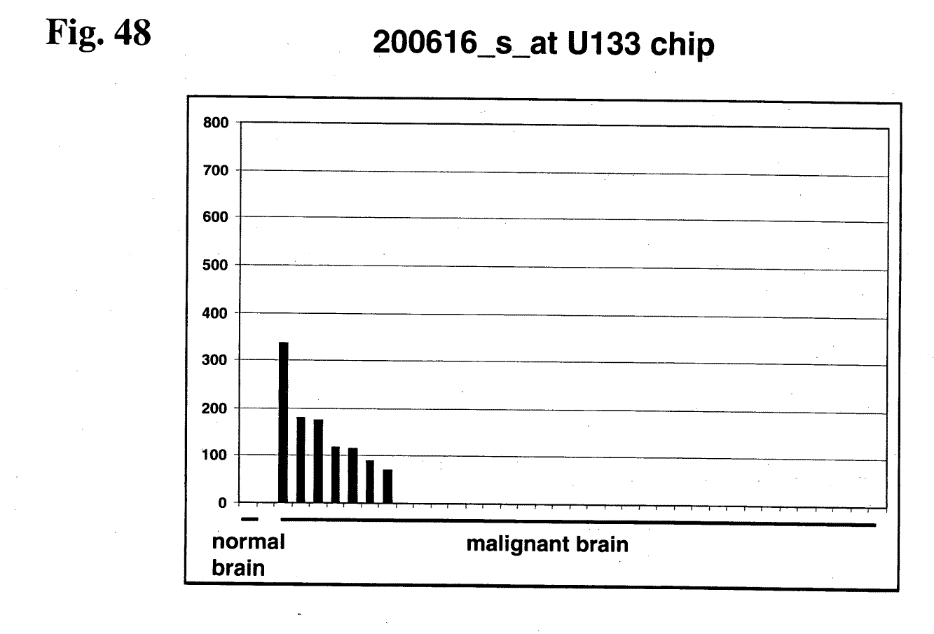

FIG. 48 shows the results of interrogating the GeneLogic database as described in FIG. 47. KIAA0152 was expressed in seven of the 35 malignant brain tissues examined and was not expressed in the normal brain specimen examined.

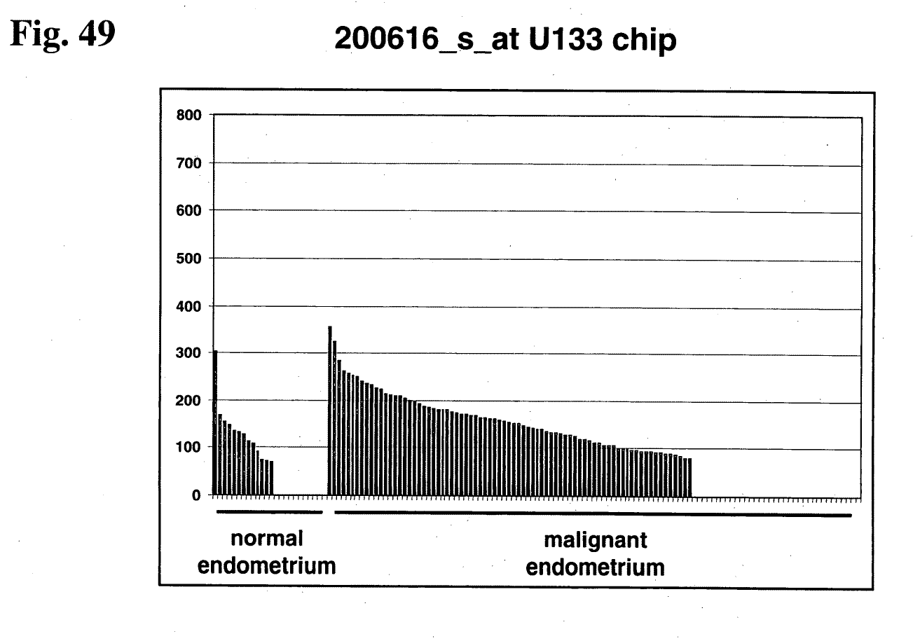

FIG. 49 shows the results of interrogating the GeneLogic database as described in FIG. 47. KIAA0152 was expressed in 77 of the 112 malignant endometrium tissues examined and in 13 of the 23 normal endometrium tissues examined.

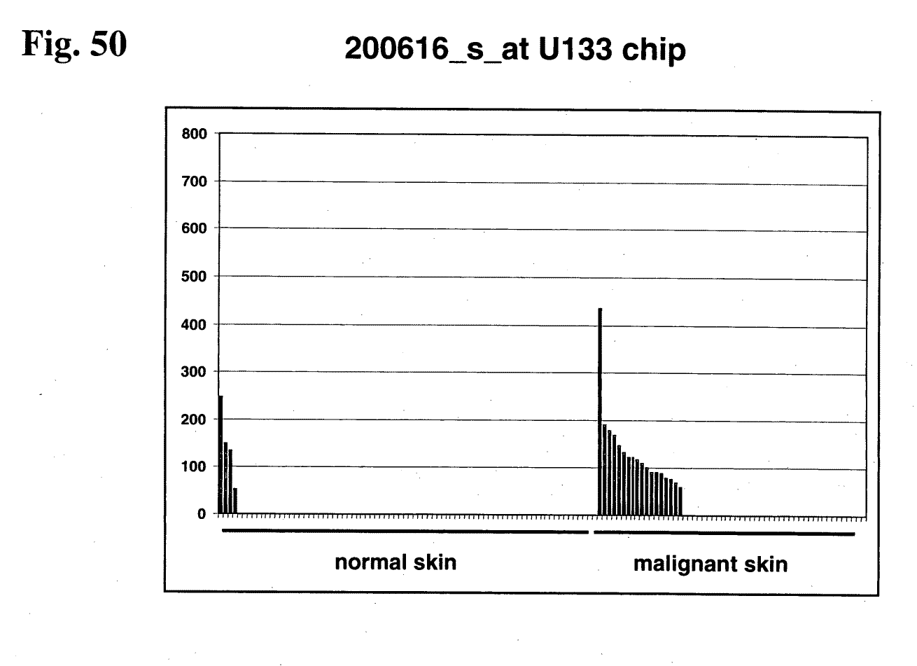

FIG. 50 shows the results of interrogating the GeneLogic database as described in FIG. 47. KIAA0152 was expressed in 18 of the malignant skin tissues examined and in four of the normal skin tissues examined.

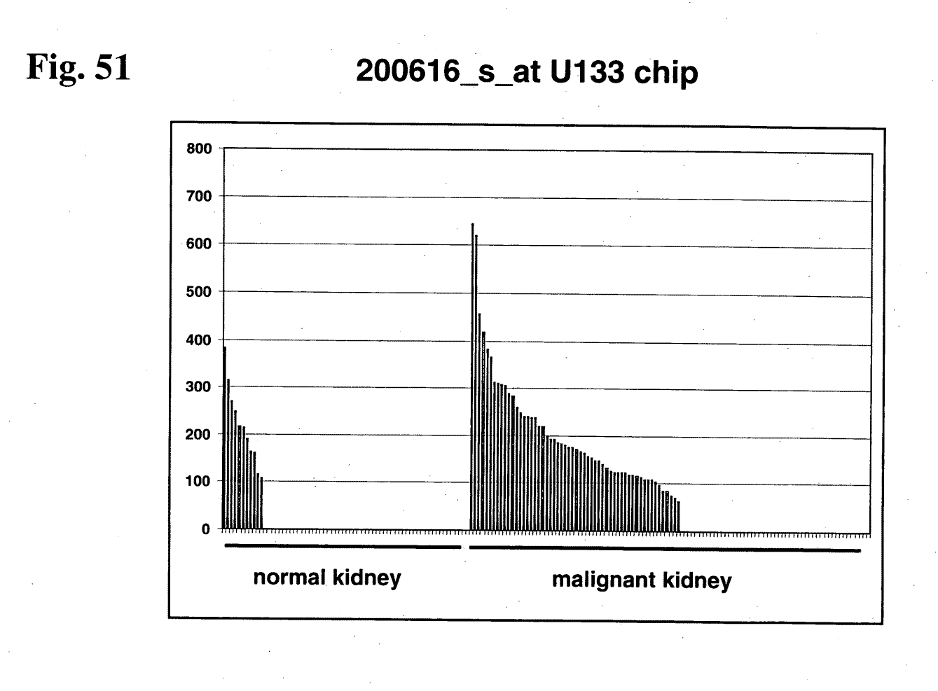

FIG. 51 shows the results of interrogating the GeneLogic database as described in FIG. 47. KIAA0152 was expressed in 56 of the 106 malignant kidney tissues examined and 11 of the 65 normal kidney tissues examined.

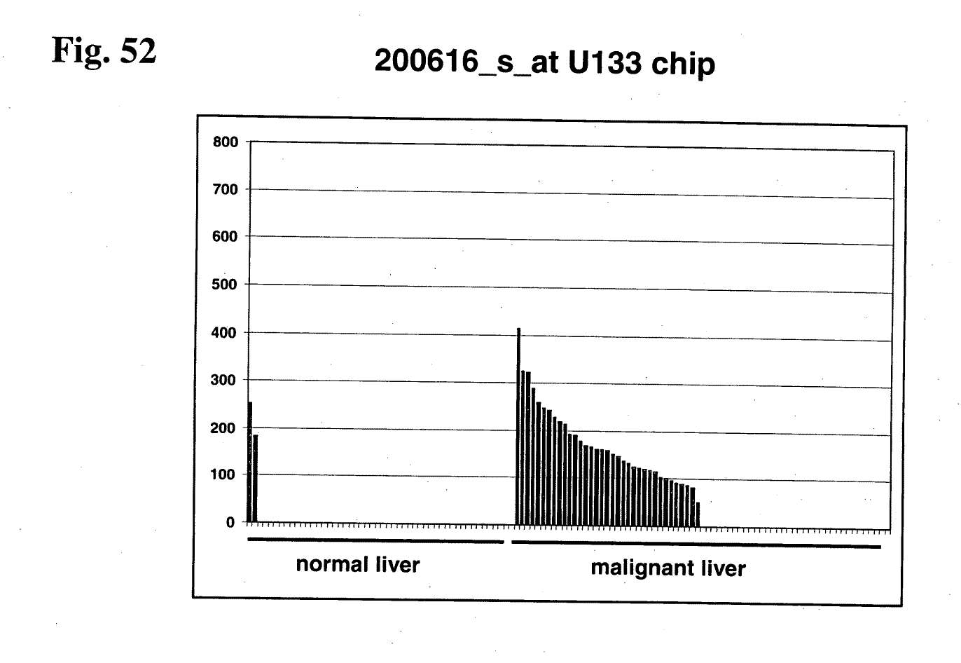

FIG. 52 shows the results of interrogating the GeneLogic database as described in FIG. 47. KIAA0152 was expressed in 35 of the 70 malignant liver tissues examined and in two of the 49 normal liver tissues examined.

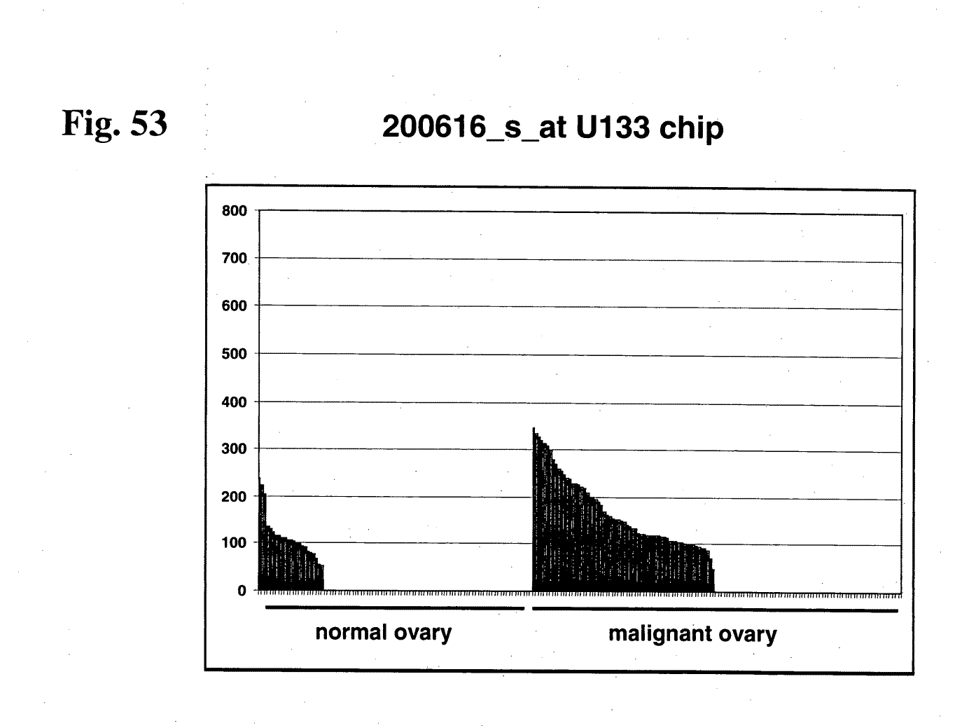

FIG. 53 shows the results of interrogating the GeneLogic database as described in FIG. 47. KIAA0152 was expressed in 64 of the 128 malignant ovary tissues examined and 23 of the 95 normal ovary tissues examined.

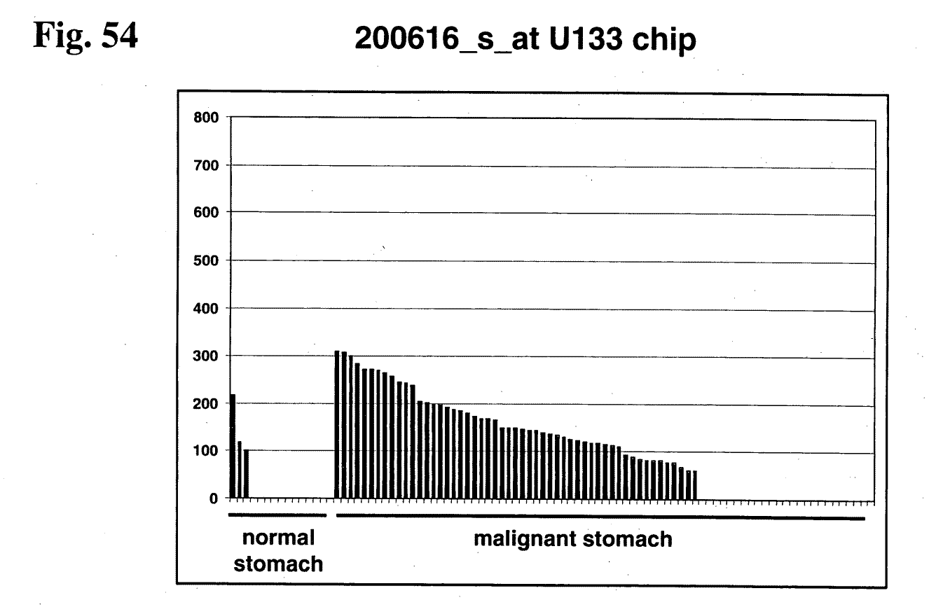

FIG. 54 shows the results of interrogating the GeneLogic database as described in FIG. 47. KIAA0152 was expressed in 53 of the malignant stomach tissues examined and in three of the normal stomach tissues examined.

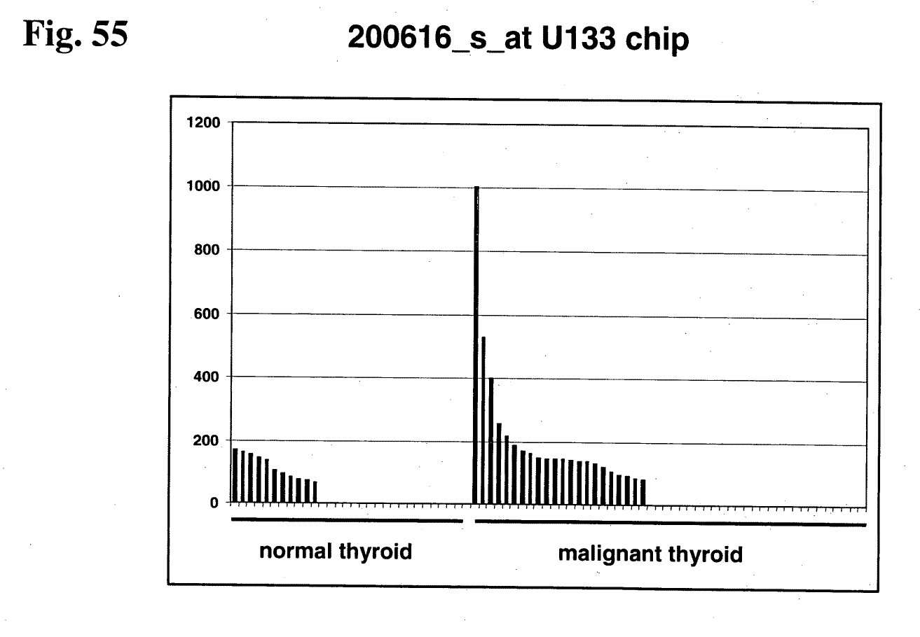

FIG. 55 shows the results of interrogating the GeneLogic database as described in FIG. 47. KIAA0152 was expressed in 22 of the malignant thyroid tissues examined and in 11 of the normal thyroid tissues examined.

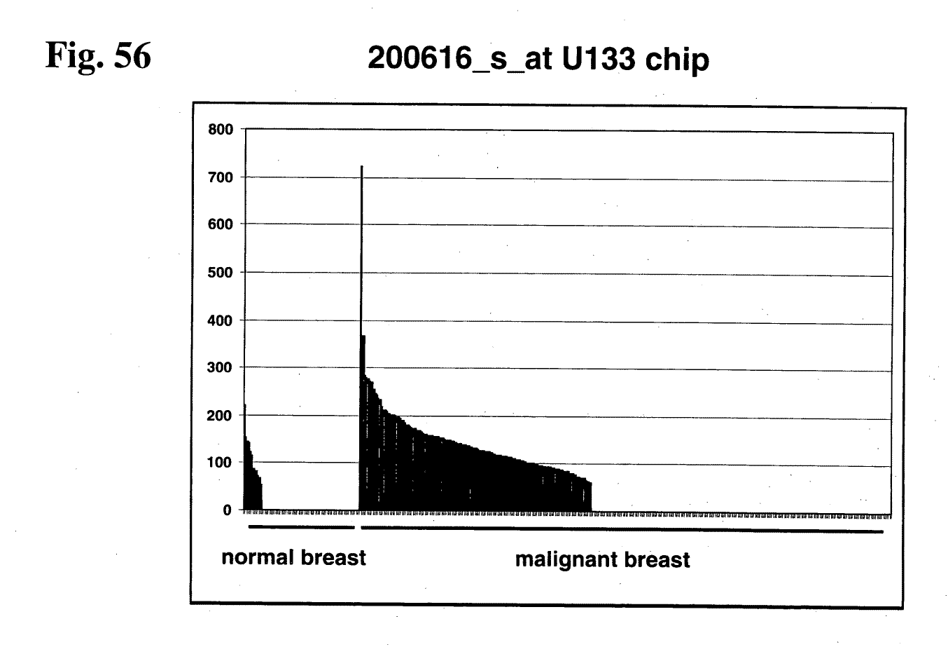

FIG. 56 shows the results of interrogating the GeneLogic database as described in FIG. 47. KIAA0152 was expressed in 153 of the 348 malignant breast tissues examined and in 12 of the 75 normal breast tissues examined.

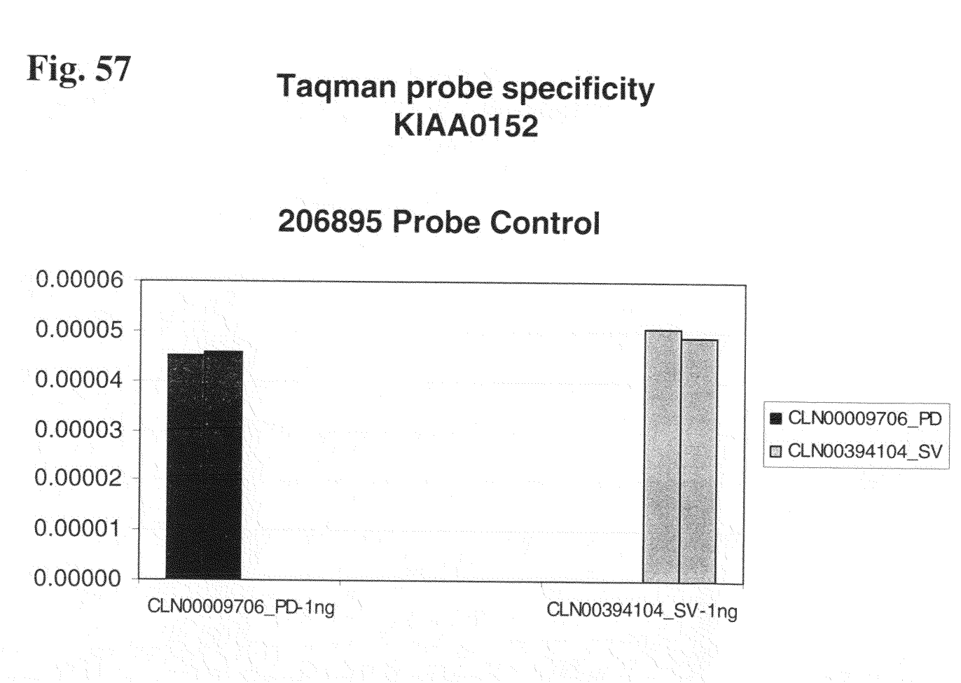

FIG. 57 shows the specificity of real-time polymerase chain reaction (RT-PCR) primers/probes for two KIAA0152 clones, CLN00009706_PD and CLN00394104_SV. These primers/probes were designed for use in RT-PCR to specifically detect KIAA0152, and were used in a Taqman primer test.

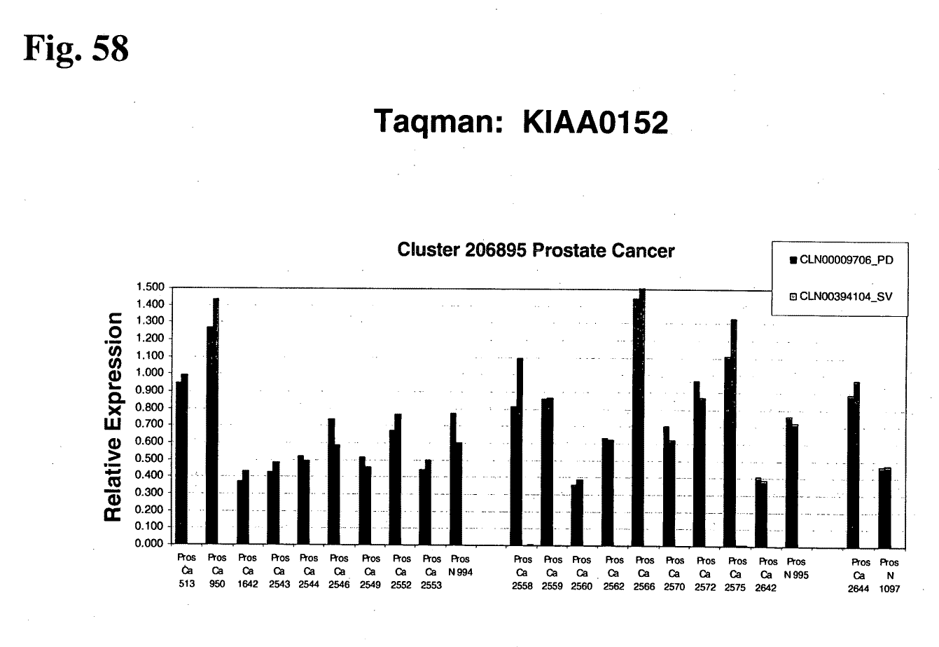

FIG. 58 shows the relative expression of two KIAA0152 clones, CLN00009706_PD and CLN00394104_SV, in prostate cancer, as determined by Taqman RT-PCR. These results demonstrate CLN00009706_PD but not CLN00394104_SV was overexpressed in many prostate cancer tissues.

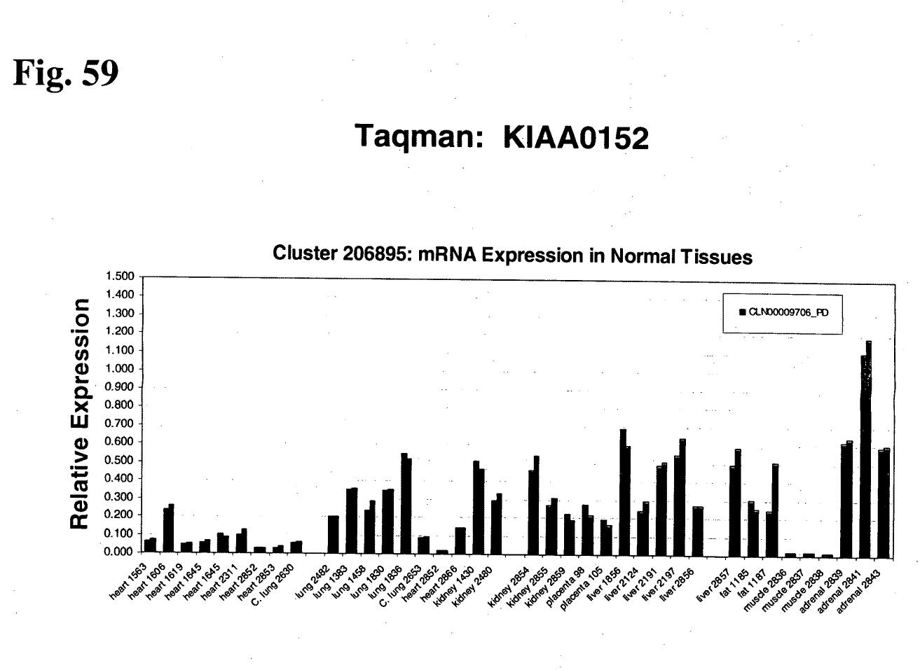

FIG. 59 shows the relative expression of KIAA0152 in RNA specimen from normal tissues, including placenta, heart, lung, kidney, liver, fat tissue, muscle, and adrenal gland, as determined by Taqman RT-PCR.

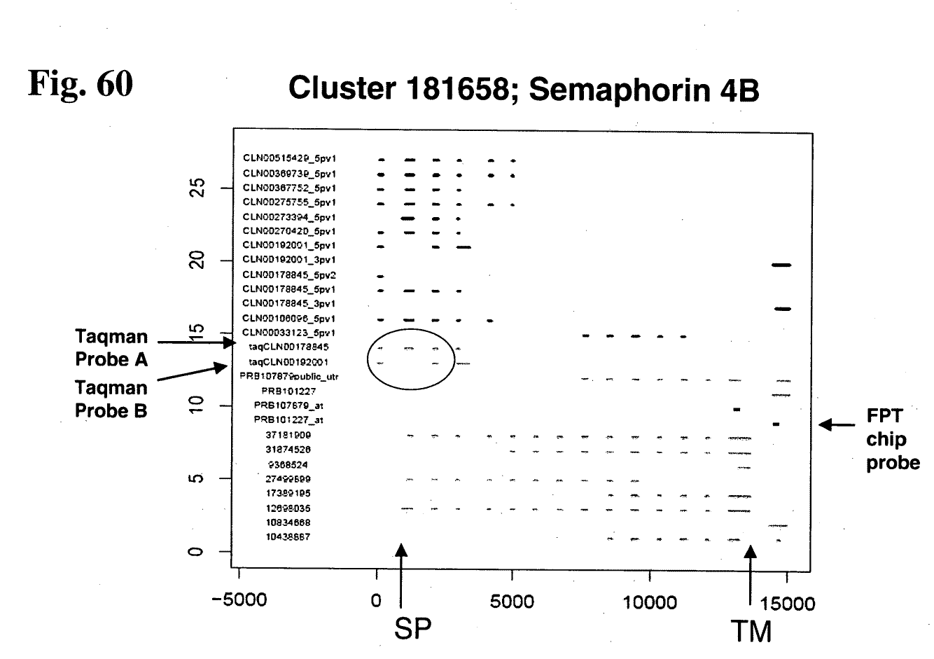

FIG. 60 shows an exon map of public and proprietary clones in cluster 181658, semaphorin 4B; the location of the microarray hybridization probe PRB101227_at; and the relative location of the RT-PCR primers and probes for CLN00178845 (Taqman probe A) and CLN00192001 (Taqman probe B). SP indicates the location of the signal peptide sequence and TM indicates the location of the transmembrane domain. The horizontal axis is a scaled version of the genome which considers all introns to have equal lengths.

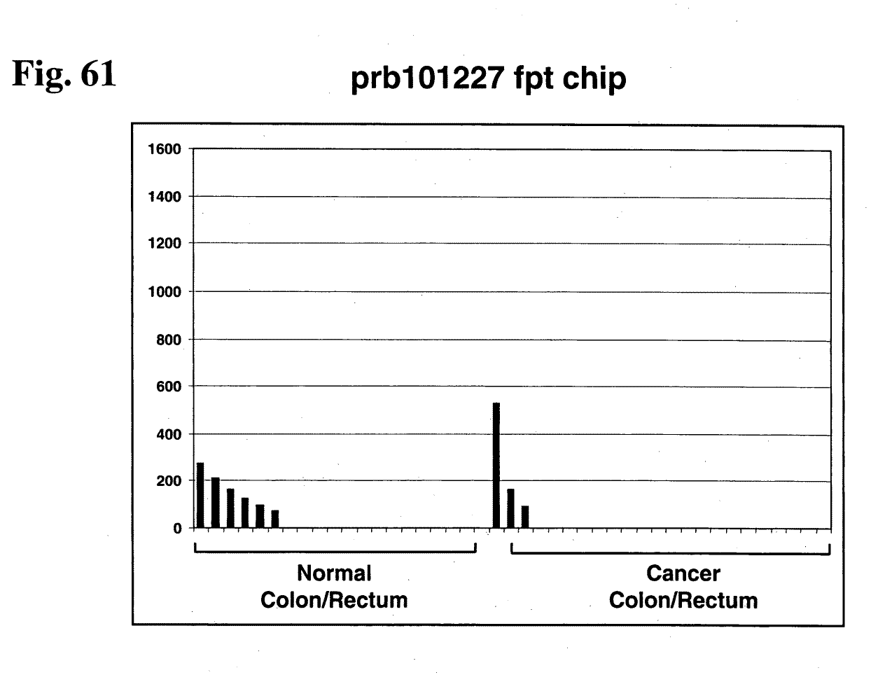

FIG. 61 shows the expression level of semaphorin 4B as detected by PRB101227 (black bars) and measured by microarray hybridization, using a Five Prime chip, of colorectal adenocarcinoma samples, and normal human colorectal specimens. The results show that semaphorin 4B was expressed in three of the 23 tumor samples examined and in six of the 19 normal samples examined. High-level expression of semaphorin 4B, i.e., in excess of the highest level of expression observed in a normal sample, was observed in one of the tumor samples.

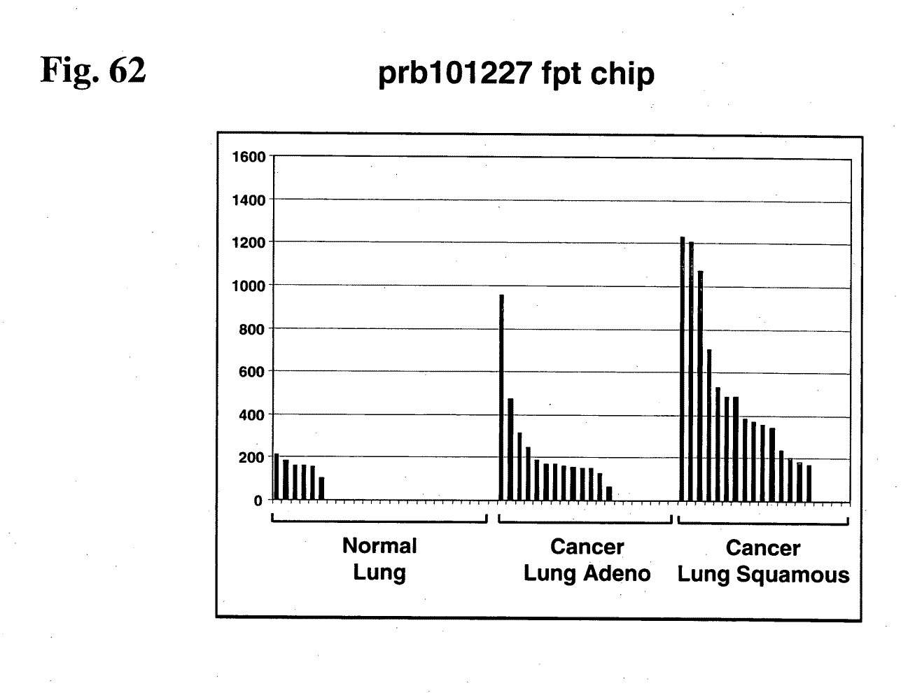

FIG. 62 shows the expression level of semaphorin 4B as detected by PRB101227 (black bars) and measured by microarray hybridization, using a Five Prime chip, of lung squamous cell cancer samples, human lung adenocarcinoma samples, and normal lung samples The results show that semaphorin 4B was expressed in 15 of the 19 lung squamous carcinoma samples, in 13 of 19 lung adenocarcinoma samples, and in six of 24 normal lung samples. High-level expression of semaphorin 4B, i.e. in excess of the highest level of expression observed in a normal sample, was observed in about 12 of 19 lung squamous carcinoma samples and about four of 19 lung adenocarcinoma samples.

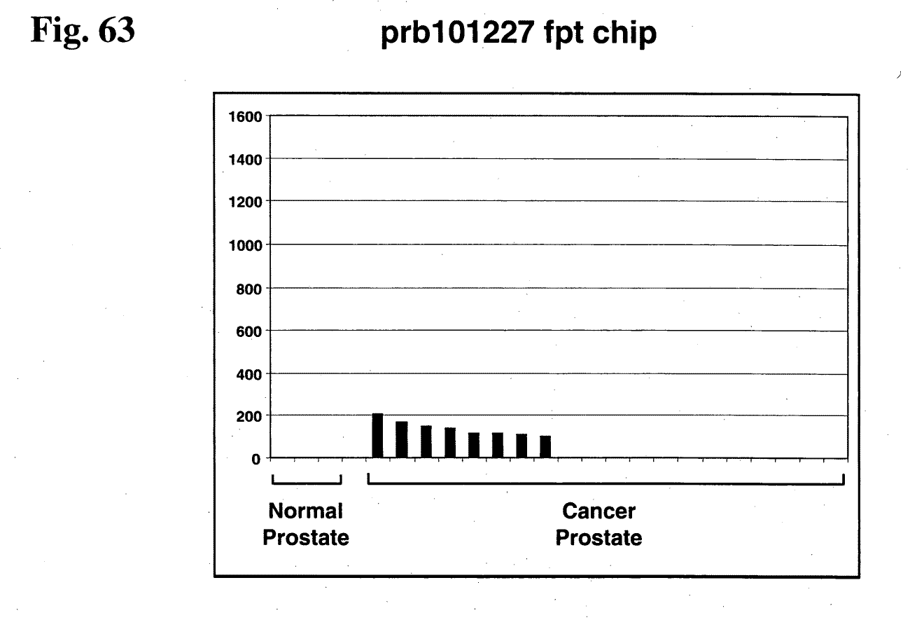

FIG. 63 shows the expression level of semaphorin 4B as detected by PRB101227 (black bars) and measured by microarray hybridization, using a Five Prime chip, of prostate cancer specimens and normal prostate specimens. The results show that semaphorin 4B was expressed in eight of the 20 prostate cancer samples but was not detected in any of the four normal prostate samples examined.

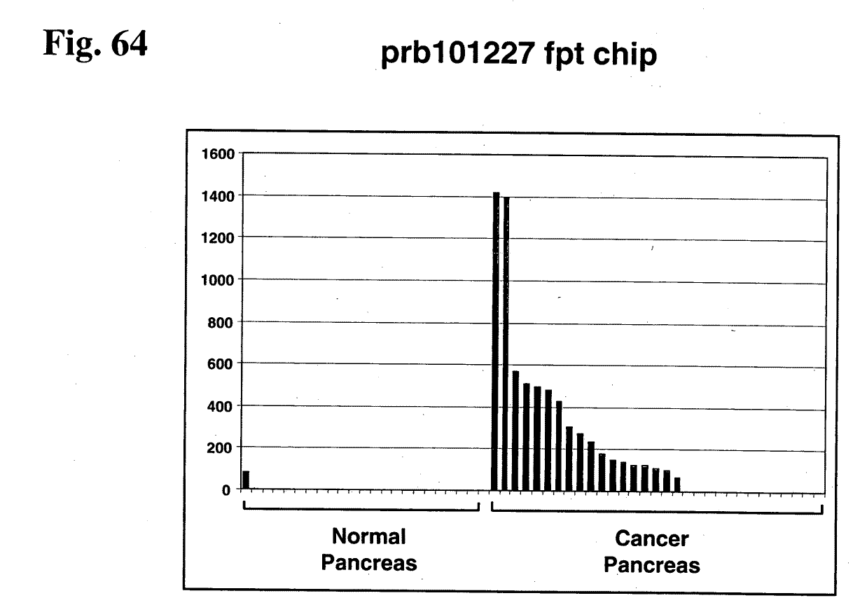

FIG. 64 shows the expression level of semaphorin 4B as detected by PRB101227 (black bars) and measured by microarray hybridization, using a Five Prime chip, of pancreatic cancer samples and normal pancreas samples. The results show that semaphorin 4B was expressed in 18 of the 31 pancreatic cancer samples and in one of the 22 normal pancreas samples. High-level expression of semaphorin 4B, i.e. in excess of the highest level of expression observed in a normal sample, was observed in about 17 of the 31 tumor samples.

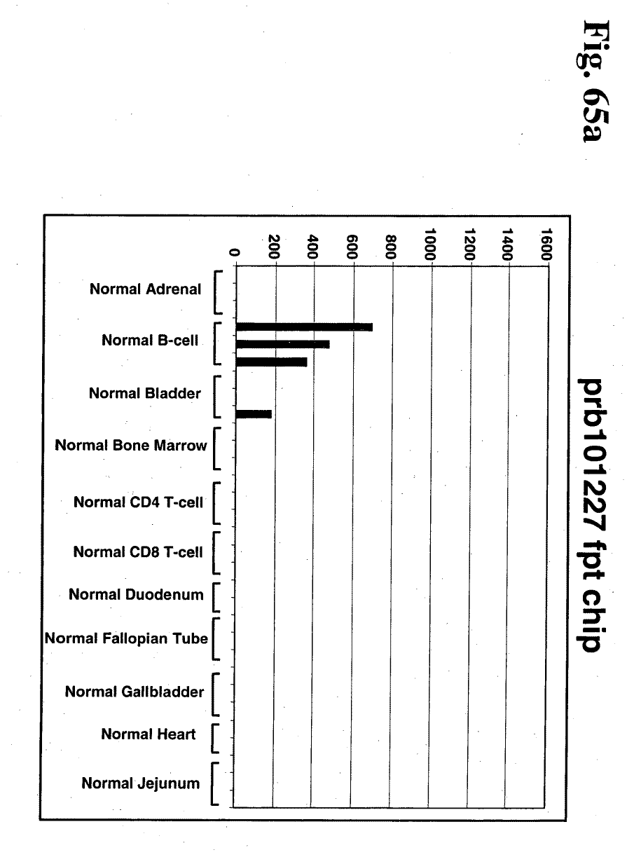

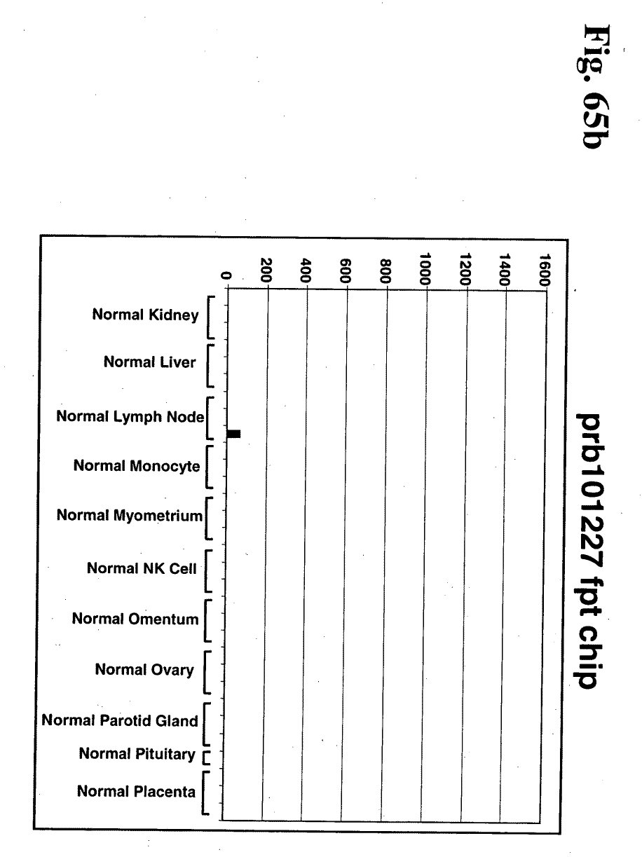

FIG. 65 shows the expression level of semaphorin 4B as detected by PRB101227 (black bars) and measured by microarray hybridization, using a Five Prime chip, of normal tissue specimens. FIG. 65a shows semaphorin 4B expression in normal adrenal, B-cell, bladder, bone marrow, CD4⁺ T-cell, CD8⁺ T-cell, duodenum, fallopian tube, gallbladder, heart, and jejunum. FIG. 65b shows semaphorin 4B expression in normal kidney, liver, lymph node, monocyte, myometrium, NK cell, omentum, ovary, parotid gland, pituitary, and placenta. FIG. 65c shows semaphorin 4B expression in normal skeletal muscle, skin, small intestine, soft tissues, spleen, stem cell, adipose tissue, testis, thymus, thyroid, uterus, and white blood cells (WBC).

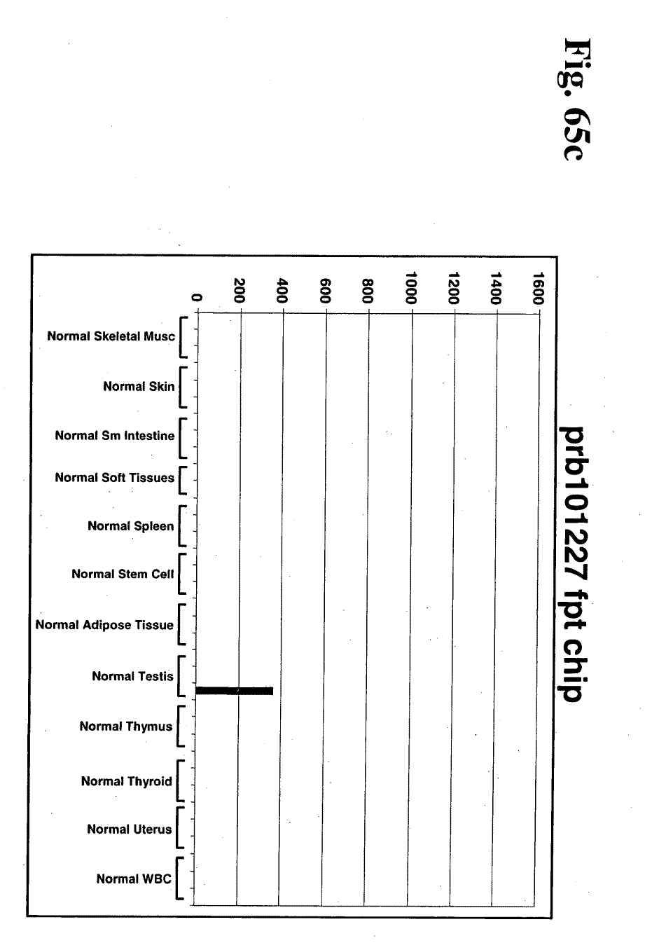

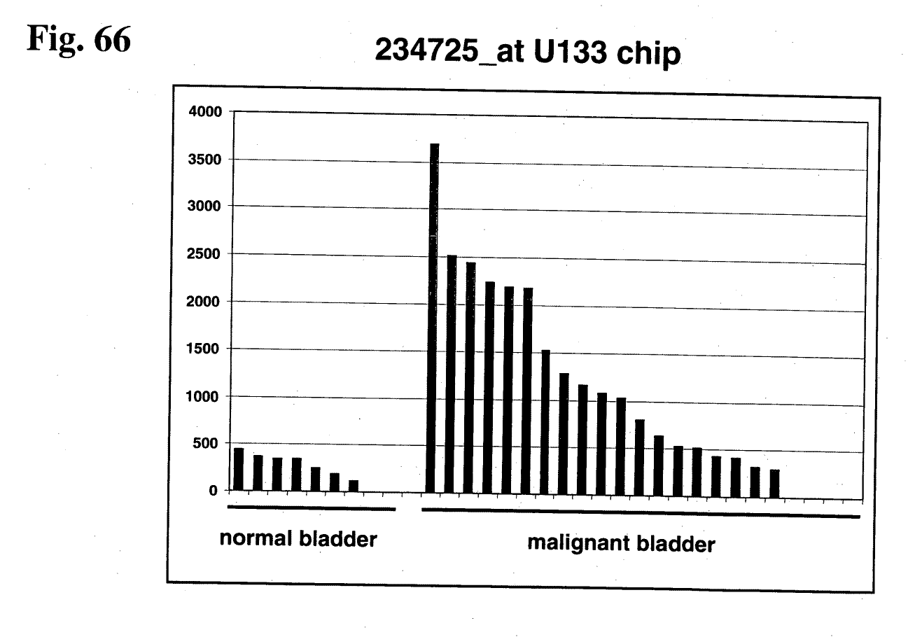

FIG. 66 shows the results of interrogating a proprietary oncology database from GeneLogic by probing an Affymetrix U133 chip with a probe 234725_at corresponding to semaphorin 4B in order to determine the expression of the sequences in normal and malignant bladder tissues. Semaphorin 4B was expressed in 19 of the 23 malignant bladder tissues examined and in seven of the nine normal bladder tissues examined.

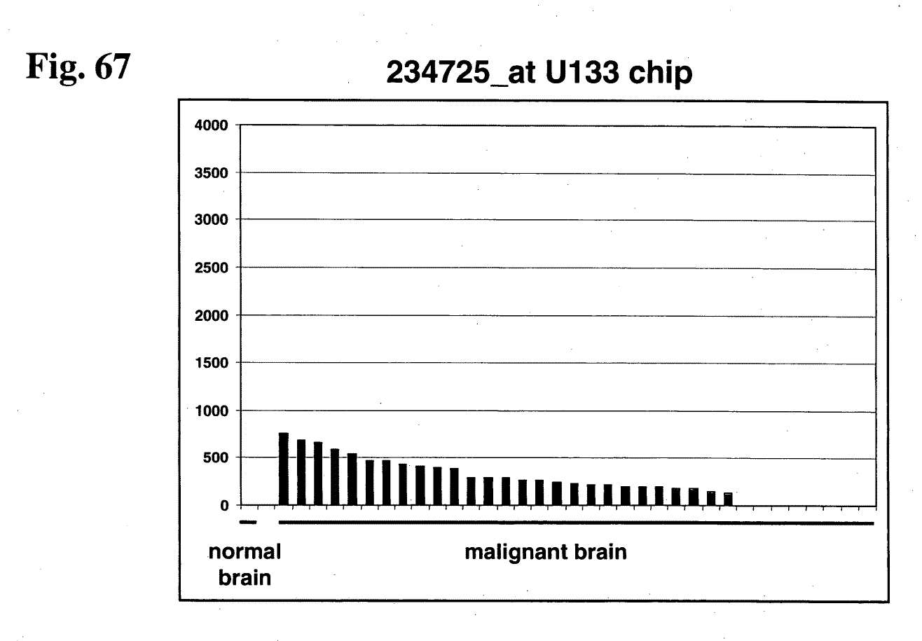

FIG. 67 shows the results of interrogating the GeneLogic database as described in FIG. 66. Semaphorin 4B was expressed in 27 of the 35 malignant brain tissues examined and was not expressed in the normal brain specimen examined.

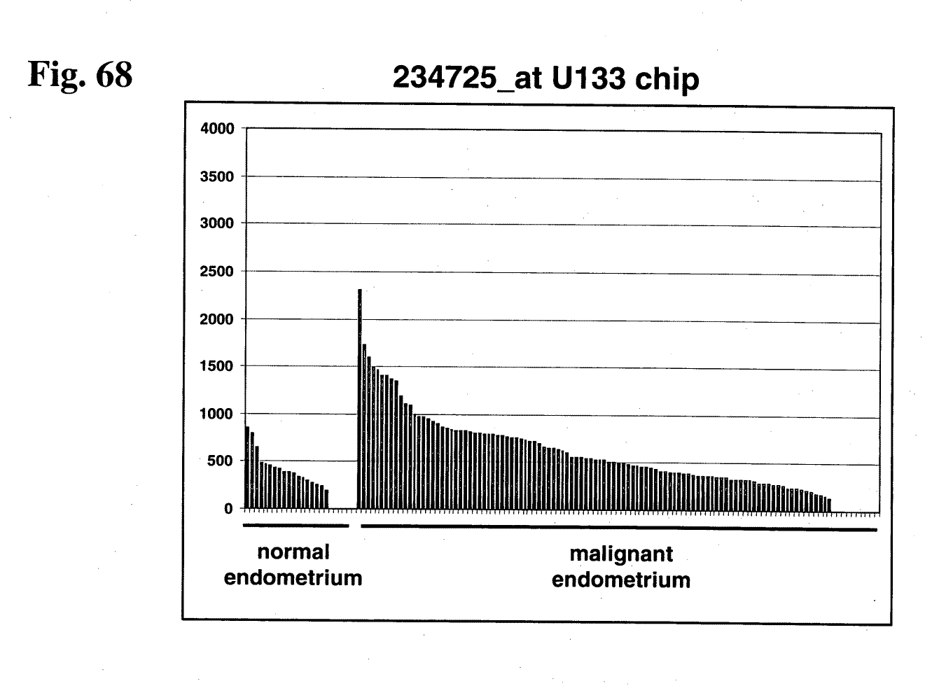

FIG. 68 shows the results of interrogating the GeneLogic database as described in FIG. 66. Semaphorin 4B was expressed in 102 of the 112 malignant endometrium tissues examined and in 18 of the 23 normal endometrium tissues examined.

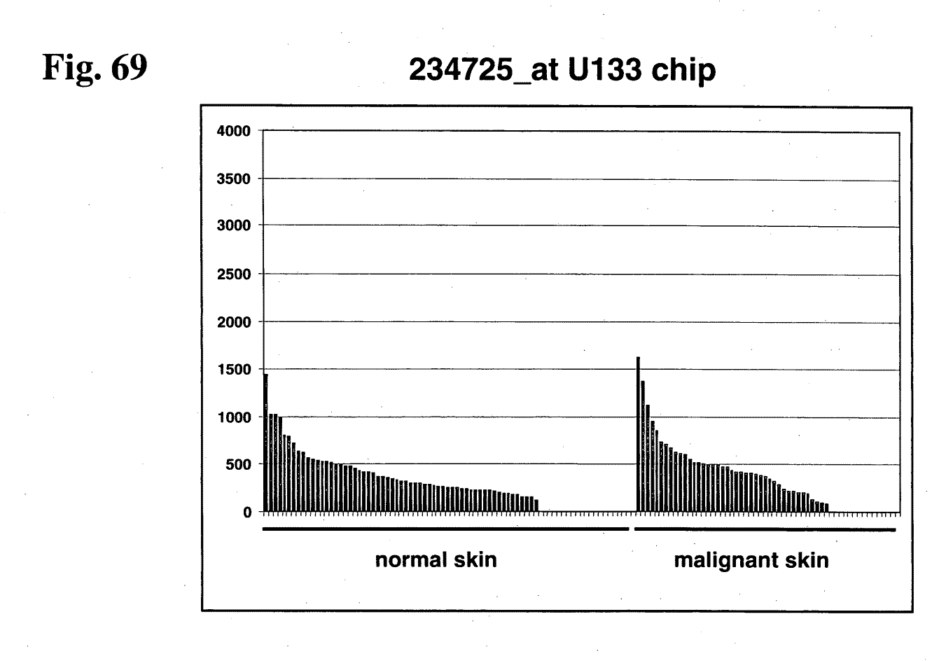

FIG. 69 shows the results of interrogating the GeneLogic database as described in FIG. 66. Semaphorin 4B was expressed in 41 of the malignant skin tissues examined and in 59 of the normal skin tissues examined.

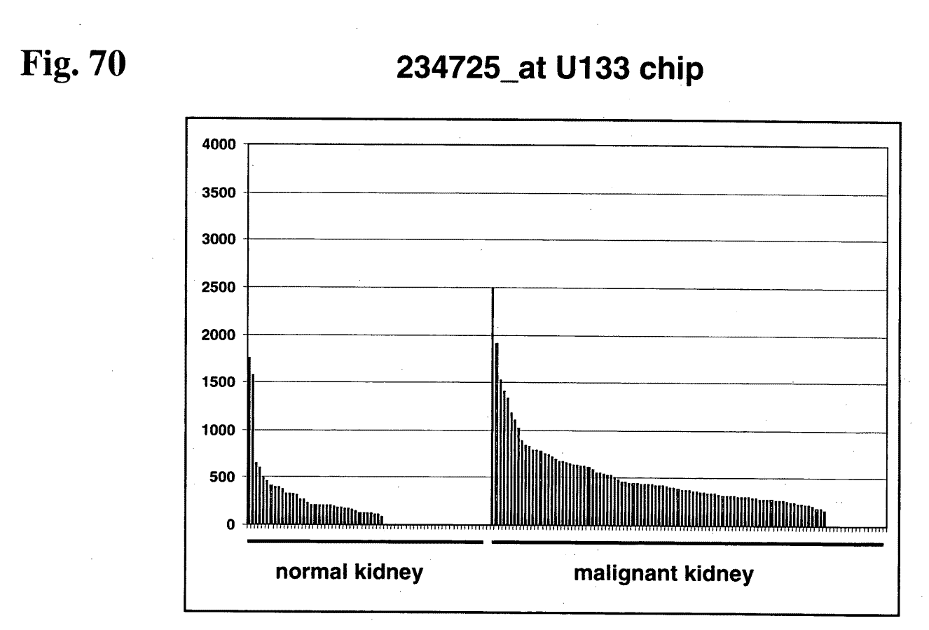

FIG. 70 shows the results of interrogating the GeneLogic database as described in FIG. 66. Semaphorin 4B was expressed in 90 of the 106 malignant kidney tissues examined and in 37 of the 65 normal kidney tissues examined.

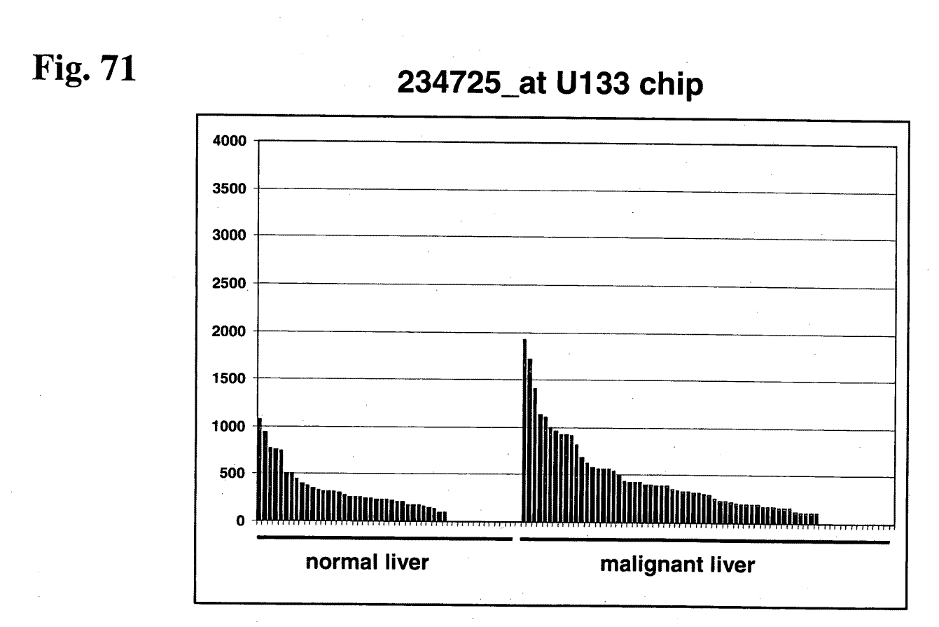

FIG. 71 shows the results of interrogating the GeneLogic database as described in FIG. 66. Semaphorin 4B was expressed in 56 of the 70 malignant liver tissues examined and in 36 of the 49 normal liver tissues examined.

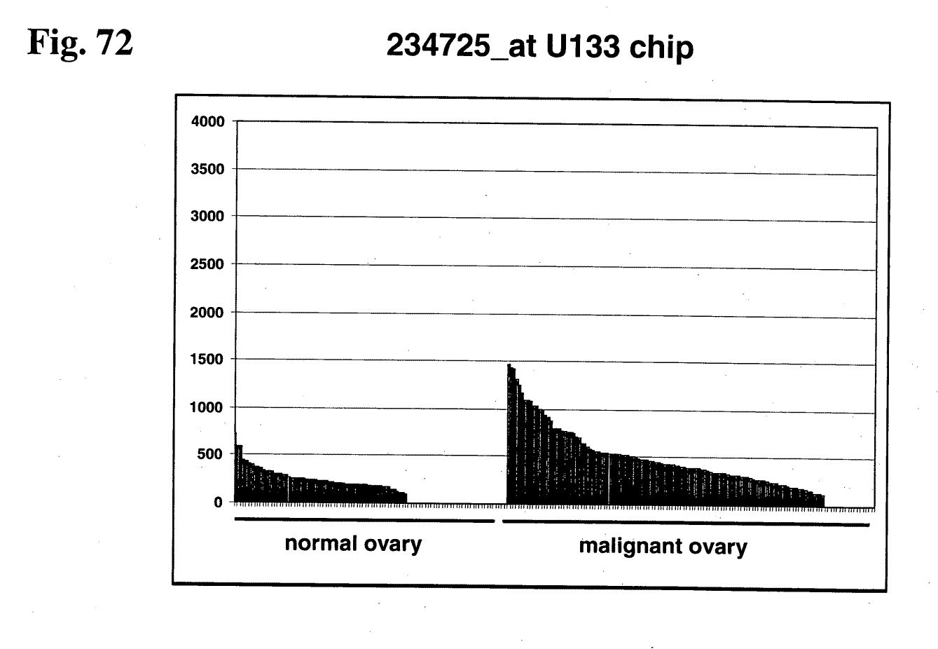

FIG. 72 shows the results of interrogating the GeneLogic database as described in FIG. 66. Semaphorin 4B was expressed in 111 of the 128 malignant ovary tissues examined and in 61 of the 95 normal ovary tissues examined.

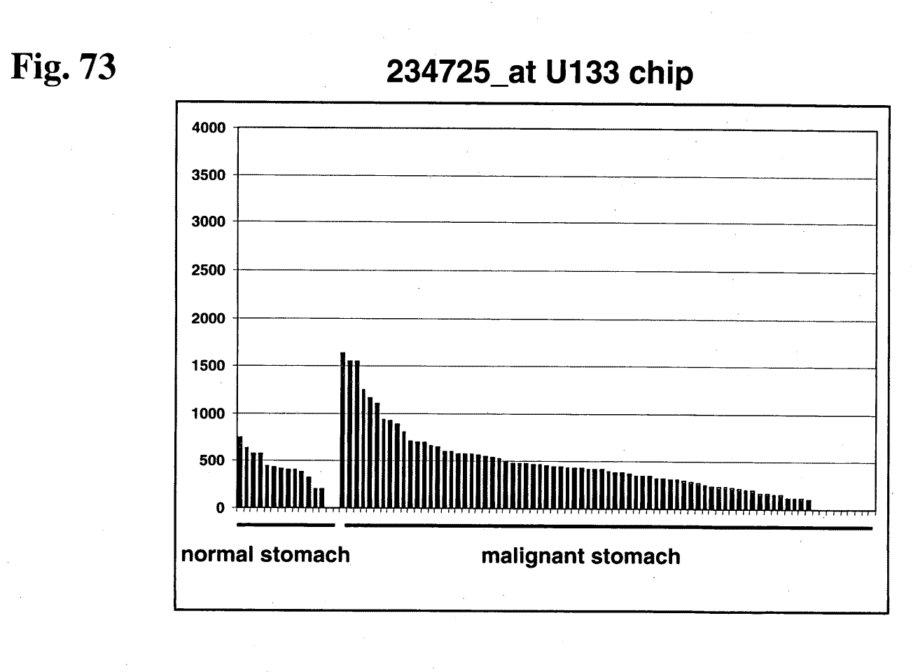

FIG. 73 shows the results of interrogating the GeneLogic database as described in FIG. 66. Semaphorin 4B was expressed in 69 of the 78 malignant stomach tissues examined and in all 14 normal stomach tissues examined.

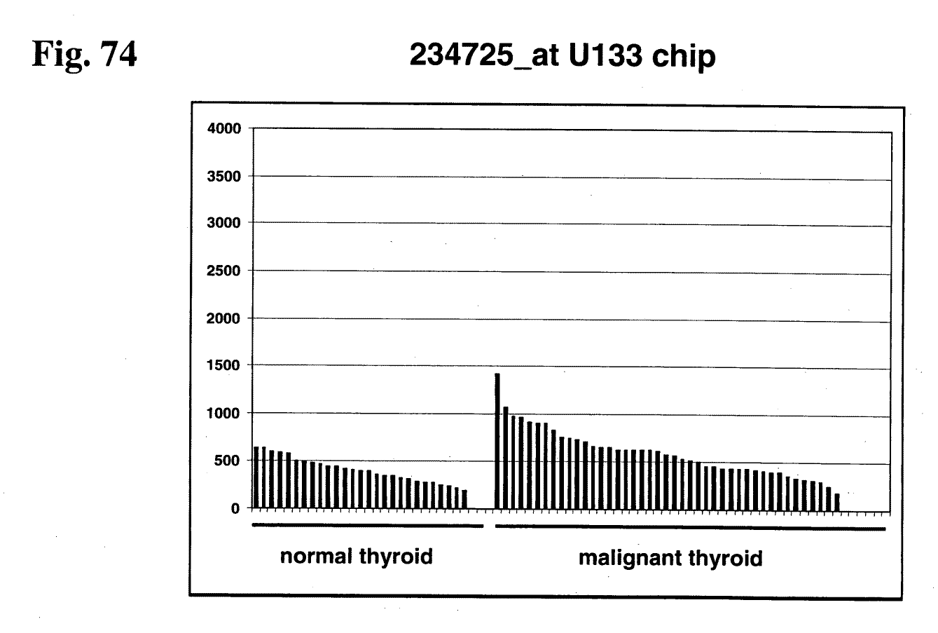

FIG. 74 shows the results of interrogating the GeneLogic database as described in FIG. 66. Semaphorin 4B was expressed in 43 of the 49 malignant thyroid tissue examined and in 27 of the 29 normal thyroid tissues examined.

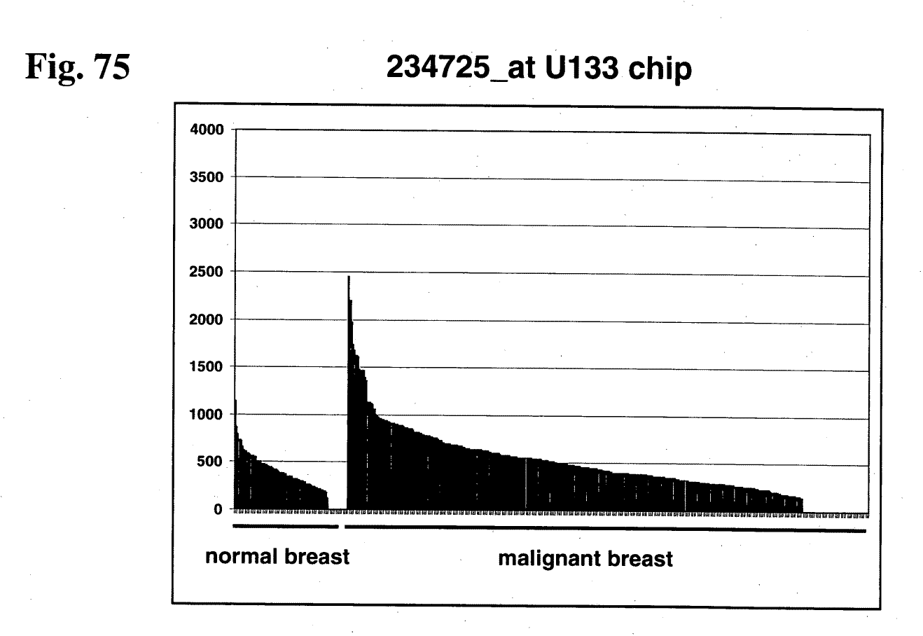

FIG. 75 shows the results of interrogating the GeneLogic database as described in FIG. 66. Semaphorin 4B was expressed in 305 of the 348 malignant breast tissues examined and in 63 of the 75 normal breast tissues examined.

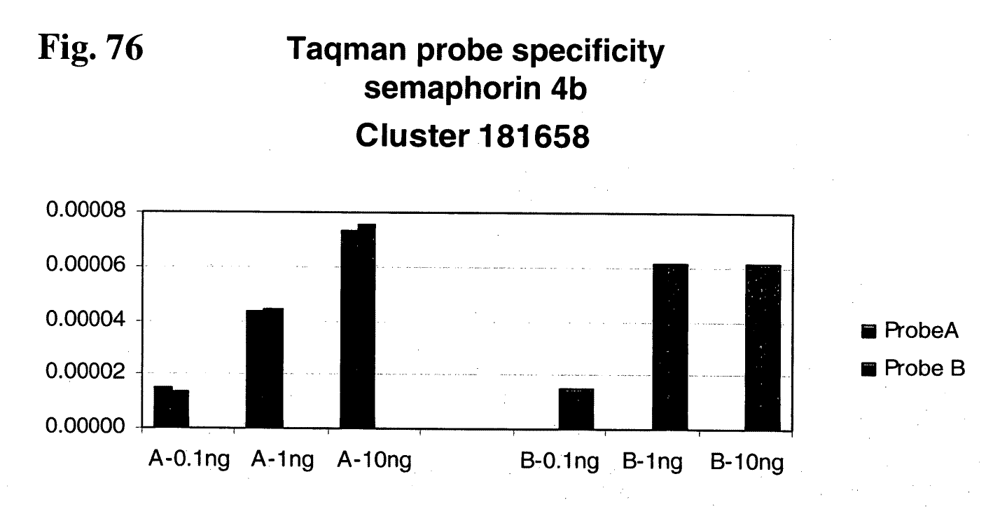

FIG. 76 shows the specificity of real-time polymerase chain reaction (RT-PCR) primers/probes for two semaphorin 4B clones, CLN00178845 (Taqman probe A) and CLN00192001 (Taqman probe B). These primers/probes were designed for use in RT-PCR to specifically detect the two different splice forms of semaphorin 4B. The probes were used in amounts of 0.1 ng, 1 ng, and 10 ng. The results show that primer/probe A is specific for CLN00178845, and primer/probe B is specific for CLN00192001.

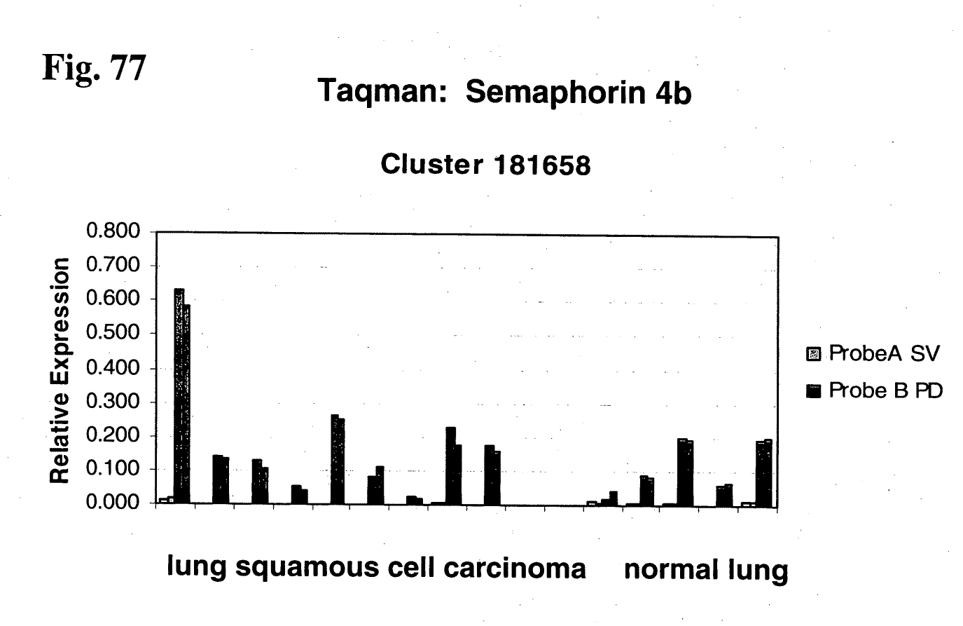

FIG. 77 shows the relative expression of the two different forms of semaphorin 4B in lung squamous cell cancer and normal adjacent RNA specimens, as determined by Taqman RT-PCR. The results show high-level expression of CLN00192001 (detected by probe B) in normal and lung squamous cell carcinoma as compared to CLN00178845 (detected by probe A), and at least one out of nine lung squamous cell carcinoma samples showed at least two-fold higher expression of CLN00192001 than normal lung.

### BRIEF DESCRIPTION OF THE TABLES

Table 1 provides information regarding sequences listed in the Sequence Listing that relate to cluster 192303, IgSF9. Column 1 shows an internally designated identification number (FP ID). Column 2 shows the nucleotide sequence ID number for the nucleic acids of the open reading frames that encode the polypeptides of the invention (SEQ. ID. NO. (N1)). Column 3 shows the amino acid sequence ID number for polypeptide sequences (SEQ. ID. NO. (P1)). Column 4 shows the nucleotide sequence ID number for nucleic acids that may include both coding and non-coding regions (SEQ. ID. NO. (N0)). Column 5 shows the NCBI accession number or an internal designation for the nucleic acids and polypeptides specified in columns 2-4 (Clone ID).

Table 2 provides information regarding NCBI sequences belonging to cluster 192303, IgSF9, identified by probe PRB103989_s_at. Column 1 shows the internally designated identification number (FP ID). Column 2 shows the NCBI accession number (Clone ID). Column 3 shows the predicted number of amino acids encoded by the sequence (Predicted Protein Length). Column 4 shows the name and species origin of the sequence as listed in the NCBI database (Annotation).

Table 3 provides information regarding the polypeptides encoded by the NCBI sequences belonging to cluster 192303, IgSF9. Column 1 shows the internally designated identification number (FP ID). Column 2 shows the NCBI accession number (Clone ID). Column 3 shows the predicted length of the polypeptide encoded by each clone (Pred Prot Len). Column 4 (Tree-vote) shows the result of an algorithm that predicts whether the predicted amino acid sequence is secreted. A Tree-vote at or near 0 indicates a low probability that the protein is secreted. A Tree vote at or near 1.00 indicates a high probability that the protein is secreted. Column 5 shows the predicted signal peptide coordinates (Signal Peptide Coords). Column 6 shows the mature protein coordinates, which refer to the coordinates of the amino acid residues of the mature polypeptide after cleavage of the secretory leader or signal peptide sequence (Mature Protein Coords). Column 7 shows alternate predictions of the signal peptide coordinates (Altern Signal Peptide Coords). Column 8 specifies the coordinates of an alternative form of the mature protein (Altern Mature Protein Coords). The alternate mature protein coordinates result from alternative predictions of the signal peptide cleavage site; their presence may, for example, depend on the host used to express the polypeptides. Column 9 specifies the number of transmembrane domains (TM). Columns 10 and 11 provide the coordinates of the transmembrane and non-transmembrane sequences of the polypeptides. The transmembrane coordinates (TM Coords) designate the transmembrane domains of the molecule. The non-transmembrane coordinates (non-TM Coords) refer to the protein segments not located in the membrane; these can include extracellular, cytoplasmic, and luminal sequences. Coordinates are listed in terms of the amino acid residues beginning with “1” for the first amino acid residue at the N-terminus of the full-length polypeptide. Finally, column 12 provides a list of Pfam domains present in each of the identified clones.

Table 4 shows the coordinates of predicted functional domains (Pfam domains) within IgSF9 polypeptides identified by probe PRB103989_s_at. Column 1 shows the internally designated identification number (FP ID). Column 2 shows the NCBI accession number for the polypeptide (Clone ID). Column 3 shows the names of the predicted functional domains (Pfam). Column 4 shows the coordinates of the beginning and ending amino acid residues spanning the functional domains in the polypeptide (Coordinates).

Table 5 provides information regarding sequences listed in the Sequence Listing that relate to cluster 301014, nectin 4. Column 1 shows an internally designated identification number (FP ID). Column 2 shows the nucleotide sequence ID number for the nucleic acids of the open reading frames that encode the polypeptides of the invention (SEQ. ID. NO. (N1)). Column 3 shows the amino acid sequence ID number for polypeptide sequences (SEQ. ID. NO. (P1)). Column 4 shows the nucleotide sequence ID number for nucleic acids that may include both coding and non-coding regions (SEQ. ID. NO. (N0)). Column 5 shows the NCBI accession number or an internal designation for the nucleic acids and polypeptides specified in columns 2-4 (Clone ID).

Table 6 provides information regarding NCBI sequences belonging to cluster 301014, nectin 4, identified by probe PRB103018_s_at. Column 1 shows the internally designated identification number (FP ID). Column 2 shows the NCBI accession number (Clone ID). Column 3 shows the predicted number of amino acids encoded by the sequence (Predicted Protein Length). Column 4 shows the name and species origin of the sequence as listed in the NCBI database (Annotation).

Table 7 provides information regarding the polypeptides encoded by the NCBI sequences belonging to cluster 301014, nectin 4. Column 1 shows the internally designated identification number (FP ID). Column 2 shows the NCBI accession number (Clone ID). Column 3 shows the predicted length of the polypeptide encoded by each clone (Pred Prot Len). Column 4 (Tree-vote) shows the result of an algorithm that predicts whether the predicted amino acid sequence is secreted. Column 5 shows the predicted signal peptide coordinates (Signal Peptide Coords). Column 6 shows the mature protein coordinates, which refer to the coordinates of the amino acid residues of the mature polypeptide after cleavage of the secretory leader or signal peptide sequence (Mature Protein Coords). Column 7 shows alternate predictions of the signal peptide coordinates (Altern Signal Peptide Coords). Column 8 specifies the coordinates of an alternative form of the mature protein (Altern Mature Protein Coords). The alternative mature protein coordinates result from alternative predictions of the signal peptide cleavage site; their presence may, for example, depend on the host used to express the polypeptides. Column 9 specifies the number of transmembrane domains (TM). Columns 10 and 11 provide the coordinates of the transmembrane and non-transmembrane sequences of the polypeptides. The transmembrane coordinates (TM Coords) designate the transmembrane domains of the molecule. The non-transmembrane coordinates (non-TM Coords) refer to the protein segments not located in the membrane; these can include extracellular, cytoplasmic, and luminal sequences. Coordinates are listed in terms of the amino acid residues beginning with “1” for the first amino acid residue at the N-terminus of the full-length polypeptide. Finally, column 12 provides a list of Pfam and/or Prosite domains present in each of the identified clones.

Table 8 shows the coordinates of predicted functional domains (Pfam and Prosite domains) within nectin 4 polypeptides identified by probe PRB103018_s_at. Column 1 shows the internally designated identification number (FP ID). Column 2 shows the NCBI accession number for the polypeptide (Clone ID). Columns 3 and 4 show the names of the predicted functional domains (Pfam and Prosite). Column 5 shows the coordinates of the beginning and ending amino acid residues spanning the functional domains in the polypeptide (Coordinates).

Table 9 provides information regarding sequences listed in the Sequence Listing that relate to cluster 206895, KIAA0152. Column 1 shows an internally designated identification number (FP ID). Column 2 shows the nucleotide sequence ID number for the nucleic acids of the open reading frames that encode the polypeptides of the invention (SEQ. ID. NO. (N1)). Column 3 shows the amino acid sequence ID number for polypeptide sequences (SEQ. ID. NO. (P1)). Column 4 shows the nucleotide sequence ID number for nucleic acids that may include both coding and non-coding regions (SEQ. ID. NO. (N0)). Column 5 shows the NCBI accession number or an internal designation for the nucleic acids and polypeptides specified in columns 2-4 (Clone ID).

Table 10 provides information regarding an NCBI sequence belonging to cluster 206895, KIAA0152, identified by probe PRB105610_at. Column 1 shows the internally designated identification number (FP ID). Column 2 shows the NCBI accession number (Clone ID). Column 3 shows the predicted number of amino acids encoded by the sequence (Predicted Protein Length). Column 4 shows the name and species origin of the sequence as listed in the NCBI database (Annotation).

Table 11 provides information regarding the polypeptide encoded by an NCBI sequence belonging to cluster 206895, KIAA0152. Column 1 shows the internally designated identification number (FP ID). Column 2 shows the NCBI accession number (Clone ID). Column 3 shows the predicted length of the polypeptide encoded by each clone (Pred Prot Len). Column 4 (Tree-vote) shows the result of an algorithm that predicts whether the predicted amino acid sequence is secreted. Column 5 shows the predicted signal peptide coordinates (Signal Peptide Coords). Column 6 shows the mature protein coordinates, which refer to the coordinates of the amino acid residues of the mature polypeptide after cleavage of the secretory leader or signal peptide sequence (Mature Protein Coords). Column 7 shows alternate predictions of the signal peptide coordinates (Altern Signal Peptide Coords). Column 8 specifies the coordinates of an alternative form of the mature protein (Altern Mature Protein Coords). The alternative mature protein coordinates result from alternative predictions of the signal peptide cleavage site; their presence may, for example, depend on the host used to express the polypeptides. Column 9 specifies the number of transmembrane domains (TM). Columns 10 and 11 provide the coordinates of the transmembrane and non-transmembrane sequences of the polypeptides. The transmembrane coordinates (TM Coords) designate the transmembrane domains of the molecule. The non-transmembrane coordinates (non-TM Coords) refer to the protein segments not located in the membrane; these can include extracellular, cytoplasmic, and luminal sequences. Coordinates are listed in terms of the amino acid residues beginning with “1” for the first amino acid residue at the N-terminus of the full-length polypeptide.

Table 12 provides information regarding sequences listed in the Sequence Listing that relate to cluster 181658, semaphorin 4B. Column 1 shows an internally designated identification number (FP ID). Column 2 shows the nucleotide sequence ID number for the nucleic acids of the open reading frames that encode the polypeptides of the invention (SEQ. ID. NO. (N1)). Column 3 shows the amino acid sequence ID number for polypeptide sequences (SEQ. ID. NO. (P1)). Column 4 shows the nucleotide sequence ID number for nucleic acids that may include both coding and non-coding regions (SEQ. ID. NO. (N0)). Column 5 shows the NCBI accession number or an internal designation for the nucleic acids and polypeptides specified in columns 2-4 (Clone ID).

Table 13 provides information regarding NCBI sequences belonging to cluster 181658, semaphorin 4B, identified by probe PRB101227_at. Column 1 shows the internally designated identification number (FP ID). Column 2 shows the NCBI accession number (Clone ID). Column 3 shows the predicted number of amino acids encoded by the sequence (Predicted Protein Length). Column 4 shows the name and species origin of the sequence as listed in the NCBI database (Annotation).

Table 14 provides information regarding polypeptides encoded by the NCBI sequences belonging to cluster 181658, semaphorin 4B. Column 1 shows the internally designated identification number (FP ID). Column 2 shows the NCBI accession number (Clone ID). Column 3 shows the predicted length of the polypeptide encoded by each clone (Pred Protein Length). Column 4 (Tree-vote) shows the result of an algorithm that predicts whether the predicted amino acid sequence is secreted. Column 5 shows the predicted signal peptide coordinates (Signal Peptide Coords). Column 6 shows the mature protein coordinates, which refer to the coordinates of the amino acid residues of the mature polypeptide after cleavage of the secretory leader or signal peptide sequence (Mature Protein Coords). Column 7 specifies the number of transmembrane domains (TM). Columns 8 and 9 provide the coordinates of the transmembrane and non-transmembrane sequences of the polypeptides. The transmembrane coordinates (TM Coords) designate the transmembrane domains of the molecule. The non-transmembrane coordinates (non-TM Coords) refer to the protein segments not located in the membrane; these can include extracellular, cytoplasmic, and luminal sequences. Coordinates are listed in terms of the amino acid residues beginning with “1” for the first amino acid residue at the N-terminus of the full-length polypeptide. Finally, column 10 provides a list of Pfam and/or Prosite domains present in each of the identified clones.

Table 15 shows the coordinates of predicted functional domains (Pfam domains) within semaphorin 4B polypeptides identified by probe PRB101227_at. Column 1 shows the internally designated identification number (FP ID). Column 2 shows the NCBI accession number for the polypeptide (Clone ID). Column 3 shows the names of the predicted functional domains (Pfam). Column 4 shows the coordinates of the beginning and ending amino acid residues spanning the functional domains in the polypeptide (Coordinates).

### DETAILED DESCRIPTION OF THE INVENTION

The invention provides polynucleotides and polypeptides useful for diagnosing and treating proliferative disease. It also provides probes that detect the overexpression of IgSF9, nectin 4, and semaphorin 4B in cancer. KIAA0152 is overexpressed in normal and cancerous prostate tissue, compared to other normal tissues. As a “non-critical” tissue, normal prostate can be therapeutically ablated along with cancerous prostate tissue.

The invention further provides modulators, such as antibodies, that may function as either agonists or antagonists, and/or may specifically bind to or interfere with the activity of IgSF9, nectin 4, KIAA0152, or semaphorin 4B, or fragments of these proteins. For example, polypeptides described herein can be used as immunogens to produce specific antibody modulators directed against the polypeptide targets. These antibodies can bind to and modulate polypeptides on cell surfaces, such as the extracellular or secreted domain of a transmembrane protein, for example, by inducing antibody-dependent cell cytotoxicity (ADCC) or complement-dependent cytotoxicity (CDC), carry a payload, such as a radioisotope or a cytotoxic molecule, or act as agonist or antagonist antibodies, for example by affecting ligand/receptor interactions, affecting cofactor interactions, interfering with cell signaling, inducing an apoptotic factor, or blocking the action, production, or release of growth factors or survival factors, such as blocking the cleavage of heparin-bound EGF (HB-EGF) to inhibit release of EGF that signals through an EGF receptor, or the release of other growth factors which signal through one or more corresponding growth factor receptors. The modulators of the invention include not only antibodies, but also small molecule drugs, RNAi molecules, ribozymes, antisense molecules, soluble receptors, and extracellular fragments of receptors or transmembrane proteins.

IgSF9, nectin 4, KIAA0152, and semaphorin 4B is screening assays can identify modulators with a desired biologic or therapeutic effect. Modulators of the invention include therapeutic agents that can be used to treat proliferative diseases, including cancer and psoriasis. The polypeptides and polynucleotides herein are highly expressed in tumor tissues compared to normal tissue, especially normal tissues vulnerable to unwanted side effects of drugs.

### DEFINITIONS

The terms used herein have their ordinary meanings, as set forth below, and can be further understood in the context of the specification.

The terms “polynucleotide,” “nucleotide,” “nucleic acid,” “nucleic acid molecule,” “nucleic acid sequence,” “polynucleotide sequence,” and “nucleotide sequence” are used interchangeably herein to refer to polymeric forms of nucleotides of any length. The polynucleotides can contain deoxyribonucleotides, ribonucleotides, and/or their analogs or derivatives.

“Interfering RNA (RNAi)” refers to the effector molecules of RNA interference, a cellular mechanism of sequence-specific gene silencing that involves inhibition of gene transcription and/or translation. Interfering RNAs (RNAi) are short double-stranded RNA molecules that include, for example, small interfering RNAs (siRNAs) and microRNAs (miRNAs).

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term includes single chain protein as well as multimers. The term also includes conjugated proteins, fusion proteins, including, but not limited to, glutathione S-transferase (GST) fusion proteins, fusion proteins with a heterologous amino acid sequence, fusion proteins with heterologous and homologous leader sequences, fusion proteins with or without N-terminal methionine residues, pegolyated proteins, and immunologically tagged, or his-tagged proteins. The term also includes peptide aptamers.

“Transmembrane proteins” extend into or through the cell membrane's lipid bilayer; they can span the membrane once, or more than once. Transmembrane proteins, having part of their molecules on either side of the bilayer have many and widely variant biological functions. Transmembrane proteins are often involved in cell signaling events; they can comprise signaling molecules, or can interact with signaling molecules. Extracellular domains of transmembrane proteins may be cleaved to produce soluble receptors.

“Secreted proteins” are generally capable of being directed to the endoplasmic reticulum (ER), secretory vesicles, or the extracellular space as a result of a secretory leader, signal peptide, or leader sequence. They may be released into the extracellular space, for example, by exocytosis or proteolytic cleavage, regardless of whether they comprise a signal sequence. A secreted protein may in some circumstances undergo processing to a mature polypeptide. Secreted proteins may comprise leader sequences of amino acid residues, located at the amino terminus of the polypeptide and extending to a cleavage site, which, upon proteolytic cleavage, result in the formation of a mature protein.

A “Pfam domain” is a protein or a portion of a protein with a tertiary structure. Pfams may have characteristic functional activities, such as enzymatic or binding activities. Multiple Pfam domains can be connected by flexible polypeptide regions within a protein. Pfam domains can comprise the N-terminus or the C-terminus of a protein, or can be situated at any point between.

A “Prosite domain” is a protein or portion of a protein comprising one or more biologically meaningful motifs described as patterns or profiles. Prosite domains are linked to documentation related to the SWISS-PROT database.

A “non-transmembrane domain” is a portion of a transmembrane protein that does not span the membrane. It may be extracellular, cytoplasmic, or luminal.

A “soluble receptor” is a receptor that lacks a membrane anchor domain, such as a transmembrane domain, and may include naturally occurring splice variants of a wild-type transmembrane protein receptor in which the transmembrane domain is spliced out and the extracellular domains or any fragment of the extracellular domain of the transmembrane protein receptor. Soluble receptors can modulate a target protein. They can, for example, compete with wild-type receptors for ligand binding and participate in ligand/receptor interactions, thus modulating the activity of or the number of the receptors and/or the cellular activity downstream from the receptors. This modulation may trigger intracellular responses, for example, signal transduction events which activate cells, signal transduction events which inhibit cells, or events that modulate cellular growth, proliferation, differentiation, and/or death, or induce the production of other factors that, in turn, mediate such activities.

A “biologically active” entity, or an entity having “biological activity,” is one or more entities having structural, regulatory, or biochemical functions of a naturally occurring molecule or any function related to or associated with a metabolic or physiological process. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, such as hybridization, when it has therapeutic value in alleviating a disease condition, when it has prophylactic value in inducing an immune response, when it has diagnostic value in determining the presence of a molecule, such as a biologically active fragment of a polynucleotide that can, for example, be detected as unique for the polynucleotide molecule, or that can be used as a primer in a polymerase chain reaction. A biologically active polypeptide or fragment thereof includes one that can participate in a biological reaction, for example, one that can serve as an epitope or immunogen to stimulate an immune response, such as production of antibodies, or that can participate in stimulating or inhibiting signal transduction by binding to ligands receptors or other proteins, or nucleic acids; or activating enzymes or substrates.

The terms “antibody” and “immunoglobulin” refer to a protein, for example, one generated by the immune system, synthetically, or recombinantly, that is capable of recognizing and binding to a specific antigen; antibodies are commonly known in the art. Antibodies may recognize polypeptide or polynucleotide antigens. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid antibody molecules, F(ab′)₂ and F(ab) fragments; Fv molecules (for example, noncovalent heterodimers), dimeric and trimeric antibody fragment constructs; minibodies, humanized antibody molecules, and any functional fragments obtained from such molecules, wherein such fragments retain specific binding.

A “humanized” antibody is a non-human immunoglobulin that contains human immunoglobulin sequences. This term is generally used to refer to an immunoglobulin that has been modified to incorporate a human framework region with the hypervariable regions of a non-human immunoglobulin. The non-human regions of a humanized antibody may extend beyond the hypervariable regions into the variable regions and beyond the variable regions into the framework regions to achieve the desired antigen-binding properties.

An “epitope” is a molecule to which an antibody binds, which may or may not be a contiguous sequence of amino acid residues in a polypeptide, and which may comprise sugars and/or molecules having other chemical structures.

The term “antibody target” or “cancer target” refers to a polypeptide, polynucleotide, or carbohydrate that can be used as an immunogen in the production of antibodies that specifically bind to such a polypeptide, polynucleotide, or carbohydrate, or a small molecule drug that modulates the activity of such polypeptide, polynucleotide, or carbohydrate.

A “target cell” is a cell affected, either directly or indirectly, by an administered composition, including those comprising polynucleotides of the invention, polypeptides of the invention, fragments thereof, or modulators thereof.

“Antibody-dependent cell cytotoxicity” (ADCC) is a form of cell mediated cytotoxicity in which an effector cell, such as a lymphocyte, NK cell, granulocyte, neutrophil, eosinophil, basophil, mast cell, or macrophage, mediates the killing of a cell to which an antibody is attached. ADCC can involve humoral and/or cell-dependent mechanisms.

“Complement dependent cytotoxicity” (CDC) is an adverse effect on a cell that can result from activation of the complement pathway. It includes actions mediated through the classical complement pathway.

The term “binds specifically,” in the context of antibody binding, refers to high avidity and/or high affinity binding of an antibody to a specific epitope. Hence, an antibody that binds specifically to one epitope (a “first epitope”) and not to another (a “second epitope”) is a “specific antibody.” An antibody specific to a first epitope may cross react with and bind to a second epitope if the two epitopes share homology or other similarity.

The term “binds specifically,” in the context of a polynucleotide, refers to hybridization under stringent conditions. Conditions that increase stringency of both DNA/DNA and DNA/RNA hybridization reactions are widely known and published in the art. See, for example, Sambrook, J., et al. (2000) Molecular Cloning, A Laboratory Manual. 3^{nd} ed. Cold Spring Harbor Laboratory Press.

An “isolated,” “purified,” “substantially isolated,” or “substantially purified” molecule (such as a polypeptide, polynucleotide, or antibody) is one that has been manipulated to exist in a higher concentration than in nature. For example, a subject antibody is isolated, purified, substantially isolated, or substantially purified when at least 10%, or 20%, or 40%, or 50%, or 70%, or 90% of non-subject-antibody materials with which it is associated in nature have been removed. As used herein, an “isolated,” “purified,” “substantially isolated,” or “substantially purified” molecule includes recombinant molecules.

A “host cell” is an individual cell or cell culture which can be or has been a recipient of any recombinant vector(s) or isolated polynucleotide. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention may be called a “recombinant host cell.”

“Patient,” “individual,” “host,” and “subject” are used interchangeably herein to refer to mammals, including, but not limited to, rodents, simians, humans, felines, canines, equines, bovines, porcines, ovines, caprines, mammalian laboratory animals, mammalian farm animals, mammalian sport animals, and mammalian pets.

A “patient sample” is any biological specimen derived from a patient; the term includes, but is not limited to, biological fluids such as blood, serum, plasma, urine, cerebrospinal fluid, tears, saliva, lymph, dialysis fluid, lavage fluid, semen, and other liquid samples, as well as cell and tissues of biological origin. The term also includes cells or cells derived therefrom and the progeny thereof, including cells in culture, cell supernatants, and cell lysates. It further includes organ or tissue culture-derived fluids, tissue biopsy samples, tumor biopsy samples, stool samples, and fluids extracted from physiological tissues, as well as cells dissociated from solid tissues, tissue sections, and cell lysates. This definition encompasses samples that have been manipulated in any way after their procurement, such as by treatment with reagents, solubilization, or enrichment for certain components, such as polynucleotides or polypeptides. Also included in the term are derivatives and fractions of patient samples. A patient sample may be used in a diagnostic, prognostic, or other monitoring assay.

The term “receptor” refers to a polypeptide that binds to a specific ligand. The ligand is usually an extracellular molecule which, upon binding to the receptor, usually initiates a cellular response such as initiation of a signal transduction pathway.

The term “ligand” refers to a molecule that binds to a specific site on another molecule, usually a receptor.

The term “modulate” refers to the production, either directly or indirectly, of an increase or a decrease, a stimulation, inhibition, interference, or blockage in a measured activity when compared to a suitable control. A “modulator” of a polypeptide or polynucleotide or an “agent” are terms used interchangeably herein to refer to a substance that affects, for example, increases, decreases, stimulates, inhibits, interferes with, or blocks a measured activity of the polypeptide or polynucleotide, when compared to a suitable control.

The term “agonist” refers to a substance that mimics or enhances the function of an active molecule. Agonists include, but are not limited to, antibodies, growth factors, cytokines, lymphokines, small molecule drugs, hormones, and neurotransmitters, as well as analogues and fragments thereof.

The term “antagonist” refers to a molecule that interferes with the activity or binding of another molecule such as an agonist, for example, by competing for the one or more binding sites of an agonist, but does not induce an active response.

An “antibody modulator of a polypeptide” is a modulator that recognizes and binds specifically to the polypeptide. Such an antibody may, for example, induce ADCC, CDC, or apoptosis, or may block or otherwise interfere with the activity of a polypeptide.

“Modulating a level of an active subject polypeptide” includes increasing or decreasing, blocking, or interfering with the expression or activity of a subject polypeptide, increasing or decreasing a level of an active polypeptide, and increasing or decreasing the level of mRNA encoding an active subject polypeptide. Modulation can occur directly or indirectly.

The term “overexpressed” or “highly expressed” refers to a state wherein there exists any measurable increase in expression over normal or baseline levels. For example, a molecule that is overexpressed in a disease is one that is manifest in a measurably higher level in the presence of the disease than in the absence of the disease. Such an increase can be, for example, at least two-fold, or at least three-fold, or more.

“Treatment,” as used herein, covers any administration or application of remedies for disease in a mammal, including a human, and includes inhibiting the disease, arresting its development, or relieving the disease, for example, by causing regression, or restoring or repairing a lost, missing, or defective function; or stimulating an inefficient process.

“Prophylaxis,” as used herein, includes preventing a disease from occurring or recurring in a subject that may be predisposed to the disease but has not yet been diagnosed with the disease. Treatment and prophylaxis can be administered to an organism, or to a cell in vivo, in vitro, or ex vivo, and the cell subsequently administered to the subject.

A “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

A “composition” herein refers to a composition that usually contains a carrier, such as a pharmaceutically acceptable carrier or excipient that is conventional in the art and which is suitable for administration into a subject for therapeutic, diagnostic, or prophylactic purposes. It may include a cell culture in which the polypeptide or polynucleotide is present in the cells or in the culture medium. For example, compositions for oral administration can form solutions, suspensions, tablets, pills, capsules, sustained release formulations, oral rinses, or powders.

“Disease” refers to any condition, infection, disorder, or syndrome that requires medical intervention or for which medical intervention is desirable. Such medical intervention can include treatment, diagnosis, and/or prevention.

“Tumor” refers to any abnormal cell or tissue growth, whether malignant, pre-malignant, or non-malignant.

“Cancer” is any malignant growth or tumor. Cancer is characterized by the loss of normal control mechanisms for cell growth, including for cell proliferation. Cancer cells may or may not invade the surrounding tissue and, hence, may or may not metastasize to new body sites. Cancer encompasses carcinomas, which are cancers of epithelial cells; carcinomas include squamous cell carcinomas, adenocarcinomas, melanomas, and hepatomas. Cancer also encompasses sarcomas, which are tumors of mesenchymal origin; sarcomas include osteogenic sarcomas, leukemias, and lymphomas. Cancers may involve one or more neoplastic cell type.

### Target Molecules

Both the nucleic acid molecules encoding the proteins described below as well as the encoded proteins serve as target molecules of the invention. These target molecules are overexpressed in tumor tissues as compared to normal tissues, as set forth in the Figures and Examples. Each of the targets corresponds to a probe that exhibited a “hit” when hybridized to the cRNA on a FivePrime microarray chip, leading to their identification.

Among the targets of the invention is the semaphorin 4B protein which is encoded by the SEMA4B gene. Semaphorin 4B includes an extracellular domain, a transmembrane domain, and a short cytoplasmic domain (http://www.ncbi.nlm.nih. gov/entrez/viewer.fcgi?db=protein&val=29840873). Semaphorin 4B is a member of the semaphorin family of proteins that have been described as intercellular signaling proteins with regulatory roles in, for example, development, regeneration, and immune function (Raper, et al., 2000). Some semaphorins are secreted proteins, while others are transmembrane proteins. Semaphorins can act as ligands for neuropilins/plexins, and can act as receptors themselves. Semaphorins are thus capabale of mediating bidirectional signaling. Various semaphorin family members have been reported to be involved in growth cone guidance and collapse during development, in co-stimulating lymphocyte proliferation, in induction of neuronal apoptosis, and as mediators of chemotactic or chemorepulsive activity for neuritis. In one report, semaphorin 4D mediated activation of the c-met protooncogene gene product. In another report, semaphorin 4D induced chemotaxis and tubulogenesis in endothelial cells and enhanced blood vessel formation in an in vivo mouse model. While some semaphorins seem capable of potentiating cancer and tumorigenesis, others may have the opposite effect. Semaphorin 3F, for example, has been reported to inhibit VEGF and bFGF induced proliferation of HUVEC cells, while another report suggests that semaphorin 3F may suppress NGF-induced activation of PI3K-AKT-MEK-ERK pathways.

Among the targets of the invention is the KIAA0152 protein encoded by the KIAA0152 gene. This protein is predicted to include an extracellular domain, a transmembrane domain, and a very short cytoplasmic domain (http://www.ncbi.nlm. nih.gov/entrez/viewer.fcgi?db=protein&val=2495712). The biological function of KIAA0152 is currently unknown.

Among the targets of the invention is the nectin 4 protein which is encoded by the PVRLA gene. Nectin 4 includes an extracellular domain, a transmembrane domain, and a cytoplasmic domain (Takai and Nakanishi, 2003). Nectin 4 is a member of the nectin family of proteins which consists of calcium-independent immunoglobulin-like intercellular adhesion molecules. Some family members also serve as virus receptors. Nectin 4 can interact with nectin1a. The nectin 4/nectin1a complex can be localized to adherens junctions together with the E-cadherin tumor suppressor molecule. The cytoplasmic tail of nectin 4 can interact with afadins, which in turn interacts with the actin cytoskeleton and with signaling molecules such as the Ras protooncogene. Ras plays a role in regulating cell proliferation, and dysregulation of Ras function is implicated in the development and potentiation of cancer. A shed form of nectin 4 may be found in serum of patients with metastatic breast cancer.

Among the targets of the invention is the IgSF9 (immunoglobulin superfamily member 2) protein which is encoded by the IgSF9 gene. IgSF9 includes an extracellular domain, a transmembrane domain, and a cytoplasmic domain (Doudney et al., 2002). IgSF9 is a member of the immunoglobulin superfamily. Members of this superfamily have diverse physiologic functions, including regulation of cell growth and proliferation, cell activation, cell adhesion, cell migration, and cell survival.

### Microarray Hybridization

The nucleic acid molecules and encoded proteins described in the Tables, Figures, and Sequence Listing, may serve as targets of modulators, including antibodies, that affect their activity, the activity of cells expressing them, or the activity of secondary target cells. They may also serve as target molecules for the selection and production of such modulators, including antibodies. These modulators of the invention can be used to diagnose or treat diseases, including cancers, in which a target molecule was expressed at higher than normal levels.

In Example 1 and FIGS. 5-21, IgSF9 probes hybridized at higher intensities to selected tumor tissues than to normal tissues. Expression profiling analysis with the proprietary Five Prime chip using a probe (PRB103989_s_at) against the cytoplasmic domain of IgSF9 (FIG. 5) revealed that IgSF9 mRNA was overexpressed in lung cancers compared to normal lung tissues, in breast cancers compared to normal breast tissues, in prostate cancers compared to normal prostate tissues, and in pancreatic cancers compared to normal pancreas tissues (FIGS. 6-10). Furthermore, IgSF9 was not expressed at detectable levels in most normal tissues (FIG. 11).

Expression profiling analysis with the Affymetrix U133 chip revealed additionally that IgSF9 mRNA was overexpressed in various other cancers as compared to the respective normal tissues, including malignant cancers of the bladder, endometrium, skin, kidney, liver, ovary, breast, and thyroid gland (FIGS. 12-21).

Similar microarray expression analyses were performed with probes against nectin 4, KIAA0152, and semaphorin 4B.

In Example 3 and FIGS. 24-39, nectin 4 probes hybridized at higher intensities to selected tumor tissues than to normal tissues. Expression profiling analysis with the proprietary Five Prime chip using a probe (PRB103018_s_at) against nectin 4 (FIG. 24) revealed that nectin 4 mRNA was overexpressed in lung adenocarcinomas and lung squamous cell carcinomas compared to normal lung tissues, in colon/colorectal cancers compared to normal colon/colorectal tissues, in prostate cancers compared to normal prostate tissues, and in pancreatic cancers compared to normal pancreas tissues (FIGS. 25-28). Furthermore, nectin 4 was not expressed at detectable levels in most normal tissues, including important tissues such as heart, liver and kidney (FIG. 29).

Expression profiling analysis with the Affymetrix U133 chip revealed additionally that nectin 4 mRNA was overexpressed in various other cancers as compared to the respective normal tissues, including malignant cancers of the bladder, endometrium, kidney, liver, ovary, breast, and thyroid gland (FIGS. 30-39). In Example 4 and FIGS. 40-56 KIAA0152 probes hybridized at higher intensities to selected tumor tissues than to normal tissues. Expression profiling analysis with the proprietary Five Prime chip using a probe (PRB105610_at) against KIAA0152 (FIG. 40) revealed that KIAA0152 mRNA was overexpressed in lung cancers compared to normal lung tissues, in colon/colorectal cancers compared to normal colon/colorectal tissues, in breast cancers compared to normal breast tissues, in prostate cancers compared to normal prostate tissues, and in pancreatic cancers compared to normal pancreas tissues (FIGS. 41-45). KIAA0152 was also expressed at relatively low levels in many but not all normal tissues (FIG. 46).

Expression profiling analysis with the Affymetrix U133 chip revealed additionally that KIAA0152 mRNA was overexpressed in various other cancers as compared to the respective normal tissues, including malignant cancers of the bladder, brain, kidney, liver, ovary, stomach, breast, skin, stomach, and thyroid gland (FIGS. 47-56).

In Example 6 and FIGS. 60-75 semaphorin 4B probes hybridized at higher intensities to selected tumor tissues than to normal tissues. Expression profiling analysis with the proprietary Five Prime chip using a probe (PRB101227_s_at) against semaphorin 4B (FIG. 60) revealed that semaphorin 4B mRNA was overexpressed in lung adenocarcinomas and lung squamous cell carcinomas compared to normal lung tissues, in colon/colorectal cancers compared to normal colon/colorectal tissues, in prostate cancers compared to normal prostate tissues, and in pancreatic cancers compared to normal pancreas tissues (FIGS. 61-64). Furthermore, semaphorin 4B was expressed at low or undetectable levels in most normal tissues, including important tissues such as heart, liver and kidney (FIG. 65). Expression profiling analysis with the Affymetrix U133 chip revealed additionally that semaphorin 4B mRNA was overexpressed in various other cancers as compared to the respective normal tissues, including malignant cancers of the bladder, brain, endometrium, liver, ovary, stomach, and thyroid gland (FIGS. 66-75).

Microarray hybridization was performed under high stringency conditions. Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where 1×SSC is 0.15 M NaCl and 15 mM citrate buffer); and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. For example, high stringency conditions include hybridization in 50% formamide, 5×SSC, 0.2 μg/μl poly(dA), 0.2 μg/μl human cot1 DNA, and 0.5% SDS, in a humid oven at 42° C. overnight, followed by successive washes in 1×SSC, 0.2% SDS at 55° C. for 5 minutes, followed by washing at 0.1×SSC, 0.2% SDS at 55° C. for 20 minutes. Further examples of high stringency conditions include hybridization at 50° C. and 0.1×SSC (15 mM sodium chloride/1.5 mM sodium citrate); overnight incubation at 42° C. in a solution containing 50% formamide, 1×SSC, 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. High stringency conditions also include aqueous hybridization (for example, free of formamide) in 6×SSC, 1% sodium dodecyl sulfate (SDS) at 65° C. for about 8 hours (or more), followed by one or more washes in 0.2×SSC, 0.1% SDS at 65° C. Highly stringent hybridization conditions are hybridization conditions that are at least as stringent as any one of the above representative conditions. Other stringent hybridization conditions are known in the art and can also be employed to identify nucleic acids of this particular embodiment of the invention.

Conditions of reduced stringency, suitable for hybridization to molecules encoding structurally and functionally related proteins, or otherwise serving related or associated functions, are the same as those for high stringency conditions but with a reduction in temperature for hybridization and washing to lower temperatures (for example, room temperature or about 22° C. to 25° C.). For example, moderate stringency conditions include aqueous hybridization (for example, free of formamide) in 6×SSC, 1% SDS at 65° C. for about 8 hours (or more), followed by one or more washes in 2×SSC, 0.1% SDS at room temperature. Low stringency conditions include, for example, aqueous hybridization at 50° C. and 6×SSC and washing at 25° C. in 1×SSC.

The specificity of a hybridization reaction allows any single-stranded sequence of nucleotides to be labeled with a radioisotope or chemical and used as a probe to find a complementary strand, even in a cell or cell extract that contains millions of different DNA and RNA sequences. Probes of this type are widely used to detect the nucleic acids corresponding to specific genes, both to facilitate the purification and characterization of the genes after cell lysis and to localize them in cells, tissues, and organisms.

### Real Time PCR

The microarray hybridization results were validated and further characterized by quantitative real time-PCR, as set forth in Example 2, 5 and 7. Specific primers and probes for each target were designed and tested for their specificity (FIGS. 22, 57, and 76). In contrast to the microarray probes, which targeted the 3′-UTR non-coding region or a region of the mRNA encoding a carboxy-terminal portion of the proteins, the RT-PCR primer-probes targeted regions of the mRNA encoding amino-terminal portions of the proteins. The results obtained with these probes confirm that the expression profiles observed by the microarray hybridizations can be extrapolated to the mRNA region that encodes the extracellular domains, thus providing evidence that tumor cells produce full length and not truncated forms of the target proteins.

Quantitative RT-PCR (Taqman) analysis of lung squamous cell carcinoma and normal lung tissues confirmed the overexpression of IgSF9 in lung squamous cell carcinoma (FIG. 25). Quantitative RT-PCR (Taqman) analysis of prostate cancer, normal prostate tissues, and other normal tissues confirmed the overexpression of KIAA0152 in a fraction of prostate cancers and the detection of KIAA0152 in some other normal tissues at relatively low levels (FIGS. 58 and 59). Quantitative RT-PCR (Taqman) analysis of lung squamous cell carcinoma and normal lung tissues also confirmed the overexpression of semaphorin 4B in a fraction of lung squamous cell carcinomas (FIG. 77).

### Antibody Targets

IgSF9, nectin 4, KIAA0152, and semaphorin 4B are therapeutic targets for cancer, since they are transmembrane proteins overexpressed on the surface of cancer tissues compared to normal tissues. Antibodies are particularly suited to be used as therapeutic agents when their targets are transmembrane proteins expressed on the surface of cancer cells. Thus, in one aspect of the invention, the nucleic acids and proteins are antibody targets or markers or biomarkers identified by binding to an antibody.

Antibodies binding to the extracellular domains of the identified targets are therapeutic for cancers, including lung squamous cell carcinoma, lung adenocarcinoma, colon/colorectal cancer, bladder cancer, pancreatic cancer, stomach cytotoxic and others. Such antibodies can be used as monotherapy if they mediate ADCC or CDC, or if they modify the underlying function of the target molecule. Such antibodies can also be used in the form of antibody conjugates to directly deliver cancer agents with a lethal effect on the tumor. Such agents include radionuclides, toxins, and chemotherapeutics.

Such antibodies can also be used in combination with standard chemotherapeutic or radiation regimens to treat cancers. In this case, the antibodies can act to sensitize the cancer cells to chemotherapy or radiation, allowing for more efficient tumor killing. Alternatively, the antibodies can act in synergy with chemotherapy or radiation treatment, such that lower doses of either may be used, decreasing the overall toxicity to normal cells while maintaining equivalent efficacy in treating the tumor.

Antibodies having a therapeutic effect on cancers include those binding to amino acids sequences involved in function of the target proteins, including functionally important sites in the extracellular domains that are accessible for antibody binding. Examples of epitopes targeted by the therapeutic antibodies of the invention are provided in the Sequence Listing.

### Protein Families

The sequences of the invention encompass nucleic acids and polypeptides with different structures and functions, embodied in different molecular domains. They can encode or comprise polypeptides belonging to different protein families, for example, those described by the Pfam database and those having different biologically meaningful motifs, as described by the Prosite database. The Pfam system is an organization of protein sequence classification and analysis, based on conserved protein domains; it can be publicly accessed in a number of ways, for example, at http://Pfam.wustl.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families (Bateman et al., 2002). The Prosite system provides a classification of sequence motifs that are described as patterns or profiles, in conjunction with the SWISS-PROT protein database (Sigrist et al., 2002). It can be accessed publicly, for example, at http://www.expasy.org/prosite (Copyright© 2005 Oxford University Press).

Sequences of the invention can encode or be comprised of more than one Pfam or Prosite. Sequences encompassed by the invention include, but are not limited to, the polypeptide and polynucleotide sequences of the molecules shown in the Figures, Tables and Sequence Listing and corresponding molecular sequences found at all developmental stages of an organism. Sequences of the invention can comprise genes or gene segments designated in the Figures, Tables, and Sequence Listing, and their gene products, i.e., RNA and polypeptides. They also include variants of those presented in the Figures, Tables, and Sequence Listing that are present in the normal physiological state, for example, variant alleles such as SNPs, splice variants, as well as variants that are affected in pathological states, such as disease-related mutations or sequences with alterations that lead to pathology, and variants with conservative amino acid changes. Some sequences of the invention are categorized below with respect to one or more protein family. Any given sequence can belong to one or more than one category.

Semaphorin 4B comprises Pfam and/or Prosite domains, including SEMA and PSI domains. The Sema domain is a 500 amino acid domain that serves as a receptor recognition and binding module and is found near the N-terminus of eukaryotic and viral proteins. The Sema domain is characterized by a conserved set of cysteine residues, which form four disulfide bonds to stabilize its structure (http://pfam.wustl.edu/cgi-bin/getdesc?acc=PF01403). The PSI domain is a cysteine-rich repeat with unknown function that is found in several different extracellular receptors, including Plexin (http://pfam.wustl.edu/cgi-bin/getdesc?name=PSI). Nectin-4 comprises Pfam and/or Prosite domains, including V-set and Ig domains. The ig (immunoglobulin-like) domain is a very widespread domain that can be considered as an heterogeneous group built on a common fold. The well conserved fold consists of a β-sandwich formed of 7-10 strands in 2 sheets with a Greek-key topology. All ig domains appear to be involved in protein-protein and protein-ligand interactions (http://pfam.wustl.edu/cgi-bin/getdesc?name=ig). The V-set domain is found in antibodies as well as in various other proteins (http://pfam.wustl.edu/cgi-bin/getdesc?name=V-set).

Immunoglobulin superfamily member 9 comprises Pfam and/or Prosite domains, including previously discussed V-set and Ig domains as well as fn3 domains. The fn3 domain is a fibronectin type III repeat region of approximately 100 amino acid residues, different tandem repeats of which contain binding sites for DNA, heparin and the cell surface. Fn3 domains are found in many different protein families, the majority of which are cell surface binding proteins, receptor protein tyrosine kinases, or cytokine receptors (http://pfam.wustl.edu/cgi-bin/getdesc?name=fn3).

### Polypeptide Expression

The target polypeptides described herein can be expressed using methods known in the art. The polymerase chain reaction, cell-based methods, and cell-free methods are all suitable for producing polypeptides of the invention. The use of the polymerase chain reaction has been described (Saiki et al., 1985) and current techniques have been reviewed (Sambrook et al., 2000; McPherson et al. 2000; Dieffenbach and Dveksler, 1995). Cell-based methods generally involve introducing a nucleic acid construct into a host cell in vitro and culturing the host cell under conditions suitable for expression, then harvesting the polypeptide, either from the culture medium or from the host cell, (for example, by disrupting the host cell), or both, as described in detail above. The invention also provides methods of producing a polypeptide using cell-free in vitro transcription/translation methods, which are well known in the art.

The target polypeptides can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography (HPLC) can be employed for purification. Target polypeptides include products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Typically, a heterologous polypeptide, whether modified or unmodified, may be expressed as described above, or as a fusion protein, and may include not only secretion signals, but also a secretory leader sequence. A secretory leader sequence of the invention may direct certain proteins to the ER. The ER separates the membrane-bound proteins from other proteins. Once localized to the ER, proteins can be further directed to the Golgi apparatus for distribution to vesicles; including secretory vesicles; the plasma membrane, lysosomes, and other organelles.

Proteins targeted to the ER by a secretory leader sequence can be released into the extracellular space as a secreted protein. For example, vesicles containing secreted proteins can fuse with the cell membrane and release their contents into the extracellular space in a process called exocytosis. Exocytosis can occur constitutively or in response to a triggering signal. In the latter case, the proteins may be stored in secretory vesicles (or secretory granules) until exocytosis is triggered. Similarly, proteins residing on the cell membrane can also be secreted into the extracellular space by proteolytic cleavage of a linker holding the protein to the membrane.

Additionally, peptide moieties and/or purification tags may be added to the polypeptide to facilitate purification. Such regions may be removed prior to final preparation of the polypeptide. The addition of peptide moieties to polypeptides to engender secretion or excretion, to improve stability, and to facilitate purification, among other reasons, are familiar and routine techniques in the art. Suitable purification tags include, for example, V5, polyhistidines, avidin, and biotin.

Protein expression systems known in the art can produce fusion proteins that incorporate the polypeptides of the invention. Target protein fusions can facilitate production, secretion, and/or purification. They can confer a longer half-life when administered to an animal. Fusion partners suitable for use in the invention include, for example, polyethylene glycol (PEG), fetuin, human serum albumin, immunoglobulin F_{c}, and/or one or more of their fragments. Such modified polypeptides can show, for example, enhanced activity or increased stability. In addition, they may be purified in higher yields and show better solubility than the corresponding natural polypeptide, at least under certain purification and storage conditions.

### Kits

Detection of cancer cell-specific biomarkers provides an effective cancer screening strategy. Early detection provides not only early diagnosis, but also the ability to screen for polymorphism and detect post-operative residual tumor cells and occult metastases, an early indicator of tumor recurrence. Early detection of cancer cell-specific biomarkers can thus improve survival in patients before diagnosis, while undergoing treatment, and while in remission.

IgSF9, nectin 4, KIAA0152, and Semaphorin 4B are overexpressed in cancer patients. Since these polypeptides are not normally expressed at high levels in healthy, non-pregnant individuals, their elevated presence can be used as a diagnostic or prognostic marker for diseases, including cancer, such as in identifying a patient population appropriate for treatment. Diagnostic antibodies can be used in a number of ways, including but not limited to ELISA, Western blot, immunofluorescence, or immunohistochemistry, for these purposes.

The invention provides methods for diagnosing disease states based on the detected presence and/or level of target polynucleotides, polypeptides, or antibodies in a biological sample, and/or the detected presence and/or level of biological activity of the polynucleotide or polypeptide. These detection methods can be provided as part of a kit. Thus, the invention further provides kits for detecting the presence and/or a level of a polynucleotide, polypeptide, or antibody of interest in a biological sample, or for detecting the presence and/or a level of biological activity of a polynucleotide or polypeptide in a biological sample.

Where the kit provides for polynucleotide detection, it can include one or more nucleotide sequences that hybridize specifically to a target nucleotide sequence of interest. Examples of such nucleotide sequences are described in the Examples and provided in the Sequence Listing.

Where the kit provides for polypeptide detection, it can include one or more specific antibodies. In some embodiments, the antibody specific to the polypeptide of interest is detectably labeled. In other embodiments, the antibody specific to the polypeptide is not labeled; instead, a second, detectably labeled antibody is provided that binds to the specific antibody. The kit may further include blocking reagents, buffers, and reagents for developing and/or detecting the detectable marker. The kit may further include instructions for use, controls, and interpretive information.

The invention also provides for therapeutic kits with unit doses of an active agent. In some embodiments, the agent is provided in oral or injectable doses, as described in further detail below. Such kits can comprise containers containing the unit doses and an informational package insert describing the use and attendant benefits of the drugs in treating a condition of interest.

### Panel

The tumor targets or markers listed in the Tables can be used separately or in combination for diagnostic purposes, for example, in a panel that comprises two or more of such. It is expected that almost all lung, prostate or colon cancers will overexpress at least one of these genes, and that combining these markers into a panel will provide an effective screen for certain cancers.

### Gene Expression of the Target Molecules in Cancer

Genes that are uniquely or differentially expressed in cancerous cells or tissues may potentially serve as cancer cell markers in bodily fluids, for example serum, or in cancer cells or tissues. A reliable marker must be specific to cancer, and expressed only when the patient has cancer.

The present invention utilized probes that were designed by and purchased from Affymetrix, Inc. (Santa Clara, Calif.). Eleven matching probes, each about 25 nucleotides in length, were designed to correspond to a target sequence for selected clones from tumor or normal tissues. Eleven other target probes were designed for each target sequence, each with a single nucleotide mismatch. These probes were spotted on a microarray chip (i.e., the FivePrime Chip) and hybridized to cDNA made complementary to RNA from tumor tissues or normal tissues. After hybridization, using an Affymetrix protocol, the results were read, again using Affymetrix's equipment and protocol. Results were reported as being present/absent and as a value representing intensity of hybridization. For example, if the ratio of intensity of a matched probe and a mismatched probe was high, this would generate a “present” call. If such a ratio was low, this would generate an “absent” call, reflecting non-specific hybridization. Thus, even if two of the 11 probes for a target sequence lit up, for example, with high intensity but nine of the remaining 11 probes were considered “absent,” this probe set would not be considered a “hit.” We considered a probe set a “hit” when the probe set was “present” and when the intensity was high in tumor tissues and low in normal tissues. The targets of the invention were identified by this method as positive hits.

### Active Agents (or Modulators)

The nucleic acid, polypeptide, and modulator compositions of the subject invention find use as therapeutic agents in situations where one wishes to modulate an activity of a subject polypeptide, or to provide or inhibit the activity at a particular anatomical site. The active agents of the invention are useful in the diagnosis and treatment of proliferative diseases, for example, lung, breast, bladder, pancreatic, ovarian, prostate, skin, kidney, liver, endometrial, thyroid, and stomach cancer; and psoriasis. Modulators of the invention include, for example, polypeptide variants, whether agonist or antagonist; aptamers or antibodies, whether agonist or antagonist; soluble receptors, usually antagonists; small molecule drugs, whether agonist or antagonist; interfering RNAs (RNAi), usually antagonists; antisense molecules, usually antagonists; and ribozymes, usually antagonists.

In some embodiments, modulators of the invention bind to subject polypeptides. They may directly modulate the targeted subject polypeptides as a result of their binding. They may also indirectly modulate a biological process by interacting with the targeted subject polypeptides. Modulators of the invention may bind to subject polypeptides in a manner that may or may not interfere with the function of the targeted molecules; the modulator may be therapeutically efficacious whether or not the modulator interferes with the function of the targeted molecules. For example, a modulator may form a complex with a subject polypeptide and an effector molecule or effector cell.

In some embodiments, an agent is a subject polypeptide which is administered to an individual. In some embodiments, an agent is an antibody specific for a subject target polypeptide. In some embodiments, an agent is a chemical compound, such as a small molecule, that may be useful as an orally available drug. Such modulation may include the recruitment of other molecules that directly effect the modulation. For example, an antibody that modulates the activity of a subject polypeptide that is a receptor on a cell surface may bind to the receptor and fix complement, activating the complement cascade and result in lysis of the cell. An agent which modulates a biological activity of a subject polypeptide or polynucleotide increases or decreases the activity or binding at least about 10%, at least about 15%, at least about 20%, at least about 25%, at least about 50%, at least about 100%, or at least about 2-fold, at least about 5-fold, or at least about 10-fold or more when compared to a suitable control.

The invention provides a method of identifying a modulator of the biological activity of a polypeptide of the invention by providing at least one polypeptide chosen from the sequences listed in the Tables, Figures, and Sequence Listing, and active fragments thereof; allowing at least one agent to contact the polypeptide; and selecting an agent that binds the polypeptide or affects the biological activity of the polypeptide. In an embodiment, the modulator is an antibody.

The invention provides compositions comprising modulators obtained by this method and a pharmaceutically acceptable carrier. For example, the invention provides modulator compositions comprising a pharmaceutically acceptable carrier and a modulator, wherein the modulator is a soluble receptor that competes for ligand binding or cofactor binding to an isolated polypeptide comprising an amino acid sequence chosen from the Tables, Figures, and Sequence Listing, and biologically active fragments thereof. The invention also provides a modulator composition comprising a pharmaceutically acceptable carrier and a modulator, wherein the modulator is an extracellular fragment that competes for ligand binding or cofactor binding to an isolated polypeptide comprising an amino acid sequence chosen from the Tables, Figures, and Sequence Listing, and biologically active fragments thereof.

### Antisense Oligonucleotides

In certain embodiments of the invention, the agent is an antisense molecule that modulates, and generally decreases or down regulates, polypeptide expression in a host (Agrawal et al., 1998; Hartmann et al., 1999; Phillips et al., 1999a; Phillips et al., 1999b; Stein et al., 1998). Accordingly, the invention provides a modulator composition comprising a pharmaceutically acceptable carrier and a modulator, wherein the modulator is an antisense molecule that inhibits the transcription or translation of an isolated polynucleotide or an isolated polypeptide comprising an amino acid sequence encoded by a polynucleotide chosen from the Tables, Figures, and Sequence Listing, and biologically active fragments thereof. The invention also provides a modulator composition comprising a pharmaceutically acceptable carrier and a modulator, wherein the modulator is a ribozyme that inhibits the transcription or translation of an isolated polynucleotide or an isolated polypeptide comprising an amino acid sequence encoded by a polynucleotide chosen from the Figures, Tables and Sequence Listing, and biologically active fragments thereof.

Antisense reagents of the invention include antisense oligonucleotides (ODN), i.e., synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit target gene expression through various mechanisms, for example, by reducing the amount of mRNA available for translation, through activation of RNase H, or steric hindrance. One or a combination of antisense molecules can be administered, where a combination can comprise multiple different sequences.

Antisense molecules can be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides can be chemically synthesized by methods known in the art (Wagner et al., 1993; Milligan et al., 1993). Antisense oligonucleotides will generally be at least about 7, at least about 12, or at least about 20 nucleotides in length, and not more than about 500, not more than about 50, or not more than about 35 nucleotides in length, where the length is governed by efficiency of inhibition, and specificity, including absence of cross-reactivity, and the like. Short oligonucleotides, of from about 7 to about 8 bases in length, can be strong and selective inhibitors of gene expression (Wagner et al., 1996).

A specific region or regions of the endogenous sense strand of target mRNA sequence is chosen to be complemented by the antisense sequence. Selection of a specific sequence for the oligonucleotide can use an empirical method, where several candidate sequences are assayed for inhibition of expression of the target gene in an in vitro or animal model. As noted above, a combination of sequences can also be used, where several regions of the mRNA sequence are chosen for antisense complementation.

As an alternative to antisense inhibitors, catalytic nucleic acid compounds, for example, ribozymes, or antisense conjugates can be used to inhibit gene expression. Ribozymes can be synthesized in vitro and administered to the patient, or can be encoded in an expression vector, from which the ribozyme is synthesized in the targeted cell (WO 9523225; Beigelman et al., 1995). Examples of oligonucleotides with catalytic activity are described in WO 9506764. Conjugates of antisense ODN with a metal complex, for example, terpyridyl Cu(II), capable of mediating mRNA hydrolysis are described in Bashkin et al., 1995.

### Interfering RNA (RNAi)

In some embodiments, the active agent is an interfering RNA (RNAi). RNA interference provides a method of silencing eukaryotic genes. Use of RNAi to reduce a level of a particular mRNA and/or protein is based on the interfering properties of RNA, e.g., double-stranded RNA (dsRNA), derived from the coding regions of a gene. The technique is an efficient high-throughput method for disrupting gene function (O'Neil, 2001). RNAi can also help identify the biochemical mode of action of a drug and to identify other genes encoding products that can respond or interact with specific compounds. Accordingly, the invention provides a modulator composition comprising a pharmaceutically acceptable carrier and a modulator, wherein the modulator is an RNAi molecule that inhibits the transcription or translation of an isolated polynucleotide or an isolated polypeptide comprising an amino acid sequence encoded by a polynucleotide chosen from the Tables, Figures, and Sequence Listing, and biologically active fragments thereof.

In one embodiment of the invention, complementary sense and antisense RNAs derived from a substantial portion of a subject polynucleotide are synthesized in vitro. The resulting sense and antisense RNAs are annealed in an injection buffer, and the double-stranded RNA injected or otherwise introduced into the subject, for example, in food or by immersion in buffer containing the RNA (Gaudilliere et al., 2002; O'Neil et al., 2001; WO99/32619). In an embodiment, dsRNA derived from a subject polynucleotide is generated in vivo by simultaneously expressing both sense and antisense RNA from appropriately positioned promoters operably linked to sequences in both sense and antisense orientations. The expressed sequences can be derived from the translated portion of a mRNA encoding a polypeptide of the invention, or from the 3′ or 5′ untranslated regions of such a mRNA.

### Aptamers

Another suitable agent for modulating an activity of a subject polypeptide is an aptamer. Aptamers of the invention include both nucleotide and peptide aptamers that bind to polypeptides comprising an amino acid sequence encoded by a polynucleotide chosen from the Figures, Tables, and Sequence Listing, and biologically active fragments thereof. Nucleotide aptamers of the invention include double stranded DNA and single stranded RNA molecules. Peptide aptamers are peptides or small polypeptides that act as dominant inhibitors of protein function. Peptide aptamers specifically bind to target proteins, blocking their functional ability (Kolonin and Finley, 1998). Due to the highly selective nature of peptide aptamers, they can be used not only to target a specific protein, but also to target specific functions of a given protein (for example, a signaling function). Further, peptide aptamers can be expressed in a controlled fashion by use of promoters which regulate expression in a temporal, spatial, or inducible manner. Peptide aptamers act dominantly, therefore, they can be used to analyze proteins for which loss-of-function mutants are not available. Aptamers of the invention may bind nucleotide cofactors (Latham et al., 1994).

Peptide aptamers that bind with high affinity and specificity to a target protein can be isolated by a variety of techniques known in the art. Peptide aptamers can be isolated from random peptide libraries by yeast two-hybrid screens (Xu et al., 1997). They can also be isolated from phage libraries (Hoogenboom et al., 1998) or chemically generated peptides/libraries.

### Peptides and Modified Peptides

Polypeptides of the invention include full length proteins that include a signal peptide or leader sequence, if present, or a mature protein after cleavage of the signal peptide or leader sequence, the signal peptide or leader sequence, or portions or fragments of the full length or mature protein. Also included in this term are biologically active variations of naturally occurring proteins, where such variations are homologous or substantially similar to the naturally occurring protein, as well as corresponding homologs from different species. Variants of polypeptide sequences may include insertions, additions, deletions, or substitutions compared with the subject polypeptides. Variants of polypeptide sequences include biologically active polymorphic variants.

In some embodiments of the present invention, the active agent is a peptide. Suitable peptides include peptides of from about 3 amino acids to about 50, from about 5 to about 30, or from about 10 to about 25 amino acids in length which may, but need not, correspond to the sequence of the naturally-occurring protein. In some embodiments, a peptide has a sequence of from about 7 amino acids to about 45, from about 9 to about 35, or from about 12 to about 25 amino acids of corresponding naturally-occurring protein. In some embodiments, a peptide exhibits one or more of the following activities: inhibits binding of a subject polypeptide to an interacting protein or other molecule; inhibits subject polypeptide binding to a second polypeptide molecule; inhibits a signal transduction activity of a subject polypeptide; inhibits an enzymatic activity of a subject polypeptide; or inhibits a DNA binding activity of a subject polypeptide.

Peptides of the invention can include naturally-occurring and non-naturally occurring amino acids. Peptides can comprise D-amino acids, a combination of D- and L-amino acids, and various “designer” or “synthetic” amino acids (for example, β-methyl amino acids, Cα-methyl amino acids, and Nα-methyl amino acids, etc.) to convey special properties. Additionally, peptides can be cyclic. Peptides can include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics can be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; α-turn inducing analogs; α-helix inducing analogs; γ-turn inducing analogs; Gly-Ala turn analogs; amide bond isostere; or tetrazol, and the like.

Peptides of the invention can be a depsipeptide, which can be linear or cyclic (Kuisle et al., 1999). Linear depsipeptides can comprise rings formed through S—S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. Cyclic depsipeptides contain at least one ring formed only through peptide or ester links, derived from hydroxy carboxylic acids.

Peptides of the invention can be cyclic or bicyclic. For example, the C-terminal carboxyl group or a C-terminal ester can be induced to cyclize by internal displacement of the (—OH) or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art.

A desamino or descarboxy residue can be incorporated at the terminal ends of the peptide, so that there is no terminal amino or carboxyl group, to decrease susceptibility to proteases or to restrict conformation. C-terminal functional groups include amide, amide lower alkyl, amide di (lower alkyl), lower alkoxy, hydroxy, and carboxy, and the lower ester derivatives thereof, and the pharmaceutically acceptable salts thereof.

In addition to the foregoing N-terminal and C-terminal modifications, peptides or peptidomimetics of the invention can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran, and dextran derivatives. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, (1995); Monfardini et al., (1995); U.S. Pat. Nos. 4,640,835; 4,496,689; 4,301,144; 4,670,417; 4,791,192; 4,179,337, or WO 95/34326.

### Soluble Receptors

Extracellular fragments of cell surface receptors can be soluble, and can modulate a target protein. These fragments can act as ligands for binding to receptors on cell surfaces in ligand/receptor interactions, and can modulate receptor interactions with other molecules and cellular activity downstream of the receptors. This modulation can trigger certain intracellular responses, such as inducing signal transduction, and can stimulate or inhibit cellular growth, proliferation, differentiation, adhesion, migration, or programmed cell death, or induce the production of other factors that, in turn, mediate such activities.

### Small Molecules

Small molecules, modulators such as those commonly used as therapeutic drugs, can be used as modulators in the invention. Small molecule agents include chemical compounds that bind the polypeptide and modulate activity of the polypeptide or cell containing the polypeptide. Small molecule modulators may permeate the cell, and/or may exert their action at the extracellular surface or on non-cellular structures, such as the extracellular matrix.

### Antibodies

Modulators of the invention may be antibodies. The invention provides isolated antibodies that specifically recognize, bind to, interfere with, and/or otherwise modulate the biological activity of at least one polypeptide comprising an amino acid sequence encoded by a polynucleotide chosen from the Tables, Figures, and Sequence Listing, and biologically active fragments thereof. For example, an antibody of the invention may be directed to a polypeptide comprising part or all of a non-transmembrane domain and/or an extracellular domain, a part or all of a Pfam or Prosite domain, or part or all of another functionally or structurally relevant domain.

Useful antibodies bind to or react with antigens comprising one or more discrete epitope or a combination of nested epitopes. A single antibody can interact with one or more epitopes. Further, the antibody can be used alone or in combination with different antibodies that recognize either a single or multiple epitopes.

The production and use of antibodies is well-known in the art (Harlow et al., 1998; Harlow and Lane, 1998; Howard et al., 2000). This antibody may be a monoclonal antibody; a polyclonal antibody; a single chain antibody; an antibody comprising a backbone of a molecule with an Ig domain or a T cell receptor backbone; a targeting antibody; a neutralizing antibody; a stabilizing antibody; an enhancing antibody; an antibody agonist; an antibody antagonist; an antibody that promotes endocytosis of a target antigen; a cytotoxic antibody; an antibody that mediates antibody dependent cell cytotoxicity; a human antibody; a non-human primate antibody; a non-primate animal antibody; an antibody that mediates complement dependent cytotoxicity.

An antibody of the invention can be a human antibody, a non-human primate antibody, a non-primate animal antibody, a rabbit antibody, a mouse antibody, a rat antibody, a sheep antibody, a goat antibody, a horse antibody, a porcine antibody, a cow antibody, a chicken antibody, a humanized antibody, a primatized antibody, and/or a chimeric antibody. Antibodies of the invention can comprise a cytotoxic antibody with one or more cytotoxic component chosen from a radioisotope, a microbial toxin, a plant toxin, and a chemical compound. The chemical compound can, for example, be chosen from doxorubicin and cisplatin. Antibodies of the invention include antigen binding fragments; fragments comprising a variable region of a heavy chain or a light chain of an immunoglobulin; fragments comprising a complementarity determining region or a framework region of an immunoglobulin; and one or more active fragments, analogues, and/or antagonists.

The isolated antibodies of the invention can be produced in a variety of cells. Host cells of the invention can be genetically modified to produce an antibody of the invention; these include bacterial cells, fungal cells, plant cells, insect cells, and mammalian cells. For example, isolated antibodies of the invention may be produced in yeast cells, Aspergillus cells, SF9 cells, High Five cells, cereal plant cells, tobacco cells, tomato cells, human kidney embryonic kidney 293 cells, myeloma cells, including mouse myeloma NS0 cells, human fetal Per C6 cells, and CHO cells.

In another aspect, the invention provides antibody targets. The polynucleotides and polypeptides described herein comprise nucleic acid and amino acid sequences that can be recognized by antibodies. A target sequence can be any polynucleotide or amino acid sequence of approximately eighteen or more contiguous nucleotides or approximately six or more amino acids. A variety of comparing means can be used to accomplish comparison of sequence information from a sample (for example, to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art. A target sequence includes an antibody target sequence, which refers to an amino acid sequence that can be used as an immunogen for injection into animals for production of antibodies or for screening against a phage display or antibody library for identification of binding partners.

The invention provides target structural motifs and target functional motifs, i.e., any rationally selected sequences or combination of sequences in which the sequence(s) are chosen based on a three-dimensional configuration formed upon the folding of the target motif, or on consensus sequences of regulatory or active sites. There are a variety of target motifs known in the art. Protein target motifs include, but are not limited to, enzyme active sites and signal sequences. Nucleic acid target motifs include, but are not limited to, hairpin structures, promoter sequences, and other expression elements, such as binding sites for transcription factors.

Antibodies of the invention bind specifically to their targets. Specific binding, in the context of antibody binding, refers to high avidity and/or high affinity binding of an antibody to a specific polypeptide, or more accurately, to an epitope of a specific polypeptide. Antibody binding to such an epitope on a polypeptide can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide, which does not comprise the epitope. Antibodies that bind specifically to a subject polypeptide may be capable of binding other polypeptides at a weak, yet detectable, level (for example, 10% or less of the binding shown to the polypeptide of interest). Such weak binding, or background binding, is readily discernible from the specific antibody binding to a subject polypeptide, for example, by use of appropriate controls. In general, antibodies of the invention bind to a specific polypeptide with a binding affinity of 10⁷ M⁻¹ or greater (for example, 10 M⁻¹, 10⁹ M⁻¹, 10¹¹ M⁻¹, 10¹¹ M⁻¹, etc.).

The invention provides antibodies that can distinguish variant target sequences from one another. These antibodies can distinguish polypeptides that differ by no more than one amino acid (U.S. Pat. No. 6,656,467). They have high affinity constants, i.e., in the range of approximately 10¹⁰ M⁻¹, and are produced, for example, by genetically engineering appropriate antibody gene sequences, according to the method described by Young et al., in U.S. Pat. No. 6,656,467.

Antibodies of the invention can be provided as matrices, i.e., as geometric networks of antibody molecules and their antigens, as found in immunoprecipitation and flocculation reactions. An antibody matrix can exist in solution or on a solid phase support.

Antibodies of the invention can be provided as a library of antibodies or fragments thereof, wherein at least one antibody or fragment thereof specifically binds to at least a portion of a polypeptide comprising an amino acid sequence or fragment thereof described in the Figures, Tables and Sequence Listing, and/or wherein at least one antibody or fragment thereof interferes with at least one activity of the polypeptide or fragment thereof. In certain embodiments, the antibody library comprises at least one antibody or fragment thereof that specifically inhibits the binding of a semaphorin 4B polypeptide to its ligand or other interaction partner, or that specifically inhibits binding of a semaphorin 4B polypeptide as a ligand to a semaphorin receptor. In certain embodiments, the antibody library comprises combinatorial complementarity determining regions, heavy chains, and light chains. The present invention also features corresponding polynucleotide libraries comprising at least one polynucleotide sequence that encodes an antibody or antibody fragment of the invention. In specific embodiments, the library is provided on a nucleic acid array or in computer-readable format.

The invention provides a method of making an antibody by introducing an antigen chosen from an isolated nucleic acid molecule comprising at least one polynucleotide sequence chosen from the Figures, Tables and Sequence Listing; sequences that hybridize to these sequences under high stringency conditions; sequences having at least 80% sequence identity to these sequences, or sequences that hybridize to them under high stringency conditions; complements of any of these sequences; or biologically active fragments of any of the above-listed sequences or an isolated polypeptide comprising an amino acid sequence, wherein the amino acid sequence is chosen from the Figures, Tables and Sequence Listing, or a biologically active fragment thereof, or is encoded by a polynucleotide sequence chosen from the Figures, Tables and Sequence Listing, or a biologically active fragment thereof, into an animal in an amount sufficient to elicit generation of antibodies specific to the antigen, and recovering the antibodies therefrom.

The immunogen can comprise a nucleic acid, a complete protein, or fragments and derivatives thereof, or proteins expressed on cell surfaces. Protein domains, for example, Pfam domains, or extracellular, cytoplasmic, or luminal domains can be used as immunogens. Immunogens can comprise all or a part of a subject polypeptide, where the amino acids contain post-translational modifications, such as glycosylation, found on the native target protein. Immunogens comprising protein extracellular domains are produced in a variety of ways known in the art, for example, expression of cloned genes using conventional recombinant methods, or isolation from tumor cell culture supernatants, etc. The immunogen can also be expressed in vivo from a polynucleotide encoding the immunogenic peptide introduced into the host animal.

Polyclonal antibodies of the invention are prepared by conventional techniques. These include immunizing the host animal in vivo with the target protein (or immunogen) in substantially pure form, for example, comprising less than about 1% contaminant. The immunogen can comprise the complete target protein, fragments, or derivatives thereof. To increase the immune response of the host animal, the target protein can be combined with an adjuvant; suitable adjuvants include alum, dextran, sulfate, large polymeric anions, and oil and water emulsions, for example, Freund's adjuvant (complete or incomplete). The target protein can also be conjugated to synthetic carrier proteins or synthetic antigens. The target protein is administered to the host, usually intradermally, with an initial dosage followed by one or more, usually at least two, additional booster dosages. Following immunization, blood from the host is collected, followed by separation of the serum from blood cells. The immunoglobulin present in the resultant antiserum can be further fractionated using known methods, such as ammonium salt fractionation, or DEAE chromatography and the like.

Monoclonal antibodies of the invention are also produced by conventional techniques, such as fusing an antibody-producing plasma cell with an immortal cell to produce hybridomas. Suitable animals will be used, for example, to raise antibodies against a mouse polypeptide of the invention, the host animal will generally be a hamster, guinea pig, goat, chicken, or rabbit, or the like. Generally, the spleen and/or lymph nodes of an immunized host animal provide the source of plasma cells, which are immortalized by fusion with myeloma cells to produce hybridoma cells. Culture supernatants from individual hybridomas are screened using standard techniques to identify clones producing antibodies with the desired specificity. The antibody can be purified from the hybridoma cell supernatants or from ascites fluid present in the host by conventional techniques, for example, affinity chromatography using antigen, for example, the subject protein, bound to an insoluble support, for example, protein A Sepharose®, etc.

The antibody can be produced as a single chain, instead of the normal multimeric structure of the immunoglobulin molecule. Single chain antibodies have been previously described (for example, Jost et al., 1994). DNA sequences encoding parts of the immunoglobulin, for example, the variable region of the heavy chain and the variable region of the light chain are ligated to a spacer, such as one encoding at least about four small neutral amino acids, i.e., glycine or serine. The protein encoded by this fusion allows the assembly of a functional variable region that retains the specificity and affinity of the original antibody.

The invention also provides intrabodies that are intracellularly expressed single-chain antibody molecules designed to specifically bind and inactivate target molecules inside cells. Intrabodies have been used in cell assays and in whole organisms (Chen et al., 1994; Hassanzadeh et al., 1998). Inducible expression vectors can be constructed with intrabodies that react specifically with a protein of the invention. These vectors can be introduced into host cells and model organisms.

The invention provides artificial antibodies, i.e., antibodies and antibody fragments produced and selected in vitro. In some embodiments, these antibodies, or fragments thereof are displayed on the surface of a bacteriophage or other viral particle, as described above. Suitable fragments include single chain variable region antibodies. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected, for example, on the basis of phage binding to selected antigens, can be fused to a Fc fragment of an immunoglobulin for use as a therapeutic, as described, for example, in U.S. Pat. No. 5,116,964 or WO 99/61630.

In an embodiment, artificial antibodies of the invention include genetically engineered antibodies. Single chain variable region antibodies are within the scope of such an embodiment. Engineered antibodies may incorporate non-antibody domains, including, for example, coiled coil domains for dimerization, linkers, or other such useful modifications. Genetically engineered antibodies of the invention include proteins with predetermined ligand specificity based on a known or predicted epitope, for example anticalins (Schlehuber et al., 2001), which are suitable for use in the invention when an immunogenic, cross-linking, or effector property of an antibody is undesirable.

For in vivo use, particularly for injection into humans, in some embodiments it is desirable to decrease the antigenicity of the antibody. An immune response of a recipient against the antibody may potentially decrease the period of time that the therapy is effective. Methods of humanizing antibodies are known in the art. The humanized antibody can be the product of an animal having transgenic human immunoglobulin genes, for example, constant region genes (for example, Grosveld and Kolias, 1992; Murphy and Carter, 1993; Pinkert, 1994; and International Patent Applications WO 90/10077 and WO 90/04036). Alternatively, the antibody of interest can be engineered by recombinant DNA techniques to substitute the CH1, CH2, CH3, hinge domains, and/or the framework domain with the corresponding human sequence (see, for example, WO 92/02190).

Thus, antibodies of the invention can be partially human or fully human antibodies. For example, xenogenic antibodies, which are produced in animals that are transgenic for human antibody genes, can be employed to make a fully human antibody. By xenogenic human antibodies is meant antibodies that are fully human antibodies, with the exception that they are produced in a non-human host that has been genetically engineered to express human antibodies (for example, WO 98/50433; WO 98/24893 and WO 99/53049).

Humanized antibodies can be produced by immunizing mice that make human antibodies. Abgenix's XenoMouse (for example, U.S. Pat. Nos. 5,939,598; 6,075,181; 6,091,001; 6,114,598; 6,150,584; 6,162,963; 6,657,103; 6,673,986; 6,682,736) Medarex's mice (for example, U.S. Pat. Nos. 5,922,845; 6,111,166; 6,410,690; 6,680,209) and Kirin's mice (for example, U.S. Pat. Nos. 6,320,099; 6,632,976) are suitable for use in the invention. Humanized antibodies can be made, for example, using the technology of Protein Design Labs, Inc. (Fremont, Calif.) (for example, Coligan, 2002). Both polyclonal and monoclonal antibodies made in non-human animals may be humanized before administration to human subjects.

Chimeric immunoglobulin genes constructed with immunoglobulin cDNA are known in the art (Liu et al. 1987a; Liu et al. 1987b). Messenger RNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant (C) regions genes are known in the art (Kabat et al., 1991). Human C region genes are readily available from known clones. The choice of isotype will be guided by the desired effector functions, such as complement fixation, or antibody-dependent cellular cytotoxicity. IgG1, IgG2, IgG3, and IgG4 isotypes, and either of the kappa or lambda human light chain constant regions can be used. The chimeric, humanized antibody is then expressed by conventional methods.

Consensus sequences of heavy (H) and light (L) J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

A convenient expression vector for producing antibodies is one that encodes a functionally complete human CH or CL immunoglobulin sequence, with appropriate restriction sites engineered so that any VH or VL sequence can be easily inserted and expressed, such as plasmids, retroviruses, YACs, or EBV derived episomes, and the like. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, for example, SV-40 early promoter (Okayama, et al. 1983), Rous sarcoma virus LTR (Gorman et al. 1982), and Moloney murine leukemia virus LTR (Grosschedl et al. 1985), or native immunoglobulin promoters.

Antibody fragments, such as Fv, F(ab′)2, and Fab can be prepared by cleavage of the intact protein, for example, by protease or chemical cleavage. These fragments can include heavy and light chain variable regions. Alternatively, a truncated gene can be designed, for example, a chimeric gene encoding a portion of the F(ab′)₂ fragment that includes DNA sequences encoding the CH1 domain and hinge region of the H chain, followed by a translational stop codon.

Antibodies may be administered by injection systemically, such as by intravenous injection; or by injection or application to the relevant site, such as by direct injection into a tumor, or direct application to the site when the site is exposed in surgery; or by topical application, such as if the disorder is on the skin, for example.

The antibodies of the present invention may be administered alone or in combination with other molecules for use as a therapeutic, for example, by linking the antibody to radioactive molecules or other cytotoxic agents. Radioactive antibodies and antibodies comprising a cytotoxic microbial, plant, or chemical compound that are specific to a cancer cell, diseased cell, or other target cell may be able to deliver a sufficient dose of radioactivity or toxin to kill the cell.

Radiolabeled antibodies of the invention can be used clinically to detect tumor cells, including latent metastases. Radionuclide imaging can be performed according to well-known methods, including this described in Kufe et al., 2003. In vivo diagnostic imaging methods of the invention include single photon and positron imaging, and may include the use of scanners and cameras, including, but not limited to computed tomography scanners and gamma cameras.

Antibodies of the invention can be used to modulate biological activity of cells, either directly or indirectly. An antibody can modulate the activity of a target cell, with which it has primary interaction, or it can modulate the activity of other cells by exerting secondary effects, i.e., when the primary targets interact or communicate with other cells. An antibody can also modulate the activity of a target cell by primarily interacting with an antigen, which then exerts an effect, whether direct, or indirect, on a target cell. Antibodies of the invention may specifically inhibit the binding of a subject polypeptide to a ligand, specifically inhibit the binding of a subject polypeptide to a substrate, specifically inhibit the binding of a subject polypeptide as a ligand, specifically inhibit the binding of a subject polypeptide as a substrate, specifically inhibit cofactor binding, induce apoptosis, induce ADCC, induce CDC, inhibit protease activity, inhibit adhesion, inhibit migration, inhibit proliferation, inhibit ligand/receptor interaction, and/or inhibit enzyme/substrate interaction.

The antibodies of the invention can be administered to mammals, and the present invention includes such administration, for example, for therapeutic and/or diagnostic purposes in humans. Accordingly, the invention provides a pharmaceutical composition comprising a pharmaceutically acceptable carrier and an antibody of the invention.

The antibodies of the present invention can also be used in assays to detect subject polypeptides. In some embodiments, the assay is a binding assay that detects binding of a polypeptide with an antibody specific for the polypeptide; the subject polypeptide or antibody can be immobilized, while the subject polypeptide and/or antibody can be detectably labeled. For example, the antibody can be directly labeled or detected with a labeled secondary antibody. That is, suitable, detectable labels for antibodies include direct labels, which label the antibody to the protein of interest, and indirect labels, which label an antibody that recognizes the antibody to the protein of interest.

These labels include radioisotopes, including, but not limited to ⁶⁴Cu, ⁶⁷CU, ⁹⁰Y, ^{99m}Tc, ¹¹¹In, ¹²⁴I, ¹²⁵I, ¹³¹I, ¹³⁷Cs, ¹⁸⁶Re, ²¹¹At, ²¹²Bi, ²¹³Bi, ²²³Ra, ²⁴¹Am, and ²⁴⁴ Cm; enzymes having detectable products (for example, luciferase, peroxidase, alkaline phosphatase, β-galactosidase, and the like); fluorescers and fluorescent labels, for example, as provided herein; fluorescence emitting metals, for example, ¹⁵²Eu, or others of the lanthanide series, attached to the antibody through metal chelating groups such as EDTA; chemiluminescent compounds, for example, luminol, isoluminol, or acridinium salts; and bioluminescent compounds, for example, luciferin, or aequorin (green fluorescent protein), specific binding molecules, for example, magnetic particles, microspheres, nanospheres, luminescent quantum dot nanocrystals, and the like.

Alternatively, specific-binding pairs may be used, involving, for example, a second stage antibody or reagent that is detectably labeled and that can amplify the signal. For example, a primary antibody can be conjugated to biotin, and horseradish peroxidase-conjugated strepavidin added as a second stage reagent. Digoxin and antidigoxin provide another such pair. In other embodiments, the secondary antibody can be conjugated to an enzyme such as peroxidase in combination with a substrate that undergoes a color change in the presence of the peroxidase. The absence or presence of antibody binding can be determined by various methods, including flow cytometry of dissociated cells, microscopy, radiography, or scintillation counting. Such reagents and their methods of use are well known in the art.

Antibodies of the invention can be provided in the form of arrays, i.e., collections of plural biological molecules having locatable addresses that may be separately detectable. Generally, a microarray encompasses use of submicrogram quantities of biological molecules. The antibodies may be affixed to a substrate or may be in solution or suspension. The substrate can be porous or solid, planar or non-planar, unitary or distributed, such as a glass slide, a 96 well plate, with or without the use of microbeads or nanobeads. Antibody microarrays of the invention include arrays of antibodies obtained by purification, as fusion proteins, and or recombinantly, and can be used for specific binding studies (Zhu and Snyder, 2003; Houseman et al., 2002; Schaeferling et al., 2002; Weng et al., 2002; Winssinger et al., 2002; Zhu et al., 2001; and MacBeath and Schreiber, 2000).

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, for example, adjuvants or cytokines.

### Vaccine Therapy

IgSF9, nectin 4, KIAA0152, and semaphorin 4B are overexpressed at the surface of cancer cells and are not normally expressed at high levels in healthy, non-pregnant individuals. Polypeptide, such as the extracellular domains of these target proteins, or portions of them, can be formulated and administered as a vaccine. Such a vaccine can be used to treat patients overexpressing the target at the surface of cancer cells, inducing antibody or cell mediated immune responses against the cancer cells, including antibody-dependent cell cytotoxicity (ADCC) or complement dependent cytotoxicity (CDC).

The invention also provides a method for prophylaxis or therapeutic treatment of a subject needing or desiring such treatment by providing a vaccine and administering the vaccine to the subject. The vaccine may comprise one or more of a polynucleotide, polypeptide, or modulator of the invention, for example an antibody vaccine composition, a polypeptide vaccine composition, or a polynucleotide vaccine composition. It may comprise a complement, biologically active fragment, or variant of any of these. For example, the vaccine can be a cancer vaccine, and the polypeptide can concomitantly be a cancer antigen. The vaccine can be administered with or without an adjuvant.

Vaccine therapy involves the use of polynucleotides, polypeptides, or agents of the invention as immunogens for tumor antigens (Machiels et al., 2002; Shinnick et al., 1983). For example, peptide-based vaccines of the invention include unmodified subject polypeptides, fragments thereof, and MHC class I and class II-restricted peptide (Knutson et al., 2001), comprising, for example, the disclosed sequences with universal, nonspecific MHC class II-restricted epitopes. Peptide-based vaccines comprising a tumor antigen can be given directly, either alone or in conjunction with other molecules. The vaccines can also be delivered orally by producing the antigens in transgenic plants that can be subsequently ingested (U.S. Pat. No. 6,395,964).

In some embodiments, antibodies themselves can be used as antigens in anti-idiotype vaccines. That is, administering an antibody to a tumor antigen stimulates B cells to make antibodies to that antibody, which in turn recognize the tumor cells.

Nucleic acid-based vaccines can deliver tumor antigens as polynucleotide constructs encoding the antigen. Vaccines comprising genetic material, such as DNA or RNA, can be given directly, either alone or in conjunction with other molecules. Administration of a vaccine expressing a molecule of the invention, for example, as plasmid DNA, leads to persistent expression and release of the therapeutic immunogen over a period of time, helping to control unwanted tumor growth.

In some embodiments, nucleic acid-based vaccines encode subject antibodies. In such embodiments, the vaccines (for example, DNA vaccines) can include post-transcriptional regulatory elements, such as the post-transcriptional regulatory acting RNA element (WPRE) derived from Woodchuck Hepatitis Virus. These post-transcriptional regulatory elements can be used to target the antibody, or a fusion protein comprising the antibody and a co-stimulatory molecule, to the tumor microenvironment (Pertl et al., 2003).

Cytokines can be used to help stimulate immune response. Cytokines act as chemical messengers, stimulating optimal responses from immune cells. An example of a cytokine is granulocyte-macrophage colony-stimulating factor (GM-CSF), which stimulates the proliferation of antigen-presenting cells, thus boosting an organism's response to a cancer vaccine. As with adjuvants, cytokines can be used in conjunction with the antibodies and vaccines disclosed herein. For example, they can be incorporated into the antigen-encoding plasmid or introduced via a separate plasmid, and in some embodiments, a viral vector can be engineered to display cytokines on its surface.

Besides stimulating anti-tumor immune responses by inducing humoral responses, vaccines of the invention can also induce cellular responses, including stimulating T-cells that recognize and kill tumor cells directly. For example, nucleotide-based vaccines of the invention encoding tumor antigens can be used to activate the CD8⁺ cytotoxic T lymphocyte arm of the immune system.

In some embodiments, the vaccines activate T-cells directly, and in others they enlist antigen-presenting cells to activate T-cells. Killer T-cells are primed, in part, by interacting with antigen-presenting cells, for example, dendritic cells. In some embodiments, plasmids comprising the nucleic acid molecules of the invention enter antigen-presenting cells, which in turn display the encoded tumor-antigens that contribute to killer T-cell activation. Again, the tumor antigens can be delivered as plasmid DNA constructs, either alone or with other molecules.

In further embodiments, RNA can be used. For example, antigen-presenting cells can be transfected or transduced with RNA encoding tumor antigens (Heiser et al., 2002; Mitchell and Nair, 2000). This approach overcomes the limitations of obtaining sufficient quantities of tumor material, extending therapy to patients otherwise excluded from clinical trials. For example, a subject RNA molecule isolated from tumors can be amplified using RT-PCR. In some embodiments, the RNA molecule of the invention is directly isolated from tumors and transfected into antigen-presenting cells or dendritic cells with no intervening cloning steps.

In some embodiments the molecules of the invention are altered such that the peptide antigens are more highly antigenic than in their native state. These embodiments address the need in the art to overcome the poor in vivo immunogenicity of most tumor antigens by enhancing tumor antigen immunogenicity via modification of epitope sequences (Yu and Restifo, 2002).

Another recognized problem of cancer vaccines is the presence of preexisting neutralizing antibodies. Some embodiments of the present invention overcome this problem by using viral vectors from non-mammalian natural hosts, i.e., avian pox viruses. Alternative embodiments that also circumvent preexisting neutralizing antibodies include genetically engineered influenza viruses, and the use of “naked” plasmid DNA vaccines that contain DNA with no associated protein. (Yu and Restifo, 2002).

### Carriers, Excipients and Formulations

In some embodiments, compositions related to one or more of the target molecules IgSF9, nectin 4, KIAA0152, and Semaphorin 4B, are provided in formulation with pharmaceutically acceptable excipients, a wide variety of which are known in the art (Gennaro, 2003; Ansel et al., 2004; Kibbe et al., 2000). Pharmaceutically acceptable excipients, such as vehicles, adjuvants, carriers, or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers such as Azone can also be included.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration. Administration of the agents can be achieved in various ways, including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, etc., or otherwise by implantation or inhalation. Thus, the subject compositions can be formulated into preparations in solid, semi-solid, liquid or gaseous forms, such as tablets, capsules, powders, granules, ointments, solutions, suppositories, injections, inhalants and aerosols. The following methods and excipients are merely exemplary and are in no way limiting.

Compositions for oral administration can form solutions, suspensions, tablets, pills, granules, capsules, sustained release formulations, oral rinses, or powders. For oral preparations, the agents, polynucleotides, and polypeptides can be used alone or in combination with appropriate additives, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art (Gennaro, 2003). The composition or formulation to be administered will, in any event, contain a quantity of the agent adequate to achieve the desired state in the subject being treated.

The antibodies, other agents, polynucleotides, and polypeptides can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. Other formulations for oral or parenteral delivery can also be used, as conventional in the art.

The antibodies, other agents, polynucleotides, and polypeptides can be utilized in aerosol formulation to be administered via inhalation. The compounds of the present invention can be formulated into pressurized acceptable propellants such as dichlorodifluoromethane, propane, nitrogen, and the like. Further, the agent, polynucleotides, or polypeptide composition may be converted to powder form for administration intranasally or by inhalation, as conventional in the art.

Furthermore, the antibodies, other agents, polypeptides, and polynucleotides can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

A polynucleotide, polypeptide, antibody, or other agent can also be introduced into tissues or host cells by other routes, such as viral infection, microinjection, or vesicle fusion. For example, expression vectors can be used to introduce nucleic acid compositions into a cell as described above. Further, jet injection can be used for intramuscular administration (Furth et al., 1992). The DNA can be coated onto gold microparticles, and delivered intradermally by a particle bombardment device, or “gene gun” as described in the literature (Tang et al., 1992), where gold microprojectiles are coated with the DNA, then bombarded into skin cells.

The agents can be provided in unit dosage forms, i.e., physically discrete units suitable as unitary dosages for human and animal subjects, each unit containing a predetermined quantity of compounds of the present invention calculated in an amount sufficient to produce the desired effect in association with a pharmaceutically acceptable diluent, carrier, or vehicle. The specifications for the novel unit dosage forms of the present invention depend on the particular compound employed and the effect to be achieved, and the pharmacodynamics associated with each compound in the host.

Unit dosage forms for oral or rectal administration such as syrups, elixirs, and suspensions can be provided wherein each dosage unit, for example, teaspoonful, tablespoonful, tablet, or suppository, contains a predetermined amount of the composition containing one or more agents. Similarly, unit dosage forms for injection or intravenous administration can comprise the agent(s) in a composition as a solution in sterile water, normal saline or another pharmaceutically acceptable carrier.

### Therapeutic Applications

The invention provides various therapeutic methods. In some embodiments, methods of modulating, including increasing and inhibiting, a biological activity of a target protein are provided. In other embodiments, methods of modulating a signal transduction activity of a target protein are provided. In further embodiments, methods of modulating interaction of a target protein with another, interacting protein or other macromolecule (for example a DNA, carbohydrate, or lipid), are provided.

Thus, in an embodiment, the therapeutic compositions herein are administered to subjects for treatment of a proliferative disease, such as a tumor or psoriasis. In an embodiment, the therapeutic compositions herein are administered to subjects to modulate immune related diseases. In a further embodiment, the therapeutic compositions herein are administered to subjects for modulation of apoptosis-related diseases.

As mentioned above, an effective amount of an agent of the invention is administered to the host, at a dosage sufficient to produce a desired result. In some embodiments, the desired result is at least a modification of a given biological activity of a subject polypeptide (in the individual, or in a localized anatomical site in the individual), as compared to a control. In other embodiments, the desired result is at least a modification of the level of an active subject polypeptide (in the individual, or in a localized anatomical site in the individual), as compared to a control. In yet other embodiments, the desired result is at least a modification of the cellular activity of a primary and/or a secondary target cell, as compared to a control.

Typically, the compositions of the instant invention will contain from less than 1% to about 95% of the active ingredient, in some embodiments, about 10% to about 50%. Generally, between about 100 mg and 500 mg of the compositions will be administered to a child and between about 500 mg and 5 grams will be administered to an adult. Administration is generally by injection and often by injection to a localized area. The frequency of administration will be determined by the care given based on patient responsiveness. Other effective dosages can be readily determined by one of ordinary skill in the art through trials establishing dose response curves.

In order to calculate the amount of therapeutic agent to be administered, those skilled in the art could use readily available information with respect to the amount of agent necessary to have the desired effect. The amount of an agent necessary to increase or decrease a level of an active target molecule can be calculated from in vitro experimentation. The amount of agent will, of course, vary depending upon the particular agent used.

Other effective dosages can be readily determined by one of ordinary skill in the art through routine trials establishing dose response curves, for example, the amount of agent necessary to increase or decrease a level of an active target molecule or a level of a cellular activity of a target cell can be calculated from in vitro experimentation. Those of skill will readily appreciate that dose levels can vary as a function of the specific compound, the severity of the symptoms, and the susceptibility of the subject to side effects, and preferred dosages for a given compound are readily determinable by those of skill in the art by a variety of means. For example, in order to calculate the polypeptide, polynucleotide, or modulator dose, those skilled in the art can use readily available information with respect to the amount necessary to have the desired effect, depending upon the particular agent used.

### Proliferative Conditions

In some embodiments, IgSF9, nectin 4, KIAA0152, or Semaphorin 4B are involved in the control of cell proliferation, and an agent of the invention inhibits undesirable cell proliferation. Such agents are useful for treating disorders that involve abnormal cell proliferation, including, but not limited to, cancer. The polypeptides, polynucleotides, antibodies, and other agents of the invention are useful for treating various types of cancer, as described in the Examples and Figures. Whether a particular agent and/or therapeutic regimen of the invention is effective in reducing unwanted cellular proliferation, for example, in the context of treating cancer or psoriasis, can be determined using standard methods.

In an embodiment, the invention provides a method of modulating the biological survival of a first human or non-human animal host cell comprising providing an antibody of the invention and contacting the antibody with the first host cell, wherein the activity of the first host cell, and/or a second host cell, is modulated either directly or indirectly. A polypeptide of the invention can modulate a survival signal to a cell which would otherwise die. This modulation may occur either directly or indirectly, for example, through a signaling pathway. When an abnormal number of cells survive, they may contribute to tumor formation. In an embodiment, the invention provides the abrogation of such a survival signal, providing a therapeutic benefit.

The therapeutic compositions and methods of the invention can be used in the treatment of cancer, i.e., an abnormal malignant cell or tissue growth, for example, a tumor. In an embodiment, the compositions and methods of the invention kill tumor cells. In an embodiment, they inhibit tumor development. Cancer is characterized by the proliferation of abnormal cells that tend to invade the surrounding tissue and metastasize to new body sites. The growth of cancer cells exceeds that of and is uncoordinated with the normal cells and tissues. In an embodiment, the compositions and methods of the invention inhibit the progression of premalignant lesions to malignant tumors.

Cancer encompasses carcinomas, which are cancers of epithelial cells, and are the most common forms of human cancer; carcinomas include squamous cell carcinoma, adenocarcinoma, melanomas, and hepatomas. Cancer also encompasses sarcomas, which are tumors of mesenchymal origin, and includes osteogenic sarcomas, leukemias, and lymphomas. Cancers can have one or more than one neoplastic cell type. Some characteristics that can, in some instances, apply to cancer cells are that they are morphologically different from norm al cells, and may appear anaplastic; they have a decreased sensitivity to contact inhibition, and may be less likely than normal cells to stop moving when surrounded by other cells; and they have lost their dependence on anchorage for cell growth, and may continue to divide in liquid or semisolid surroundings, whereas normal cells must be attached to a solid surface to grow.

Treatment herein refers to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. Thus, the invention provides both treatment and prophylaxis. It includes (1) preventing the disorder from occurring or recurring in a subject who may be predisposed to the disorder but has not yet been diagnosed as having it, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, and/or tumor size.

The polynucleotides, polypeptides, and antibodies described above can be used to treat cancer. In an embodiment, a fusion protein or conjugate can additionally comprise a tumor-targeting moiety. Suitable moieties include those that enhance delivery of an therapeutic molecule to a tumor. For example, compounds that selectively bind to cancer cells compared to normal cells, selectively bind to tumor vasculature, selectively bind to the tumor type undergoing treatment, or enhance penetration into a solid tumor are included in the invention. Tumor targeting moieties of the invention can be peptides. Nucleic acid and amino acid molecules of the invention can be used alone or as an adjunct to cancer treatment. For example, a nucleic acid or amino acid molecules of the invention may be added to a standard chemotherapy regimen. It may be combined with one or more of the wide variety of drugs that have been employed in cancer treatment, including, but are not limited to, cisplatin, taxol, etoposide, Novantrone (mitoxantrone), actinomycin D, camptothecin (or water soluble derivatives thereof), methotrexate, mitomycins (for example, mitomycin C), dacarbazine (DTIC), and anti-neoplastic antibiotics such as doxorubicin and daunomycin, or others, described, for example, in De Vita et al., 2001.

Drugs employed in cancer therapy may have a cytotoxic or cytostatic effect on cancer cells, or may reduce proliferation of the malignant cells. Drugs employed in cancer treatment can also be peptides. A nucleic acid or amino acid molecules of the invention can be combined with radiation therapy. A nucleic acid or amino acid molecules of the invention may be used adjunctively with therapeutic approaches described in De Vita et al., 2001. For those combinations in which a nucleic acid or amino acid molecule of the invention and a second anti-cancer agent exert a synergistic effect against cancer cells, the dosage of the second agent may be reduced, compared to the standard dosage of the second agent when administered alone. A method for increasing the sensitivity of cancer cells comprises co-administering a nucleic acid or amino acid molecule of the invention with an amount of a chemotherapeutic anti-cancer drug that is effective in enhancing sensitivity of cancer cells. Co-administration may be simultaneous or non-simultaneous administration. A nucleic acid or amino acid molecule of the invention may be administered along with other therapeutic agents, during the course of a treatment regimen. In one embodiment, administration of a nucleic acid or amino acid molecule of the invention and other therapeutic agents is sequential. An appropriate time course may be chosen by the physician, according to such factors as the nature of a patient's illness, and the patient's condition.

The invention also provides a method for prophylactic or therapeutic treatment of a subject needing or desiring such treatment by providing a vaccine that can be administered to the subject. The vaccine may comprise one or more agent of the invention, for example an antibody vaccine composition, a polypeptide vaccine composition, or a polynucleotide vaccine composition, useful for preventing or treating proliferative disorders, obesity, cardiac hypertrophy, or liver disease.

Whether a particular agent and/or therapeutic regimen of the invention is effective in reducing unwanted cellular proliferation, for example, in the context of treating cancer, can be determined using standard methods. For example, the number of cancer cells in a biological sample (for example, blood, a biopsy sample, and the like), can be determined. The tumor mass can be determined using standard radiological or biochemical methods.

### Apoptosis and Cell Death

The control of cell numbers in mammals is believed to be determined, in part, by a balance between cell proliferation and cell death. One form of cell death, sometimes referred to as necrotic cell death, is typically characterized as pathologic, resulting from trauma or injury. In contrast, there is another physiologic form of cell death that usually proceeds in an orderly or controlled manner. This orderly or controlled form of cell death is often referred to as apoptosis (Barr et al., 1994; Steller, 1995).

Apoptotic cell death naturally occurs in many physiological processes, including embryonic development and clonal selection in the immune system (Itoh et al., 1991). Decreased levels of apoptotic cell death have been associated with a variety of pathological conditions, including cancer and immune disease (Thompson et al., 1995). Antibodies specific to IgSF9, nectin 4, KIAA0152, or Semaphorin 4B may induce the apoptotic death of cancer cells by binding to the extracellular domains.

Apoptosis can be assayed using any known method. Assays can be conducted on cell populations or an individual cell, and include morphological assays and biochemical assays. Procedures to detect cell death based on the TUNEL method are available commercially, for example, from Boehringer Mannheim (Cell Death Kit) and Oncor (Apoptag Plus).

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not per se limitations to the claimed invention.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims. The claims do not encompass embodiments in the public domain.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

### MODES FOR CARRYING OUT THE INVENTION

The invention provides an isolated first nucleic acid molecule comprising a first polynucleotide sequence which encodes a polypeptide, a complement thereof, or a biologically active fragment thereof, wherein the sequence is shown in Tables 1-15, FIGS. 1-4, and/or SEQ. ID. NOS.:1-380. For example, this nucleic acid may encode IgSF9, nectin 4, KIAA0152, semaphorin 4B, or a fragment, variant, and/or an analogue of any of these. These polynucleotides may comprise an RNAi molecule, a ribozyme, or a nucleotide aptamer.

The invention also provides a pharmaceutical composition comprising a pharmaceutically acceptable carrier and an isolated first nucleic acid molecule described above. In an embodiment, the invention provides a non-human animal injected with one or more of these polynucleotides and/or their encoded polypeptides.

The invention further provides an isolated antibody that specifically recognizes, binds to, interferes with, and/or otherwise modulates the biological activity of at least one polypeptide and/or polynucleotide chosen from Tables 1-15, FIGS. 1-4, SEQ. ID. NOS.:1-380, and a biologically active fragment of any of these, and wherein the antibody is not currently in the public domain. The antibody specificity may be directed to a non-transmembrane domain and/or an extracellular domain of a polypeptide chosen from the non-TM coordinates shown in the Tables. The antibody specificity may also be directed to a Pfam domain or a Prosite domain chosen from the functional domain coordinates of the Tables and/or the protein domain coordinates of the Tables.

In an embodiment, the invention provides a pharmaceutical composition comprising a pharmaceutically acceptable carrier and any of these antibodies. The antibodies of the invention may further comprise one or more cytotoxic component, for example, a radioisotope, a microbial toxin, a plant toxin, or a chemical compound.

The invention yet further provides that any of the antibodies of the invention may have the function of specifically inhibiting the binding of the polypeptide to a ligand, specifically inhibiting the binding of the polypeptide to a substrate, specifically inhibiting the binding of the polypeptide as a ligand, specifically inhibiting the binding of the polypeptide as a substrate, specifically inhibiting cofactor binding (for example, zinc, calcium, magnesium, manganese, or other divalent cation), inducing apoptosis, inducing antibody dependent cell cytoxicity, inducing complement dependent cytotoxicity, inhibiting protease activity, inhibiting adhesion, modulating ligand/receptor interaction, and/or modulating enzyme/substrate interaction.

An antibody of the invention may be a monoclonal antibody; a polyclonal antibody; a single chain antibody; an antibody comprising a backbone of a molecule with an Ig domain or a T cell receptor backbone; a targeting antibody; a neutralizing antibody; a stabilizing antibody; an enhancing antibody; an antibody agonist; an antibody antagonist; an antibody that promotes endocytosis of a target antigen; a cytotoxic antibody; an antibody that mediates antibody dependent cell cytotoxicity; an antibody that mediates complement dependent cytotoxicity; a human antibody; a non-human primate antibody; a non-primate animal antibody; a rabbit antibody; a mouse antibody; a rat antibody; a sheep antibody; a goat antibody; a horse antibody; a porcine antibody; a cow antibody; a chicken antibody; a humanized antibody; a primatized antibody; a chimeric antibody; an antigen binding fragment; a fragment comprising a variable region of a heavy chain or a light chain of an immunoglobulin; a fragment comprising a complementarity determining region or a framework region of an immunoglobulin; and one or more active fragment, analogue, and/or antagonist of one or more of these antibodies.

Antibodies of the invention may be produced in a plant, an animal, a cell, or a virus. For example, they may be produced in a bacterial cell, a fungal cell, a plant cell, an insect cell, and/or a mammalian cell. Suitable cells include, but are not limited to yeast cells, Aspergillus cells, SF9 cells, High Five cells, cereal plant cells, tobacco cells, tomato cells, 293 cells, myeloma cells, NS0 cells, PerC6 cells, and CHO cells. The invention provides a host cell genetically modified to produce an antibody of the invention. It also provides a bacteriophage displaying an antibody of the invention and/or a fragment thereof.

The invention provides an isolated first nucleic acid molecule comprising the first polynucleotide sequence SEQ. ID. NOS.:1, 2, 92, 93, 95, 96, 221, 222, 224, 225, 248, 249, 250, 379, and/or 380; the polynucleotide sequence encoding a polypeptide of from SEQ. ID. NOS.:55, 56, 164, 165, 246, 247, 314 and/or 315; or biologically active fragments of any of these. This nucleic acid molecule may be chosen from a cDNA molecule, a genomic DNA molecule, a cRNA molecule, a siRNA molecule, a RNAi molecule, or a mRNA molecule. The invention also provides a double-stranded isolated nucleic acid molecule comprising this first nucleic acid molecule and its complement.

The invention further provides a second nucleic acid molecule comprising a second polynucleotide sequence complementary to the first nucleic acid molecule. This second nucleic acid molecule may be a RNAi molecule, an anti-sense molecule, or a ribozyme.

The invention provides an isolated polypeptide comprising an amino acid sequence from SEQ ID NOS.:55, 56, 164, 165, 246, 247, 314, 315, or biologically active fragments of any of these. This polypeptide may be present in a cell culture, for example, a bacterial cell culture, a mammalian cell culture, and/or an insect cell culture. The isolated polypeptide may be encoded by the first nucleic acid molecule described above.

In another aspect, the invention provides a method of modulating the biological activity of a first human or non-human animal host cell comprising providing at least one modulator, wherein the modulator is an antibody of claim 11, a soluble receptor that competes for ligand binding to a polypeptide encoded by a polynucleotide of the invention, an extracellular fragment that competes for ligand binding to a polypeptide encoded by a polynucleotide of the invention, or an aptamer, small molecule drug, RNA, anti-sense molecule, or ribozyme that inhibits the transcription or translation of a polynucleotide of the invention; and contact the modulator with the first target cell, wherein the activity of the first target cell, and/or a second target cell, is modulated. This method may be performed such that the modulation of biological activity is that of inhibiting cell activity directly, inhibiting cell activity indirectly, inducing antibody dependent cell cytotoxicity, or inducing complement dependent cytotoxicity. The modulated cell activity may be, for example, receptor binding, signal transduction, transcription, translation, protein-protein interaction, proteolysis, adhesion, migration, invasion, metastasis, cell growth, proliferation, cell death, or cell survival. In practicing the method, contacting the antibody with a first target cell may result in recruitment of at least one second target cell. The invention provides that the first target cell may be a cancer cell. The first or second host cell may be chosen from a T cell, B cell, NK cell, dendritic cell, macrophage, muscle cell, stem cell, skin cell, fat cell, blood cell, brain cell, bone marrow cell, endothelial cell, retinal cell, bone cell, kidney cell, pancreatic cell, liver cell, spleen cell, prostate cell, cervical cell, ovarian cell, breast cell, lung cell, soft tissue cell, colorectal cell, and a cell of the gastrointestinal tract.

The invention also provides a method of identifying a modulator of the biological activity of a polypeptide comprising providing at least one polypeptide of Tables 1-15, FIGS. 1-4, SEQ. ID. NOS.:1-380, and active fragments of any of these, allowing at least one agent to contact the polypeptide, and selecting an agent that binds the polypeptide and/or affects the biological activity of the polypeptide. The modulator may, for example, be an antibody.

The invention further provides a method of identifying a modulator that modulates the biological activity of a polypeptide comprising providing at least one polypeptide chosen from sequences listed in Tables 1-15, FIGS. 1-4, SEQ. ID. NOS.:1-380, and active fragments, variants, or analogues thereof; allowing at least one agent to contact the polypeptide; and selecting an agent that binds the polypeptide or affects the biological activity of the polypeptide wherein the selection is based on assays described herein or known in the art. The modulator identified by this method may, for example, comprise an antibody of the invention.

The invention further provides a modulator composition comprising a pharmaceutically acceptable carrier and a modulator, wherein the modulator is obtainable by the method described above. The invention provides that the modulator composition may comprise a pharmaceutically acceptable carrier and a modulator, wherein the modulator is an antibody of the invention. The modulator composition may comprise a pharmaceutically acceptable carrier and a modulator, wherein the modulator is a soluble receptor that competes for ligand binding to an isolated polypeptide comprising an amino acid sequence of Tables 1-15, FIGS. 1-4, SEQ. ID. NOS.: 1-380, and biologically active fragments of any of these. In an embodiment, the modulator composition comprises a pharmaceutically acceptable carrier and a modulator, wherein the modulator is an extracellular fragment that competes for ligand binding to an isolated polypeptide comprising an amino acid sequence of Tables 1-15, FIGS. 1-4, SEQ. ID. NOS.:1-380, and biologically active fragments of any of these.

In an embodiment, the modulator composition comprises a pharmaceutically acceptable carrier and a modulator, wherein the modulator is an RNAi molecule that inhibits the transcription or translation of an isolated polynucleotide or an isolated polypeptide comprising an amino acid sequence encoded by a polynucleotide of Tables 1-15, FIGS. 1-4, SEQ. ID. NOS.:1-380, and biologically active fragments of any of these.

In an embodiment, the modulator composition comprises a pharmaceutically acceptable carrier and a modulator, wherein the modulator is an antisense molecule that inhibits the transcription or translation of an isolated polynucleotide or an isolated polypeptide comprising an amino acid sequence encoded by a polynucleotide of Tables 1-15, FIGS. 1-4, SEQ. ID. NOS.: 1-380, and biologically active fragments of any of these.

In an embodiment, the modulator composition comprises a pharmaceutically acceptable carrier and a modulator, wherein the modulator is a ribozyme that inhibits the transcription or translation of an isolated polynucleotide or an isolated polypeptide comprising an amino acid sequence encoded by a polynucleotide of Tables 1-15, FIGS. 1-4, SEQ. ID. NOS.:1-380, and biologically active fragments any of these.

In an embodiment, the modulator composition comprises a pharmaceutically acceptable carrier and a modulator, wherein the modulator is an aptamer that inhibits the function of an isolated polynucleotide or an isolated polypeptide comprising an amino acid sequence encoded by a polynucleotide of Tables 1-15, FIGS. 1-4, SEQ. ID. NOS.:1-380, and biologically active fragments of any of these.

In another aspect, the invention provides a method of determining the presence of a polypeptide specifically binding to an antibody in a sample, comprising allowing an antibody of the invention to interact with the sample and determining whether interaction between the antibody and the polypeptide has occurred.

The invention also provides a method of determining the presence of an antibody specifically binding to a polypeptide or a polynucleotide in a sample, comprising allowing an isolated polynucleotide encoding a polypeptide or an isolated polypeptide encoded by a polynucleotide, wherein the polypeptide comprises an amino acid sequence from Tables 1-15, FIGS. 1-4, SEQ. ID. NOS.:1-380, and/or biologically active fragments of any of these, to interact with the sample; and determining whether interaction between the antibody and the polypeptide or polynucleotide has occurred.

The invention further provides a method of diagnosing cancer in a patient, comprising providing an antibody of the invention, allowing the polypeptide to contact a patient sample (for example, a blood sample), and detecting specific binding between the polypeptide and any interacting molecule in the sample to determine whether the patient has cancer. This method may be detect a cancer is chosen from lung, colorectal, breast, prostate, bladder, pancreatic, endometrial, skin, kidney, liver, thyroid, ovarian, and stomach cancer. The invention provides a kit comprising an antibody of the invention and instructions for performing the diagnostic methods described above.

The invention provides a method of treating hyperproliferative growth in a patient comprising administering a modulator which binds to or interferes with the activity of an isolated polynucleotide encoding a polypeptide or an isolated polypeptide encoded by a polynucleotide, wherein the polypeptide comprises an amino acid sequence of Tables 1-15, FIGS. 1-4, SEQ. ID. NOS.:1-380, and biologically active fragments of any of these, to a patient. In an embodiment, the modulator is an antibody of the invention. In an embodiment, the uncontrolled proliferative growth is a tumor, for example, a tumor is chosen from a lung tumor, a colorectal tumor, a breast tumor, a prostate tumor, a bladder tumor, a pancreatic tumor, an endometrial tumor, a skin tumor, a kidney tumor, a liver tumor, a thyroid tumor, an ovarian tumor, and a stomach tumor.

The invention also provides a method of treating a tumor in a patient comprising a modulator composition as described herein and administering the modulator composition to the patient. In an embodiment, the modulator is an antibody, which, for example, may specifically recognize, bind to, or modulate the biological activity of a polypeptide and wherein the polypeptide comprises an amino acid sequence of Tables 1-15, FIGS. 1-4, SEQ. ID. NOS.:1-380 or is encoded by a polynucleotide of Tables 1-15, FIGS. 1-4, SEQ. ID. NOS.:1-380, and biologically active fragments of any of these.

The invention further provides a method of treating a lung, colorectal, breast, prostate, bladder, pancreatic, endometrial, skin, kidney, liver, thyroid, ovarian, or stomach tumor in a patient comprising providing a modulator composition described herein and administering the modulator composition to the patient. In an embodiment, the modulator is an antibody, which, for example, may specifically recognize, bind to, or modulate the biological activity of a polypeptide and wherein the polypeptide comprises an amino acid sequence in the Tables and Sequence Listing or is encoded by a polynucleotide in the Tables and Sequence Listing, and biologically active fragments of any of these. The invention yet further provides a kit comprising one or more antibody of the invention and instructions for performing the methods of treatment described herein.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### INDUSTRIAL APPLICABILITY

Nectin 4, semaphorin 4b, IgSF9, and KIAA0152 polypeptides, polynucleotides, and modulators, for example, antibodies, find use in a number of diagnostic, prophylactic, and therapeutic applications relating to proliferative disorders, for example, cancer and psoriasis. These therapeutics include nucleic acid and polypeptide antibodies and vaccines, such as cancer vaccines, which may be administered alone, such as naked DNA, or may be facilitated, such as via viral vectors, microsomes, liposomes, or nanoparticles. Therapeutic antibodies include, for example, monoclonal antibodies or binding fragments. They may be administered alone or in combination with cytotoxic agents, such as radioactive or chemotherapeutic agents.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### IgSF9 Microarray Expression in Normal and Cancerous Tissues

The present invention utilized probes that were designed by and purchased from Affymetrix, Inc. (Santa Clara, Calif.) to identify specific targets. Eleven matching probes, each about 25 nucleotides in length, were designed to correspond to a target sequence for selected clones from tumor or normal tissues. Eleven other target probes were designed for each target sequence, each with a single nucleotide mismatch. These probes were spotted on a microarray chip designed by Five Prime Therapeutics, Inc., with the nucleotide sequences of approximately 30,000 human genes “the Five Prime chip,” and hybridized to cRNA made complementary to RNA from tumor tissues or normal tissues.

FIG. 5 shows an exon map of the cluster containing the IgSF9 gene. The corresponding position of the Five Prime chip's probe used to identify IgSF9 as a target of the invention is also shown (FPT chip probe).

After hybridization, using an Affymetrix protocol, the results were read, again using Affymetrix's equipment and protocol. Results were reported as being either present or absent on the chip and also as a value representing the intensity of the hybridization. A probe set was a “hit” when the probe set was “present” and when the intensity was high in tumor tissues and low in normal tissues. A probe set was a “hit” when the probes matching the designated sequence hybridized to the RNA and the mismatched probes did not hybridize.

RNA was prepared from tumor tissue resected from patients with different types of cancer, and from normal-appearing adjacent tissue resected from the same patients. RNA was also prepared from other normal tissue specimens. Tissues were flash frozen in liquid nitrogen, transported on dry ice, and stored at minus 180° C. in liquid nitrogen. Histology was performed on a sample of each frozen tissue specimen and reviewed by a pathologist to confirm the cancer diagnosis or the tissue's normality. Only confirmed specimens were used for microarray hybridization or real time PCR experiments.

RNA was isolated from the tissues by grinding them to a fine powder under liquid nitrogen with a pre-chilled mortar and pestle. Total RNA was extracted using TRIzol reagent (Invitrogen, Carlsbad, Calif., USA) according to the manufacturer's protocol. It was treated with DNase in a final volume of 500 μl using 350 μg total RNA, 35 U DNase I, 50 μL DNase buffer and 280 U RNaseOUT (all from Invitrogen). Following incubation at 37° C. for 30 min., 500 μl phenol:chloroform:isoamyl alcohol (Invitrogen) was added, and the mixture vortexed, spun at 14,000 rpm for 5 min., and the aqueous phase transferred to a new 2 ml tube. The RNA was then ethanol precipitated by adding 80 mL 5 M NH₄OAc, 1.5 ml EtOH, incubated at −20° C. for 30 min., then spun at 14,000 rpm for 30 min. The pellet was washed with 75% EtOH and resuspended with 20 μL H₂0. The quality and concentration of the RNA were determined spectrophotometrically at 260 and 280 nm wavelengths and by agarose gel electrophoresis.

The resulting RNA was used as a template to prepare cDNA. First-strand cDNA synthesis was performed in a final volume of 20 μl with 10 μg total RNA, 5 μM T7-linked oligo(dT)₂₄ primer, 4 μl of 5× first-strand cDNA buffer, 10 mM DTT, 0.5 mM dNTP mix and 400 U Superscript B reverse transcriptase (all from Invitrogen). This mixture was incubated at 42° C. for 80 min. Second-strand cDNA synthesis was performed in a final volume of 150 μl using 20 μl of the first strand synthesis mixture, 30 μL 5× second-strand reaction buffer, 0.2 mM dNTP mix, 10 U E. coli DNA ligase, 40 U E. Coli DNA polymerase I, and 2 U E. coli RNase H. This mixture was incubated at 16° C. for 2 h. Then 20 U DNA polymerase was added and incubation at 16° C. continued for an additional 5 min.

In vitro transcription was performed with biotinylated UTP and CTP (Enzo Life Sciences, Inc., Farmingdale, N.Y.), resulting in an approximately 40-fold linear amplification of the RNA. Thirty five micrograms of biotinylated RNA was fragmented to a size of approximately 50 to 150 nucleotides before overnight hybridization to a chip microarray designed by Five Prime Therapeutics, Inc. (South San Francisco, Calif.) and custom built by Affymetrix (Santa Clara, Calif.). The array contained probe sets for approximately 30,000 human genes, including a specific probe (PRB103989_s_at for IgSF9 (NCBI accession number NP_{—}065840:NM_{—}020789). After washing, arrays were stained with streptavidin-phycoerythrin (Molecular Probes) and scanned with an Affymetrix GeneChip 3000 high-resolution scanner. Intensity values were scaled such that overall intensity for each chip of the same type was equivalent. Intensity for each feature of the array was captured by using Genechip Software (GCOS) (Affymetrix, Santa Clara, Calif.), and a single raw expression level for each gene was derived from 11 probe pairs representing each gene by using a trimmed mean algorithm.

The results of these microarray hybridization experiments are shown in FIGS. 6-11. IgSF9 probes hybridized at higher intensities to selected tumor tissues than to normal tissues. Expression profiling analysis with the proprietary Five Prime chip using a probe (PRB103989_s_at) against the cytoplasmic domain of IgSF9 (FIG. 5) revealed that IgSF9 mRNA was overexpressed in lung cancers compared to normal lung tissues, in breast cancers compared to normal breast tissues, in prostate cancers compared to normal prostate tissues, and in pancreatic cancers compared to normal pancreas tissues (FIGS. 6-10). Furthermore, IgSF9 was not expressed at detectable levels in most normal tissues (FIG. 11).

### Example 2

### Expression of IgSF9 Quantified by Real-Time PCR

RNA was prepared from the normal and cancerous tissues described in Example 1 and a subset of these tissues were used to perform real time PCR. Complementary DNA was prepared by reverse transcription, performed in a final volume of 100 μl with 2 μg of the isolated RNA, 125 U Multiscribe reverse transcriptase, 10 μL reverse transcription buffer, 22 μL 25 mM MgCl₂, 20 μL 10 mM dNTP, random hexamers, and oligo(dT)₁₆ at a final concentration of 2 mM each, and 40 U RNase inhibitor (all from Applied Biosystems, Foster City, Calif., USA). This mixture was incubated at 25° C. for 10 min. at 42° C. for 60 min. then at 95° C. for 5 mm.

Five Prime PCR primers and probes were designed using Primer Express™ software (Applied Biosystems, Foster City, Calif., USA). The locations of the PCR probes for IgSF9 are shown in the context of the IgSF9 exon map, shown in FIG. 5.

The primers and probes were used to quantitatively amplify IgSF9 in a polymerase chain reaction (PCR) performed on duplicate samples in a 25 μl reaction volume containing 2× TaqMan Universal PCR Master Mix (Applied Biosystems), primers at a final concentration of 900 nM each, 250 nM probe, water to a 20 μl final volume, and 5 μl of the cDNA. This PCR-based quantification analysis was performed with an ABI Prism 7000 Sequence Detection System (Applied Biosystems) using the following amplification parameters: 2 min. at 50° C., 10 min. at 95° C., 40 cycles of 15 sec. at 95° C. and 1 min. at 60° C.

To confirm that the RT-PCR primer-probes were specific each set of probes and primers was tested on cDNA plasmid clones encoding IgSF9 (FIG. 22).

These specific primer-probes were then used for quantitative RT-PCR (Taqman) analysis of lung squamous cell carcinoma and normal lung tissues. The results confirmed the overexpression of IgSF9 in lung squamous cell carcinoma as compared to normal lung tissue (FIG. 25).

### Example 3

### Nectin 4 Microarray Expression in Normal and Cancerous Tissues

Microarray expression analysis of nectin 4 (NCBI accession number NP_{—}112178:NM_{—}030916) was performed essentially as described for IgSF9 in Example 1. FIG. 24 shows an exon map of the cluster containing the nectin 4 gene. The corresponding position of the Five Prime chip's probe (PRB103018_s_at) used to identify nectin 4 as a target of the invention is also shown (FPT chip probe). The results of these microarray hybridization experiments are shown in FIGS. 25-29. Nectin 4 mRNA was overexpressed in lung adenocarcinomas and lung squamous cell carcinomas compared to normal lung tissues, in colon/colorectal cancers compared to normal colon/colorectal tissues, in prostate cancers compared to normal prostate tissues, and in pancreatic cancers compared to normal pancreas tissues (FIGS. 25-28). Furthermore, nectin 4 was not expressed at detectable levels in most normal tissues, including important tissues such as heart, liver and kidney (FIG. 29).

### Example 4

### KIAA0152 Microarray Expression in Normal and Cancerous Tissues

Microarray expression analysis of KIAA0152 (NCBI accession number NP_{—}055545:NM_{—}014730) was performed essentially as described for IgSF9 in Example 1. FIG. 40 shows an exon map of the cluster containing the KLAA0152 gene. The corresponding position of the Five Prime chip's probe (PRB105610 at) used to identify KIAA0152 as a target of the invention is also shown (FPT chip probe). The results of these microarray hybridization experiments are shown in FIGS. 41-46. KIAA0152 mRNA was overexpressed in lung cancers compared to normal lung tissues, in colon/colorectal cancers compared to normal colon/colorectal tissues, in breast cancers compared to normal breast tissues, in prostate cancers compared to normal prostate tissues, and in pancreatic cancers compared to normal pancreas tissues (FIGS. 41-45). KIAA0152 was also expressed at relatively low levels in many, but not all, normal tissues (FIG. 46).

### Example 5

### Expression of KIAA0152 Quantified by Real-Time PCR

Real-time PCR analysis of KIAA0152 was performed essentially as described for IgSF9 in Example 2. RNA was prepared from the normal and cancerous tissues described in Example 1 and a subset of these tissues were used to perform real-time PCR analysis. The locations of the PCR primer-probes for KIAA0152 are shown in the context of the KIAA0152 exon map, shown in FIG. 40. The specificity of the primer-probes was tested and confirmed on cDNA plasmid clones encoding KIAA0152 (FIG. 57). These specific primer-probes were then used for quantitative RT-PCR (Taqman) analysis of prostate cancer tissues, normal prostate tissues, and other normal tissues. The results confirmed the overexpression of KIAA0152 in a fraction of prostate cancers and the detection of KIAA0152 in some other normal tissues at relatively low levels (FIGS. 58 and 59).

### Example 6

### Semaphorin 4B Microarray Expression in Normal and Cancerous Tissues

Microarray Expression analysis of semaphorin 4B (NCBI accession number 39777608:39777607) was performed essentially as described for IgSF9 in Example 1. FIG. 60 shows an exon map of the cluster containing the semaphorin 4B gene. The corresponding position of the Five Prime chip's probe (PRB101227_s_at) used to identify semaphorin 4B as a target of the invention is also shown (FPT chip probe). The results of these microarray hybridization experiments are shown in FIGS. 61-65. Semaphorin 4B mRNA was overexpressed in lung adenocarcinomas and lung squamous cell carcinomas compared to normal lung tissues, in colon/colorectal cancers compared to normal colon/colorectal tissues, in prostate cancers compared to normal prostate tissues, and in pancreatic cancers compared to normal pancreas tissues (FIGS. 61-64). Furthermore, semaphorin 4B was expressed at low or undetectable levels in most normal tissues, including important tissues such as heart, liver and kidney (FIG. 65).

### Example 7

### Expression of Semaphorin 4B Quantified by Real-Time PCR

Real-time PCR analysis of semaphorin 4B was performed essentially as described for IgSF9 in Example 2. RNA was prepared from the normal and cancerous tissues described in Example 1 and a subset of these tissues were used to perform real-time PCR analysis. The locations of the PCR primer-probes for semaphorin 4B are shown in the context of the semaphorin 4B exon map, shown in FIG. 60. The specificity of the primer-probes was tested and confirmed on cDNA plasmid clones encoding semaphorin 4B (FIG. 76). These specific primer-probes were then used for quantitative RT-PCR (Taqman) analysis of lung squamous cell carcinoma and normal lung tissues. The results confirmed the overexpression of semaphorin 4B in a fraction of lung squamous cell carcinomas (FIG. 77).

TABLE 1
SEQ. ID. NOS.: 1-94 (Related to Cluster 192303, IgSF9)

| | SEQ. ID. NO. | SEQ. ID. NO. | SEQ. ID. NO. | |
|---|---|---|---|---|
| FP ID | (N1) | (P1) | (N0) | Clone ID |
| HG1015801 | SEQ. ID. NO. 1 | SEQ. ID. NO. 55 | SEQ. ID. NO. 92 | 37181362:37181361 |
| HG1015821 | SEQ. ID. NO. 2 | SEQ. ID. NO. 56 | SEQ. ID. NO. 93 | 7243091:7243090 |
| HG1015845 | SEQ. ID. NO. 3 | SEQ. ID. NO. 57 | SEQ. ID. NO. 94 | NP_065840:NM_020789 |
| HG1016214 | SEQ. ID. NO. 4 | SEQ. ID. NO. 58 | | 37181362:37181361.ig.1 |
| HG1016215 | SEQ. ID. NO. 5 | SEQ. ID. NO. 59 | | 37181362:37181361.ig.2 |
| HG1016216 | SEQ. ID. NO. 6 | SEQ. ID. NO. 60 | | 37181362:37181361.ig.3 |
| HG1016241 | SEQ. ID. NO. 7 | SEQ. ID. NO. 61 | | 7243091:7243090.fn3.1 |
| HG1016242 | SEQ. ID. NO. 8 | SEQ. ID. NO. 62 | | 7243091:7243090.fn3.2 |
| HG1016243 | SEQ. ID. NO. 9 | SEQ. ID. NO. 63 | | 7243091:7243090.ig.1 |
| HG1016244 | SEQ. ID. NO. 10 | SEQ. ID. NO. 64 | | 7243091:7243090.ig.2 |
| HG1016245 | SEQ. ID. NO. 11 | SEQ. ID. NO. 65 | | 7243091:7243090.ig.3 |
| HG1016246 | SEQ. ID. NO. 12 | SEQ. ID. NO. 66 | | 7243091:7243090.ig.4 |
| HG1016311 | SEQ. ID. NO. 13 | SEQ. ID. NO. 67 | | NP_065840:NM_020789.fn3.1 |
| HG1016312 | SEQ. ID. NO. 14 | SEQ. ID. NO. 68 | | NP_065840:NM_020789.fn3.2 |
| HG1016313 | SEQ. ID. NO. 15 | SEQ. ID. NO. 69 | | NP_065840:NM_020789.ig.1 |
| HG1016314 | SEQ. ID. NO. 16 | SEQ. ID. NO. 70 | | NP_065840:NM_020789.ig.2 |
| HG1016315 | SEQ. ID. NO. 17 | SEQ. ID. NO. 71 | | NP_065840:NM_020789.ig.3 |
| HG1016420 | SEQ. ID. NO. 18 | | | PRB103989_s_at |
| HG1017286 | SEQ. ID. NO. 19 | | | PRB103989_s_at:1 |
| HG1017287 | SEQ. ID. NO. 20 | | | PRB103989_s_at:10 |
| HG1017288 | SEQ. ID. NO. 21 | | | PRB103989_s_at:11 |
| HG1017289 | SEQ. ID. NO. 22 | | | PRB103989_s_at:2 |
| HG1017290 | SEQ. ID. NO. 23 | | | PRB103989_s_at:3 |
| HG1017291 | SEQ. ID. NO. 24 | | | PRB103989_s_at:4 |
| HG1017292 | SEQ. ID. NO. 25 | | | PRB103989_s_at:5 |
| HG1017293 | SEQ. ID. NO. 26 | | | PRB103989_s_at:6 |
| HG1017294 | SEQ. ID. NO. 27 | | | PRB103989_s_at:7 |
| HG1017295 | SEQ. ID. NO. 28 | | | PRB103989_s_at:8 |
| HG1017296 | SEQ. ID. NO. 29 | | | PRB103989_s_at:9 |
| HG1019532 | SEQ. ID. NO. 30 | | | NP_065840_taqman |
| HG1019533 | SEQ. ID. NO. 31 | | | CLN00162030_taqman |
| HG1019534 | SEQ. ID. NO. 32 | | | 192303_set1F |
| HG1019535 | SEQ. ID. NO. 33 | | | 192303_set1R |
| HG1019536 | SEQ. ID. NO. 34 | | | 192303_set1Probe |
| HG1019537 | SEQ. ID. NO. 35 | | | 192303_set2F |
| HG1019538 | SEQ. ID. NO. 36 | | | 192303_set2R |
| HG1019539 | SEQ. ID. NO. 37 | | | 192303_set2Probe |
| HG1019540 | SEQ. ID. NO. 38 | | | 192303_set3F |
| HG1019541 | SEQ. ID. NO. 39 | | | 192303_set3R |
| HG1019542 | SEQ. ID. NO. 40 | | | 192303_set3Probe |
| HG1019543 | SEQ. ID. NO. 41 | SEQ. ID. NO. 72 | | 7243091_ECD |
| HG1019544 | SEQ. ID. NO. 42 | SEQ. ID. NO. 73 | | NP_065840_ECD |
| HG1019545 | SEQ. ID. NO. 43 | SEQ. ID. NO. 74 | | NP_065840_frag1 |
| HG1019546 | | SEQ. ID. NO. 75 | | 7243091_frag1 |
| HG1019547 | | SEQ. ID. NO. 76 | | NP_065840_frag2 |
| HG1019548 | | SEQ. ID. NO. 77 | | NP_065840_frag3 |
| HG1019549 | | SEQ. ID. NO.78 | | NP_065840_frag4 |
| HG1019550 | | SEQ. ID. NO. 79 | | NP_065840_frag5 |
| HG1019551 | | SEQ. ID. NO. 80 | | NP_065840_frag6 |
| HG1019610 | SEQ. ID. NO. 44 | SEQ. ID. NO. 81 | | 37181362:37181361.I- |
| | | | | set.1 |
| HG1019611 | SEQ. ID. NO. 45 | SEQ. ID. NO. 82 | | 37181362:37181361.I- |
| | | | | set.2 |
| HG1019612 | SEQ. ID. NO. 46 | SEQ. ID. NO. 83 | | 37181362:37181361.V- |
| | | | | set.1 |
| HG1019613 | SEQ. ID. NO. 47 | SEQ. ID. NO. 84 | | 37181362:37181361.V- |
| | | | | set.2 |
| HG1019614 | SEQ. ID. NO. 48 | SEQ. ID. NO. 85 | | 7243091:7243090.I- |
| | | | | set.1 |
| HG1019615 | SEQ. ID. NO. 49 | SEQ. ID. NO. 86 | | 7243091:7243090.I- |
| | | | | set.2 |
| HG1019616 | SEQ. ID. NO. 50 | SEQ. ID. NO. 87 | | 7243091:7243090.V- |
| | | | | set.1 |
| HG1019617 | SEQ. ID. NO. 51 | SEQ. ID. NO. 88 | | 7243091:7243090.V- |
| | | | | set.2 |
| HG1019618 | SEQ. ID. NO. 52 | SEQ. ID. NO. 89 | | NP_065840:NM_020789.I-set.1 |
| HG1019619 | SEQ. ID. NO. 53 | SEQ. ID. NO. 90 | | NP_065840:NM_020789.I-set.2 |
| HG1019620 | SEQ. ID. NO. 54 | SEQ. ID. NO. 91 | | NP_065840:NM_020789.V-set.1 |

TABLE 2
Annotation of NCBI Sequences Identified by PRB103989_s_at

| | | Predicted | |
|---|---|---|---|
| | | Protein | |
| FP ID | Clone ID | Length | Annotation |
| HG1015801 | 37181362:37181361 | 717 | IgSF9 |
| | | | [Homo sapiens] |
| HG1015821 | 7243091:7243090 | 1189 | KIAA1355 |
| | | | protein |
| | | | [Homo sapiens] |
| HG1015845 | NP_065840:NM_020789 | 1163 | Immunoglobulin |
| | | | superfamily, |
| | | | member |
| | | | 9 [Homo sapiens] |

TABLE 3
Characterization of Polypeptides Encoded by NCBI Sequences of
Cluster 192303, IgSF9

| | | | | | | Altern | Altern | | | | |
|---|---|---|---|---|---|---|---|---|---|---|---|
| | | Pred | | Signal | Mature | Signal | Mature | | | | |
| | | Prot | Tree- | Peptide | Protein | Peptide | Protein | | TM | non-TM | |
| FP ID | Clone ID | Len | vote | Coords | Coords | Coords | Coords | TM | Coords | Coords | Pfam |
| HG1015801 | 37181362: | 717 | 1 | (1-20) | (21-717) | (5-17) | (18-717) | 0 | | (1-717) | I-set; |
| | 37181361 | | | | | (3-15) | (16-717) | | | | ig; |
| | | | | | | | | | | | V-set |
| HG1015821 | 7243091: | 1189 | 0 | (11-30) | (31-1189) | (9-21) | (22-1189) | 1 | (748-770) | (1-747) | I-set |
| | 7243090 | | | | | (15-27) | (28-1189) | | | (771-1189) | fn3; |
| | | | | | | (13-25) | (26-1189) | | | | ig |
| | | | | | | | | | | | V-set |
| HG1015845 | NP_065840: | 1163 | 0 | (1-20) | (21-1163) | (5-17) | (18-1163) | 1 | (722-744) | (1-721) | I-set |
| | NM_020789 | | | | | (3-15) | (16-1163) | | | (745-1163) | fn3 ig |
| | | | | | | | | | | | V-set |

TABLE 4
Pfam Domains of Polypeptides Encoded by Sequences of
Cluster 192303, IgSF9

| FP ID | Clone ID | Pfam | Coordinates |
|---|---|---|---|
| HG1015801 | 37181362:37181361 | I-set | (136-224) |
| HG1015801 | 37181362:37181361 | I-set | (227-302) |
| HG1015801 | 37181362:37181361 | V-set | (226-320) |
| HG1015801 | 37181362:37181361 | V-set | (11-133) |
| HG1015801 | 37181362:37181361 | ig | (151-208) |
| HG1015801 | 37181362:37181361 | ig | (241-303) |
| HG1015821 | 7243091:7243090 | I-set | (146-234) |
| HG1015821 | 7243091:7243090 | I-set | (237-312) |
| HG1015821 | 7243091:7243090 | V-set | (236-330) |
| HG1015821 | 7243091:7243090 | V-set | (21-143) |
| HG1015821 | 7243091:7243090 | fn3 | (518-606) |
| HG1015821 | 7243091:7243090 | fn3 | (634-718) |
| HG1015821 | 7243091:7243090 | ig | (161-218) |
| HG1015821 | 7243091:7243090 | ig | (443-498) |
| HG1015821 | 7243091:7243090 | ig | (251-313) |
| HG1015845 | NP_065840:NM_020789 | I-set | (136-224) |
| HG1015845 | NP_065840:NM_020789 | I-set | (227-302) |
| HG1015845 | NP_065840:NM_020789 | V-set | (226-320) |
| HG1015845 | NP_065840:NM_020789 | fn3 | (492-580) |
| HG1015845 | NP_065840:NM_020789 | fn3 | (608-692) |
| HG1015845 | NP_065840:NM_020789 | ig | (151-208) |
| HG1015845 | NP_065840:NM_020789 | ig | (417-472) |
| HG1015845 | NP_065840:NM_020789 | ig | (241-303) |

TABLE 5
SEQ. ID. NOS.: 95-223 (Related to Cluster 301014, Nectin 4)

| | SEQ. ID. NO. | SEQ. ID. NO. | SEQ. ID. NO. | |
|---|---|---|---|---|
| FP ID | (N1) | (P1) | (N0) | Clone ID |
| HG1015749 | SEQ. ID. NO. 95 | SEQ. ID. NO. 164 | SEQ. ID. NO. 221 | 14714574:14714573 |
| HG1015825 | SEQ. ID. NO. 96 | SEQ. ID. NO. 165 | SEQ. ID. NO. 222 | 9049508:9049507 |
| HG1015860 | SEQ. ID. NO. 97 | SEQ. ID. NO. 166 | SEQ. ID. NO. 223 | NP_112178: |
| | | | | NM_030916 |
| HG1016134 | SEQ. ID. NO. 98 | SEQ. ID. NO. 167 | | 14714574: |
| | | | | 14714573.ig.1 |
| HG1016135 | SEQ. ID. NO. 99 | SEQ. ID. NO. 168 | | 14714574: |
| | | | | 14714573.ig.2 |
| HG1016250 | SEQ. ID. NO. 100 | SEQ. ID. NO. 169 | | 9049508:9049507.ig.1 |
| HG1016251 | SEQ. ID. NO. 101 | SEQ. ID. NO. 170 | | 9049508:9049507.ig.2 |
| HG1016329 | SEQ. ID. NO. 102 | SEQ. ID. NO. 171 | | NP_112178: |
| | | | | NM_030916.ig.1 |
| HG1016330 | SEQ. ID. NO. 103 | SEQ. ID. NO. 172 | | NP_112178: |
| | | | | NM_030916.ig.2 |
| HG1016403 | SEQ. ID. NO. 104 | | | PRB103018_s_at |
| HG1017099 | SEQ. ID. NO. 105 | | | PRB103018_s_at:1 |
| HG1017100 | SEQ. ID. NO. 106 | | | PRB103018_s_at:10 |
| HG1017101 | SEQ. ID. NO. 107 | | | PRB103018_s_at:11 |
| HG1017102 | SEQ. ID. NO. 108 | | | PRB103018_s_at:2 |
| HG1017103 | SEQ. ID. NO. 109 | | | PRB103018_s_at:3 |
| HG1017104 | SEQ. ID. NO. 110 | | | PRB103018_s_at:4 |
| HG1017105 | SEQ. ID. NO. 111 | | | PRB103018_s_at:5 |
| HG1017106 | SEQ. ID. NO. 112 | | | PRB103018_s_at:6 |
| HG1017107 | SEQ. ID. NO. 113 | | | PRB103018_s_at:7 |
| HG1017108 | SEQ. ID. NO. 114 | | | PRB103018_s_at:8 |
| HG1017109 | SEQ. ID. NO. 115 | | | PRB103018_s_at:9 |
| HG1019621 | SEQ. ID. NO. 116 | SEQ. ID. NO. 173 | | 14714574:14714573.V- |
| | | | | set.1 |
| HG1019622 | SEQ. ID. NO. 117 | SEQ. ID. NO. 174 | | 9049508:9049507.V- |
| | | | | set.1 |
| HG1019623 | SEQ. ID. NO. 118 | SEQ. ID. NO. 175 | | NP_112178:NM_030916.V- |
| | | | | set.1 |
| HG1019624 | SEQ. ID. NO. 119 | SEQ. ID. NO. 176 | | 14714574:14714573_ECD |
| HG1019625 | SEQ. ID. NO. 120 | SEQ. ID. NO. 177 | | 9049508:9049507_ECD |
| HG1019626 | SEQ. ID. NO. 121 | SEQ. ID. NO. 178 | | NP_112178:NM_030916_ECD |
| HG1019696 | SEQ. ID. NO. 122 | SEQ. ID. NO. 179 | | NP_112178_49-69 |
| HG1019697 | SEQ. ID. NO. 123 | SEQ. ID. NO. 180 | | NP_112178_49-54 |
| HG1019698 | SEQ. ID. NO. 124 | SEQ. ID. NO. 181 | | NP_112178_50-55 |
| HG1019699 | SEQ. ID. NO. 125 | SEQ. ID. NO. 182 | | NP_112178_51-56 |
| HG1019700 | SEQ. ID. NO. 126 | SEQ. ID. NO. 183 | | NP_112178_52-57 |
| HG1019701 | SEQ. ID. NO. 127 | SEQ. ID. NO. 184 | | NP_112178_53-58 |
| HG1019702 | SEQ. ID. NO. 128 | SEQ. ID. NO. 185 | | NP_112178_54-59 |
| HG1019703 | SEQ. ID. NO. 129 | SEQ. ID. NO. 186 | | NP_112178_55-60 |
| HG1019704 | SEQ. ID. NO. 130 | SEQ. ID. NO. 187 | | NP_112178_56-61 |
| HG109705 | SEQ. ID. NO. 131 | SEQ. ID. NO. 188 | | NP_112178_57-62 |
| HG1019706 | SEQ. ID. NO. 132 | SEQ. ID. NO. 189 | | NP_112178_58-63 |
| HG1019707 | SEQ. ID. NO. 133 | SEQ. ID. NO. 190 | | NP_112178_59-64 |
| HG1019708 | SEQ. ID. NO. 134 | SEQ. ID. NO. 191 | | NP_112178_60-65 |
| HG1019709 | SEQ. ID. NO. 135 | SEQ. ID. NO. 192 | | NP_112178_61-66 |
| HG1019710 | SEQ. ID. NO. 136 | SEQ. ID. NO. 193 | | NP_112178_62-67 |
| HG1019711 | SEQ. ID. NO. 137 | SEQ. ID. NO. 194 | | NP_112178_63-68 |
| HG1019712 | SEQ. ID. NO. 138 | SEQ. ID. NO. 195 | | NP_112178_64-69 |
| HG1019713 | SEQ. ID. NO. 139 | SEQ. ID. NO. 196 | | NP_112178_88-115 |
| HG1019714 | SEQ. ID. NO. 140 | SEQ. ID. NO. 197 | | NP_112178_88-93 |
| HG1019715 | SEQ. ID. NO. 141 | SEQ. ID. NO. 198 | | NP_112178_89-94 |
| HG1019716 | SEQ. ID. NO. 142 | SEQ. ID. NO. 199 | | NP_112178_90-95 |
| HG1019717 | SEQ. ID. NO. 143 | SEQ. ID. NO. 200 | | NP_112178_91-96 |
| HG1019718 | SEQ. ID. NO. 144 | SEQ. ID. NO. 201 | | NP_112178_92-97 |
| HG1019719 | SEQ. ID. NO. 145 | SEQ. ID. NO. 202 | | NP_112178_93-98 |
| HG1019720 | SEQ. ID. NO. 146 | SEQ. ID. NO. 203 | | NP_112178_94-99 |
| HG1019721 | SEQ. ID. NO. 147 | SEQ. ID. NO. 204 | | NP_112178_95-100 |
| HG1019722 | SEQ. ID. NO. 148 | SEQ. ID. NO. 205 | | NP_112178_96-101 |
| HG1019723 | SEQ. ID. NO. 149 | SEQ. ID. NO. 206 | | NP_112178_97-102 |
| HG1019724 | SEQ. ID. NO. 150 | SEQ. ID. NO. 207 | | NP_112178_98-103 |
| HG1019725 | SEQ. ID. NO. 151 | SEQ. ID. NO. 208 | | NP_112178_99-104 |
| HG1019726 | SEQ. ID. NO. 152 | SEQ. ID. NO. 209 | | NP_112178_100-105 |
| HG1019727 | SEQ. ID. NO. 153 | SEQ. ID. NO. 210 | | NP_112178_101-106 |
| HG1019728 | SEQ. ID. NO. 154 | SEQ. ID. NO. 211 | | NP_112178_102-107 |
| HG1019729 | SEQ. ID. NO. 155 | SEQ. ID. NO. 212 | | NP_112178_103-108 |
| HG1019730 | SEQ. ID. NO. 156 | SEQ. ID. NO. 213 | | NP_112178_104-109 |
| HG1019731 | SEQ. ID. NO. 157 | SEQ. ID. NO. 214 | | NP_112178_105-110 |
| HG1019732 | SEQ. ID. NO. 158 | SEQ. ID. NO. 215 | | NP_112178_106-111 |
| HG1019733 | SEQ. ID. NO. 159 | SEQ. ID. NO. 216 | | NP_112178_107-112 |
| HG109734 | SEQ. ID. NO. 160 | SEQ. ID. NO. 217 | | NP_112178_108-113 |
| HG1019735 | SEQ. ID. NO. 161 | SEQ. ID. NO. 218 | | NP_112178_109-114 |
| HG1019736 | SEQ. ID. NO. 162 | SEQ. ID. NO. 219 | | NP_112178_110-115 |
| HG1019737 | SEQ. ID. NO. 163 | SEQ. ID. NO. 220 | | NP_112178_148-237 |

TABLE 6
Annotation of NCBI sequences identified by PRB103018_s_at

| | | Predicted | |
|---|---|---|---|
| | | Protein | |
| FP ID | Clone ID | Length | Annotation |
| HG1015749 | 14714574: | 510 | PVRL4 protein |
| | 14714573 | | [Homo sapiens] |
| HG1015825 | 9049508: | 510 | Ig superfamily receptor LNIR |
| | 9049507 | | precursor [Homo sapiens] |
| HG1015860 | NP_112178: | 510 | poliovirus receptor-related 4 |
| | NM_030916 | | [Homo sapiens] |

TABLE 7
Characterization of Polypeptides Encoded by NCBI Sequences of Cluster 301014, Nectin 4

| | | | | | | Altern | Altern | | | | |
|---|---|---|---|---|---|---|---|---|---|---|---|
| | | Pred | | Signal | Mature | Signal | Mature | | | | |
| | | Prot | Tree- | Peptide | Protein | Peptide | Protein | | TM | non-TM | Pfam |
| FP ID | Clone ID | Len | vote | Coords | Coords | Coords | Coords | TM | Coords | Coords | Prosite |
| HG1015749 | 14714574: | 510 | 0.02 | (9-31) | (32-510) | (17-29) | (30-510) | 1 | (350-372) | (1-349) | ig |
| | 14714573 | | | | | (14-26) | (27-510) | | | (373-510) | V-set |
| | | | | | | | | | | | Ig-like |
| HG1015825 | 9049508: | 510 | 0.02 | (9-31) | (32-510) | (17-29) | (30-510) | 1 | (350-372) | (1-349) | ig |
| | 9049507 | | | | | (14-26) | (27-510) | | | (373-510) | V-set |
| | | | | | | | | | | | Ig-like |
| HG1015860 | NP_112178: | 510 | 0.02 | (9-31) | (32-510) | (17-29) | (30-510) | 1 | (350-372) | (1-349) | ig |
| | NM_030916 | | | | | (14-26) | (27-510) | | | (373-510) | V-set |
| | | | | | | | | | | | Ig-like |

TABLE 8
Pfam and Prosite Domains of Polypeptides Encoded by Sequences
of Cluster 301014, Nectin 4

| FP ID | Clone ID | Pfam | Prosite | Coordinates |
|---|---|---|---|---|
| HG1015749 | 14714574:14714573 | V-set | | (6-146) |
| HG1015749 | 14714574:14714573 | ig | | (263-317) |
| HG1015749 | 14714574:14714573 | | Ig-like | (148-237) |
| HG1015825 | 9049508:9049507 | V-set | | (6-146) |
| HG1015825 | 9049508:9049507 | ig | | (263-317) |
| HG1015825 | 9049508:9049507 | | Ig-like | (148-237) |
| HG1015860 | NP_112178:NM_030916 | V-set | | (6-146) |
| HG1015860 | NP_112178:NM_030916 | ig | | (263-317) |
| HG1015860 | NP_112178:NM_030916 | | Ig-like | (148-237) |

TABLE 9
SEQ. ID. NOS.: 224-248 (Related to Cluster 206895, KIAA0152)

| | SEQ. ID. NO. | SEQ. ID. NO. | SEQ. ID. NO. | |
|---|---|---|---|---|
| FP ID | (N1) | (P1) | (N0) | Clone ID |
| HG1019552 | SEQ. ID. NO. 224 | SEQ. ID. NO. 246 | SEQ. ID. NO. 248 | NP_055545:NM_014730 |
| HG1019554 | SEQ. ID. NO. 225 | SEQ. ID. NO. 247 | | NP_055545_ECD |
| HG1019555 | SEQ. ID. NO. 226 | | | PRB105610_at |
| HG1019556 | SEQ. ID. NO. 227 | | | PRB105610_at:1 |
| HG1019557 | SEQ. ID. NO. 228 | | | PRB105610_at:2 |
| HG1019558 | SEQ. ID. NO. 229 | | | PRB105610_at:3 |
| HG1019559 | SEQ. ID. NO. 230 | | | PRB105610_at:4 |
| HG1019560 | SEQ. ID. NO. 231 | | | PRB105610_at:5 |
| HG1019561 | SEQ. ID. NO. 232 | | | PRB105610_at:6 |
| HG1019562 | SEQ. ID. NO. 233 | | | PRB105610_at:7 |
| HG1019563 | SEQ. ID. NO. 234 | | | PRB105610_at:8 |
| HG1019564 | SEQ. ID. NO. 235 | | | PRB105610_at:9 |
| HG1019565 | SEQ. ID. NO. 236 | | | PRB105610_at:10 |
| HG1019566 | SEQ. ID. NO. 237 | | | PRB105610_at:11 |
| HG1019567 | SEQ. ID. NO. 238 | | | CLN00009706_taqman |
| HG1019568 | SEQ. ID. NO. 239 | | | CLN00394104_taqman |
| HG1019569 | SEQ. ID. NO. 240 | | | CLN00009706_taqmanF |
| HG1019570 | SEQ. ID. NO. 241 | | | CLN00009706_taqmanR |
| HG1019571 | SEQ. ID. NO. 242 | | | CLN00009706_taqman- |
| | | | | Probe |
| HG1019572 | SEQ. ID. NO. 243 | | | CLN00394104_taqmanF |
| HG1019573 | SEQ. ID. NO. 244 | | | CLN00394104_taqmanR |
| HG1019574 | SEQ. ID. NO. 245 | | | CLN00394104_taqman- |
| | | | | Probe |

TABLE 10
Annotation of NCBI Sequence Identified by PRB105610_at

| | | Predicted | |
|---|---|---|---|
| | | Protein | |
| FP ID | Clone ID | Length | Annotation |
| HG1019552 | NP_055545:NM_014730 | 292 | KIAA0152 |
| | | | gene product |
| | | | [Homo sapiens] |

TABLE 11
Characterization of a Polypeptide Encoded by NCBI Sequences of Cluster 206895, KIAA0152

| | | | | | | Altern | Alten | | | |
|---|---|---|---|---|---|---|---|---|---|---|
| | | Pred | | Signal | Mature | Signal | Mature | | | |
| | | Prot | Tree- | Peptide | Protein | Peptide | Protein | | TM | non-TM |
| FP ID | Clone ID | Len | vote | Coords | Coords | Coords | Coords | TM | Coords | Coords |
| HG1019552 | NP_055545: | 292 | 0.12 | (14-32) | (33-292) | (16-28) | (29-292) | 1 | (271-290) | (1-270) |
| | NM_014730 | | | | | | | | | (291-292) |

TABLE 12
SEQ. ID. NOS.: 249-380 (Related to Cluster 181658, Semaphorin 4B)

| | SEQ. ID. NO. | SEQ. ID. NO. | SEQ. ID. NO. | |
|---|---|---|---|---|
| FP ID | (N1) | (P1) | (N0) | Clone ID |
| HG1019631 | SEQ. ID. NO. 249 | SEQ. ID. NO. 314 | SEQ. ID. NO. 379 | 39777608:39777607 |
| HG1019632 | SEQ. ID. NO. 250 | SEQ. ID. NO. 315 | SEQ. ID. NO. 380 | 10438887:10438886 |
| HG1019633 | SEQ. ID. NO. 251 | SEQ. ID. NO. 316 | | 10438887_347-352 |
| HG1019634 | SEQ. ID. NO. 252 | SEQ. ID. NO. 317 | | 10438887_348-353 |
| HG1019635 | SEQ. ID. NO. 253 | SEQ. ID. NO. 318 | | 10438887_349-354 |
| HG1019636 | SEQ. ID. NO. 254 | SEQ. ID. NO. 319 | | 10438887_350-355 |
| HG1019637 | SEQ. ID. NO. 255 | SEQ. ID. NO. 320 | | 10438887_351-356 |
| HG1019638 | SEQ. ID. NO. 256 | SEQ. ID. NO. 321 | | 39777608_261-284 |
| HG1019639 | SEQ. ID. NO. 257 | SEQ. ID. NO. 322 | | 39777608_261-266 |
| HG1019640 | SEQ. ID. NO. 258 | SEQ. ID. NO. 323 | | 39777608_262-267 |
| HG1019641 | SEQ. ID. NO. 259 | SEQ. ID. NO. 324 | | 39777608_263-268 |
| HG1019642 | SEQ. ID. NO. 260 | SEQ. ID. NO. 325 | | 39777608_264-269 |
| HG1019643 | SEQ. ID. NO. 261 | SEQ. ID. NO. 326 | | 39777608_265-270 |
| HG1019644 | SEQ. ID. NO. 262 | SEQ. ID. NO. 327 | | 39777608_266-271 |
| HG1019645 | SEQ. ID. NO. 263 | SEQ. ID. NO. 328 | | 39777608_267-272 |
| HG1019646 | SEQ. ID. NO. 264 | SEQ. ID. NO. 329 | | 39777608_268-273 |
| HG1019647 | SEQ. ID. NO. 265 | SEQ. ID. NO. 330 | | 39777608_269-274 |
| HG1019648 | SEQ. ID. NO. 266 | SEQ. ID. NO. 331 | | 39777608_270-275 |
| HG1019649 | SEQ. ID. NO. 267 | SEQ. ID. NO. 332 | | 39777608_271-276 |
| HG1019650 | SEQ. ID. NO. 268 | SEQ. ID. NO. 333 | | 39777608_272-277 |
| HG1019651 | SEQ. ID. NO. 269 | SEQ. ID. NO. 334 | | 39777608_273-278 |
| HG1019652 | SEQ. ID. NO. 270 | SEQ. ID. NO. 335 | | 39777608_274-279 |
| HG1019653 | SEQ. ID. NO. 271 | SEQ. ID. NO. 336 | | 39777608_275-280 |
| HG1019654 | SEQ. ID. NO. 272 | SEQ. ID. NO. 337 | | 39777608_276-281 |
| HG1019655 | SEQ. ID. NO. 273 | SEQ. ID. NO. 338 | | 39777608_277-282 |
| HG1019656 | SEQ. ID. NO. 274 | SEQ. ID. NO. 339 | | 39777608_278-283 |
| HG1019657 | SEQ. ID. NO. 275 | SEQ. ID. NO. 340 | | 39777608_279-284 |
| HG1019658 | SEQ. ID. NO. 276 | SEQ. ID. NO. 341 | | 39777608_304-328 |
| HG1019659 | SEQ. ID. NO. 277 | SEQ. ID. NO. 342 | | 39777608_304-309 |
| HG1019660 | SEQ. ID. NO. 278 | SEQ. ID. NO. 343 | | 39777608_305-310 |
| HG1019661 | SEQ. ID. NO. 279 | SEQ. ID. NO. 344 | | 39777608_306-311 |
| HG1019662 | SEQ. ID. NO. 280 | SEQ. ID. NO. 345 | | 39777608_307-312 |
| HG1019663 | SEQ. ID. NO. 281 | SEQ. ID. NO. 346 | | 39777608_308-313 |
| HG1019664 | SEQ. ID. NO. 282 | SEQ. ID. NO. 347 | | 39777608_309-314 |
| HG1019665 | SEQ. ID. NO. 283 | SEQ. ID. NO. 348 | | 39777608_310-315 |
| HG1019666 | SEQ. ID. NO. 284 | SEQ. ID. NO. 349 | | 39777608_311-316 |
| HG1019667 | SEQ. ID. NO. 285 | SEQ. ID. NO. 350 | | 39777608_312-317 |
| HG1019668 | SEQ. ID. NO. 286 | SEQ. ID. NO. 351 | | 39777608_313-318 |
| HG1019669 | SEQ. ID. NO. 287 | SEQ. ID. NO. 352 | | 39777608_314-319 |
| HG1019670 | SEQ. ID. NO. 288 | SEQ. ID. NO. 353 | | 39777608_315-320 |
| HG1019671 | SEQ. ID. NO. 289 | SEQ. ID. NO. 354 | | 39777608_316-321 |
| HG1019672 | SEQ. ID. NO. 290 | SEQ. ID. NO. 355 | | 39777608_317-322 |
| HG1019673 | SEQ. ID. NO. 291 | SEQ. ID. NO. 356 | | 39777608_318-323 |
| HG1019674 | SEQ. ID. NO. 292 | SEQ. ID. NO. 357 | | 39777608_319-324 |
| HG1019675 | SEQ. ID. NO. 293 | SEQ. ID. NO. 358 | | 39777608_320-325 |
| HG1019676 | SEQ. ID. NO. 294 | SEQ. ID. NO. 359 | | 39777608_321-326 |
| HG1019677 | SEQ. ID. NO. 295 | SEQ. ID. NO. 360 | | 39777608_322-327 |
| HG1019678 | SEQ. ID. NO. 296 | SEQ. ID. NO. 361 | | 39777608_323-328 |
| HG1019679 | SEQ. ID. NO. 297 | SEQ. ID. NO. 362 | | 39777608_339-359 |
| HG1019680 | SEQ. ID. NO. 298 | SEQ. ID. NO. 363 | | 39777608_339-344 |
| HG1019681 | SEQ. ID. NO. 299 | SEQ. ID. NO. 364 | | 39777608_340-345 |
| HG1019682 | SEQ. ID. NO. 300 | SEQ. ID. NO. 365 | | 39777608_341-346 |
| HG1019683 | SEQ. ID. NO. 301 | SEQ. ID. NO. 366 | | 39777608_342-347 |
| HG1019684 | SEQ. ID. NO. 302 | SEQ. ID. NO. 367 | | 39777608_343-348 |
| HG1019685 | SEQ. ID. NO. 303 | SEQ. ID. NO. 368 | | 39777608_344-349 |
| HG1019686 | SEQ. ID. NO. 304 | SEQ. ID. NO. 369 | | 39777608_345-350 |
| HG1019687 | SEQ. ID. NO. 305 | SEQ. ID. NO. 370 | | 39777608_346-351 |
| HG1019688 | SEQ. ID. NO. 306 | SEQ. ID. NO. 371 | | 39777608_347-352 |
| HG1019689 | SEQ. ID. NO. 307 | SEQ. ID. NO. 372 | | 39777608_348-353 |
| HG1019690 | SEQ. ID. NO. 308 | SEQ. ID. NO. 373 | | 39777608_349-354 |
| HG1019691 | SEQ. ID. NO. 309 | SEQ. ID. NO. 374 | | 39777608_350-355 |
| HG1019692 | SEQ. ID. NO. 310 | SEQ. ID. NO. 375 | | 39777608_351-356 |
| HG1019693 | SEQ. ID. NO. 311 | SEQ. ID. NO. 376 | | 39777608_352-357 |
| HG1019694 | SEQ. ID. NO. 312 | SEQ. ID. NO. 377 | | 39777608_353-358 |
| HG1019695 | SEQ. ID. NO. 313 | SEQ. ID. NO. 378 | | 39777608_354-359 |

TABLE 13
Annotation of NCBI sequences identified by PRB101227_at

| | | Predicted | |
|---|---|---|---|
| | | Protein | |
| FP ID | Clone ID | Length | Annotation |
| HG1019631 | 39777608:39777607 | 837 | semaphorin 4B precursor |
| | | | [Homo sapiens] |
| HG1019632 | 10438887:10438886 | 380 | unnamed protein product |
| | | | [Homo sapiens] |

TABLE 14
Characterization of Polypeptides Encoded by NCBI Sequences of
Cluster 181658, Semaphorin 4B

| | | Pred | | Signal | Mature | | | | |
|---|---|---|---|---|---|---|---|---|---|
| | | Protein | Tree- | Peptide | Protein | | TM | non-TM | |
| FP ID | Clone ID | Length | vote | Coords | Coords | TM | Coords | Coords | Pfam |
| HG1019631 | 39777608: | 837 | 0 | (19-43) | (44-837) | 1 | (717-739) | (1-716) | PSI |
| | 39777607 | | | | | | | (740-837) | Sema |
| HG1019632 | 10438887: | 380 | 0.02 | | (1-380) | 0 | | | PSI |
| | 10438886 | | | | | | | | Sema |

TABLE 15
Pfam Domains of Polypeptides Encoded by Sequences of Cluster
181658, Semaphorin 4B

| FP ID | Clone ID | Pfam | Coordinates |
|---|---|---|---|
| HG1019631 | 39777608:39777607 | PSI | (525-577) |
| HG1019631 | 39777608:39777607 | Sema | (70-507) |
| HG1019632 | 10438887:10438886 | PSI | (163-215) |
| HG1019632 | 10438887:10438886 | Sema | (1-145) |

### REFERENCES

The specification is most thoroughly understood in light of the following references, all of which are hereby incorporated in their entireties. The disclosures of the patents and other references cited above are also hereby incorporated by reference in their entireties.
1. Agrawal, S., et al., eds. (1998) Antisense Research and Application. Handbook of Experimental Pharmaco., Vol. 131. Springer-Verlag NY, Inc.
2. American Cancer Society (2005) Cancer Facts & Figures. (http://www. cancer.org/downloads/STT/CAFF2005f4PWSecured.pdf).
3. Ansel, H. C., et al., eds. (2004) Pharmaceutical Dosage Forms and Drug Delivery Systems. 8th ed. Lippincott Williams & Wilkins.
4. Barr, P. J. and Tomei, L. D. (1994) Bio/Technology 12:487-493.
5. Bateman, A., et al. (2000) Nucleic Acids Research 30:276-280.
6. Beigelman, L., et al. (1995) Nucleic Acids Res. 23:4434-4442.
7. Chen, S. Y., et al. (1994) Hum. Gene Ther. 5:595-601.
8. Coligan, J. E. et al., eds. (2002) Current Protocols in Immunology, Vols. 1-4 (including quarterly suppl.). John Wiley and Sons, Inc.
9. De Vita, V. T., Jr., et al., eds. (2001) Cancer: Principles & Practice of Oncol.
10. Dieffenbach, C. W., et al. eds. (1995) PCR Primer: A Laboratory Manual. Cold Spring Harbor Laboratory Press.
11. Furth, P. A., et al. (1992) Anal. Biochem. 205:365-368.
12. Gaudilliere, B., et al. (2002) J. Biol. Chem. 277:46,442-46, 446.
13. Gennaro, A. R. (2003) Remington: The Sci. and Pract. of Pharm. with Facts and Comparisons: DrugfactsPlus. 20th ed. Lippincott Williams & Wilkins.
14. Gorman, et al. (1982) Proc. Natl. Acad. Sci. 79:6777-6781.
15. Grosschedl, R. and Baltimore, D. (1985) Cell 41:885-897.
16. Grosveld, F. and Kollias, G., eds. (1992) Transgenic Animals. 1^{st} ed. Academic Press.
17. Harlow, E., et al., eds. (1998) Using Antibodies: A Laboratory Manual: Portable Protocol NO. I. Cold Spring Harbor Laboratory.
18. Harlow, E. and Lane, D., eds. (1988) Antibodies: A Laboratory Manual. Cold Spring Harbor Laboratory.
19. Hartmann, G. and Endres, S., eds. (1999) Manual of Antisense Methodology (Perspectives in Antisense Science). 1^{st} ed. Kluwer Law International.
20. Hassanzadeh, G. H. G., et al. (1998) FEBS Lett. 437:75-80.
21. Heiser, A., et al. (2002) J. Clin. Invest. 109:409-417.
22. Hoogenboom, H. R., et al. (1998) Immunotechnology 4:1-20.
23. Houseman, B. T., et al. (2002) Nature Biotechnol. 20:270-274.
24. Howard, G. C. and Bethell, D. R. (2000) Basic Methods in Antibody Production and Characterization. CRC Press.
25. Itoh, N., et al. (1991) Cell 66:233-243.
26. Jost, C. R., et al. (1994) J. Biol. Chem. 269:26,267-26, 273.
27. Kabat, E. A. and Wu, T. T. (1991) J. Immunol. 147:1709-1719.

28. Kibbe, A. H., ed. (2000) Handbook of Pharmaceutical Excipients. 3^{rd} ed. Pharmaceutical Press.
29. Knutson, K. L., et al. (2001) J. Clin. Invest. 107:477-484.
30. Kolonin, M. G., et al. (1998) Proc. Natl. Acad. Sci. 95:14,266-14, 271.
31. Kuisle, O., et al., (1999) Tetrahedron Lett. 40:1203-1206.
32. Latham, J. A., et al. (1994) Nucl. Acids Res. 22:2817-2822.
33. Liu A. Y., et al. (1987a) Proc. Natl. Acad. Sci. USA 84:3439-3443.
34. Liu, A. Y., et al. (1987b) J. Immunol. 139:3521-3526.
35. MacBeath, G. and Schreiber, S. L. (2000) Science 289:1760-1763.
36. Machiels, J. P., et al. (2002) Semin. Oncol. 29:494-502.
37. McPherson, M. J., et al. (2000) PCR Basics. Springer Verlag.
38. Milligan, J. F., et al. (1993) J. Med. Chem. 36:1923-1937.
39. Mitchell, D. A., et al. (2000) J. Clin. Invest. 106:1065-1069.
40. Monfardini, C., et al. (1995) Bioconjugate Chem. 6:62-69.
41. Murphy, D., and Carter, D. A., eds. (1993) Transgenesis Techniques: Principles and Protocols. Humana Press.
42. Takai, Y., and Nakanishi, H. (2003) J. Cell Sci. 1116:17-27.
43. Okayama, H., and Berg, P. (1983) Mol. Cell. Biol. 3:280-289.
44. O'Neil, N. J., et al., (2001) Am. J. Pharmacogenomics 1:45-53.
45. Pertl, U., et al. (2003) Blood 101:649-654.
46. Phillips, M. I., ed. (1999a) Antisense Technology, Part A. Methods in Enzymology, Vol. 313. Academic Press, Inc.
47. Phillips, M. I., ed. (1999b) Antisense Technology, Part B. Methods in Enzymology, Vol. 314. Academic Press, Inc.
48. Pinkert, C. A., ed. (1994) Transgenic Animal Technology: A Laboratory Handbook. Academic Press.
49. Raper, J. A. (2000) Curr. Opin. Neurobiol. 10:88-94.
50. Sambrook, J., Russell, D. W., (2000) Molecular Cloning, A Laboratory Manual. 3^{nd} ed. Cold Spring Harbor Laboratory Press.
51. Schaeferling, M., et al. (2002) Electrophoresis 23:3097-3105.
52. Schlehuber, S., and Skerra, A. (2001) Biol. Chem. 382:1335-1342.
53. Shinnick, T. M., et al. (198.3) Ann. Rev. Microbiol. 37:425-446.
54. Sigrist et al., et al. (2002) Brief Bioinform. 3:265-274.
55. Shinnick, T. M., et al. (1983) Ann. Rev. Microbiol. 37:425-446.
56. Stein, C. A., et al., eds. (1998) Appl. Antisense Oligonucl. Technol. Wiley-Liss.
57. Steller, H. (1995) Science, 267:1445-1449.

58. Takai, Y. and Nakanishi, H. (2003) J. Cell Sci. 116:17-27.
59. Tang, D. C., et al. (1992) Nature 356:152-154.
60. Thompson, C. B. (1995) Science 267:1456-1462.
61. Wagner, R. W., et al. (1993) Science 260:1510-1513.
62. Wagner, R. W., et al. (1996) Nat. Biotechnol. 14:840-844.
63. Weng, S., et al. (2002) Proteomics 2:48-57.
64. Winssinger, N., et al. (2002) Proc. Natl. Acad. Sci. 99:11,139-11, 144.
65. Xu, C. W., et al. (1997) Proc. Natl. Acad. Sci. 94:12,473-12, 478.
66. Yu, Z. and Restifo, N. P. (2002) J. Clin. Invest. 110:289-294.
67. Zallipsky, S. (1995) Bioconjugate Chem., 6:150-165.
68. Zhu, H., et al. (2001) Science 293:2101-2105.
69. Zhu, H. and Snyder, M. (2003) Curr. Opin. Chem. Biol. 7:55-63.

1.-93. (canceled)

94. A method of treating cancer comprising administering to a patient an antibody that binds to nectin 4.

95. The method of claim 94, wherein the cancer is selected from lung adenocarcinoma, lung squamous cell carcinoma, colon/colorectal cancer, prostate cancer, pancreatic cancer, bladder cancer, endometrial cancer, kidney cancer, liver cancer, ovarian cancer, breast cancer, and thyroid cancer.

96. The method of claim 94, wherein the antibody mediates antibody-dependent cell cytotoxicity (ADCC) or complement-dependent cytotoxicity (CDC).

97. The method of claim 94, wherein the antibody is an antibody conjugate.

98. The method of claim 97, wherein the antibody is conjugated to an agent selected from a radionuclide, a toxin, and a chemotherapeutic.

99. The method of claim 98, wherein the toxin is a microbial toxin or a plant toxin.

100. The method of claim 98, wherein the chemotherapeutic is selected from doxorubicin and cisplatin.
